(19) United States
(12) Patent Application Publication
(10) Pub. No.: US 2023/0220328 A1
(43) Pub. Date: Jul. 13, 2023

(54) METHODS AND COMPOSITIONS FOR CULTURING HEMOGLOBIN-DEPENDENT BACTERIA

(71) Applicant: Evelo Biosciences, Inc., Cambridge, MA (US)

(72) Inventors: Valeria Kravitz, Norwood, MA (US); Manas Ray, Cambridge, MA (US); Maria Sizova, Roslindale, MA (US)

(21) Appl. No.: 17/780,819

(22) PCT Filed: Nov. 25, 2020
(86) PCT No.: PCT/US2020/062167

§ 371 Date: May 27, 2022

Related U.S. Application Data
(60) Provisional application No. 62/941,076, filed on Nov. 27, 2019.
(60) Provisional application No. 63/008,233, filed on Apr. 10, 2020.

(51) Int. Cl. C12N 1/20; C12N 1/38

(52) U.S. Cl. CPC C12N 1/20; C12N 1/38; C12N 2501/998

(57) ABSTRACT

Provided herein are compositions and methods related to culturing bacteria (such as hemoglobin-dependent bacteria) with a heme-containing polypeptide (such as a heme-containing polypeptide that is not sourced from an animal and/or can be recombinantly produced).

### CROSS-REFERENCE TO RELATED APPLICATIONS

This application is a 371 national-stage application based on International Patent Application No. PCT/US20/62167, filed Nov. 25, 2020, which claims the benefit of U.S. Provisional Application No. 62/941,076 filed on Nov. 27, 2019; and U.S. Provisional Application No. 63/008,233 filed on Apr. 10, 2020; the entire contents of each of which are incorporated by reference herein in their entirety.

### REFERENCE TO A SEQUENCE LISTING

This application contains a Sequence Listing in computer readable form. The computer readable form is incorporated herein by reference. Said ASCII copy, created on Nov. 15, 2022, is named ETB-04901 SL.txt and is 337,850 bytes in size.

### BACKGROUND

The composition of a person's microbiome can play an important role in their health and well-being. Indeed, disruption of an individual's microbiome has been implicated in numerous diseases, including inflammatory bowel diseases, immune disorders, type 2 diabetes, neurodegenerative disorders, cardiovascular diseases, and cancers. Thus, microbiome modulation is an attractive therapeutic strategy for such diseases.

### SUMMARY

One way to modulate a person's microbiome is by orally administering to them one or more strains of beneficial bacteria, e.g., formulated for therapeutic use, e.g., in a bacterial composition (e.g., a bacterial composition (e.g., a pharmaceutical composition)). However, development of such therapies has been hindered by the fact that large-scale production of many bacterial strains has proven challenging, particularly for bacterial strains that require hemoglobin (or its derivatives such as hemin) for growth.

Hemoglobin is an iron-containing metalloprotein in red blood cells that captures atmospheric oxygen in the lungs and carries it to the rest of the body. Iron is an essential nutrient for almost all forms of life, including bacteria. As hemoglobin is the most abundant reservoir of iron within humans, much of the bacteria that make up the human microbiome use hemoglobin or its derivatives as their primary source of iron. Often, such hemoglobin-dependent bacteria require the presence of hemoglobin or hemin for optimal in vitro growth. However, commercial hemoglobin and its derivatives are typically purified from animal sources, such as from porcine blood, which, inter alia, results in purified hemoglobin being costly. Moreover, GMP (good manufacturing practice)-grade hemoglobin is not easily sourced, making the large-scale manufacture and/or GMP grade manufacture of hemoglobin-dependent bacteria for pharmaceutical purposes particularly challenging.

Accordingly, there is a great need for improved compositions and methods for growing hemoglobin-dependent bacteria, e.g., without the need for hemoglobin sourced from an animal (e.g., without the need for porcine or bovine hemoglobin).

In certain aspects, provided herein are heme-containing polypeptides (e.g., that are not sourced from an animal) that can be used in growth media to facilitate the in vitro culturing of hemoglobin-dependent bacteria, including bacteria of the genus Prevotella, such as Prevotella histicola. The heme-containing polypeptides (e.g., that are not sourced from an animal) provided herein can allow for large-scale manufacture and/or GMP grade manufacture of hemoglobin-dependent bacteria, e.g., for therapeutic use and/or in a bacterial composition (e.g., a bacterial composition (e.g., a pharmaceutical composition)). In certain embodiments, the heme-containing polypeptide may be a piscine (i.e., fish) polypeptide or an avian (i.e., bird) polypeptide. In some embodiments, the heme-containing polypeptide may be a non-animal-derived polypeptide, such as a plant polypeptide (e.g., a grain or legume polypeptide), a bacteria polypeptide, a cyanobacteria polypeptide, a fungus polypeptide, an algae polypeptide, or a protozoa polypeptide. In some embodiments, the heme-containing polypeptide may be a legume polypeptide (e.g., leghemoglobin, e.g., soy leghemoglobin). In certain embodiments, the heme-containing polypeptides provided herein can be obtained from their natural source (e.g., isolated from fish, birds, plants, bacteria, cyanobacteria, fungus, algae, or protozoa). In some embodiments, the heme-containing polypeptides provided herein are recombinantly expressed. Notably, recombinant expression of the disclosed heme-containing polypeptides facilitates, inter alia, the cost-effective, vegetarian, kosher, and GMP-grade production of heme-containing polypeptides; it is an attractive way to produce heme-containing polypeptides for use in bacterial cell culture applications.

In certain aspects, provided herein are methods and compositions that facilitate the culturing of hemoglobin-dependent bacteria (e.g., for use in a bacterial composition (e.g., a pharmaceutical composition) and/or for therapeutic use). Growth of hemoglobin-dependent bacteria is accomplished through the inclusion in the cell culture media of a heme-containing polypeptide provided herein. In certain embodiments, the heme-containing polypeptide is a hemoglobin or a variant thereof, such as symbiotic hemoglobin, non-symbiotic hemoglobin, and/or truncated hemoglobin that is able to support growth of hemoglobin-dependent bacteria. In certain embodiments, the heme-containing polypeptide is a leghemoglobin (e.g., soy leghemoglobin (recombinantly expressed (e.g., in Pichia pastoris) or naturally-sourced)) or myoglobin that is able to support growth of hemoglobin-dependent bacteria. In certain embodiments, the heme-containing polypeptide is a cytochrome or peroxidase that is able to substitute for hemoglobin to support growth of otherwise hemoglobin-dependent bacteria. Non-limiting examples of classes of heme-containing polypeptides provided herein include androglobins, cytoglobins, globin E, globin X, globin Y, hemoglobins, myoglobins, erythrocruorins, beta hemoglobins, alpha hemoglobins, protoglobins, cyanoglobins, cytoglobins, histoglobins, neuroglobins, chlorocruorins, truncated 2/2 globins, hemoglobin 3 (e.g., Glb3), cytochromes, and/or peroxidases (e.g., cytochrome c peroxidase) that are able to support growth of hemoglobin-dependent bacteria.

Thus, in certain aspects, provided herein are methods and compositions for culturing hemoglobin-dependent bacteria (e.g., for use in a bacterial composition (e.g., a pharmaceutical composition) and/or for therapeutic use) in growth media that includes a heme-containing polypeptide provided herein. In some aspects, provided herein are compositions (e.g., growth media) comprising a heme-containing polypeptide provided herein that are useful for culturing hemoglobin-dependent bacteria in conditions free from conventional hemoglobin (e.g., porcine hemoglobin) (e.g., conditions not comprising a hemoglobin sourced from an animal) or a derivative thereof, as well as methods of making and/or using such compositions.

In some embodiments, the heme-containing polypeptide used in the methods and compositions provided herein is a piscine polypeptide. In certain embodiments, the piscine heme-containing polypeptide is purified from fish, fish meals, and/or fish protein hydrolysate. In some embodiments, the piscine heme-containing polypeptide is recombinantly expressed. In some embodiments, the piscine heme-containing polypeptide is a heme-containing polypeptide of a fish of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes. Accordingly, provided herein are methods and compositions for culturing hemoglobin-dependent bacteria (e.g., for use in a bacterial composition (e.g., a pharmaceutical composition) and/or for therapeutic use) in growth media that includes a recombinant or native piscine heme-containing polypeptide. In some aspects, provided herein are compositions (e.g., growth media) comprising a recombinant or native piscine heme-containing polypeptide that are useful for culturing hemoglobin-dependent bacteria (e.g., for use in a bacterial composition (e.g., a pharmaceutical composition) and/or for therapeutic use) in conditions free from conventional hemoglobin (e.g., porcine hemoglobin) (e.g., conditions not comprising a hemoglobin sourced from an animal), as well as methods of making and/or using such compositions.

In some embodiments, the heme-containing polypeptide used in the methods and compositions provided herein is an avian polypeptide. In certain embodiments, the avian heme-containing polypeptide is purified from birds, bird meals (e.g., chicken meal), and/or bird protein hydrolysate. In some embodiments, the avian heme-containing polypeptide is recombinantly expressed. In certain embodiments, the avian heme-containing polypeptide is a heme-containing polypeptide of a bird of the genus Gallus, Meleagris, Anas, Anser, Branta, Chen, Agelastes, Numida, Guttera, Acryllium, or Columba. Accordingly, provided herein are methods and compositions for culturing hemoglobin-dependent bacteria (e.g., for use in a bacterial composition (e.g., a pharmaceutical composition) and/or for therapeutic use) in growth media that includes a recombinant or native avian heme-containing polypeptide. In some aspects, provided herein are compositions (e.g., growth media) comprising a recombinant or native avian heme-containing polypeptide that are useful for culturing hemoglobin-dependent bacteria (e.g., for use in a bacterial composition (e.g., a pharmaceutical composition) and/or for therapeutic use) in conditions free from conventional hemoglobin (e.g., porcine hemoglobin) (e.g., conditions not comprising a hemoglobin sourced from an animal), as well as methods of making and/or using such compositions.

In certain embodiments, the heme-containing polypeptide used in the methods and compositions provided herein is not animal derived. In certain embodiments, the non-animal-derived heme-containing polypeptide is purified from a source such as plants (e.g., grain, legume) bacteria, cyanobacteria, fungus, algae, and/or protozoa. In some embodiments, the non-animal-derived heme-containing polypeptide is recombinantly expressed. In some embodiments, the non-animal-derived heme-containing polypeptide is a heme-containing polypeptide of an organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium. In some embodiments, the non-animal-derived polypeptide may be purified from a processed form of these organisms (e.g., soy flour, pea flour, mung bean flour, etc.). Accordingly, provided herein are methods and compositions for culturing hemoglobin-dependent bacteria (e.g., for use in a bacterial composition (e.g., a pharmaceutical composition) and/or for therapeutic use) in growth media that includes a recombinant or native non-animal-derived heme-containing polypeptide. In some aspects, provided herein are compositions (e.g., growth media) comprising a recombinant or native non-animal-derived heme-containing polypeptide that are useful for culturing hemoglobin-dependent bacteria (e.g., for use in a bacterial composition (e.g., a pharmaceutical composition) and/or for therapeutic use) in conditions free from conventional hemoglobin (e.g., porcine hemoglobin) (e.g., conditions not comprising a hemoglobin sourced from an animal), as well as methods of making and/or using such compositions.

Exemplary heme-containing polypeptide and nucleic acid sequences useful in the methods and compositions provided herein are listed, for example, in Table 3. In some embodiments, the heme-containing polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity to an amino acid sequence in any of SEQ ID NO: 1-29, 31, 33, 35, 37, 39, 41, 43, or 114. In some embodiments, the heme-containing polypeptide comprises the amino acid sequence of any of SEQ ID NO: 1-29, 31, 33, 35, 37, 39, 41, 43, or 114.

In some embodiments, the heme-containing polypeptide comprises an amino acid sequence with at least 70% identity to the amino acid sequence of SEQ ID NO: 4.

In some embodiments, the heme-containing polypeptide comprises an amino acid sequence with at least 90% identity to the amino acid sequence of SEQ ID NO: 4.

In some embodiments, the heme-containing polypeptide comprises the amino acid sequence of SEQ ID NO: 4.

In some embodiments, the heme-containing polypeptide comprises an amino acid sequence with at least 70% identity to the amino acid sequence of SEQ ID NO: 114 or UniProtKB—P02236 (LGB2_SOYBN).

In some embodiments, the heme-containing polypeptide comprises an amino acid sequence with at least 90% identity to the amino acid sequence of SEQ ID NO: 114 or UniProtKB—P02236 (LGB2_SOYBN).

In some embodiments, the heme-containing polypeptide comprises the amino acid sequence of SEQ ID NO: 114 or UniProtKB—P02236 (LGB2_SOYBN).

In certain embodiments, the heme-containing polypeptide provided herein is recombinantly expressed. In some embodiments, the recombinant heme-containing polypeptide provided herein further comprises a heterologous polypeptide, such as a histidine tag, TAP (tandem affinity purification) tag, TEV cleavage site, a FLAG tag, a GST tag, and/or an immunoglobulin domain. In some embodiments, such heterologous polypeptide enhances or facilitates purification and/or detection of the heme-containing polypeptide. In some embodiments, the recombinant polypeptide is expressed in a host cell (e.g., from an exogenous nucleic acid, such as an expression vector present in the host cell). In some embodiments, the host cells are bacteria cells, yeast cells, insect cells, or mammalian cells (e.g., a mammalian cell line). In some embodiments, the host cells are yeast cells. In some embodiments, the host cell is Pichia Pastoris. It will be understood that Pichia pastoris has been reclassified as Komagataella species, such as Komagataella phaffii, Komagataella pastoris, or Komagataella pseudopastoris, though the term “Pichia pastoris” is still in use and may refer to any appropriate Komagataella species. Representative recombinant heme-containing polypeptides using Pichia Pastoris host cells are disclosed in U.S. Pat. Nos. 9,938,327; 10,273,492; 10,798,958, which are hereby incorporated by reference herein in their entireties, and in particular for the heme-containing polypeptide constructs disclosed therein. In some embodiments, the host cell is Escherichia coli.

In certain aspects, provided herein is a growth medium for use in culturing hemoglobin-dependent bacteria (e.g., for use in a bacterial composition (e.g., a pharmaceutical composition) and/or for therapeutic use), the growth medium comprising a heme-containing polypeptide provided herein (e.g., a heme-containing polypeptide not sourced from an animal). In some embodiments, the growth medium comprises hemoglobin-dependent bacteria (e.g., for use in a bacterial composition (e.g., a pharmaceutical composition) and/or for therapeutic use). In certain embodiments, provided herein is a heme-containing polypeptide (e.g., a heme-containing polypeptide not sourced from an animal) for use as a substitute for conventional hemoglobin or a derivative thereof in a growth medium for hemoglobin-dependent bacteria.

In certain aspects, provided herein is a method of culturing hemoglobin-dependent bacteria (e.g., for use in a bacterial composition (e.g., a pharmaceutical composition) and/or for therapeutic use), the method comprising incubating the hemoglobin-dependent bacteria in a growth medium that comprises a heme-containing polypeptide provided herein (e.g., a heme-containing polypeptide not sourced from an animal). In some aspects, provided herein is a method of culturing hemoglobin-dependent bacteria (e.g., for use in a bacterial composition (e.g., a pharmaceutical composition) and/or for therapeutic use), the method comprising (a) adding a heme-containing polypeptide provided herein (e.g., a heme-containing polypeptide not sourced from an animal) and hemoglobin-dependent bacteria to a growth medium; and (b) incubating the hemoglobin-dependent bacteria in the growth medium.

In certain aspects, provided herein is a composition comprising a growth medium comprising a heme-containing polypeptide provided herein and hemoglobin-dependent bacteria.

In certain aspects, provided herein is a bioreactor comprising hemoglobin-dependent bacteria (e.g., for use in a bacterial composition (e.g., a pharmaceutical composition) and/or for therapeutic use) in a growth medium comprising a heme-containing polypeptide provided herein (e.g., a heme-containing polypeptide not sourced from an animal). In some embodiments, provided herein is a method of culturing hemoglobin-dependent bacteria (e.g., for use in a bacterial composition (e.g., a pharmaceutical composition) and/or for therapeutic use), the method comprising comprises incubating the hemoglobin-dependent bacteria in a bioreactor provided herein.

In some embodiments, the growth medium comprises at least 0.001 g/L, at least 0.005 g/L, at least 0.01 g/L, at least 0.02 g/L, at least 0.03 g/L, at least 0.04 g/L, at least 0.05 g/L, at least 0.06 g/L, at least 0.07 g/L, at least 0.08 g/L, at least 0.09 g/L, at least 0.1 g/L, at least 0.2 g/L, at least 0.3 g/L, at least 0.4 g/L, at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of a heme-containing polypeptide provided herein. In some embodiments, the growth medium comprises at least 0.005 g/L and no more than 1 g/L of a heme-containing polypeptide. In some embodiments, the growth medium comprises about 0.02 g/L of a heme-containing polypeptide. In some embodiments, the growth medium comprises about 0.05 g/L of a heme-containing polypeptide. In some embodiments, the growth medium comprises about 0.1 g/L of a heme-containing polypeptide. In some embodiments, the growth medium comprises about 0.2 g/L of a heme-containing polypeptide. In some embodiments, the growth medium comprises about 0.5 g/L of a heme-containing polypeptide. In some embodiments, the growth medium comprises about 1 g/L of a heme-containing polypeptide. In some embodiments, the growth medium comprises yeast extract, soy peptone A2SC 19649, Soy peptone E110 19885, dipotassium phosphate, monopotassium phosphate, L-cysteine-HCl, ammonium chloride, glucidex 21 D, and/or glucose. In some embodiments, the growth media comprises about 5 g/L glucose, about 10 g/L yeast extract 19512, about 10 g/L soy peptone A2 SC 19649, about 10 g/L soypeptone E110 19885, about 2.5 g/L dipotassium phosphate K2HPO4, and about 0.5 g/L L-cysteine-HCl. In some embodiments, the growth medium is at a pH of 5.5 to 7.5. In certain embodiments, the growth medium is at a pH of about 6.5. In some embodiments of the methods and compositions provided herein, the growth medium does not comprise hemoglobin or a derivative thereof. In certain embodiments, the growth medium does not comprise animal products. In certain embodiments, the growth medium does not comprise a heme-containing polypeptide sourced from an animal.

In some embodiments of the methods and compositions provided herein, the hemoglobin-dependent bacteria (e.g., for use in a bacterial composition (e.g., a pharmaceutical composition) and/or for therapeutic use) can be any bacteria that require the presence of hemoglobin or a hemoglobin derivative for optimal growth (i.e., for optimal growth in the absence of a heme-containing polypeptide provided herein). In some embodiments of the methods and compositions provided herein, the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella. In some embodiments, the hemoglobin-dependent bacteria are of the genus Prevotella. In some embodiments, the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis. In some embodiments, the hemoglobin-dependent bacteria are Alistipes indistinctus, Alistipes shahii, Alistipes timonensis, Bacillus coagulans, Bacteroides acidifaciens, Bacteroides cellulosilyticus, Bacteroides eggerthii, Bacteroides intestinalis, Bacteroides uniformis, Collinsella aerofaciens, Cloacibacillus evryensis, Clostridium cadaveris, Clostridium cocleatum, Cutibacterium acnes, Eisenbergiella sp., Erysipelotrichaceae sp., Eubacterium hallii/Anaerobutyricum halii, Eubacterium infirmum, Megasphaera micronuciformis, Parabacteroides distasonis, Peptoniphilus lacrimalis, Rarimicrobium hominis, Shuttleworthia satelles, or Turicibacter sanguinis.

In some embodiments of the methods and compositions provided herein, the hemoglobin-dependent bacteria (e.g., for use in a bacterial composition (e.g., a pharmaceutical composition) and/or for therapeutic use) are a strain of the species Prevotella histicola. In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain B 50329. In certain embodiments, the Prevotella histicola strain is a strain that comprises at least at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain B 50329 (NRRL accession number B 50329). In certain embodiments, the Prevotella histicola strain is a strain that comprises at least at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain B 50329 (NRRL accession number B 50329). In certain embodiments, the Prevotella histicola strain is Prevotella Strain B 50329 (NRRL accession number B 50329).

In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain C (ATCC Deposit Number PTA-126140, deposited on Sep. 10, 2019). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain C (PTA-126140). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain C (PTA-126140). In certain embodiments, the Prevotella histicola strain is Prevotella Strain C (PTA-126140).

In some embodiments, the hemoglobin-dependent bacteria (e.g., for use in a bacterial composition (e.g., a pharmaceutical composition) and/or for therapeutic use) are a strain of Prevotella bacteria comprising one or more proteins listed in Table 1. In some embodiments, the hemoglobin-dependent bacteria (e.g., for use in a bacterial composition (e.g., a pharmaceutical composition) and/or for therapeutic use) are from a strain of Prevotella substantially free of one or more of the proteins listed in Table 2.

In some embodiments, the growth medium comprises at least 0.001 g/L, at least 0.005 g/L, at least 0.01 g/L, at least 0.02 g/L, at least 0.03 g/L, at least 0.04 g/L, at least 0.05 g/L, at least 0.06 g/L, at least 0.07 g/L, at least 0.08 g/L, at least 0.09 g/L, at least 0.1 g/L, at least 0.2 g/L, at least 0.3 g/L, at least 0.4 g/L, at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of a heme-containing polypeptide (e.g., a heme-containing polypeptide not sourced from an animal, e.g., a heme-containing polypeptide described herein). In some embodiments, the growth medium comprises at least 0.005 g/L and no more than 1 g/L of a heme-containing polypeptide. In some embodiments, the growth medium comprises about 0.05 g/L of a heme-containing polypeptide. In some embodiments, the growth medium comprises about 0.1 g/L of a heme-containing polypeptide. In some embodiments, the growth medium comprises about 0.02 g/L of a heme-containing polypeptide. In some embodiments, the growth medium comprises about 0.2 g/L of a heme-containing polypeptide. In some embodiments, the growth medium comprises about 0.5 g/L of a heme-containing polypeptide. In some embodiments, the growth medium comprises about 1 g/L of a heme-containing polypeptide provided herein. In some embodiments, the growth medium comprises yeast extract, soy peptone A2SC 19649, Soy peptone E110 19885, dipotassium phosphate, monopotassium phosphate, L-cysteine-HCl, ammonium chloride, glucidex 21 D, and/or glucose. In some embodiments, the growth media comprises about 5 g/L glucose, about 10 g/L yeast extract 19512, about 10 g/L soy peptone A2 SC 19649, about 10 g/L soypeptone E110 19885, about 2.5 g/L dipotassium phosphate K2HPO4, and about 0.5 g/L L-cysteine-HCl. In some embodiments, the growth medium is at a pH of 5.5 to 7.5. In certain embodiments, the growth medium is at a pH of about 6.5.

In some embodiments of the methods and compositions provided herein, the growth medium does not comprise conventional hemoglobin (e.g., porcine hemoglobin) (e.g., not comprising a hemoglobin sourced from an animal) or a derivative thereof. In certain embodiments, the growth medium does not comprise animal products. In certain embodiments, the growth medium does not comprise a heme-containing polypeptide sourced from an animal.

In some embodiments of the methods and compositions provided herein, the hemoglobin-dependent bacteria (e.g., for use in a bacterial composition (e.g., a pharmaceutical composition) and/or for therapeutic use) grow at an increased rate in the growth medium comprising a heme-containing polypeptide provided herein (e.g., a heme-containing polypeptide not sourced from an animal) compared to the rate at which the hemoglobin-dependent bacteria grow in the same growth medium but without the heme-containing polypeptide. In some embodiments, the rate at which the hemoglobin-dependent bacteria grow in the growth medium comprising a heme-containing polypeptide provided herein is at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400% higher than the rate at which the hemoglobin-dependent bacteria grow in the same growth medium but without the heme-containing polypeptide. In some embodiments, the growth rate is increased by 200% to 400%.

In certain embodiments of the methods and compositions provided herein the hemoglobin-dependent bacteria (e.g., for use in a bacterial composition (e.g., a pharmaceutical composition) and/or for therapeutic use) grow to a higher cell density in the growth medium comprising a heme-containing polypeptide provided herein, compared to the cell density to which the hemoglobin-dependent bacteria grow in the same growth medium but without the heme-containing polypeptide. In some embodiments, the hemoglobin-dependent bacteria grow to a cell density in the growth medium comprising a heme-containing polypeptide provided herein that is at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400% higher than the cell density to which the hemoglobin-dependent bacteria grow in the same growth medium but without the heme-containing polypeptide. In some embodiments, the bacterial cell density is 200% to 400% higher.

In certain aspects, provided herein is a composition comprising hemoglobin-dependent bacteria (e.g., for use in a bacterial composition (e.g., a pharmaceutical composition) and/or for therapeutic use) disclosed herein and a heme-containing polypeptide disclosed herein.

### BRIEF DESCRIPTION OF FIGURES

FIG. 1 shows the growth curves for Strain A (Prevotella histicola strain B 50329 (NRRL accession number B 50329)) with various sources of hemoglobin.

FIG. 2 shows the growth curves for Strain A (Prevotella histicola strain B 50329 (NRRL accession number B 50329)) with various sources and concentrations of hemoglobin.

FIG. 3 shows the growth curves for Parabacteroides Strain A with various sources and concentrations of hemoglobin.

FIG. 4 shows the growth curves for Bacteroides Strain B with various sources and concentrations of hemoglobin.

FIG. 5 shows the growth curves for Alistipes Strain C with various sources and concentrations of hemoglobin.

### DETAILED DESCRIPTION

In certain aspects, provided herein are methods and compositions that allow for the culturing of hemoglobin-dependent bacteria (e.g., for use in a bacterial composition (e.g., a pharmaceutical composition) and/or for therapeutic use). Specifically, disclosed herein are heme-containing polypeptides that can be added to culture media to facilitate the growth of hemoglobin-dependent bacteria. In certain embodiments, the heme-containing polypeptide can be a piscine polypeptide, an avian polypeptide, or a non-animal-derived polypeptide. These heme-containing polypeptides may be purified directly from various organisms, or a processed form thereof (e.g., flour, protein hydrolysate, meals, etc.). Alternatively, these heme-containing polypeptides may be recombinantly produced, e.g., recombinantly expressed in a host cell.

Thus, in certain aspects, provided herein are methods and compositions for culturing hemoglobin-dependent bacteria in growth media that includes a heme-containing polypeptide provided herein. In some aspects, provided herein are compositions (e.g., growth media) comprising a heme-containing polypeptide provided herein that are useful for culturing hemoglobin-dependent bacteria, as well as methods of making and/or using such compositions.

### Definitions

As used herein, “anaerobic conditions” are conditions with reduced levels of oxygen compared to normal atmospheric conditions. For example, in some embodiments anaerobic conditions are conditions wherein the oxygen levels are partial pressure of oxygen (pO₂) no more than 8%. In some instances, anaerobic conditions are conditions wherein the pO₂ is no more than 2%. In some instances, anaerobic conditions are conditions wherein the pO₂ is no more than 0.5%. In certain embodiments, anaerobic conditions may be achieved by purging a bioreactor and/or a culture flask with a gas other than oxygen such as, for example, nitrogen and/or carbon dioxide (CO₂).

As used herein, the term “bacterial composition” includes a pharmaceutical composition and/or a composition for therapeutic use. The bacterial composition can be a pharmaceutical composition. The bacterial composition can be a medicinal product, medical food, a food product, or a dietary supplement.

As used herein, “derivatives” of hemoglobin include compounds that are derived from hemoglobin that can facilitate growth of hemoglobin-dependent bacteria. Examples of derivatives of hemoglobin include hemin and protoporphyrin.

The term “gene” is used broadly to refer to any nucleic acid associated with a biological function. The term “gene” applies to a specific genomic sequence, as well as to a cDNA or an mRNA encoded by that genomic sequence.

As used herein, “heme-containing polypeptide” refers to a polypeptide that covalently or noncovalently binds to a heme moiety.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

“Non-animal-derived polypeptide” refers to a polypeptide that is not purified from an animal source. A non-animal-derived polypeptide includes both polypeptides purified from non-animal sources and polypeptides that are recombinantly expressed. Thus, for example, the term “non-animal-derived polypeptide” would include a polypeptide that is recombinantly expressed from a sequence that encodes an animal polypeptide, e.g., corresponds to a polypeptide encoded by an animal gene.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

Hemoglobin-Dependent Bacteria

In some aspects, provided herein are methods and compositions for culturing hemoglobin-dependent bacteria. The hemoglobin-dependent bacteria may be for use in a bacterial composition (e.g., a pharmaceutical composition) and/or for therapeutic use (e.g., in a subject, e.g., human). As used herein, “hemoglobin dependent bacteria” refers to bacteria for which growth rate is slowed and/or maximum cell density is reduced when cultured in growth media lacking conventional hemoglobin (e.g., porcine hemoglobin), a hemoglobin derivative or a heme-containing polypeptide disclosed herein when compared to the same growth media containing conventional hemoglobin (e.g., animal hemoglobin, e.g., porcine hemoglobin), a hemoglobin derivative or a heme-containing polypeptide disclosed herein. In some embodiments, the hemoglobin-dependent bacteria are selected from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

In some embodiments, the hemoglobin-dependent bacteria are of the genus Prevotella. In some embodiments, the hemoglobin-dependent bacteria are of the species Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella melanogenica, Prevotella intermedia, Prevotella maculosa, Prevotella marshii, Prevotella melaninogenica, Prevotella micans, Prevotella multiformis, Prevotella nigrescens, Prevotella orails, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

In some embodiments, the hemoglobin-dependent bacteria are Alistipes indistinctus, Alistipes shahii, Alistipes timonensis, Bacillus coagulans, Bacteroides acidifaciens, Bacteroides cellulosilyticus, Bacteroides eggerthii, Bacteroides intestinalis, Bacteroides uniformis, Collinsella aerofaciens, Cloacibacillus evryensis, Clostridium cadaveris, Clostridium cocleatum, Cutibacterium acnes, Eisenbergiella sp., Erysipelotrichaceae sp., Eubacterium hallii/Anaerobutyricum halii, Eubacterium infirmum, Megasphaera micronuciformis, Parabacteroides distasonis, Peptoniphilus lacrimalis, Rarimicrobium hominis, Shuttleworthia satelles, or Turicibacter sanguinis.

In some embodiments, the hemoglobin-dependent Prevotella strain is Prevotella Strain B 50329 (NRRL accession number B 50329). In some embodiments, the hemoglobin-dependent Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain C (ATCC Deposit Number PTA-126140, deposited on Sep. 10, 2019). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain C (PTA-126140). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain C (PTA-126140). In certain embodiments, the Prevotella histicola strain is Prevotella Strain C (PTA-126140).

In some embodiments, the hemoglobin-dependent Prevotella strain is a strain of Prevotella bacteria comprising one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) proteins listed in Table 1 and/or one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) genes encoding proteins listed in Table 1. In some embodiments, the hemoglobin-dependent Prevotella strain comprises all of the proteins listed in Table 1 and/or all of the genes encoding the proteins listed in Table 1.

TABLE 1
Exemplary Prevotella proteins

| SEQ | | | |
|---|---|---|---|
| ID | | | |
| NO. | Name | Uniprot ID | Amino Acid Sequence |
| 45 | Cluster: | G6ADE1 | MNLKTFTKTVLCFALFAVSAITAKAADHLAIVG |
| | Uncharacterized | | EAVWGGWDLVKATAMVKSPNNPDVFMATVHLNA |
| | protein | | GKGFKFLTEREWGKLEYRSGASDVVLKSGIRYK |
| | | | LYASIGASEDGKFKVSESANYEIICDLARKTVE |
| | | | VKKVAYQAKEIRYAALWMIGDATAGDWDYNNGV |
| | | | LLSQDSGNPTCYTATVELKEGEFKFTTNKQWGY |
| | | | DHSVYIFRDVNDQNKIVFGGEDNKWRITEDGMY |
| | | | NVTVDVPTKTISIKQIDDPAGHKPQFGNDVILV |
| | | | GDATIAGWNLDNAIYLEHTGQAGRVFKTTTYLE |
| | | | AGKGFKFLSMLSYDDIDYRPANNTVLNPGVPGT |
| | | | FVPSLPSSTDTKFSVERSGNYDIVCNMNNRTVV |
| | | | VTLSENQVLVNYPALWLIGSATSAGWNPGKAVE |
| | | | LKRSEADPAVYTARVQLKKGEFKILTSKNVGFD |
| | | | QPTYYRDSTNEHRIVFGVDGDEVAKKDCKWTLS |
| | | | ENAEGTYDVTVDIEAMTIFCDKVNMDEPSVEST |
| | | | DKELILIGDATYSAWDLPKSIVMTPVGPTTFKA |
| | | | VTHLEAGKEFKFLTELAWKRYEYRAESLRKELQ |
| | | | EGSMSMLVPYRYTNDKDDKDHDFKFVVKESGNY |
| | | | EIVCDLYIPALIIRKVRYQDTPVTYSSLWIVGS |
| | | | ATPGGWTIERGIKMTQDENYPTKFTAKANLVPG |
| | | | ELKFATNKFADFTQDFFFRGKDDYTAVLGGNDN |
| | | | KWNITEAGTYSVTIDVASKRVTITKPARNAPTG |
| | | | ISTVDSSDEAPAEYFTLNGIKVTTPSSGIYIKR |
| | | | QGGRTTKVVMK |
| 46 | Nicotinamide_ | P24520 | MDTYQILDIIGCIVGLIYIYQEYKASIWLWMTG |
| | riboside_transporter_ | | IIMPVIYMFVYYEAGLYADFGMQIYYTLAAIYG |
| | PnuC | | YLYWKLGKKKGTEDKEIPITHFPRRYIIPAIIV |
| | | | FFVLWIALYYILICFTNSTVPVLDSFGNALSFI |
| | | | GLWALAKKYLEQWWIWIVVDAELSALYIYKGIP |
| | | | FTAMLYALYTVIAVAGYFKWRRYIKQQK |
| 47 | Pectate_trisaccharide- | Q8GCB2 | MRVRLYKNILLFLFLWVNTLACVSADTSRTVES |
| | lyase | | QPIENGLIITESKGWLETIYAKWKPVAEADGYY |
| | | | VYVKGGQYADYSKVDSELIRVYNGYVRVDIPGL |
| | | | KAGTYSLKIVAVKGGKETQSSEVTGLKVLNYVR |
| | | | EGFAHKNYSGVGAYNDDGTLKSGAVVIYVNKDN |
| | | | AKTVSAHLGKTTFIGLQAILNAYQKGNITTPLS |
| | | | VRILGLLRNGDTDTFGSSTEGIQIKGKQADSEM |
| | | | NITIEGIGEDASIYGFGFLVRNAKSVEFRNLGI |
| | | | MRAMDDGVSLDTNNSNIWIHHMDLFYGKASGGD |
| | | | HIKGDGSIDVKTDSKYVTIDNCHFWDTGKTSMC |
| | | | GMKKETGPNYITYHHNWFDHSDSRHARVRTMSV |
| | | | HLWNNYYDGCAKYGIGATMGCSVFSENNYFRAT |
| | | | KNPILISKQGSDAKGTGKFSGEPGGMVKEYGSL |
| | | | FTEKGAESTYTPISYADNNSSFDFYHAISRNEK |
| | | | VPASVKTLNGGNIYNNFDTDAALMYSYTPDATA |
| | | | LVPSQVTGFYGAGRLNHGSLQFKFNNAVEDTNS |
| | | | TPIPALEALIDAYSGK |
| 48 | Glycosyltransferase_ | Q9AET5 | MKYNIAYCIEGFYNHGGMERILSVCANLLSDIY |
| | Gtf1 | | SITIIVANQRGREHAYNLAQNVNVVDLGVSCKN |
| | | | YKEEYKKSLTRYLQDHQFSVVISLAGLELFFLP |
| | | | QIKDGSKKVMWFHFAFDVSKMFLSERFHGWKLN |
| | | | LLYYIHTIRRIYFAKKFDTIVVLSKSDCDSWSR |
| | | | FCNNVKYIYNPITIDRKVISNLSEESVIAVGRL |
| | | | GWQKGFDFLIDSWVLVDDKHPDWHLDIFGEGPD |
| | | | RLELQHQIDRKGLHDKVRLCGVTKQIEEEYGKH |
| | | | SIYVMSSRAEGFPLALLEASSCGLPMISFNCHQ |
| | | | GPNEIIQEGENGFLVDKVGDIYTLSDRICKLIE |
| | | | DNNLRNMMGKKALDSSFRFEGEVIKKDWISLLK |
| | | | QLI |
| 49 | Cluster: Protein | A0A096B759 | MKRLFFMFLFLGTITMNSLAQEEKPIKYETKNF |
| | TonB | | SLPDKMPLYPGGDGALRAFLSLNLHYPEKAQAF |
| | | | GVEGRSLMKFCVSSDGSIKDISAVDCKITNYNR |
| | | | TEFNKLPLSKQESLKKECAKAFAKEAARVIRLM |
| | | | PKWEPAELNGKKMNVYYSLPFTFKLR |
| 50 | Cluster: | G6AEN6 | MNYPLFIARKIYNGGDRTRKVSKPAIRIATIGV |
| | Uncharacterized | | AIGLAVMIISVGVVLGFKHTIRNKVVGFGSDIT |
| | protein | | VANFLTLQSSEQYPIQITDSLVKSLQITPGIKH |
| | | | VQRYDYTQGILKTDNDFLGVLLKGVGPDFDSTF |
| | | | IHENMVEGSLPHFHDNESQQKIVISKTIADKLN |
| | | | LKVGQRIFAYFINKQGVRTRKFTITGIYATNMK |
| | | | QFDSQICFTDIYTTNKLNGWEPDQYSGAELQVD |
| | | | NFSQLTPISMRVLNKVKNTVDHYGGTYSSENII |
| | | | EQNPQIFSWLDLMDMNVWIILALMISVAGVTMI |
| | | | SGLLIIILERTQMIGILKALGSRNRQIRHIFLW |
| | | | FATFIIGKGLLWGNIIGLGCILFQSWTGLVKLD |
| | | | PQTYYVNTVPVEINIPLIIALNMVTMLVCLVIL |
| | | | IAPSYLISHIHPAKSMHYE |
| 51 | Bifunctional_ | P9WHG9 | MEDKFIYTDKERKLSYQILDELKDTLDKSFLEN |
| | (p)ppGpp_synthase/ | | DLPMLQVQLKDSVAKNTIHRNVFGLNPILCSLQ |
| | hydrolase_RelA | | TAAIAVKDIGLKRDSVIAILLHQSVQDGYITLE |
| | | | DIDNRFGKSVAKIIHGLIRIQTLYQKNPIIESE |
| | | | NFRNLLLSFAEDMRVILIMIADRVNLMRQIRDA |
| | | | EDKEAQHKVAEEASYLYAPLAHKLGLYQLKREL |
| | | | EDLSLKYLEHDAYYLIKDKLNATKASRDAYINQ |
| | | | FIAPVRERLTAGGLRFHIKGRTKSIHSIWQKMK |
| | | | KQKCGFEGIYDLFAIRIILDAPLEKEKIQCWQA |
| | | | YSIVTDMYQPNPKRLRDWLSVPKSNGYECLHIT |
| | | | VLGPEKKWVEVQIRTERMDEIAEHGLAAHWRYK |
| | | | GIKEEGGLDDWLASIRAALEAGDNLEVMDQFKS |
| | | | DLYEKEIYVFTPKGDLLKFPKGATILDFAYHIH |
| | | | SKVGNQCVGGKINAKNVSLRTELHSGDTVEILT |
| | | | SATQKPKAEWLKIVKSSRAKAKIRLALKETQIK |
| | | | DGLYAKELLERRFKNKKIEIEESTMGHLLRKLG |
| | | | FKEVSEFYKQVADEKLDPNYIIEEYQKVYNHDH |
| | | | NLNQPKETESAENFEFENPTNEFLKKNDDVLVI |
| | | | DKNLKGLDFSLAKCCHPIYGDPVFGFVTVNGG1 |
| | | | KIHRTDCPNAPEMRKRFGYRIVKARWSGKGSSQ |
| | | | YAITLRVIGNDDIGIVSNITNVISKDEKIVMRS |
| | | | INIDSHDGLFSGNLVVLLDDNSKLNMLIKKLRT |
| | | | VKGVKQVTRI |
| 52 | VitaminB 12_import_ | P06609 | MKRRIFLFVALSVSIVILFGLNLIIGSVHIPLS |
| | system_permease_ | | DILTILSGSFTGKESWRFIIWDSRLPQALTAML |
| | protein_BtuC | | CGSSLAVCGLMLQTAFRNPLAGPDVFGISSGAS |
| | | | LGVALVMLLLGGTVETSMFTASGFLAILIVAFA |
| | | | GAILVTAFILFLSSVVRNSVLLLIVGIMVGYVA |
| | | | SSAVTLLNFFSSEDGVKGYIVWGMGNFGGVSMS |
| | | | HIPLFAFLCLAGIIASFLLVKPLNILLLGPQYA |
| | | | ESLGISIRRIRNILLVVVGILTAVTTAFCGPIS |
| | | | FIGLAAPHVARLLFRTENHQKLLPGTLLVGTVV |
| | | | ALLCNLICFLPRESGMIPLNAVTPLIGAPIIIY |
| | | | VIMKRH |
| 53 | NADH- | P33599 | MKLENKEFGFDSFATEMARLKNEKHFDYLVTVV |
| | quinone_oxidoreductase_ | | GEDFGTEEGLGCIYILENTSTHERCSVKQLAKK |
| | subunit_C/ | | VGEEFVIPSVIKLWADADLLEREVYDFYGIKFL |
| | D | | GHPDMRRLFLRNDFKGYPLRKDYDMDPAKNMYT |
| | | | TEDDVELDTTTEWNLDKNGELVGTQHALFTDDN |
| | | | FVVNIGPQHPSTHGVLRLQTVLDGETVTNIYPH |
| | | | LGYIHRGIEKLCEQFTYPQTLALTDRMNYLSAM |
| | | | MNRHALVGVIEEGMGIELSERILYIRTIMDELQ |
| | | | RIDNHLLYTACCAQDLGALTAFLYGMRDREHVL |
| | | | NVMEETTGGRLIQNYYRIGGLQADIDPNFVSNV |
| | | | KELCKYLRPMIQEYVDVFGDNVITHQRFEGVGV |
| | | | MDEKDCISYGVTGPAGRASGWKNDVRKYHPYAM |
| | | | YDKVNFEEITLTNGDSMDRYFCHIKEIYQSLNI |
| | | | IEQLIDNIPEGEFYIKQKPIIKVPEGQWYFSVE |
| | | | GASGEFGAYLDSRGDKTAYRLKFRPMGLTLVGA |
| | | | MDKMLRGQKIADLVTTGAALDFVIPDIDR |
| 54 | FKBP- | P45523 | MRTSTQSKDMGKKQEYKLRNEEFLHNISKKDSI |
| | type_peptidyl- | | KTLPHGIFYEIIKEGSGEGTVQPRSIVICNYRG |
| | prolyl_cis- | | SLISGQVFDDSWQKPTPEAFRLNELITGLQIAL |
| | trans_isomerase | | CAMHKGDSWRIYIPYQEGYGSKRNADIPAFSTL |
| | | | IFDIELINIA |
| 55 | Putative_acetolactate_ | P9WKJ3 | MADNKIAKESVKREVIAGERLYTLLVYSENVAG |
| | synthase_small_ | | VLNQIAAVFTRRQVNIESLNVSASSIEGIHKYT |
| | subunit | | ITAWSDAATIEKITKQVEKKIDVIKADYYEDSD |
| | | | LFIHEVGLYKIATPILLENAEVSRAIRKRNARM |
| | | | MEVNPTYSTVLLAGMTDEVTALYHDLKNFDCLL |
| | | | QYSRSGRVAVTRGFSEPVSDFLKSEEESSVL |
| 56 | Serine/threonine_ | P0AGE4 | MKKKVKIGLLPRVIIAILLGIFFGYFMPTPLAR |
| | transporter_SstT | | VFLTFNGIFSQFLGFMIPLIIIGLVTPAIADIG |
| | | | KGAGKLLLVTVIIAYVDTVVAGGLAYGTGLCLF |
| | | | PSMIASTGGAMPHIDKATELAPYFSINIPAMAD |
| | | | VMSGLVFSFMLGLGIAYGGLTATKNIFNEFKYV |
| | | | IEKVIAKAIIPLLPLYIFGVFLNMAHNGQAQQ1 |
| | | | LLVFSQIIIVILVLHVFILVYQFCIAGAIIRRN |
| | | | PFRLLWNMMPAYLTALGTSSSAATIPVTLEQTM |
| | | | KNGVGKEIAGFVVPLCATIHLSGSAMKITACAL |
| | | | TICLLVGLPHDPALFIYFILMLSIIMVAAPGVP |
| | | | GGAIMAALAPLASILGFNSEAQALMIALYIAMD |
| | | | SFGTACNVTGDGAIALVVNKMFGKKER |
| 57 | Cluster: | G6AJ07 | MKKLLLLVCAAVMSLSASAQAGDKALGAQLVFG |
| | Uncharacterized | | SETNSLGFGVKGQYYFTDHIRGEGSFDYFLKNK |
| | protein | | GISMWDINANVHYLFDVADKFKVYPLAGLGYTN |
| | | | WSYKYEYAGAPVVEGSDGRLAVNLGGGVEYELT |
| | | | KNLNVNAEAKYQIISNYNQLVLGVGVAYKF |
| 58 | Heterocyst_ | P22638 | MHFYCTKSSLDTMSERYVKRMIAKLASQGKTVI |
| | differentiation_ATP- | | SIAHRFSTIMDAKHIILLAKGKVVAEGTHQELL |
| | binding_protein | | KTSEDYRKLWSDQNDEID |
| 59 | UDP-2,3- | Q9I2V0 | MKNVYFLSDAHLGSLAIAHRRTQERRLVRFLDS |
| | diacylglucosamine_ | | IKHKASAVYLLGDMFDFWDEYKYVVPKGFTRFL |
| | hydrolase | | GKVSELTDMGVEVHFFTGNHDLWTYGYLEEECG |
| | | | VILHRKPVTMEIYGKVFYLAHGDGLGDPDPMFQ |
| | | | FLRKVFHNRVCQRLLNFFHPWWGMQLGLNWAKK |
| | | | SRLKRADGKEMPYLGEDKEYLVRYTKDYMRSHK |
| | | | DIDYYIYGHRHIELDLTLSGKVRMLILGDWIWQ |
| | | | FTYAVFDGEHMFLEEYIEGESKP |
| 60 | Anacrobic_glycerol- | P0A9C0 | MNSKQNDNYDVIIIGGGITGAGTARDCALRGLK |
| | 3- | | VLLVEKFDFTNGATGRNHGLLHSGARYAVTDPE |
| | phosphate_dehydrogenase | | SATECIKENMVLRRIAKHCIEETDGLFITLPED |
| | | | DINYQKTFVEACARAGISANIISPEEALRLDPS |
| | | | VNPDLLGAVRVPDASVDPFHLTTANVLDARQHG |
| | | | ADVLTYHEVVAILTSNGRVEGVRLRNNHTGEEI |
| | | | EKHAVLVINAAGIWGHDIAKMADIKINMFPAKG |
| | | | TLLVFGHRVNKMVINRCRKPANADILVPDDAVC |
| | | | VIGTTSDRVPYDTVDNLKITSEEVDTLIREGEK |
| | | | LAPSLATTRILRAYAGVRPLVAADNDPTGRSIS |
| | | | RGIVCLDHEKRDGLTGMITITGGKMMTYRLMAE |
| | | | QATDLACKKLGINKTCETATTPLPGTAGKDSDN |
| | | | PHHTYSTAHKAAKGRQGNRVKEIDERTEDDRAL |
| | | | ICECEEVSVGEAKYAIEELHVHDLLNLRRRTRV |
| | | | GMGTCQGELCACRAAGVMCENGVKVDKAMTDLT |
| | | | KFINERWKGMRPVAWGSTLDEAQLTTIIYQGLC |
| | | | GLGI |
| 61 | Anaerobic_glycerol- | P13033 | MRYDTIIIGGGLSGLTAGITLAKAGQKVCIVSA |
| | 3- | | GQSSLHFHSGSFDLLGYDADGEVVTHPLQAIAD |
| | phosphate_dehydrogenase | | LKAEHPYSKIGISNIEHLASQAKTLLCEAGISV |
| | | | MGNYEQNHYRVTPLGTLKPAWLTTEGYAMIDDP |
| | | | EILPWKKVELLNIQGFMDFPTQFIAENLRMMGV |
| | | | ECQIKTFTTDELSTARQSPTEMRATNIAKVLAN |
| | | | KDALSKVSERINAISGDPDALLLPAVLGFSNAE |
| | | | SLDEMKQWIKKPVQYIATLPPSVSGVRTTILLK |
| | | | RLFAQAGGTLLIGDSATTGQFSGNHLVSITTDH |
| | | | LPDEKLYADHFILASGSFMSHGIRSNYAGVYEP |
| | | | VFKLDVDAAEKRDDWSVTNAFEAQPYMEFGVHT |
| | | | DKDFHATKDGKNIENLYAIGSVLSGHNSIKHAD |
| | | | GTGVSLLTALYVAKKITGKG |
| 62 | Anacrobic_glycerol- | P0A996 | MAEGIQLKNISGNNLEQCLKCSICTAYCPVSAV |
| | 3- | | EPKYPGPKQSGPDQERYRLKDSKFFDEALKMCL |
| | phosphate_dehydrogenase | | NCKRCEVACPSGVRIADIIQASRITYSTHRPIP |
| | | | RDIMLANTDFVGTMANMVAPIVNATLGLKPVKA |
| | | | VLHGVMGIDKHRTFPAYSSQKFETWYKRMAAKK |
| | | | QDSYSKHVSYFHGCYVNYNFPQLGKDLVKIMNA |
| | | | VGYGVHLLEKEKCCGVALIANGLSGQARRQGKV |
| | | | NIRSIRKAAEQNRIVLTTSSTCTFTMRDEYEHL |
| | | | LDIKTDDVRENITLATRFLYRLIEKGDIKLAFR |
| | | | KDFKMRTAYHSACHMEKMGWIIYSTELLKMIPG |
| | | | LELIMLDSQCCGIAGTYGFKKENYQRSQEIGEG |
| | | | LFKQIKELNPDCVSTDCETCKWQIEMSTGYEVK |
| | | | NPISILADALDVEETIKLNQ |
| 63 | Glycerol_uptake_ | P18156 | MMIKNIVLSIPISLIIYLNHLIMEYSMTTQFLM |
| | facilitator_protein | | ELIGTLILVLFGDGVCACVTLNKSKGQKAGWVV |
| | | | ITIAWGLAVCMGVLVAGPYTGAHLNPAVSIGLA |
| | | | VAGMFPWSSVPYYIVAQMIGGFLGGLLVWFFYK |
| | | | DHYDATDDEAAKLGTFCTSPAIRNYKMNFLSEV |
| | | | IATLVLVFIIISFSVDGNTGDAEHFKFGLAALG |
| | | | PIPVTLLIIALGMSLGGTTGYAMNPARDLSPRL |
| | | | AHAVCMKGDNDWSYSWIPVLGPIIGAIIAGFCG |
| | | | AALLLV |
| 64 | Serine/threonine- | Q97PA9 | MSEKIIPSNEPAQAASEPIKASYTEYTVIPSQG |
| | protein_kinase_StkP | | YCQFVKCKKGDQPVVLKGLKEAYRERVLLRNAL |
| | | | KREFKQCQRLNHPGIVRYQGLVDVEGYGLCIEE |
| | | | EYVDGRTLQAYLKESHTDDEKITIVNQIADALR |
| | | | YAHQQGVAHRNLKPSNILITKQGDHVKLIDFNV |
| | | | LSLDDVKPTADTTRFMAPELKDETMTADGTADI |
| | | | YSLGTIMKVMGLTLAYSEVIKRCCAFKRSDRYS |
| | | | DIDEFLADFNHDGSSFSMPKIGKGTVVIGFIAV |
| | | | VVIALAALAYNYGGALVDQVGKIDVTSIFKSDA |
| | | | ETAPEDSAMVKSVEQNNNDSVADEAPATGKLAF |
| | | | MNTMKPALYKDLDRLFAKHSDDRAKLNRAIKVY |
| | | | YRGLIQANDTLDNEQRAELDRVFGNYVKQKKAA |
| | | | LK |
| 65 | Cluster: D-alanyl- | G6AHI1 | MLVAQLFVGVLQAQKPVQNRRQAVGQSMERQGL |
| | D-alanine | | VNVKAVVPSIKVALMYARTDNFCHRMALS |
| | dipeptidase | | |
| 66 | Anaerobic_C4- | P0ABN5 | MITGLVIIQLLIVLALIFIGARVGGIGLGIYGM |
| | dicarboxylate_ | | IGVFILVYGFGLAPGSAPIDVMMIIVAVITAAS |
| | transporter_DcuA | | ALQASGGLEYLVGVAAKFLQKHPDHITYFGPIT |
| | | | CWLFCVVAGTAHTSYSLMPIIAEIAQTNKIRPE |
| | | | RPLSLSVIAASLGITCSPVSAATAALISQDLLG |
| | | | AKGIELGTVLMICIPTAFISILVAAFVENHIGK |
| | | | ELEDDPEYKRRVAAGLINPEAACEEVQKAENEH |
| | | | DPSAKHAVWAFLFGVALVILFGFLPQLRPEGVS |
| | | | MSQTIEMIMMSDAALILLVGKGKVGDAVNGNIF |
| | | | KAGMNAVVAIFGIAWMGNTFYVGNEKILDAALS |
| | | | SMISSTPILFAVALFLLSIMLFSQAATVTTLYP |
| | | | VGIALGINPLLLIAMFPACNGYFFLPNYPTEVA |
| | | | AIDFDRTGTTRVGKYVINHSFQIPGFITTIVSI |
| | | | LLGVLMVQFFR |
| 67 | L-asparaginase_2 | P00805 | MRILKITFVTVLALVMSTVVFAQKPKIRIIATG |
| | | | GTIAGVSASATSSAYGAGQVGVQTLIDAVPQIK |
| | | | DIADVSGEQLVNIGSQDMNDEVWLKLAKRINDL |
| | | | LNKEGYDGVLITHGTDTMEETAYFLSLTVHTDK |
| | | | PVVMVGSMRPSTAISADGPANLYNGICTLVDPS |
| | | | SKGHGVMVCMNNELFEAKSVIKTHTTDVSTFKG |
| | | | GLYGEMGYVYNGKPYFLHKPVAKQGLTSEFNVD |
| | | | NLTSLPKVGIVYGYANCSPLPIQAFVNAKFDGI |
| | | | VLAGVGDGNFYKDVFDVALKAQNSGIQIVRSSR |
| | | | VPFGPTNLNGEVDDAKYHFVASLNLNPQKARVL |
| | | | LMLALTKTKDWQKIQQYFNEY |
| 68 | Trehalose_synthase/ | P9WQ19 | MALACAMTMSASAQMGTNPKWLGDAIFYQIYPS |
| | amylase_TreS | | SYMDTDGNGIGDLPGITQKLDYIKSLGVNAIWL |
| | | | NPVFESGWFDGGYDVIDFYKIDPRFGTNTDMVN |
| | | | LVKEAHKRGIKVCLDLVAGHTSTKCPWFKESAN |
| | | | GDRNSRYSDYFIWTDSISEADKKEIAERHKEAN |
| | | | PASSTHGRYVEMNAKRGKYYEKNFFECQPALNY |
| | | | GFAKPDPNQPWEQPVTAPGPQAVRREMRNIMAE |
| | | | WFDKGVDGFRVDMASSLVKNDWGKKEVSKLWNE |
| | | | MREWKDKNYPECVLISEWSDPAVAIPAGFNIDF |
| | | | MIHFGIKGYPSLFFDRNTPWGKPWPGQDISKDY |
| | | | KFCYFDKAGKGEVKEFVDNFSEAYNATKNLGYI |
| | | | AIPSANHDYQRPNIGTRNTPEQLKVAMTFFLTM |
| | | | PGVPFIYYGDEIGMKYQMDLPSKEGSNERAGTR |
| | | | TPMQWTSGPTAGFSTCNPSQLYFPVDTEKGKLT |
| | | | VEAQQNDPRSLLNYTRELTRLRHSQPALRGNGE |
| | | | WILVSKESQPYPMVYKRTSGGETVVVAINPSDK |
| | | | KVSANIAHLGKAKSLIMTGKASYKTGKTEDAVE |
| | | | LNGVSAAVFKIAE |
| 69 | Ribitol-5 - | Q720Y7 | MNIAVIFAGGSGLRMHTKSRPKQFLDLNGKPII |
| | phosphate_ | | IYTLELFDNHPGIDAIVVACIESWIPFLEKQLR |
| | cytidylyltransferase | | KFEINKVVKIVPGGESGQASIYNGLCAAEAYIK |
| | | | SKNVASEDTTVLIHDGVRPLITEETITDNINKV |
| | | | AEVGSCITCIPATETLVVKQHDGSLEIPSRADS |
| | | | LIARAPQSFLLSDILTAHRRAIDEKKNDFIDSC |
| | | | TMMSHYGYRLGTIIGPMENIKITTPTDFFVLRA |
| | | | MVKVHEDQQIFGL |
| 70 | UDP-Glc: alpha-D- | B5L3F2 | MTEKKSVSIVLCTYNGTKYLQEQLDSILAQTYP |
| | GlcNAc- | | LHEIIIQDDGSTDNTWQILEKYEEKYPLIHIYH |
| | diphosphoundecaprenol | | NEGTHGVNANFLSAMHRTTGDFIAIADQDDIWE |
| | | | TDKIANQMTTIGNKLLCSGLTRPFSSDGSFAYF |
| | | | DNRPRNVSIFRMMFLGLPGHTMLFRRELLRMMP |
| | | | PVTHSFFNVSLYDAALSILAASHDSIAFCNKVL |
| | | | VNFRRHADATTYNDYSRSLPSWQNGLYELLWGL |
| | | | RHYHQARSIALPIYRGKLALMEGITTNYHDFIE |
| | | | AKAIMRLETQKGLWAFLRLQYLLTKNHQRLFQT |
| | | | SGGSFIKMIRAWLYPVMQLYMYHHALRRCK |
| 71 | UDP-N- | P33038 | MESFIIEGGHRLSGTIAPQGAKNEALEVICATL |
| | acetylglucosamine | | LTTEEVIIRNIPNILDVNNLIKLLQDIGVKVKK |
| | | | LGANDFSFQADEVKLDYLESIDFVKKCSSLRGS |
| | | | VLMIGPLLGRFGKATIAKPGGDKIGRRRLDTHF |
| | | | LGFKNLGARFVRIEDRDVYEIQADKLVGDYMLL |
| | | | DEASVTGTANIIMSAVMAEGTTTIYNAACEPYI |
| | | | QQLCHLLNAMGAKITGIASNLITIEGVTSLHGA |
| | | | EHRILPDMIEVGSFIGMAAMVGDGVRIKDVSIP |
| | | | NLGLILDTFRRLGVQIIEDEDDLIIPRQDHYVI |
| | | | DSFIDGTIMTISDAPWPGLTPDLISVLLVVATQ |
| | | | AQGSVLFHQKMFESRLFFVDKLIDMGAQIILCD |
| | | | PHRAVVVGHDHAKKLRAGRMSSPDIRAGIALLI |
| | | | AALTAEGTSRIDNIAQIDRGYENIEGRLNALGA |
| | | | KVQRVEIC |
| 72 | Sensor_protein_EvgS | P30855 | MERSGNFYKAIRLGYILISILIGCMAYNSLYEW |
| | | | QEIEALELGNKKIDELRKEINNINIQMIKFSLL |
| | | | GETILEWNDKDIEHYHARRMAMDSMLCRFKATY |
| | | | PAERIDSVRHLLEDKERQMCQIVQILEQQQAIN |
| | | | DKITSQVPVIVQKSVQEQPKKSKRKGFLGIFGK |
| | | | KEEAKPTVTTTMHRSFNRNMRTEQQAQSRRLSV |
| | | | HADSLAARNAELNRQLQGLVVQIDGKVQTDLQK |
| | | | REAEITAMRERSFIQIGGLTGFVILLLVISYII |
| | | | IHRNANRIKRYKQETADLIERLQQMAKRNEALI |
| | | | TSRKKAVHTITHELRTPLTAITGYAGLIQKNFN |
| | | | ADKTGMYIRNIQQSSDRMREMLNTLLSFFRLDD |
| | | | GKEQPNFSTCRISSIAHTLESEFMPIAINKGLA |
| | | | LTVTNHTDAVVLTDKERILQIGNNLLSNAIKFT |
| | | | ENGAVSLTMGYDNGMLKLIVKDTGSGMTEEEQQ |
| | | | RVFGAFERLSNAAAKDGFGLGLSIVQRIVTMLG |
| | | | GTIQLKSEKGKGSRFTVEIPMQSAEELPERINK |
| | | | TQIHHNRTLHDIVAIDNDKVLLLMLKEMYAQEG |
| | | | IHCDTCTNAAELMEMIRRKEYSLLLTDLNMPDI |
| | | | NGFELLELLRTSNVGNSRIIPIIVTTASGSCNR |
| | | | EELLERGFSDCLLKPFSISELMEVSDKCAMKGK |
| | | | QNEKPDFSSLLSYGNESVMLDKLIAETEKEMQS |
| | | | VRDGEQRKDFQELDALTHHLRSSWEILRADQPL |
| | | | RELYKQLHGSAVPDYEALNNAVTAVLDKGSEII |
| | | | RLAKEERRKYENG |
| 73 | Phosphate- | Q7A5Q2 | MKRSRFYITVGLILSLTLLMSACGQKKAKDGRT |
| | binding_protein_ | | DTPTSGTIKFASDESFSPIVEELLQNYQFRYPQ |
| | PstS | | AHLLPIYTDDNTGMKLLLDQKVNLFITSHAMTK |
| | | | GEDAILRGKGPIPEVFPIGYDGIAFIVNRSNPD |
| | | | SCITVDDVKKILQGKIAKWNQLNPKNNRGSIEV |
| | | | VFDNKASATLHYVVDSILGGKNIKSENIVAAKN |
| | | | SKSVIDYVNKTPNAIGVIGSNWLNDHRDTTNTT |
| | | | FKKDVTVASISKATVASPSNSWQPYQAYLLDGR |
| | | | YPFVRTIYALLADPHKALPYAFANYIANPIGQM |
| | | | IIFKAGLLPYRGNINIREVEVKNQ |
| 74 | Bifunctional_purine_ | P9WHM7 | MAGTKRIKTALISVFHKDGLDDLLKKLDEEGVQ |
| | biosynthesis_ | | FLSTGGTQQFIESLGYECQKVEDVTSYPSILGG |
| | protein_PurH | | RVKTLHPKIFGGILARRDNEEDQKQMVEYTIPA |
| | | | IDLVIVDLYPFEQTVASGASAQDIIEKIDIGGI |
| | | | SLIRAGAKNFKDVVIVPSKAEYPVLLQLLNTKG |
| | | | AETEIEDRKMFAERAFGVSSHYDTAIHSWFAAE |
| 75 | Multidrug_efflux_ | P0AE06 | MEEEKGGRIGQRPYILKIITERNYIIIIDMKKA |
| | pump_subunit_AcrA | | KILLFVTALVAVLTSCGGGQKGLPTSDEYPVIT |
| | | | IGASNAQLKTTYPATIKGVQDVEVRPKVSGFIT |
| | | | KLNIHEGEYVHAGQVLFVIDNSTYQAAVRQAQA |
| | | | QVNSAQSAVAQAKANVVQANASLNSANAQAATS |
| | | | RLTYNNSQNLYNNKVIGDYELQSAKNTYETAQA |
| | | | SVRQAQSGIASAQAAVKQAEAGVRQAQAMLSTA |
| | | | KDNLGFCYVKSPASGYVGSLPFKEDALVSASSA |
| | | | QPVTTISNTSTIEVYFSMTEADVLKLSRTDDGL |
| | | | SNAIKKFPAVSLLLADGSTYNHEGAIVKTSGMI |
| | | | DATTGTINVIARFPNPEHLLKSGGSGKIVIAKN |
| | | | NNRALLIPQEAVTQVQNKMFVYKVDAKDKVHYS |
| | | | EITVDPQNDGINYIVTSGLKMGERIVSKGVSSL |
| | | | EDGAKIKALTPAEYEEAIKKAEKLGENQSSASG |
| | | | FLKTMKGDSK |
| 76 | Cell_division_protein_ | Q81X30 | MAKRRNKARSHHSLQVVTLCISTAMVLILIGMV |
| | FtsX | | VLTVFTSRNLSSYVKENLTVTMILQPDMSTEES |
| | | | AALCQRIRSLHYINSLNFISKEQALKEGTRELG |
| | | | ANPAEFAGQNPFTGEIELQLKANYANNDSIKNI |
| | | | ERELRTYRGVSDITYPQNLVESVNHTLGKISLV |
| | | | LLVIAILLTIVSFSLMNNTIRLSIYARRFSIHT |
| | | | MKLVGASWGFIRAPFLRRAVMEGLVSALLAIAV |
| | | | LGVGLCLLYDYEPDITKVLSWDVLVITAGVMLA |
| | | | FGVLIATFCSWLSVNKFLRMKAGDLYKI |
| 77 | Fe(2+)_transporter_ | Q9PMQ9 | MKLSDLKTGETGVIVKVLGHGGFRKRIIEMGFI |
| | FeoB | | QGKQVEVLLNAPLRDPVKYKIMGYEVSLRHSEA |
| | | | DQIEVISAEEARQLEQAKADNEPQQGALSNNIP |
| | | | DESDHALTPFELTDAANRKSKVINVALVGNPNC |
| | | | GKTSLFNFASGAHERVGNYSGVTVDAKVGRANY |
| | | | EGYEFHLVDLPGTYSLSAYSPEELYVRKQLVEK |
| | | | TPDVVINVIDASNLERNLYLTTQLIDMHVRMVC |
| | | | ALNMFDETEQRGDNIDYQKISELFGIPMVPTVF |
| | | | TNGRGVKELFHQVIAVYEGKEDETSQFRHIHIN |
| | | | HGHELEGGIKNIQEHLRAYPDICQRYSTRYLAI |
| | | | KLLEHDKDVEELIKPLKDSDEIFKHRDIAAQRV |
| | | | KEETGNESETAIMDAKYGFIHGALEEADYSTGQ |
| | | | KKDTYQTTHFIDQILTNKYFGFPIFFLILFIMF |
| | | | TATFVIGQYPMDWIDGGVSWLGDFISSNMPDGP |
| | | | VKDMLVDGIIGGVGAVIVFLPQILILYFFISYM |
| | | | EDSGYMARAAFIMDKLMHKMGLHGKSFIPLIMG |
| | | | FGCNVPAVMATRTIESRRSRLVTMLILPLMSCS |
| | | | ARLPIYVMITGSFFALKYRSLAMLSLYVIGILM |
| | | | SVIMSRVFSRFLVKGEDTPFVMELPPYRFPTWK |
| | | | AIGRHTWEKGKQYLKKMGGIILVASIIVWALGY |
| | | | FPLPDKPDMGQQERQEHSFIGQIGHAVEPVFRP |
| | | | QGFNWKLDVGLLAGVGAKEIVASTMGVLYSNDD |
| | | | SFKDDNSFSSEGGKYVKLHKQITQDVANLHGVS |
| | | | YNEAEPIATLTAFCFLLFVLLYFPCIATIAAIK |
| | | | GETGSWGWALFAAGYTTLLAWVVSAIVFQVGML |
| | | | FIG |
| 78 | Pneumolysin | Q04IN8 | MKKNLLKAVLPASLALFAVTFGSCSQDGQLTGT |
| | | | KEDTGERVLDNTREIQNYLRTLPLAPMMSRASD |
| | | | PVPSDDGTTVPVDEGTSKTEEKGVLNGIPGSWV |
| | | | KTTRRYKMTQAFDESFLFDPTSDIVYPGCVLKG |
| | | | GTIANGTYAIITSHETGDVTFSINLSPANPQEA |
| | | | RETSATVHNIRKSEYQEVWNKWANMQWKESPIT |
| | | | TIESVEKINSQEELATKLGVAVNSPVANGSLNF |
| | | | GFNFNKKKNHILARLIQKYFSVSTDAPKKGNIF |
| | | | ESIDKEALDGYQPVYISNINYGRIIYLSVESDE |
| | | | DEKVVDEAINFAMNQIKGVDVSVSADQSLHYRK |
| | | | VLANCDIRITVLGGGQTIQKEVLKGDIDSFQRF |
| | | | LNADIPMEQMSPISFSLRYAVDNSQARVVTSNE |
| | | | FTVTQRDFVPEFKKVRMQLQVLGFSGTNTGPFP |
| | | | NLDREAGLWGSISLSLNGQDNELVKISQSNPFF |
| | | | FNYREKKETMHPIGFGGIVTVEFDKDPNESLED |
| | | | FVDHQKMTFVSDLHSTRSIYNYNFGRTTFTHTL |
| | | | GTLYTKYKGDDPIFVLESNNKNVKIHTYVKVLD |
| | | | MKFFN |
| 79 | Cluster: | G6AG77 | MTKFIYAMSLFLLAAISIKAQPIQKTSGCLLHG |
| | Uncharacterized | | SVVSSTDATAIAGATVRLYQLKKLVGGTVSDAS |
| | protein | | GNFDVKCPSSGSLQLRITAVGFKEVDTTLNVPT |
| | | | VTPLSIYMRAGKHAMDEVTVTASEKRGMTSTTV |
| | | | IGQTAMEHLQPSSFADLLALLPGGMTKIPALGS |
| | | | ANVITLREAGPPSSQYATSSLGTKFVIDGQAIG |
| | | | TDANMQYTAGSFQGDADNSRNHVSYGVDMREIP |
| | | | TDNIEKVEVVRGIPSVKYGELTSGLINITRKRS |
| | | | QSPLLLRLKADEYGKLVSVGKGFLLSGKWNLNV |
| | | | DGGLLDARKEPRNRFETYRRLTFSARLRRKWNL |
| | | | GERYVLEWSGATDYSLNIDNVKTDPEIQIHRED |
| | | | SYRSSYLKMGMNHRLLLRRKALVGLQSVSLAYS |
| | | | ASLASDRIHQTEAVALQRDYVVPLAYEGGEYDG |
| | | | LFLPMQYLCDYRVEGKPFYSTLRGETEWLARTS |
| | | | FISHHITAGGEFLLNKNYGRGQIFDITKPLHAS |
| | | | TARRPRSYKDIPATDILSFYAEDKATMPIGKHQ |
| | | | LTVMAGLRTTQMLNIPASYAVHGKLFTDTRVNV |
| | | | QWDFPSFLGFKSFVSGGLGMMTKMPTVLDLYPD |
| | | | YVYKDITEMNYWDIRPAYKRIHIRTYKLNQVNP |
| | | | DLRPARNKKWEIRLGMDKGAHHFSVTYFHEDMK |
| | | | DGFRSTTTMRPFIYKRYDTSVINPSALTGPPSL |
| | | | ASLPVVTDTLLDGYGRTENGSRITKQGIEFQYS |
| | | | SPRIPVIQTRITVNGAWFRTLYENSIPLFRSAP |
| | | | NVVVGTVAIADRYAGYYMSTDKYDKQIFTSNFI |
| | | | FDSYVDKLGLILSATAECFWMSNTKRPATSSTP |
| | | | MGYMDITGTVHPYVEADQSDPYLRWLVLTGTAG |
| | | | QDMDYRERSYMLVNFKATKRFGRHLSLSFFADR |
| | | | VFYVAPDYEVNGFIVRRTFSPYFGMEIGLKI |
| 80 | Cell_division_ATP- | P0A9R7 | MLIDFKKVNIYQDERLILKDIDFQATEGEFIYL |
| | binding_protein_FtsE | | IGRVGSGKSSLLKTFYGELDIDQEDAEKAEVLG |
| | | | ESVLDIKQKRIPALRRQMGIIFQDFQLLHDRSV |
| | | | AKNLKFVLQATGWKDKEKIKQRIKEVLEQVGMI |
| | | | DKAAKMPSELSGGEQQRIAIARAFLNNPKIILA |
| | | | DEPTGNLDPETASNIVSILKDTCKNGTTVIMST |
| | | | HNINLLSQFPGKVYRCMEQALVPVTNEAQTKDL |
| | | | EEDSTSVEPLIEPVLEEEAQAEDSKE |
| 81 | Di-/ | P0C2U3 | MFENQPKALYALALANTGERFGYYTMIAVFALF |
| | tripeptide_transporter | | LRANFGLEPGTAGLIYSIFLGLVYFLPLIGGIM |
| | | | ADKFGYGKMVTIGIIVMFAGYLFLSVPLGGGTV |
| | | | AFGAMLAALLLISFGTGLFKGNLQVMVGNLYDT |
| | | | PELASKRDSAFSIFYMAINIGALFAPTAAVKIK |
| | | | EWAETSLGYAGNDAYHFSFAVACVSLIVSMGIY |
| | | | YAFRSTFKHVEGGTKKTEKAAAAAVEELTPQQT |
| | | | KERIVALCLVFAVVIFFWMAFHQNGLTLTYFAD |
| | | | EFVSPTSTGVQSMAFDVVNLVMIVFIVYSIMAL |
| | | | FQSKTTKAKGIACAVILAAIAVLAYKYMNVNGQ |
| | | | VEVSAPIFQQFNPFYVVALTPISMAIFGSLAAK |
| | | | GKEPSAPRKTAYGMTVAGCAYLLMVLASQGLLT |
| | | | PHEQKLAKAAGETVPFASANWLIGTYLVLTFGE |
| | | | LLLSPMGISFVSKVAPPKYKGAMMGGWFVATAI |
| | | | GNILVSVGGYLWGDLSLTVVWTVFIVLCLVSAS |
| | | | FMFLMMKRLEKVA |
| 82 | Calcium- | Q47910 | MKKILIFVAGLCMSLAASAQIQRPKLVVGLVVD |
| | transporting_ATPase | | QMRWDYLYYYYNEYGTDGLRRLVDNGFSFENTH |
| | | | INYAPTVTAIGHSSVYTGSVPAITGIAGNYFFQ |
| | | | DDKNVYCCEDPNVKSVGSDSKEGQMSPHRLLAS |
| | | | TIGDELQISNDFRSKVIGVALKDRASILPAGHA |
| | | | ADAAYWWDTSAGHFVTSTFYTDHLPQWVIDFNE |
| | | | KNHTAPNFNIKTSTQGVTMTFKMAEAALKNENL |
| | | | GKGKETDMLAVSISSTDAIGHVYSTRGKENHDV |
| | | | YMQLDKDLAHFLKTLDEQVGKGNYLLFLTADHG |
| | | | AAHNYNYMKEHRIPAGGWDYRQSVKDLNGYLQG |
| | | | KFGIAPVMAEDDYQFFLNDSLIAASGLKKQQII |
| | | | DESVEYLKKDPRYLYVFDEERISEVTMPQWIKE |
| | | | RMINGYFRGRSGEIGVVTRPQVFGAKDSPTYKG |
| | | | TQHGQPFPYDTHIPFLLYGWNVKHGATTQQTYI |
| | | | VDIAPTVCAMLHIQMPNGCIGTARNMALGN |
| 83 | Poly-beta- 1,6-N- | Q5HKQ0 | MDRQVFQTDSRQRWNRFKWTLRVLITIAILLGV |
| | acetyl-D- | | VFVAMFALEGSPQMPFRHDYRSVVSASEPLLKD |
| | glucosamine_synthase | | NKRAEVYKSFRDFFKEQKMHSNYAKVAARQHRF |
| | | | VGHTDNVTQKYIKEWTDPRMGIRSAWYVNWDKH |
| | | | AYISLKNNLKNLNMVLPEWYFINPKTDRIEARI |
| | | | DQRALKLMRRAHIPVLPMLTNNYNSAFRPEAIG |
| | | | RIMRDSTKRMGMINELVAACKHNGFAGINLDLE |
| | | | ELNINDNALLVTLVKDFARVFHANGLYVTQAVA |
| | | | PFNEDYDMQELAKYDDYLFLMAYDEYNAGSQAG |
| | | | PVSSQRWVEKATDWAAKNVPNDKIVLGMATYGY |
| | | | NWAQGQGGTTMSFDQTMATALNAGAKVNFNDDT |
| | | | YNLNFSYQDEDDGTLHQVFFPDAVTTFNIMRFG |
| | | | ATYHLAGFGLWRLGTEDSRIWKYYGKDLSWESA |
| | | | ARMPIAKIMQLSGTDDVNFVGSGEVLNVTSEPH |
| | | | AGRIGIVLDKDNQLIIEERYLSLPATYTVQRLG |
| | | | KCKEKQLVLTFDDGPDSRWTPKVLSILKHYKVP |
| | | | AAFFMVGLQIEKNIPIVKDVFNQGCTIGNHTFT |
| | | | HHNMIENSDRRSFAELKLTRMLIESITGQSTIL |
| | | | FRAPYNADADPTDHEEIWPMIIASRRNYLFVGE |
| | | | SIDPNDWQQGVTADQIYKRVLDGVHQEYGHIIL |
| | | | LHDAGGDTREPTVTALPRIIETLQREGYQFISL |
| | | | EKYLGMSRQTLMPPIKKGKEYYAMQANLSLAEL |
| | | | IYHISDFLTALFLVFLVLGFMRLVFMYVLMIRE |
| | | | KRAENRRNYAPIDPLTAPAVSIIVPAYNEEVNI |
| | | | VRTISNLKEQDYPSLKIYLVDDGSKDNTLQRVR |
| | | | EVFENDDKVVIISKKNGGKASALNYGIAACSTD |
| | | | YIVCVDADTQLYKDAVSKLMKHFIADKTGKLGA |
| | | | VAGNVKVGNQRNMLTYWQAIEYTTSQNFDRMAY |
| | | | SNINAITVIPGAIGAFRKDVLEAVGGFTTDTLA |
| | | | EDCDLTMSINEHGYLIENENYAVAMTEAPESLR |
| | | | QFIKQRIRWCFGVMQTFWKHRASLFAPSKGGFG |
| | | | MWAMPNMLIFQY11PTFSPIADVLMLFGLFSGN |
| | | | ASQIFIYYLIFLLVDASVSIMAYIFEHESLWVL |
| | | | LWIIPQRFFYRWIMYYVLFKSYLKAIKGELQTW |
| | | | GVLKRTGHVKGAQTIS |
| 84 | ATP_synthase_ | P29707 | MSQINGRISQIIGPVIDVYFDTKGENPEKVLPN |
| | subunit_beta,_ | | IYDALRVKKADGQDLIIEVQQQIGEDTVRCVAM |
| | sodium_ion_specific | | DNTDGLQRGLEVVPTGSPIVMPAGEQIKGRMMN |
| | | | VIGQPIDGMSALQMEGAYPIHREAPKFEDLSTH |
| | | | KEMLQTGIKVIDLLEPYMKGGKIGLFGGAGVGK |
| | | | TVLIMELINNIAKGHNGYSVFAGVGERTREGND |
| | | | LIRDMLESGVIRYGEKFRKAMDEGKWDLSLVDS |
| | | | EELQKSQATLVYGQMNEPPGARASVALSGLTVA |
| | | | EEFRDHGGKNGEAADIMFFIDNIFRFTQAGSEV |
| | | | SALLGRMPSAVGYQPTLASEMGAMQERITSTKH |
| | | | GSITSVQAVYVPADDLTDPAPATTFTHLDATTE |
| | | | LSRKITELGIYPAVDPLGSTSRILDPLIVGKEH |
| | | | YDCAQRVKQLLQKYNELQDIIAILGMDELSDDD |
| | | | KLVVNRARRVQRFLSQPFTVAEQFTGVKGVMVP |
| | | | IEETIKGFNAILNGEVDDLPEQAFLNVGTIEDV |
| | | | KEKAKQLLEATKA |
| 85 | Cluster: | G6AGX5 | MNPIYKIITSILFCVLSINTMAQDLTGHVTSKA |
| | Uncharacterized | | DDKPIAYATVTLKENRLYAFTDEKGNYTIKNVP |
| | protein | | KGKYTVVFSCMGYASQTVVVMVNAGGATQNVRL |
| | | | AEDNLQLDEVQVVAHRKKDEITTSYTIDRKTLD |
| | | | NQQIMTLSDIAQLLPGGKSVNPSLMNDSKLTLR |
| | | | SGTLERGNASFGTAVEVDGIRLSNNAAMGETAG |
| | | | VSTRSVSASNIESVEVVPGIASVEYGDLTNGVV |
| | | | KVKTRRGSSPFIVEGSINQHTRQIALHKGVDLG |
| | | | GNVGLLNFSIEHARSFLDAASPYTAYQRNVLSL |
| | | | RYMNVFMKKSLPLTLEVGLNGSIGGYNSKADPD |
| | | | RSLDDYNKVKDNNVGGNIHLGWLLNKRWITNVD |
| | | | LTAAFTYADRLSESYTNESSNATQPYIHTLTEG |
| | | | YNIAEDYDRNPSANIILGPTGYWYLRGFNDSKP |
| | | | LNYSLKMKANWSKAFGKFRNRLLVGGEWTSSMN |
| | | | RGRGTYYADMRYAPSWREYRYDALPSLNNIAIY |
| | | | AEDKLSMDVNERQNAELTAGIREDITSIPGSEY |
| | | | GSVGSFSPRMNARYVFRFGQNSWLNSMTLHAGW |
| | | | GRSVKIPSFQVLYPSPSYRDMLAFASTSDADNR |
| | | | SYYAYYTYPSMARYNANLKWQRADQWDLGVEWR |
| | | | TKIADVSLSFFRSKVSNPYMATDVYTPFTYKYT |
| | | | SPAMLQRSGIAVADRRFSIDPQTGIVTVSDASG |
| | | | VKSPVTLGYEERNTYVTNTRYVNADALQRYGLE |
| | | | WIVDFKQIKTLRTQVRLDGKYYHYKAQDETLFA |
| | | | DVPVGLNTRQSDGRLYQYVGYYRGGAATTTNYT |
| | | | ANASASNGSVSGQVDLNATITTHIPKIRLIVAL |
| | | | RLESSLYAFSRATSSRGYVVSSGNEYFGVPYDD |
| | | | KTENQTVIVYPEYYSTWDAPDVLIPFAEKLRWA |
| | | | ETNDRGLFNDLAQLVVRTNYPYTLNPNRLSAYW |
| | | | SANLSVTKEIGRHVSVSFYANNFFNTLSQVHST |
| | | | QTGLETSLFGSGYVPSFYYGLSLRLKI |

In some embodiments, the Prevotella bacteria is a strain of Prevotella bacteria free or substantially free of one or more (e.g., 1, 2, 3, 4, 5, 6, 7, 8, 9, 10, 11, 12, 13, 14, 15, 16, 17, 18, 19, 20, 21, 22, 23, 24, 25 or more) proteins listed in Table 2 and/or one or more (e.g., 1, 2, 3, 4, 5, 6, 7, 8, 9, 10, 11, 12, 13, 14, 15, 16, 17, 18, 19, 20, 21, 22, 23, 24, 25 or more) genes encoding proteins listed in Table 2. In some embodiments, Prevotella bacteria is free of all of the proteins listed in Table 2 and/or all of the genes encoding the proteins listed in Table 2.

TABLE 2
Other Prevotella proteins

| SEQ | | | |
|---|---|---|---|
| ID | | Uniprot | |
| NO. | Name | ID | Amino Acid Sequence |
| 86 | UDP-Gal:alpha-D- | Q03084 | MERIDISVLMAVYKKDNPAFLRESLESIFSQ |
| | GlcNAc- | | TVEAAEVVLLEDGPLTDALYDVIKSYEAIY |
| | diphosphoundecap | | STLKVVSYPENRGLGKTLNDGLLLCKYNL |
| | renol | | VARMDADDICKPNRLEMEYNWLKSHEDY |
| | | | DVIGSWVDEFTDNKTRVKSIRKVPEAYDEI |
| | | | KNYAQYRCPINHPTAMYRKAAVLAVGGY |
| | | | LTEYFPEDYFLWLRMLNNGSKFYNIQESLL |
| | | | WFRYSEETVAKRGGWAYACDEVRILVRM |
| | | | LKMGYIPFHVFCQSVVIRFTTRVMPLPIRQR |
| | | | LYNLIRKT |
| 87 | ATP_synthase_ | A1B8P0 | MSQINGRISQIIGPVIDVYFDTKGENPEKVLP |
| | subunit_beta | | KIHDALRVKRANGQDLIIEVQQHIGEDTVR |
| | | | CVAMDNTDGLQRNLEVVPTGSPIVMPAGD |
| | | | QIKGRMMNVIGQPIDGMEALSMEGAYPIHR |
| | | | EAPKFEDLSTHKEMLQTGIKVIDLLEPYMK |
| | | | GGKIGLFGGAGVGKTVLIMELINNIAKGHN |
| | | | GYSVFAGVGERTREGNDLIRDMLESGVIRY |
| | | | GEKFRKAMDEGKWDLSLVDQEELQKSQA |
| | | | TLVYGQMNEPPGARASVALSGLTVAEEFR |
| | | | DHGGKNGEAADIMFFIDNIFRFTQAGSEVS |
| | | | ALLGRMPSAVGYQPTLASEMGTMQERITST |
| | | | KHGSITSVQAVYVPADDLTDPAPATTFTHL |
| | | | DATTELSRKITELGIYPAVDPLGSTSRILDPL |
| | | | IVGKDHYECAQRVKQLLQHYNELQDIIAIL |
| | | | GMDELSDEDKLVVNRARRVQRFLSQPFTV |
| | | | AEQFTGVKGVMVPIEETIKGFNAILNGEVD |
| | | | DLPEQAFLNVGTIEDVKEKAKRLLEATK |
| 88 | Cell_division_ | O05779 | MPIGNGQKYQLTIINHTEIIMLIDYKKVNIY |
| | ATP-binding_ | | QDERLILKDVDFQAETGEFIYLIGRVGSGKS |
| | protein_FtsE | | SLLKTIYGELDIDSEDAEKAVVLDESMPNIK |
| | | | RSRIPALRKQMGIIFQDFQLLHDRSVAKNL |
| | | | KFVLQATGWTSKQKIERRIEEVLAQVGMT |
| | | | DKKNKMPSELSGGEQQRIAIARALLNTPKII |
| | | | IADEPTGNLDPETAANIVSILKDSCQAGTTV |
| | | | IMSTHNINLIDQFPGKVYRCHEGELHQLTD |
| | | | KKEVSELAEETAPVETIDEPEQND |
| 89 | Hemin_transport_ | Q56992 | MKRNILLFICLATSILLLFGLNLTTGSVQIPF |
| | system_permease_ | | ADILDILCGRFIGKESWEYIILENRLPQTLTA |
| | protein_HmuU | | ILCGASLSVCGLMLQTAFRNPLAGPDVFGIS |
| | | | SGAGLGVALVMLLLGGTVSTSIFTVSGFLAI |
| | | | LTAAFVGAIAVTALILFLSTLVRNSVLLLIV |
| | | | GIMVGYVSSSAVSLLNFFASEEGVKSYMV |
| | | | WGMGNFGAVSMNHIPLFSILCLIGIIASFLL |
| | | | VKPLNILLLGPQYAESLGISTRQIRNILLVVV |
| | | | GLLTAITTAFCGPISFIGLAIPHIARLLFRTEN |
| | | | HQILLPGIVLSGAAIALLCNFICYLPGESGIIP |
| | | | LNAVTPLIGAPIIIYVIIQRR |
| 90 | Hexuronate_ | O34456 | MKKYYPWVLVALLWFVALLNYMDRQML |
| | transporter | | STMQEAMKVDIAELNHAEAFGALMAVFL |
| | | | WIYGIVSPFAGIIADRVNRKWLVVGSIFVW |
| | | | SAVTYLMGYAESFDQLYWLRAFMGISEAL |
| | | | YIPAALSLIADWHEGKSRSLAIGIHMTGLYV |
| | | | GQAVGGFGATLAAMFSWHAAFHWFGIIGI |
| | | | VYSLVLLLFLKENPKHGQKSVLQGETKPSK |
| | | | NPFRGLSIVFSTWAFWVILFYFAVPSLPGW |
| | | | ATKNWLPTLFANSLDIPMSSAGPMSTITIAV |
| | | | SSFIGVIMGGVISDRWVQRNLRGRVYTSAI |
| | | | GLGLTVPALMLLGFGHSLVSVVGAGLCFGI |
| | | | GYGMFDANNMPILCQFISSKYRSTAYGIMN |
| | | | MTGVFAGAAVTQVLGKWTDGGNLGNGFA |
| | | | ILGGIVVLALVLQLSCLKPTTDNME |
| 91 | 1,4-alpha- | P9WN45 | MVTKKTTTKKAPVKKTSAKTTKVKEPSHI |
| | glucan_branching_ | | GLVKNDAYLAPYEDAIRGRHEHALWKMN |
| | enzyme_GlgB | | QLTQNGKLTLSDFANGHNYYGLHQTADG |
| | | | WVFREWAPNATEIYLVGDFNGWNEQEAY |
| | | | QCHRIEGTGNWELTLPHDAMQHGQYYKM |
| | | | RVHWEGGEGERIPAWTQRVVQDEASKIFS |
| | | | AQVWAPAEPYVWEKKTFKPQTSPLLIYEC |
| | | | HIGMAQDEEKVGTYNEFREKVLPRIIKDGY |
| | | | NAIQIMAIQEHPYYGSFGYHVSSFFAASSRF |
| | | | GTPEELKALIDEAHKNGIAVIMDIVHSHAV |
| | | | KNEVEGLGNLAGDPNQYFYPGERHEHPAW |
| | | | DSLCFDYGKDEVLHFLLSNCKYWLEEYHF |
| | | | DGFRFDGVTSMLYYSHGLGEAFCNYADYF |
| | | | NGHQDDNAICYLTLANCLIHEVNKNAVTIA |
| | | | EEVSGMPGLAAKFKDGGYGFDYRMAMNIP |
| | | | DYWIKTIKELPDEAWKPSSIFWEIKNRRSDE |
| | | | KTISYCESHDQALVGDKTIIFRLVDADMYW |
| | | | HFRKGDETEMTHRGIALHKMIRLATIAAIN |
| | | | GGYLNFMGNEFGHPEWIDFPREGNGWSHK |
| | | | YARRQWNLVDNEELCYHLLGDFDRKMLE |
| | | | VITSEKKFNETPIQEIWHNDGDQILAFSRGE |
| | | | LVFVFNFSPSHSYSDYGFLVPEGSYNVVLN |
| | | | TDAREFGGFGFADDTVEHFTNSDPLYEKDH |
| | | | KGWLKLYIPARSAVVLRKK |
| 92 | Cluster: YihY | D9RW24 | MKIDIERIKYFLTVGMFMKTEHSSKRRNML |
| | family protein | | IRQFQKFYLTVKFFFVRDHAASTAQLSFSTI |
| | | | MAIVPIASMIFAIANGFGFGQFLEKQFREML |
| | | | SAQPEAATWLLKLTQSYLVHAKTGLFIGIG |
| | | | LMIMLYSVFSLIRTVETTFDNIWQVKDSRPI |
| | | | SRIVIDYTALMFLVPISIIILSGLSIYFYSFVEN |
| | | | LNGLRFLGTIASFSLRYLVPWAILTLMFIVL |
| | | | YVFMPNAKVKITKTVAPAMIASIAMLCLQA |
| | | | VYIHGQIFLTSYNAIYGSFAALPLFMLWILA |
| | | | SWYICLFCAELCYFNQNLEYYECLIDTEDIC |
| | | | HNDLLILCATVLSHICQRFANDQKPQTALQI |
| | | | KTETHIPIRVMTDILYRLKEVNLISENFSPTS |
| | | | DEVTYTPTHDTNNITVGEMIARLESTPASDF |
| | | | ALLGFSPKKAWNHDIYDRVGSIREIYLNEL |
| | | | KSINIKELISYSEN |
| 93 | Capsule_ | P19579 | MMKRPSIARVVKVIICLLTPILLSFSGIGDND |
| | biosynthesis_ | | IDKKKSTSKEVDDTLRIVITGDLLLDRGVRQ |
| | protein_CapA | | KIDMAGVDALFSPTIDSLFHSSNYVIANLEC |
| | | | PVTKIRERVFKRFIFRGEPEWLPTLRRHGIT |
| | | | HLNLANNHSIDQGRNGLLDTQEQIKKAGMI |
| | | | PIGAGKNMEEAAEPVLISTSPRHVWVISSLR |
| | | | LPLENFLYLPQKPCVSQESIDSLIMRVKRLR |
| | | | ATDKNCYILLILHWGWEHHFRATPQQRED |
| | | | AHKLIDAGADAIVGHHSHTLQTIETYRGKPI |
| | | | YYGIGNFIFDQRKPMNSRACLVELSITAEKC |
| | | | KAKALPIEIKNCTPYLSK |
| 94 | Peptidoglycan_ | B5ZA76 | MILLSFDTEEFDVPREHGVDFSLEEGMKVSI |
| | deacetylase | | EGTNRILDILKANNVCATFFCTGNFAELAPE |
| | | | VMERIKNEGHEVACHGVDHWQPKPEDVFR |
| | | | SKEIIERVTGVKVAGYRQPRMFPVSDEDIEK |
| | | | AGYLYNSSLNPAFIPGRYMHLTTSRTWFM |
| | | | QGKVMQIPASVSPHLRIPLFWLSMHNFPEW |
| | | | FYLRLVRQVLRHDGYFVTYFHPWEFYDLK |
| | | | SHPEFKMPFIIKNHSGHELEQRLDRFIKAMK |
| | | | ADKQEFITYVDFVNRQKK |
| 95 | Fumarate_ | P0AC47 | MAKNISFTIKYWKQNGPQDQGHFDTHEMK |
| | reductase_iron- | | NIPDDTSFLEMLDILNEELIAAGDEPFVFDH |
| | sulfur_subunit | | DCREGICGMCSLYINGTPHGKTERGATTCQ |
| | | | LYMRRFNDGDVITVEPWRSAGFPVIKDCM |
| | | | VDRTAFDKIIQAGGYTTIRTGQAQDANAILI |
| | | | SKDNADEAMDCATCIGCGACVAACKNGS |
| | | | AMLFVSSKVSQLALLPQGKPEAAKRAKAM |
| | | | VAKMDEVGFGNCTNTRACEAVCPKNEKIA |
| | | | NIARLNREFIKAKFAD |
| 96 | Serine/threonine- | P9WI71 | MSENKLSTNEQAQTADAPVKASYTEYKVIP |
| | protein_kinase_ | | SQGYCMIVKCRKGDQTVVLKTLKEEYRER |
| | PknH | | VLLRNALKREFKQCQRLNHSGIVRYQGLV |
| | | | EVDGYGLCIEEEYVEGRTLQAYLKENHTD |
| | | | DEKIAIINQIADALRYAHQQGVIHRNLKPSN |
| | | | VLVTTQGDYVKLIDFSVLSPEDVKPTAETT |
| | | | RFMAPEMKDETLTADATADIYSLGTIMKV |
| | | | MGLTLAYSEVIKRCCAFKRSDRYSNVDELL |
| | | | ADLNNEGSSFSMPKIGKGTVVLGLIIAVVIG |
| | | | IGALLYNYGGALIDQVGKIDVSSVFSSDAET |
| | | | APEDTVKVNTAEQSDSLSTEAEAPAIGKLA |
| | | | FMNRMKPALYKDLDNIFEKNSADKAKLTK |
| | | | AIKTYYRGLIQANDTLDNEQRAEVDRVFG |
| | | | DYVKQKKAALN |
| 97 | Carboxy- | O34666 | MRKYICLLLFYLFTFLPLSAQQGNDSPLRKL |
| | terminal_ | | QLAEMAIKNFYVDSVNEQKLVEDGIRGML |
| | processing_ | | EKLDPHSTYTDAKETKAMNEPLQGDFEGIG |
| | protease_CtpA | | VQFNMIEDTLVVIQPVVNGPSQKVGILAGD |
| | | | RIVSVNDSTIAGVKMARIDIMKMLRGKKGT |
| | | | KVKLGVVRRGVKGVLTFVVTRAKIPVHTIN |
| | | | ASYMIRPNVGYIRIESFGMKTHDEFMSAVD |
| | | | SLKKKGMKTLLLDLQDNGGGYLQSAVQIS |
| | | | NEFLKNNDMIVYTEGRRARRQNFKAIGNG |
| | | | RLQDVKVYVLVNELSASAAEIVTGAIQDND |
| | | | RGTVVGRRTFGKGLVQRPFDLPDGSMIRLT |
| | | | IAHYYTPSGRCIQKPYTKGDLKDYEMDIEK |
| | | | RFKHGELTNPDSIQFSDSLKYYTIRKHRVV |
| | | | YGGGGIMPDNFVPLDTTKFTRYHRMLAAK |
| | | | SIIINAYLKYADANRQALKAQYSSFDAFNK |
| | | | GYVVPQSLLDEIVAEGKKEKIEPKDAAELK |
| | | | ATLPNIALQIKALTARDIWDMNEYFRVWN |
| | | | TQSDIVNKAVALATGK |
| 98 | Cluster: | D9RRG3 | MKLTEQRSSMLHGVLLITLFACAAFYIGDM |
| | Uncharacterized | | GWVKALSLSPMVVGIILGMLYANSLRNNL |
| | protein | | PDTWVPGIAFCGKRVLRFGIILYGFRLTFQD |
| | | | VVAVGFPAIIVDAIIVSGTILLGVLVGRLLK |
| | | | MDRSIALLTACGSGICGAAAVLGVDGAIRP |
| | | | KPYKTAVAVATVVIFGTLSMFLYPILYRAGI |
| | | | FDLSPDAMGIFAGSTIHEVAHVVGAGNAM |
| | | | GAAVSNSAIIVKMIRVMMLVPVLLVIAFFV |
| | | | AKNVAERDDEAGGSRKINIPWFAILFLVVIG |
| | | | FNSLNLLPKELVDFINTLDTFLLTMAMSAL |
| | | | GAETSIDKFKKAGFKPFLLAAILWCWLIGG |
| | | | GYCLAKYLVPVLGVAC |
| 99 | Cluster: Cna | X6Q2J4 | MNKQFLLAALWLSPLGLYAHKANGIGAVT |
| | protein B-type | | WKNEAPKERMIRGIDEDKTHQRFTLSGYV |
| | domain protein | | KDRNGEPLINATIYDLTTRQGTMTNAYGHF |
| | | | SLTLGEGQHEIRCSYVGYKTLIETIDLSANQ |
| | | | NHDIILQNEAQLDEVVVTTDLNSPLLKTQT |
| | | | GKLSLSQKDIKTEYALLSSPDVIKTLQRTSG |
| | | | VADGMELASGLYVHGGNGDENLFLLDGTP |
| | | | LYHTNHSLGLFSSFNADVVKNVDFYKSGFP |
| | | | ARYGGRLSSVIDVRTADGDLYKTHGSYRIG |
| | | | LLDGAFHIGGPIRKGKTSYNFGLRRSWMDL |
| | | | LTRPAFAIMNHKSDNEDKLSMSYFFHDLNF |
| | | | KLTNIFNERSRMSLSVYSGEDRLDAKDEW |
| | | | HSNNSSGYNDVDIYVNRFHWGNFNAALD |
| | | | WNYQFSPKLFANFTAVYTHNRSTVSSSDE |
| | | | WRFTRPGEKEQLTLTSHGYRSSIDDIGYRA |
| | | | AFDFRPSPRHHIRFGQDYTYHRFQPQTYNR |
| | | | FDNYQTNSEAKADTIATHSYNKNVAHQLT |
| | | | FYAEDEMTLNEKWSLNGGVNADVFHISGK |
| | | | TFATLSPRLSMKFQPTERLSLKASYTLMSQF |
| | | | VHKIANSFLDLPTDYWVPTTARLHPMRSW |
| | | | QVAAGAYMKPNKHWLLSLEAYYKRSSHIL |
| | | | QYSSWAGLEPPAANWDYMVMEGDGRSYG |
| | | | VELDADYNVSNLTLHGSYTLSWTQKKFDD |
| | | | FYDGWYYDKFDNRHKLTLTGRWNITKKIA |
| | | | AFAAWTFRTGNRMTIPTQYIGLPDVPAQEQ |
| | | | GGLTFNSSDDNTLNFAYEKPNNVILPAYHR |
| | | | LDIGFDFHHTTKKGHERIWNLSFYNAYCHL |
| | | | NSLWVRVKIDSNNQMKIRNIAFIPVIPSFSY |
| | | | TFKF |
| 100 | Poly-beta-1,6-N- | P75905 | MSKQVFQTDSRQRWSYFKWTLRVILTILSL |
| | acetyl-D- | | LGIVFLAMFALEGSPQMPFRHDYRNAVTA |
| | glucosamine_ | | ASPYTKDNKTAKLYKSFRDFFKEKKMHNN |
| | synthase | | YAKATIKKQRFIGKADSVTQKYFREWDDP |
| | | | RIGVRSAWYVNWDKHAYISLKNNIKHLNM |
| | | | VLPEWFFINPKTDKVEYRIDKQALRLMRRT |
| | | | GIPVLPMLTNNYNSDFHPEAIGRIMRDEKK |
| | | | RMALINEMVRTCRHYGFAGINLDLEELNIQ |
| | | | DNDLLVELLKDFSRVFHANGLYVTQAVAP |
| | | | FNEDYNMQELAKYNDYLFLMAYDEHNIES |
| | | | QPGAVSSQRWVEKATDWAAKNVPNDKIV |
| | | | LGMATYGYDWANGEGGTTVSFDQTMAIA |
| | | | QDADAKVKFDDDTYNVNFSYQNTDDGKIH |
| | | | HVFFTDAATTFNIMRFGAEYHLAGYGLWR |
| | | | LGTEDKRIWRFYGKDMSWENVARMSVAK |
| | | | LMQLNGTDDVNFVGSGEVLEVTTEPHPGDI |
| | | | SIRIDKDNRLISEEYYRALPSTYTIQRLGKCK |
| | | | DKQLVITFDDGPDSRWTPTVLSTLKKYNVP |
| | | | AAFFMVGLQMEKNLPLVKQVYEDGHTIGN |
| | | | HTFTHHNMIENSDRRSYAELKLTRMLIESV |
| | | | TGHSTILFRAPYNADADPTEHEEIWPMIVAS |
| | | | RRNYLFVGESIDPNDWEPNVTSDQIYQRVI |
| | | | DGVHHEDGHIILLHDAGGSSRKPTLDALPRI |
| | | | IETLQHEGYQFISLEQYLGMGKQTLMPEIN |
| | | | KGKAYYAMQTNLWLAEMIYHVSDFLTALF |
| | | | LVFLALGMMRLIFMYVLMIREKRAENRRN |
| | | | YAPIDAATAPAVSIIVPGYNEEVNIVRTITTL |
| | | | KQQDYPNLHIYFVDDGSKDHTLERVHEAF |
| | | | DNDDTVTILAKKNGGKASALNYGIAACRS |
| | | | EYVVCIDADTQLKNDAVSRLMKHFIADTE |
| | | | KRVGAVAGNVKVGNQRNMLTYWQAIEYT |
| | | | SSQNFDRMAYSNINAITVVPGAIGAFRKEVI |
| | | | EAVGGFTTDTLAEDCDLTMSINEHGYIIENE |
| | | | NYAVALTEAPETLRQFVKQRIRWCFGVMQ |
| | | | AFWKHRSSLFAPSKKGFGLWAMPNMLIFQ |
| | | | YIIPTFSPLADVLMLIGLFTGNALQIFFYYLIF |
| | | | LVIDASVSIMAYIFEGERLWVLLWVIPQRFF |
| | | | YRWIMYYVLFKSYLKAIKGELQTWGVLKR |
| | | | TGHVKG |
| 101 | Cell_division_ | O34876 | MAKKRNKARSRHSLQVVTLCISTAMVLML |
| | protein_FtsX | | IGIVVLTGFTSRNLSSYVKENLTITMILQPD |
| | | | MNTEESAALCERIRTLHYINSLNFISKEQAL |
| | | | KDGTKELGANPAEFAGENPFTGEIEVQLKA |
| | | | NYANNDSIRNIVQQLRTYRGVSDITYPQSL |
| | | | VESVNQTLGKISLVLLVIAVLLTIISFSLINNT |
| | | | IRLSIYAHRFSIHTMKLVGGSWSFIRAPFLR |
| | | | RAVLEGLVSALLAIAVLGIGICLLYEKEPEIT |
| | | | KLLSWDALIITAIVMLAFGVIIATFCAWLSV |
| | | | NKFLRMKAGDLYKI |
| 102 | UDP-2,3- | P44046 | MKNIYFLSDAHLGSLAIDHRRTHERRLVRF |
| | diacylglucosamine | | LDSIKHKAAAVYLLGDMFDFWNEYKYVVP |
| | hydrolase | | KGFTRFLGKISELTDMGVEVHFFTGNHDL |
| | | | WTYGYLEKECGVILHRKPITTEIYDKVFYL |
| | | | AHGDGLGDPDPMFRFLRKVFHNRFCQRLL |
| | | | NFFHPWWGMQLGLNWAKRSRLKRKDGKE |
| | | | VPYLGEDKEYLVQYTKEYMSTHKDIDYYI |
| | | | YGHRHIELDLTLSRKARLLILGDWIWQFTY |
| | | | AVFDGEHMFLEEYVEGESKP |
| 103 | Poly-beta-1,6-N- | P75905 | MVGLDVLCYFIHAKGREKECYFERIIYQITC |
| | acetyl-D- | | HSRTKCYLCNIMKYSIIVPVFNRPDEVEELL |
| | glucosamine_ | | ESLLSQEEKDFEVVIVEDGSQIPCKEVCDKY |
| | synthase | | ADKLDLHYYSKENSGPGQSRNYGAERAKG |
| | | | EYLLILDSDVVLPKGYICAVSEELKREPADA |
| | | | FGGPDCAHESFTDTQKAISYSMTSFFTTGGI |
| | | | RGGKKKLDKFYPRSFNMGIRRDVYQELGG |
| | | | FSKMRFGEDIDFSIRIFKAGKRCRLFPEAWV |
| | | | WHKRRTDFRKFWKQVYNSGIARINLYKKY |
| | | | PESLKLVHLLPMVFTVGTALLVLMILFGLF |
| | | | LQLFPIINVFGSVFIMMGLMPLVLYSVIICV |
| | | | DSTMQNNSLNIGLLSIEAAFIQLTGYGCGFI |
| | | | SAWWKRCVCGMDEFAAYEKNFYK |
| 104 | Enolase | Q8DTS9 | MKIEKVHAREIMDSRGNPTVEVEVTLENG |
| | | | VMGRASVPSGASTGENEALELRDGDKNRF |
| | | | LGKGVLKAVENVNNLIAPALKGDCVLNQR |
| | | | AIDYKMLELDGTPTKSKLGANAILGVSLAV |
| | | | AQAAAKALNIPLYRYIGGANTYVLPVPMM |
| | | | NIINGGAHSDAPIAFQEFMIRPVGAPSEKEGI |
| | | | RMGAEVFHALAKLLKKRGLSTAVGDEGGF |
| | | | APKFDGIEDALDSIIQAIKDAGYEPGKDVKI |
| | | | AMDCAASEFAVCEDGKWFYDYRQLKNGM |
| | | | PKDPNGKKLSADEQIAYLEHLITKYPIDSIE |
| | | | DGLDENDWENWVKLTSAIGDRCQLVGDD |
| | | | LFVTNVKFLEKGIKMGAANSILIKVNQIGSL |
| | | | TETLEAIEMAHRHGYTTVTSHRSGETEDTTI |
| | | | ADIAVATNSGQIKTGSMSRTDRMAKYNQLI |
| | | | RIEEELGACAKYGYAKLK |
| 105 | Outer_membrane_ | Q8G0Y6 | MKKLFTIAMLLGVTLGIHAQEVYSLQKCRE |
| | efflux_protein_ | | LALQNNRQLKVSRMTVDVAENTRKAAKT |
| | BepC | | KYLPRVDALAGYQHFSREISLLSDDQKNAF |
| | | | SNLGTNTFGQLGGQIGQNLTSLAQQGILSP |
| | | | QMAQQLGQLFSNVATPLTQVGNNIGQSIND |
| | | | AFRSNTKNVYAGGIVVNQPIYMGGAIKAA |
| | | | NDMAAIGEQVAQNNISLKRQLVLYGVDNA |
| | | | YWLAISLKKKEALAIRYRDLAQKLNEDVK |
| | | | KMIREGVATRADGLKVEVAVNTADMQIAR |
| | | | IQSGVSLAKMALCELCGLELNGDIPLSDEG |
| | | | DADLPPTPSTQFDNYTVSSSDTTGLNEARPE |
| | | | LRLLQNAVDLSIQNTKLIRSLYMPHVLLTA |
| | | | GYSVSNPNLFNGFQKRFTDLWNIGITVQVP |
| | | | VWNWGENKYKVRASKTATTIAQLEMDDV |
| | | | RKKIDLEIEQNRLRLKDANKQLATSQKNM |
| | | | AAAEENLRCANVGFKEGVMTVTEVMAAQ |
| | | | TAWQTSRMAIIDAEISVKLAQTGLQKALGG |
| | | | L |
| 106 | Phosphoethanolamine_ | Q7CPC0 | MKRTFVTKMVKPIEENSLFFMFMLLVGAFT |
| | transferase_ | | NVSHRNVFGYIELIADVYIICFLLSLCQRTIR |
| | CptA | | QGLVIMLSSVIYVVAIIDTCCKTLFDTPITPT |
| | | | MLLLAQETTGREATEFFLQYLNLKLFFSAA |
| | | | DIILFLAFCHIVMAVKKMKFSTSYLKQPFV |
| | | | AFVLMFTIFVGMALSIYDKVQLYTVKNLSG |
| | | | LEVAVTNGFAHLYHPVERIVYGLYSNHLIA |
| | | | KQVDGVIMANQQIKVDSCSFTSPTIVLVIGE |
| | | | SANRHHSQLYGYPLPTTPYQLAMKNGKDS |
| | | | LAVFTNVVSPWNLTSKVFKQIFSLQSVDEK |
| | | | GDWSKYVLFPAVFKKAGYHVSFLSNQFPY |
| | | | GINYTPDWTNNLVGGFFLNHPQLNKQMFD |
| | | | YRNVTIHNYDEDLLNDYKEIISYKKPQLIIF |
| | | | HLLGQHFQYSLRCKSNMKKFGIKDYKRMD |
| | | | LTDKEKQTIADYDNATLYNDFVLNKIVEQF |
| | | | RNKDAIIVYLSDHGEDCYGKDVNMAGRLT |
| | | | EVEQINLKKYHEEFEIPFWIWCSPIYKQRHR |
| | | | KIFTETLMARNNKFMTDDLPHLLLYLAGIK |
| | | | TKDYCEERNVISPSFNNNRRRLVLKTIDYD |
| | | | KALYQ |
| 107 | Dipeptide_and_ | P36837 | MFKNHPKGLLQAAFSNMGERFGYYIMNAV |
| | tripeptide_ | | LALFLCSKFGLSDETSGLIASLFLAAIYVMS |
| | permease_B | | LVGGVIADRTQNYQRTIESGLVVMALGYV |
| | | | ALSIPVLATPENNSYLLAFTIFALVLIAVGN |
| | | | GLFKGNLQAIVGQMYDDFETEAAKVSPER |
| | | | LKWAQGQRDAGFQIFYVFINLGALAAPFIA |
| | | | PVLRSWWLGRNGLTYDAALPQLCHKYING |
| | | | TIGDNLGNLQELATKVGGNSADLASFCPHY |
| | | | LDVFNTGVHYSFIASVVTMLISLIIFMSSKK |
| | | | LFPMPGKKEQIVNVEYTDEEKASMAKEIKQ |
| | | | RMYALFAVLGISVFFWFSFHQNGQSLSFFA |
| | | | RDFVNTDSVAPEIWQAVNPFFVISLTPLIM |
| | | | WVFAYFTKKGKPISTPRKIAYGMGIAGFAY |
| | | | LFLMGFSLVHNYPSAEQFTSLEPAVRATMK |
| | | | AGPMILILTYFFLTVAELFISPLGLSFVSKVA |
| | | | PKNLQGLCQGLWLGATAVGNGFLWIGPLM |
| | | | YNKWSIWTCWLVFAIVCFISMVVMFGMVK |
| | | | WLERVTKS |
| 108 | C4- | Q9I4F5 | MQKKIKIGLLPRVIIAILLGLFLGYYLPDPAV |
| | dicarboxylate_ | | RVFLTFNSIFSQFLGFMIPLIIIGLVTPAIAGIG |
| | transport_protein_ | | KGAGKLLLATVAIAYVDTIVAGGLSYGTGT |
| | 2 | | WLFPSMIASTGGAIPHIDKATELTPYFTINIP |
| | | | AMVDVMSSLVFSFIAGLGIAYGGLRTMENL |
| | | | FNEFKTVIEKVIEKAIIPLLPLYIFGVFLSMT |
| | | | HNGQARQVLLVFSQIIIVILVLHVLILIYEFCI |
| | | | AGAIVKHNPFRLLWNMLPAYLTALGTSSSA |
| | | | ATIPVTLKQTVKNGVSEEVAGFVVPLCATI |
| | | | HLSGSAMKITACALTICMLTDLPHDPGLFIY |
| | | | FILMLAIIMVAAPGVPGGAIMAALAPLSSIL |
| | | | GFNEEAQALMIALYIAMDSFGTACNVTGD |
| | | | GAIALAVNKFFGKKKETSILS |
| 109 | Inner_membrane_ | P76090 | MISVYSIKPQFQRVLTPILELLHRAKVTANQ |
| | protein_YnbA | | ITLWACVLSLVIGILFWFAGDVGTWLYLCL |
| | | | PVGLLIRMALNALDGMMARRYNQITRKGE |
| | | | LLNEVGDVVSDTIIYFPLLKYHPESLYFIVA |
| | | | FIALSIINEYAGVMGKVLSAERRYDGPMGK |
| | | | SDRAFVLGLYGVVCLFGINLSGYSVYIFGVI |
| | | | DLLLVLSTWIRIKKTLKVTRNSQTPE |
| 110 | 2′,3′-cyclic- | P08331 | MKLSTILLSIMLGLSSSTMAQQKDVTIKLIE |
| | nucleotide | | TTDVHGSFFPYDFITRKPKSGSMARVYTLV |
| | | | EELRKKDGKDNVYLLDNGDILQGQPISYYY |
| | | | NYVAPEKTNIAASVLNYMGYDVATVGNH |
| | | | DIETGHKVYDKWFKELKFPILGANIIDTKTN |
| | | | KPYILPYYTIKKKNGIKVCVIGMLTPAIPNW |
| | | | LKESIWSGLRFEEMVSCAKRTMAEVKTQE |
| | | | KPDVIVGLFHSGWDGGIKTPEYDEDASKKV |
| | | | AKEVPGFDIVFFGHDHTPHSSIEKNIVGKDV |
| | | | ICLDPANNAQRVAIATLTLRPKTVKGKRQY |
| | | | TVTKATGELVDVKELKADDAFIQHFQPEID |
| | | | AVKAWSDQVIGRFENTIYSKDSYFGNSAFN |
| | | | DLILNLELEITKADIAFNAPLLFNASIKAGPI |
| | | | TVADMFNLYKYENNLCTMRLTGKEIRKHL |
| | | | EMSYDLWCNTMKSPEDHLLLLSSTQNDAQ |
| | | | RLGFKNFSFNFDSAAGIDYEVDVTKPDGQK |
| | | | VRILRMSNGEPFDENKWYTVAVNSYRANG |
| | | | GGELLTKGAGIPRDSLKSRIIWESPKDQRHY |
| | | | LMEEIKKAGVMNPQPNHNWKFIPETWTVP |
| | | | AAARDRKLLFGE |
| 111 | Fe(2+)_transporter_ | P33650 | MKLSELKTGETGVIVKVSGHGGFRKRIIEM |
| | FeoB | | GFIKGKTVEVLLNAPLQDPVKYKIMGYEVS |
| | | | LRHSEADQIEVLSDVKTHSVGNEEEQEDNQ |
| | | | LEMDSTTYDSTDKELTPEKQSDAVRRKNH |
| | | | TINVALVGNPNCGKTSLFNFASGAHERVGN |
| | | | YSGVTVDAKVGRAEFDGYVFNLVDLPGTY |
| | | | SLSAYSPEELYVRKQLVDKTPDVVINVIDSS |
| | | | NLERNLYLTTQLIDMHIRMVCALNMFDETE |
| | | | QRGDHIDAQKLSELFGVPMIPTVFTNGRGV |
| | | | KELFRQIIAVYEGKEDESLQFRHIHINHGHEI |
| | | | ENGIKEMQEHLKKYPELCHRYSTRYLAIKL |
| | | | LEHDKDVEQLVSPLGDSIEIFNHRDTAAAR |
| | | | VKEETGNDSETAIMDAKYGFINGALKEANF |
| | | | STGDKKDTYQTTHVIDHVLTNKYFGFPIFFL |
| | | | VLLVMFTATFVIGQYPMDWIEAGVGWLGE |
| | | | FISKNMPAGPVKDMIVDGIIGGVGAVIVFLP |
| | | | QILILYFFISYMEDCGYMSRAAFIMDRLMH |
| | | | KMGLHGKSFIPLIMGFGCNVPAVMATRTIE |
| | | | SRRSRLITMLILPLMSCSARLPIYVMITGSFF |
| | | | ALKYRSLAMLSLYIIGVLMAVAMSRLFSAF |
| | | | VVKGEDTPFVMELPPYRFPTWKAIGRHTW |
| | | | EKGKQYLKKMGGIILVASIIVWALGYFPLP |
| | | | DDPNMDNQARQEQSYIGRIGKAVEPVFRPQ |
| | | | GFNWKLDVGLLSGMGAKEIVASTMGVLYS |
| | | | NDGSFSDDNGYSSETGKYSKLHNLITKDVA |
| | | | TMHHISYEEAEPIATLTAFSFLLFVLLYFPC |
| | | | VATIAAIKGETGSWGWALFAAGYTTALAW |
| | | | IVSAVVFQVGMLFM |
| 112 | UDP-N- | P9WJM1 | MESFIIEGGHQLSGTIAPQGAKNEALEVICA |
| | acetylglucosamine | | TLLTSEEVIIRNVPDILDVNNLIKLLQDIGVK |
| | | | VKKLAPNEFSFQADEVNLDYLESSDFVKKC |
| | | | SSLRGSVLMIGPLLGRFGKATIAKPGGDKIG |
| | | | RRRLDTHFLGFKNLGAHFGRVEDRDVYEIQ |
| | | | ADKLVGTYMLLDEASITGTANIIMAAVLAE |
| | | | GTTTIYNAACEPYIQQLCKMLNAMGAKISG |
| | | | IASNLITIEGVKELHSADHRILPDMIEVGSFI |
| | | | GIAAMIGDGVRIKDVSVPNLGLILDTFHRLG |
| | | | VQIIVDNDDLIIPRQDHYVIDSFIDGTIMTISD |
| | | | APWPGLTPDLISVLLVVATQAQGSVLFHQK |
| | | | MFESRLFFVDKLIDMGAQIILCDPHRAVVV |
| | | | GHDNAKKLRAGRMSSPDIRAGIALLIAALT |
| | | | AQGTSRIDNIVQIDRGYENIEGRLNALGAKI |
| | | | QRAEVC |
| 113 | Ribitol-5- | Q8RKI9 | MNIAVIFAGGSGLRMHTKSRPKQFLDLNGK |
| | phosphate_ | | PIIIYTLELFDNHPNIDAIVVACIESWIPFLEK |
| | cytidylyltransferase | | QLRKFEINKVVKIIPGGKSGQESIYKGLCAA |
| | | | EEYAQSKGVSNEETTVLIHDGVRPLITEETI |
| | | | TDNIKKVEEVGSCITCIPATETLIVKQADDA |
| | | | LEIPSRADSFIARAPQSFRLIDIITAHRRSLAE |
| | | | GKADFIDSCTMMSHYGYKLGTIIGPMENIKI |
| | | | TTPTDFFVLRAMVKVHEDQQIFGL |

In some embodiments, the hemoglobin-dependent Prevotella strain is a strain of Prevotella bacteria comprising one or more of the proteins listed in Table 1 and that is free or substantially free of one or more proteins listed in Table 2. In some embodiments, the hemoglobin-dependent Prevotella strain is a strain of Prevotella bacteria that comprises all of the proteins listed in Table 1 and/or all of the genes encoding the proteins listed in Table 1 and that is free of all of the proteins listed in Table 2 and/or all of the genes encoding the proteins listed in Table 2.

Under the terms of the Budapest Treaty on the International Recognition of the Deposit of Microorganisms for the Purpose of Patent Procedure, the Prevotella Strain C. was deposited on Sep. 10, 2019, with the American Type Culture Collection (ATCC) of 10801 University Boulevard, Manassas, Va. 20110-2209 USA and was assigned ATCC Accession Number PTA-126140.

Applicant represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant acknowledges its duty to replace the deposit should the depository be unable to furnish a sample when requested due to the condition of the deposit.

Heme-Containing Polypeptides

Provided herein are heme-containing polypeptides (e.g., heme-containing polypeptides not sourced from an animal) that are able to be used in culture media to facilitate the growth of otherwise hemoglobin-dependent bacteria (e.g., for use in a bacterial composition (e.g., a pharmaceutical composition) and/or for therapeutic use). Heme-containing polypeptide is a polypeptide that is capable of binding covalently and/or noncovalently to a heme moiety. In some embodiments, the heme-containing polypeptide comprises a heme moiety.

In some embodiments, at least 1%, 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% of the heme-containing polypeptides used in the methods and/or compositions provided herein are bound by heme. In some embodiments, about 1%, 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% of the heme-containing polypeptides used in the methods and/or compositions provided herein are bound by heme.

In some embodiments, the heme-containing polypeptide is a globin and can include a globin fold, which comprises a series of seven to nine alpha helices. Globin type proteins can be of any class (e.g., class I, class II, or class III), and in some embodiments, can transport or store oxygen. For example, a heme-containing polypeptide can be a non-symbiotic type of hemoglobin or a leghemoglobin. A heme-containing polypeptide can be a monomer, i.e., a single polypeptide chain, or can be a dimer, a trimer, tetramer, and/or higher order oligomers.

Non-limiting examples of heme-containing polypeptides can include an androglobin, a cytoglobin, a globin E, a globin X, a globin Y, a hemoglobin, a leghemoglobin, a flavohemoglobin, Hell's gate globin I, a myoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin (e.g., HbN or HbO), a truncated 2/2 globin, a hemoglobin 3 (e.g., Glb3), a cytochrome, or a peroxidase. In some embodiments, the heme-containing polypeptide is a leghemoglobin. In certain embodiments, the heme-containing polypeptide is a soy leghemoglobin (e.g., recombinantly expressed (e.g., in Pichia pastoris) or naturally-sourced).

In some embodiments, the heme-containing polypeptide is a piscine polypeptide. In certain embodiments, the piscine polypeptide is that of a fish of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes. In some embodiments, the piscine heme-containing polypeptide is recombinantly expressed.

In some embodiments, the heme-containing polypeptide is an avian polypeptide. In some embodiments, the avian polypeptide is that of a bird of the genus Gallus, Meleagris, Anas, Anser, Branta, Chen, Agelastes, Numida, Guttera, Acryllium, or Columba. In some embodiments, the avian heme-containing polypeptide is recombinantly expressed.

In some embodiments, the heme-containing polypeptide is a non-animal-derived polypeptide. In some embodiments, the non-animal-derived polypeptide may be purified from a variety of organisms including but not limited to plants (e.g., grains, legumes), bacteria, cyanobacteria, fungus, algae, and/or protozoa. In some embodiments, the non-animal-derived polypeptide is that of an organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium. In some embodiments, the non-animal-derived polypeptide is recombinantly expressed.

In certain embodiments, heme-containing polypeptides can be from one or more of the following: a plant such as Nicotiana tabacum or Nicotiana sylvestris (tobacco), Zea mays (corn), Arabidopsis thaliana, a legume such as Glycine max (soybean), Cicer arietinum (garbanzo or chick pea), Pisum sativum (pea) varieties such as garden peas or sugar snap peas, Phaseolus vulgaris varieties of common beans such as green beans, black beans, navy beans, northern beans, or pinto beans, Vigna unguiculata varieties (cow peas), Vigna radiata (mung beans), Lupinus albus (lupin), or Medicago sativa (alfalfa), Brassica napus (canola), Triticum sps. (wheat, including wheat berries, and spelt), Gossypium hirsutum (cotton), Oryza sativa (rice), Zizania sps. (wild rice), Helianthus annuus (sunflower), Beta vulgaris (sugarbeet), Pennisetum glaucum (pearl millet), Chenopodium sp. (quinoa), Sesamum sp. (sesame), Linum usitatissimum (flax), or Hordeum vulgare (barley). In some embodiments, the heme-containing polypeptide is recombinantly expressed.

In certain embodiments, heme-containing polypeptides can be from mammals (e.g., farms animals such as cows, goats, sheep, pigs, ox, or rabbits), birds, plants, algae, fungi (e.g., yeast or filamentous fungi), ciliates, or bacteria. For example, a heme-containing polypeptide can be from a mammal such as a farm animal (e.g., a cow, goat, sheep, pig, fish, ox, or rabbit) or a bird such as a turkey or chicken. Heme-containing polypeptides can be from a plant such as Nicotiana tabacum or Nicotiana sylvestris (tobacco); Zea mays (corn), Arabidopsis thaliana, a legume such as Glycine max (soybean), Cicer arietinum (garbanzo or chick pea), Pisum sativum (pea) varieties such as garden peas or sugar snap peas, Phaseolus vulgaris varieties of common beans such as green beans, black beans, navy beans, northern beans, or pinto beans, Vigna unguiculata varieties (cow peas), Vigna radiata (mung beans), Lupinus albus (lupin), or Medicago sativa (alfalfa); Brassica napus (canola); Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); or Hordeum vulgare (barley). In some embodiments, the heme-containing polypeptide is recombinantly expressed.

In certain embodiments, heme-containing polypeptides can be a non-symbiotic hemoglobin. The non-symbiotic hemoglobin can be from any plant. In some embodiments, a non-symbiotic hemoglobin can be from a plant selected from the group consisting of soybean, sprouted soybean, alfalfa, golden flax, black bean, black eyed pea, northern bean, tobacco, pea, garbanzo, moong bean, cowpeas, pinto beans, pod peas, quinoa, sesame, sunflower, wheat berries, spelt, barley, wild rice, and rice. In some embodiments, the heme-containing polypeptide is recombinantly expressed.

In some embodiments, a leghemoglobin can be a soy, pea, or cowpea leghemoglobin. In some embodiments, the heme-containing polypeptide is recombinantly expressed.

In some embodiments, the leghemoglobin is a soy leghemoglobin. In some embodiments, the heme-containing polypeptide is recombinantly expressed.

In certain embodiments, recombinantly expressed heme-containing polypeptides can be isolated from fungi such as Saccharomyces cerevisiae, Pichia pastoris, Magnaporthe oryzae, Fusarium graminearum, or Fusarium oxysporum. In certain embodiments, the heme-containing polypeptide (e.g., soy leghemoglobin) is recombinantly expressed in Pichia pastoris.

In certain embodiments, recombinantly expressed heme-containing polypeptides can be isolated from one or more bacteria such as Escherichia coli, Bacillus subtilis, Bacillus megaterium, Synechocistis sp., Bacillus subtilis, Aquifex aeolicus, Methylacidiphilum infernorum (Hell's Gate), or thermophilic bacteria (e.g, that grow at temperatures greater than 45° C.) such as Thermophilus or Thermobifidafusca.

In certain embodiments, recombinantly expressed heme-containing polypeptides can be isolated from algae such as Chlamydomonas eugametos.

In certain embodiments, recombinantly expressed heme-containing polypeptides can be isolated from protozoans such as Paramecium caudatum or Tetrahymena pyriformis.

The sequences and structure of numerous heme-containing proteins are known. See for example, Reedy, et a, Nucleic Acids Research, 2008, Vol. 36, Database issue D307-D313 and the Heme Protein Database available on the world wide web at http://hemeprotein.info/heme.php. See also WO2014/110539 and WO2014/110532.

In some embodiments, the heme-containing polypeptide is symbiotic. In some embodiments, the heme-containing polypeptide is non-symbiotic. A non-symbiotic hemoglobin can be from one or more plants selected from the group consisting of soybean, sprouted soybean, alfalfa, golden flax, black bean, black eyed pea, northern, garbanzo, mung bean, cowpeas, pinto beans, pod peas, dried peas, quinoa, sesame, sunflower, wheat berries, spelt, barley, wild rice, and rice. In some embodiments, the heme-containing polypeptide is from soy. In some embodiments, the heme-containing polypeptide is recombinantly expressed.

The present disclosure also contemplates recombinantly expressed heme-containing polypeptides, such as those found in mammals such as a farm animal (e.g., a cow, goat, pig, ox, or rabbit), a bird such as chicken or turkey, or a fish such as carp or salmon.

The present disclosure also contemplates recombinantly expressed heme-containing polypeptides, such as those found in plants, bacteria, cyanobacteria, fungus, algae, grain, legume, and/or protozoa.

In certain embodiments, the heme-containing polypeptide is a polypeptide listed in Table 3. In certain embodiments, the heme-containing polypeptide is a polypeptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence listed in Table 3. In certain embodiments, the heme-containing polypeptide is a polypeptide encoded by a cDNA sequence listed in Table 3. In certain embodiments, the heme-containing polypeptide is a polypeptide encoded by a cDNA sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a cDNA sequence listed in Table 3. In some embodiments, the heme-containing polypeptide is recombinantly expressed.

In certain embodiments, the heme-containing polypeptide is a polypeptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with SEQ ID NO: 4.

In certain embodiments, the heme-containing polypeptide is a polypeptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the sequence of SEQ ID NO: 114 or UniProtKB—P02236 (LGB2_SOYBN).

In making the changes in the amino sequences of a polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

TABLE 3
cDNA and Amino Acid Sequences of Exemplary Heme-containing Polypeptides

SEQ ID NO: 1 Vigna radiate (mung bean) non-symbiotic hemoglobin amino acid sequence
(GenBank: AFB70892.1)

| | |
|---|---|
| 1 | mtttlergft eeqealvvks wnvmkknsge lglkfflkif eiapsaqklf sflrdstvpl |
| 61 | eqnpklkpha vsvfvmtcds avqlrkagkv tvresnlkkl gathfrtgva nehfevtkfa |
| 121 | lletikeavp emwspamkna wgeaydqlvd aikyemkpps s |

SEQ ID NO: 2 Methylacidiphilum infernorum (bacteria) hemoglobin-like flavoprotein
amino acid sequence (GenBank: WP_012463432.1)

| | |
|---|---|
| 1 | midqkekeli keswkriepn kneigllfya nlfkeeptvs vlfqnpissq srklmqvlgi |
| 61 | lvqgidnleg liptlqdlgr rhkqygvvds hyplvgdcll ksiqeylgqg fteeakaawt |
| 121 | kvygiaaqvm tae |

SEQ ID NO: 3 Aquifex aeolicus (thermophilic bacteria) bacitracin resistance protein
BacA amino acid sequence (GenBank: WP_010880084.1)

| | |
|---|---|
| 1 | mlseetirvi kstvpllkeh gteitarmye llfskypktk elfagaseeq pkklanaiia |
| 61 | yatyidrlee ldnaistiar shvrrnvkpe hyplvkecll qaieevlnpg eevlkaweea |
| 121 | ydflaktlit lekklysqp |

SEQ ID NO: 4 Glycine max (soybean) leghemoglobin C2 amino acid sequence (GenBank:
NP_001235248.2)

| | |
|---|---|
| 1 | mgaftekqea lvsssfeafk anipqysvvf ytsilekapa akdlfsflsn gvdpsnpklt |
| 61 | ghaeklfglv rdsagqlkan gtvvadaalg sihaqkaitd pqfvvvkeal lktikeavgd |
| 121 | kwsdelssaw evaydelaaa ikkaf |

SEQ ID NO: 5 Hordeum vulgare (barley) non-symbiotic hemoglobin amino acid sequence
(GenBank: Q42831.1)

| | |
|---|---|
| 1 | msaaegavvf seekealvlk swaimkkdsa nlglrfflki feiapsarqm fpflrdsdvp |
| 61 | letnpklkth avsvfvmtce aaaqlrkagk itvrettlkr lggthlkygv adghfevtrf |
| 121 | alletikeal padmwgpemr nawgeaydql vaaikqemkp ae |

SEQ ID NO: 6 Pyricularia oryzae (fungus) heme peroxidase amino acid sequence
(GenBank: XP_003713235.1)

| | |
|---|---|
| 1 | mdgavrldwt gldltgheih dgvpiasrvq vmvsfplfkd qhiimsskes psrksstigq |
| 61 | strnqscqad tqkgqlppvg ekpkpvkenp mkklkemsqr plptqhgdgt yptekkltqi |
| 121 | gedlkhirgy dvktllamvk sklkgeklkd dktmlmervm qlvarlptes kkraeltdsl |
| 181 | inelwesldh pplnylqpeh syrtpdqsyn hpfnpqlqaa qsryarsvip tvtppqalpd |
| 241 | pglifdsimg rtpnsyrkhp nnvssilwyw atiiihdifw tdprdintnk sssyldlapl |
| 301 | ygnsqemqds irtfkdgrmk pdcyadkrla gmppgvsvll imfnrfhnhv aenlalineg |
| 361 | grfnkpsdll egeareaawk kydndlfqva rlvtsglyin itlvdyvrni vnlnrvdttw |
| 421 | tldprqdaga hvgtadgaer gtgnavsaef nlcyrwhsci sekdskfvea qfqnifgkpa |
| 481 | sevrpdemwk gfakmeqntp adpgqrtfgg fkrgpdgkfd dddlvrcise avedvagafg |
| 541 | arnvpqamkv vetmgiiqgr kwnvaglnef rkhfhlkpys tfedinsdpg vaealrrlyd |
| 601 | hpdnvelypg lvaeedkqpm vpgvgiapty tisrvvlsda vclvrgdrfy ttdftprnlt |
| 661 | nwgykevdyd lsvnhgcvfy klfirafpnh fkqnsvyahy pmvvpsenkr ilealgradl |
| 721 | fdfeapkyip prvnitsygg aeyiletqek ykvtwheglg flmgegglkf mlsgddplha |
| 781 | qqrkcmaaql ykdgwteavk afyagmmeel lvsksyflgn nkhrhvdiir dvgnmvhvhf |
| 841 | asqvfglplk taknptgvft eqemygilaa ifttiffdld psksfplrtk trevcqklak |
| 901 | lveanvklin kipwsrgmfv gkpakdepls iygktmikgl kahglsdydi awshvvptsg |
| 961 | amvpnqaqvf aqavdyylsp agmhyipeih mvalqpstpe tdalllgyam egirlagtfg |
| 1021 | syreaavddv vkedngrqvp vkagdrvfvs fvdaardpkh fpdpevvnpr rpakkyihyg |
| 1081 | vgphaclgrd asqiaitemf rclfrrrnvr rvpgpqgelk kvprpggfyv ymredwgglf |
| 1141 | pfpvtmrvmw dde |

SEQ ID NO: 7 Fusarium oxysporum (fungus) L-ascorbate peroxidase 5, peroxisomal amino
acid sequence (GenBank: ENH74019.1)

| | |
|---|---|
| 1 | mkgsatlafa lvqfsaasql vwpskwdeve dllymqggfn krgfadalrt cefgsnvpgt |
| 61 | qntaewlrta fhdaithdak agtggldasi ywessrpenp gkafnntfgf fsgfhnprat |
| 121 | asdltalgtv lavgaengpr ipfragrida ykagpagvpe pstnlkdtfa aftkagftke |
| 181 | emtamvacgh aiggvhsvdf peivgikadp nndtnvpfqk dvssfhngiv teylagtskn |
| 241 | plvasknatf hsdkrifdnd katmkklstk agfnsmcadi ltrmidtvpk svqltpvlea |
| 301 | ydvrpyitel slnnknkihf tgsvrvritn nirdnndlai nliyvgrdgk kvtvptqqvt |
| 361 | fqggtsfgag evfanfefdt tmdakngitk ffiqevkpst katvthdnqk tggykvddtv |
| 421 | lyqlqqscav leklpnaplv vtamvrdara kdaltlrvah kkpvkgsivp rfqtaitnfk |
| 481 | atgkkssgyt gfqaktmfee qstyfdivlg gspasgvqfl tsqampsqcs |

SEQ ID NO: 8 Fusarium graminearum (fungus) cytochrome c peroxidase amino acid
sequence (GenBank: XP_011317020.1)

| | |
|---|---|
| 1 | masatrqfar aatratrngf aiaprqvirq qgrryyssep aqksssawiw ltgaavagga |
| 61 | gyyfygnsas satakvfnps kedyqkvyne iaarleekdd yddgsygpvl vrlawhasgt |
| 121 | ydketgtggs ngatmrfape sdhganagla aardflqpvk ekfpwitysd lwilagvcai |
| 181 | qemlgpaipy rpgrsdrdvs gctpdgrlpd askrqdhlrg ifgrmgfndq eivalsgaha |
| 241 | lgrchtdrsg ysgpwtfspt vltndyfrll veekwqwkkw ngpaqyedks tkslmmlpsd |
| 301 | ialiedkkfk pwvekyakdn daffkdfsnv vlrlfelgvp faqgtenqrw tfkpthqe |

SEQ ID NO: 9 Chlamydomonas moewusii (algae) globin amino acid sequence (PDB:
1UVX_A)

| | |
|---|---|
| 1 | slfaklggre aveaavdkfy nkivadptvs tyfsntdmkv qrskqfafla yalggasewk |
| 61 | gkdmrtahkd lvphlsdvhf qavarhlsdt ltelgvpped itdamavvas trtevlnmpq |
| 121 | q |

SEQ ID NO: 10 Chlamydomonas moewusii (algae) truncated hemoglobin LI637 amino acid
sequence (UniProtKB/Swiss-Prot: Q08753.1)

| | |
|---|---|
| 1 | mmrtvqlrtl rpciraqqqp vrpstsataa aatapapark cpsslfaklg greaveaavd |
| 61 | kfynkivadp tvstyfsntd mkvqrskqfa flayalggas ewkgkdmrta hkdlvphlsd |
| 121 | vhfqavarhl sdtltelgvp peditdamav vastrtevln mpqq |

SEQ ID NO: 11 Tetrahymena pyriformis (protozoan) group 1 truncated hemoglobin amino
acid sequence (UniProtKB/Swiss-Prot: P17724.1)

| | |
|---|---|
| 1 | mnkpqtiyek lggenamkaa vplfykkvla dervkhffkn tdmdhqtkqq tdfltmllgg |
| 61 | pnhykgknmt eahkgmnlqn lhfdaiienl aatlkelgvt davineaakv iehtrkdmlg |
| 121 | k |

SEQ ID NO: 12 Paramecium caudatum (protozoan) group 1 truncated hemoglobin amino
acid sequence (UniProtKB/Swiss-Prot: P15160.2)

| | |
|---|---|
| 1 | mslfeqlggq aavqavtaqf yaniqadatv atffngidmp nqtnktaafl caalggpnaw |
| 61 | tgrnlkevha nmgvsnaqft tvighlrsal tgagvaaalv eqtvavaetv rgdvvtv |

SEQ ID NO: 13 Aspergillus niger (fungus) truncated flavohemoglobin amino acid
sequence (GenBank: CAF32309.1)

| | |
|---|---|
| 1 | mpltpeqiki ikatvpvlqe ygtkittaxy mnmstvhpel navfntanqv kghqaralag |
| 61 | alfayashid dlgalgpave licnkhasly iqadeykivg kylleamkev lgdactddil |
| 121 | dawgaaywal adiminreaa lykqsqgwtn wrqfriskkv pesdeitsfy lepvdgkplp |
| 181 | afrpgqyisv svqvpeedpq arqyslsdts rsdyyrisvk ketgldprap gakrhpgyvs |
| 241 | nvlhdmikeg dlidvshpyg dfflstaeat hpivllsagv gmtpmmsiln titkksnrki |
| 301 | hfihgsrtte arafkshvqk lekeipnmqv tyflsrpgds dqlgvdyhha gridlqkldg |
| 361 | pshlyldnps teyyicgpdt fmtqmeealk aygvgddrik melfgtggvp hn |

SEQ ID NO: 14 Zea mays (corn) non-symbiotic hemoglobin amino acid sequence
(UniProtKB/Swiss-Prot: Q9FY42.1)

| | |
|---|---|
| 1 | malaeaddga vvfgeeqeal vlkswavmkk daanlglrff lkvfeiapsa eqmfsflrds |
| 61 | dvpleknpkl kthamsvfvm tceaaaqlrk agkvtvrett lkrlgathlr ygvadghfev |
| 121 | tgfalletik ealpadmwsl emkkawaeay sqlvaaikre mkpda |

SEQ ID NO: 15 Oryza sativa Japonica Group (rice) non-symbiotic hemoglobin 2 amino
acid sequence (NCBI Reference Sequence: XP_015628351.1)

| | |
|---|---|
| 1 | malvegnngv sggavsfsee qealvlkswa imkkdsanig lrfflkifev apsasqmfsf |
| 61 | lrnsdvplek npklkthams vfvmtceaaa qlrkagkvtv rdttlkrlga thfkygvgda |
| 121 | hfevtrfall etikeavpvd mwspamksaw seaynqlvaa ikqemkpae |

SEQ ID NO: 16 Arabidopsis thaliana (plant) hemoglobin 1 amino acid sequence (NCBI
Reference Sequence: NP_179204.1)

| | |
|---|---|
| 1 | mesegkivft eeqealvvks wsvmkknsae lglklfikif eiapttkkmf sflrdspipa |
| 61 | eqnpklkpha msvfvmcces avqlrktgkv tvrettlkrl gashskygvv dehfevakya |
| 121 | lletikeavp emwspemkva wgqaydhlva aikaemnlsn |

SEQ ID NO: 17 Pisum sativum (pea) Leghemoglobin Lb5-10 amino acid sequence
(UniProtKB/Swiss-Prot: O48668.3)

| | |
|---|---|
| 1 | mgftdkqeal vnsswesfkq nlsgnsilfy tiilekapaa kglfsflkdt agvedspklq |
| 61 | ahaeqvfglv rdsaaqlrtk gevvlgnatl gaihvqrgvt dphfvvvkea llqtikkasg |
| 121 | nnwseelnta wevaydglat aikkamt |

SEQ ID NO: 18 Vigna unguiculata (cowpea) leghemoglobin-1 amino acid sequence (NCBI
Reference Sequence: XP_027938137.1)

| | |
|---|---|
| 1 | mvafsdkqea lvngayeafk anipkysvvf yttilekapa aknlfsflan gvdatnpklt |
| 61 | ghaeklfglv rdsaaqlras ggvvadaalg avhsqkavnd aqfvvvkeal vktlkeavgd |
| 121 | kwsdelgtav elaydelaaa ikkay |

SEQ ID NO: 19 Bos taurus (cattle) myoglobin amino acid sequence (NCBI Reference
Sequence: NP_776306.1)

| | |
|---|---|
| 1 | mglsdgewql vlnawgkvea dvaghgqevl irlftghpet lekfdkfkhl kteaemkase |
| 61 | dlkkhgntvl talggilkkk ghheaevkhl aeshankhki pvkylefisd aiihvlhakh |
| 121 | psdfgadaqa amskalelfr ndmaaqykvl gfhg |

SEQ ID NO: 20 Sus scrofa (pig) myoglobin amino acid sequence (NCBI Reference
Sequence: NP_999401.1)

| | |
|---|---|
| 1 | mglsdgewql vlnvwgkvea dvaghgqevl irlfkghpet lekfdkfkhl ksedemkase |
| 61 | dlkkhgntvl talggilkkk ghheaeltpl aqshatkhki pvkylefise aiiqvlqskh |
| 121 | pgdfgadaqg amskalelfr ndmaakykel gfqg |

SEQ ID NO: 21 Equus caballus (horse) myoglobin amino acid sequence (NCBI Reference
Sequence: NP_001157488.1)

| | |
|---|---|
| 1 | mglsdgewqq vlnvwgkvea diaghgqevl irlftghpet lekfdkfkhl kteaemkase |
| 61 | dlkkhgtvvl talggilkkk ghheaelkpl aqshatkhki pikylefisd aiihvlhskh |
| 121 | pgdfgadaqg amtkalelfr ndiaakykel gfqg |

SEQ ID NO: 22 Nicotiana tabacum (common tobacco) non-symbiotic hemoglobin 1-like
amino acid sequence (NCBI Reference Sequence: XP_016448353.1)

| | |
|---|---|
| 1 | mssfteeqea lvlkswdsmk knagewglkl flkifeiaps akklfsflkd snvpleqnak |
| 61 | lkphaksvfv mtceaavqlr kagkvvvrds tlkklgaahf kygvadehfe vtkfalleti |
| 121 | keavpdmwsv dmknawgeaf dqlvngikte mk |

SEQ ID NO: 23 Bacillus (bacteria) group 2 truncated hemoglobin YjbI amino acid
sequence (NCBI Reference Sequence: WP_003232928.1)

| | |
|---|---|
| 1 | mgqsfnapye aigeellsql vdtfyervas hpllkpifps dltetarkqk qfltqylggp |
| 61 | plyteehghp mlrarhlpfp itneradawl scmkdamdhv glegeirefl fgrleltarh |
| 121 | mvnqteaedr ss |

SEQ ID NO: 24 Corynebacterium glutamicum (bacteria) globin amino acid sequence
(NCBI Reference Sequence: WP_011015129.1)

| | |
|---|---|
| 1 | mttsenfyds vggeetfsli vhrfyeqvpn ddilgpmypp ddfegaeqrl kmflsqywgg |
| 61 | pkdyqeqrgh prlrmrhvny pigvtaaerw lqlmsnaldg vdltaeqrea iwehmvraad |
| 121 | mlinsnpdph a |

SEQ ID NO: 25 Synechocystis (cyanobacteria) group 1 truncated hemoglobin amino acid
sequence (NCBI Reference Sequence: WP_010872616.1)

| | |
|---|---|
| 1 | mstlyeklgg ttavdlavdk fyervlqddr ikhffadvdm akqrahqkaf ltyafggtdk |
| 61 | ydgrymreah kelvenhgln gehfdavaed llatlkemgv pedliaevaa vagapahkrd |
| 121 | vlnq |

SEQ ID NO: 26 Synechococcus sp. PCC 7335 (cyanobacteria) globin domain protein
amino acid sequence (NCBI Reference Sequence: WP_006454394.1)

| | |
|---|---|
| 1 | mdvalleksf eqispraief sasfyqnlfh hhpelkplfa etsqtiqekk lifslaaiie |
| 61 | nlrnpdilqp alkslgarha evgtikshyp lvgqalietf aeylaadwte qlatawveay |
| 121 | dviastmieg adnpaaylep eltfyewldl ygeespkvrn aiatlthfhy gedpqdvqrd |
| 181 | srg |

SEQ ID NO: 27 Nostoc commune (cyanobacteria) group 1 truncated hemoglobin amino
acid sequence (UniProtKB/Swiss-Prot: Q00812.1)

| | |
|---|---|
| 1 | mstlydnigg qpaieqvvde lhkriatdsl lapvfagtdm vkqrnhlvaf laqifegpkq |
| 61 | yggrpmdkth aglnlqqphf daiakhlger mavrgvsaen tkaaldrvtn mkgailnk |

SEQ ID NO: 28 Bacillus megaterium (bacteria) globin-like protein amino acid sequence
(NCBI Reference Sequence: WP_014461570.1)

| | |
|---|---|
| 1 | mrekihspye llggehtisk lvdafytrvg qhpelapifp dnltetarkq kqfltqylgg |
| 61 | pslyteehgh pmlrarhlpf eitpsrakaw ltcmheamde inlegperde lyhrliltaq |
| 121 | hminspeqtd ekgfsh |

SEQ ID NO: 29 Salmo Salar (Salmon) hemoglobin subunit alpha amino acid sequence
(NCBI Reference Sequence: NP_001117134.1)

| | |
|---|---|
| 1 | msltardksv vnafwgkikg kadvvgaeal grmltaypqt ktyfshwadl spgsapvkkh |
| 61 | ggvimgaign avglmddlvg gmsglsdlha fklrvdpgnf kilshnilvt laihfpadft |
| 121 | pevhiavdkf laalsaalad kyr |

SEQ ID NO: 30 Salmo Salar (Salmon) hemoglobin subunit alpha cDNA sequence (NCBI
Reference Sequence: NM_001123662.1, CDS region 52-483)

| | |
|---|---|
| 1 | ttcttcagtt catttctcgt agatttaata caggtcaatc aaaggagcaa aatgagtctg |
| 61 | acagcaaggg acaaatctgt ggtcaatgcc ttctggggca agattaaagg aaaggcagat |
| 121 | gtcgtcggcg ctgaggcttt gggaaggatg ctgactgctt acccccagac taagacctac |
| 181 | ttctcccact gggctgacct gagccccggc tctgccccag tcaagaagca tggaggcgtc |
| 241 | atcatgggtg caattggtaa tgctgtcgga ctgatggacg acctcgtggg gggaatgagt |
| 301 | ggtctcagcg atctgcacgc cttcaagctg cgcgttgacc ctggaaactt caagattctg |
| 361 | tcccacaaca tccttgtcac cctggctatt cacttccctg cggatttcac tcccgaagtg |
| 421 | cacattgctg tggataaatt ccttgcagcc ttgtccgctg ccctggctga caaatacaga |
| 481 | taagaccatc atgaagtcca aaattggact ccagttcctg ctctgttgtt atcacaaaat |
| 541 | aaacaggcaa tgaatg |

SEQ ID NO: 31 Salmo Salar (Salmon) hemoglobin subunit beta-1 amino acid sequence
(NCBI Reference Sequence: NP_001265947.1)

| | |
|---|---|
| 1 | mvdwtdaeks tisavwgkvd inevgplala rvlivypwtq ryfgsfgdvs tpaaimgnpk |
| 61 | vaahgkvvcg aldkavknmg nilatyksls ethanklfvd pdnfrvladv ltivvaakfg |
| 121 | asftpeiqat wqkfmkvvva amgsryf |

SEQ ID NO: 32 Salmo Salar (Salmon) hemoglobin subunit beta-1 cDNA sequence (NCBI
Reference Sequence: NM_001279018.1, CDS region 41-484)

| | |
|---|---|
| 1 | tttaatcttg ttgcaaaacc ggagatcaaa cactgacaac atggttgact ggacagacgc |
| 61 | cgagaagagc accatcagtg ctgtctgggg aaaagtagat atcaatgagg tcggaccact |
| 121 | ggctctggca agagtcctga tcgtctaccc ctggactcag cgttatttcg gctctttcgg |
| 181 | agatgtgtct actcccgcag caatcatggg caaccccaaa gttgctgctc acggcaaggt |
| 241 | cgtgtgtgga gctctggata aagctgtgaa gaacatgggc aacatcttgg ccacatacaa |
| 301 | gtcactgagc gagacacacg ccaacaaact cttcgtcgac cctgacaatt tcagggtgtt |
| 361 | ggctgacgtc ctcacaattg tcgttgccgc caagttcgga gcctctttca ctcctgaaat |
| 421 | ccaggcaacc tggcagaagt tcatgaaagt ggttgtcgca gccatgggca gccggtactt |
| 481 | ctaaatgcac acgagacaag tgattatgtt ctgacatccc tgcctgagtg aaaataaaga |
| 541 | cgttg |

SEQ ID NO: 33 Cyprinus carpio (common carp) hemoglobin subunit alpha amino acid
sequence (NCBI Reference Sequence: XP_018973483.1)

| | |
|---|---|
| 1 | mslsdkdkaa vkalwakisp kaddigaeal grmltvypqt ktyfahwadl spgsgpvkkh |
| 61 | gkvimgavgd avskiddlvg glaalselha fklrvdpanf kilahnvivv igmlypgdfp |
| 121 | pevhmsvdkf fqnlalalse kyr |

SEQ ID NO: 34 Cyprinus carpio (common carp) hemoglobin subunit alpha cDNA
sequence (NCBI Reference Sequence: XM_019117938.1, CDS region 67-498)

| | |
|---|---|
| 1 | aaaatcctct tttcgccaac atctttttct gaagacaatc taacttgaga aaaagaagac |
| 61 | gcagcaatga gtctctctga taaggacaag gctgctgtga aagccctatg ggctaagatc |
| 121 | agccccaaag ccgatgatat cggcgctgaa gctctcggca gaatgctgac cgtctaccct |
| 181 | cagaccaaga cctacttcgc tcactgggct gacctgagcc ctgggtccgg tcctgtgaag |
| 241 | aagcatggca aggttatcat gggtgcagtc ggcgatgccg tttcaaaaat agacgacctt |
| 301 | gtgggaggtc tggctgccct gagcgaactt catgctttca agctgcgtgt tgacccggcc |
| 361 | aacttcaaga tcctcgcaca caatgtcatc gtggtcatcg gcatgctcta ccctggagac |
| 421 | ttccccccag aggttcacat gtcagttgac aagtttttcc agaacttggc tctggctctc |
| 481 | tctgagaagt accgctaaat ttccggtggg cattcatacg cgacactgtg cggcactcct |
| 541 | gaccaactca agtgtgatgt ctgaataaaa tttctcaata aaaggcagag aattaaatga |

SEQ ID NO: 35 Cyprinus carpio (common carp) hemoglobin subunit subunit beta amino
acid sequence (NCBI Reference Sequence: XP_018973478.1)

| | |
|---|---|
| 1 | mvewtdaers aiiglwgkln pdelgpqala rclivypwtq ryfasfgnls spaaimgnpk |
| 61 | vaahgrtvmg gleraiknmd nikatyapls vmhseklhvd pdnfrlladc itvcaamkfg |
| 121 | psgfnadvqe awqkflcvvv salcrqyh |

SEQ ID NO: 36 Cyprinus carpio (common carp) hemoglobin subunit beta cDNA sequence
(NCBI Reference Sequence: XM_019117933,1, CDS region 77-523)

| | |
|---|---|
| 1 | actccggttt ctgaatttct actagtgatt cctactggac ttaaacaaga acctaaggac |
| 61 | accctaaaac gtagacatgg ttgagtggac agatgctgag cgaagtgcca tcattggcct |
| 121 | gtggggaaag ctcaatcccg atgaactcgg acctcaggcc ctggccaggt gtctgatcgt |
| 181 | gtatccctgg actcagagat atttcgcctc cttcggaaac ctgtcaagcc ccgctgcgat |
| 241 | catgggtaac cccaaggtgg cagctcacgg gaggactgtg atgggaggtc tggagagagc |
| 301 | catcaagaac atggacaaca tcaaggccac ctatgcgcca ctcagtgtga tgcactctga |
| 361 | gaaattgcat gtggatcccg acaacttcag gctcctggct gattgcatca ccgtgtgcgc |
| 421 | tgccatgaag tttggcccct ctggtttcaa tgctgacgtc caggaggcct ggcagaagtt |
| 481 | tctgtgtgtc gtcgtttccg ctctgtgcag acagtaccac tagattctca tcaccgatga |
| 541 | acaccagctg tattgcagaa gattgcggga gctgttcaca caaagatcta actcttataa |
| 601 | aataagatca ataaaatatt gttaaaagca |

SEQ ID NO: 37 Meleagris gallopavo (turkey) hemoglobin subunit alpha-A amino acid
sequence (NCBI Reference Sequence: XP_003210796.1)

| | |
|---|---|
| 1 | mvlsaadknn vkgiftkiag haeeygaetl ermfityppt ktyfphfdls hgsaqikghg |
| 61 | kkvvaaliea vnhiddiagt lsklsdlhah klrvdpvnfk llgqcflvvv aihhpaaltp |
| 121 | evhasldkfl cavgtvltak yr |

SEQ ID NO: 38 Meleagris gallopavo (turkey) hemoglobin subunit alpha-A cDNA
sequence (NCBI Reference Sequence: XM_003210748.3, CDS region 53-481)

| | |
|---|---|
| 1 | agcagactgc aggggcaccc gtgctggggg ctgccaacgc ggaggtgcaa ccatggtgct |
| 61 | gtccgctgct gacaagaaca acgtcaaggg catcttcacc aaaatcgccg gccatgctga |
| 121 | ggagtatggc gccgagaccc tggagaggat gttcatcacc taccccccga ccaagaccta |
| 181 | cttcccccac tttgatctgt cacacggctc cgctcagatc aaggggcacg gcaagaaggt |
| 241 | agtggctgcc ctgatcgagg ctgtcaacca cattgatgac atcgcaggca ccctctccaa |
| 301 | gctcagcgac ctccatgccc acaagctccg cgtggaccct gtcaacttca aactcctggg |
| 361 | ccaatgcttc ctggtggtgg tggccatcca ccaccctgct gccctgaccc cagaggtcca |
| 421 | tgcttccctg gacaagttct tgtgcgctgt gggcaccgtg ctgactgcca agtaccgtta |
| 481 | agacagcatg gcagctagag ctggggccaa cccatcgcca gccttccaac agcgagcagc |
| 541 | caaatgagat gaaataaaat ctgttgcatt tgtgctcca |

SEQ ID NO: 39 Meleagris gallopavo (turkey) hemoglobin subunit subunit beta amino acid
sequence (NCBI Reference Sequence: XP_003203314.1)

| | |
|---|---|
| 1 | mvhwsaeekq litglwgkvn vadcgaeala rllivypwtq rffasfgnls sptailgnpm |
| 61 | vrahgkkvlt sfgdavknld nikntfsqls elhcdklhvd penfrllgdi liivlaahfs |
| 121 | kdftpecqaa wqklvrvvah alarkyh |

SEQ ID NO: 40 Meleagris gallopavo (turkey) hemoglobin subunit beta cDNA sequence
(NCBI Reference Sequence: XM_003203266.3, CDS region 91-534)

| | |
|---|---|
| 1 | acggccgctc accagcgtgc gatccccgcg ggagcaagag cccagacctc ctccgtaccg |
| 61 | acagccacac gctaccctcc aaccgccgcc atggtgcact ggtctgctga ggagaagcag |
| 121 | ctcatcaccg gcctctgggg caaggtcaat gtggccgact gcggggccga agccctggcc |
| 181 | aggctgctga ttgtctaccc ctggacccag aggttctttg cgtcctttgg gaacctctcc |
| 241 | agccccaccg ccatccttgg caaccccatg gtccgtgccc atggcaagaa agtgctcacc |
| 301 | tcctttgggg atgctgtgaa gaacctggac aacatcaaga acaccttctc ccaactgtcc |
| 361 | gagctgcact gtgacaagct gcatgtggac cccgagaact tcaggctcct gggtgacatc |
| 421 | ctcatcatcg tcctggccgc ccacttcagc aaggacttca ctcctgaatg ccaggctgcc |
| 481 | tggcagaagc tggtccgtgt ggtggcccat gccctggctc gcaagtacca ctaagcacca |
| 541 | gcagcaaaga tcccggagcg ccctacagca attgtatgca cctgcagaaa tgctccgagg |
| 601 | ctgacagctt gtgacaggca aataaagttc attcagtgac actc |

SEQ ID NO: 41 Gallus gallus (chicken) hemoglobin subunit alpha-A amino acid sequence
(NCBI Reference Sequence: NP_001004376.1)

| | |
|---|---|
| 1 | mvlsaadknn vkgiftkiag haeeygaetl ermfttyppt ktyfphfdls hgsaqikghg |
| 61 | kkvvaaliea anhiddiagt lsklsdlhah klrvdpvnfk llgqcflvvv aihhpaaltp |
| 121 | evhasldkfl cavgtvltak yr |

SEQ ID NO: 42 Gallus gallus (chicken) hemoglobin subunit alpha-A cDNA sequence
(NCBI Reference Sequence: NM_001004376.3, CDS region 27-455)

| | |
|---|---|
| 1 | ggggctgcca acacagaggt gcaaccatgg tgctgtccgc tgctgacaag aacaacgtca |
| 61 | agggcatctt caccaaaatc gccggccatg ctgaggagta tggcgccgag accctggaaa |
| 121 | gctccgctca gatcaagggg cacggcaaga cctacttccc ccacttcgat ctgtcacacg |
| 181 | accacattga tgacatcgcc ggcaccctct aggtagtggc tgccttgatc gaggctgcca |
| 241 | accacattga tgacatcgcc ggcaccctct ccaagctcag cgacctccat gcccacaagc |
| 301 | tccgcgtgga ccctgtcaac ttcaaactcc tgggccaatg cttcctggtg gtggtggcca |
| 361 | tccaccaccc tgctgccctg accccggagg tccatgcttc cctggacaag ttcttgtgcg |
| 421 | ccgtgggcac tgtgctgacc gccaagtacc gttaagacgg cacggtggct agagctgggg |
| 481 | ccaacccatc gccagccctc cgacagcgag cagccaaatg agatgaaata aaatctgttg |
| 541 | catttgtgct ccag |

SEQ ID NO: 43 Gallus gallus (chicken) hemoglobin subunit subunit beta amino acid
sequence (NCBI Reference Sequence: NP_990820.1)

| | |
|---|---|
| 1 | mvhwtaeekq litglwgkvn vaecgaeala rllivypwtq rffasfgnls sptailgnpm |
| 61 | vrahgkkvlt sfgdavknld nikntfsqls elhcdklhvd penfrllgdi liivlaahfs |
| 121 | kdftpecqaa wqklvrvvah alarkyh |

SEQ ID NO: 44 Gallus gallus (chicken) hemoglobin subunit beta cDNA sequence (NCBI
Reference Sequence: NM_205489.2, CDS region 69-512)

| | |
|---|---|
| 1 | tccccacggg agcaagagcc cagacctcct ccgtaccgac agccacacgc taccctccaa |
| 61 | ccgccgccat ggtgcactgg actgctgagg agaagcagct catcaccggc ctctggggca |
| 121 | aggtcaatgt ggccgaatgt ggggccgaag ccctggccag gctgctgatc gtctacccct |
| 181 | ggacccagag gttctttgcg tcctttggga acctctccag ccccactgcc atccttggca |
| 241 | accccatggt ccgcgcccac ggcaagaaag tgctcacctc ctttggggat gctgtgaaga |
| 301 | acctggacaa catcaagaac accttctccc aactgtccga actgcattgt gacaagctgc |
| 361 | atgtggaccc cgagaacttc aggctcctgg gtgacatcct catcattgtc ctggccgccc |
| 421 | acttcagcaa ggacttcact cctgaatgcc aggctgcctg gcagaagctg gtccgcgtgg |
| 481 | tggcccatgc cctggctcgc aagtaccact aagcaccagc accaaagatc acggagcacc |
| 541 | tacaaccatt gcatgcacct gcagaaatgc tccggagctg acagcttgtg acaaataaag |
| 601 | ttcattcagt gacactcaaa aaaaaaaaaa aa |

SEQ ID NO: 114 Glycine max (soybean) leghemoglobin C2 amino acid sequence
(UniProtKB-P02236 (LGB2_SOYBN))

| | |
|---|---|
| 1 | mgaftekqea lvsssfeafk anipqysvvf ytsilekapa akdlfsflsn gvdpsnpklt |
| 61 | ghaeklfglv rdsagqlkan gtvvadaalg sihaqkaitd pqfvvvkeal lktikeavgd |
| 121 | kwsdelssaw evaydelaaa ikkaf |

Polypeptide Purification

As described herein, isolated and purified heme-containing polypeptides can be derived from non-animal sources such as plants, bacteria, cyanobacteria, fungus, algae, grain, legume, and/or protozoa. In some embodiments, the isolated and purified proteins can be obtained from genetically modified organisms such as genetically modified bacteria or yeast. In some embodiments, the isolated and purified polypeptides are chemically synthesized or obtained via in vitro synthesis. In some embodiments, the non-animal-derived polypeptide is recombinantly expressed.

In some embodiments, heme-containing polypeptides can be purified from fish (e.g., carp, salmon, or any other fish that are commercially easily sourced), fish meals, or fish protein hydrolysates. In some embodiments, heme-containing polypeptides can be purified from birds (e.g., chicken, turkey, or any other birds that are commercially easily sourced).

In some embodiments, a heme-containing polypeptide described herein (e.g., piscine heme-containing polypeptides, avian heme-containing polypeptides, or non-animal-derived heme-containing polypeptides) can be recombinantly produced using polypeptide expression techniques (e.g., cloning and expressing an exogenous nucleic acid encoding a heme-containing polypeptide in host cells, such as bacterial cells (e.g., Escherichia coli), insect cells, fungal cells such as yeast cells (e.g., Pichia Pastoris), plant cells, or mammalian cells). The exogenous nucleic acid may be operably linked to a regulatory sequence (e.g., promoter). The exogenous nucleic acid may be in a vector, such as an expression vector. In some embodiments, the exogenous nucleic acid may be in a viral vector suitable for transmission of DNA into host cells (e.g., insect cells, mammalian cells). The recombinant polypeptides may be expressed and purified as an intracellular protein, a secreted protein, and/or an insoluble protein (e.g., contained in inclusion bodies), which can be solubilized using urea and slowly refolded in a native condition.

In some embodiments, the recombinant proteins comprise a heterologous sequence (e.g., a histidine tag, TAP (tandem affinity purification) tag, TEV cleavage site, a FLAG tag, a GST tag, and/or an immunoglobulin domain) that may be useful in purification and/or detection of the polypeptide.

In certain embodiments, heme-containing polypeptides can be purified on the basis of their molecular weight, for example, by size exclusion chromatography, ultrafiltration through membranes, or density centrifugation. In some embodiments, the polypeptides can be separated based on their surface charge, for example, by isoelectric precipitation, anion exchange chromatography, or cation exchange chromatography. Polypeptides can also be separated on the basis of their solubility, for example, by ammonium sulfate precipitation, isoelectric precipitation, surfactants, detergents, or solvent extraction. Polypeptides can also be separated by their affinity to another molecule, using, for example, hydrophobic interaction chromatography, reactive dyes, or hydroxyapatite. Affinity chromatography can also use antibodies having specific binding affinity for the protein of interest, nickel NTA for His-tagged recombinant proteins, lectins to bind to sugar moieties on a glycoprotein, or other molecules which specifically bind the polypeptides of interest.

In some embodiments, the heme-containing polypeptide is leghemoglobin. In certain embodiments, leghemoglobin can be purified from e.g., whole roots or root nodules (e.g., soy roots or soy root nodules). Whole roots or root nodules can be harvested and lysed, for example in 20 mM potassium phosphate pH 7.4, 100 mM potassium chloride and 5 mM EDTA using a grinder-blender. During this process, leghemoglobin is released into the buffer. Root-nodule lysate containing leghemoglobin can be cleared from cell debris by filtration through 5 μm filter. In some embodiments, filtration is followed by centrifugation (7000×g, 20 min). Clarified lysate containing leghemoglobin is then filtered through 200 nm filter and applied to an anion-exchange chromatography column (High Prep Q; High Prep DEAE, GE Healthcare) on a fast protein liquid chromatography machine (GE Healthcare). Leghemoglobin is collected in the flowthrough fractions and concentrated over 3 kDa filtration membrane to a desired concentration. Purity (partial abundance) of the purified leghemoglobin is analyzed by SDS-PAGE gel: in the lysate, leghemoglobin can be present at 20-40%, while after the anion-exchange purification, it can be present at 70-80%. In other embodiments, soybean leghemoglobin flowthrough from the anion-exchange chromatography is applied onto size-exclusion chromatography (Sephacryl S-100 HR, GE Healthcare). Soybean leghemoglobin is eluted as two fractions corresponding to dimeric and monomeric species. Purity (partial abundance) of leghemoglobin is analyzed by SDS-PAGE and can be ˜90-100%.

Proteins in legume root-nodule lysates can be transferred to 10 mM sodium carbonate pH 9.5, 50 mM sodium chloride buffer, filtered through 200 nm filter, and applied onto an anion-exchange chromatography column on a fast protein liquid chromatography instrument (GE Healthcare). Leghemoglobin can bind the anion exchange chromatography matrix and the bound leghemoglobin can be eluted using a sodium chloride gradient. Purity (partial abundance) of leghemoglobin can be analyzed by SDS-PAGE and can be ˜60-80%.

Undesired small molecules from legume roots can be removed from purified leghemoglobin by passing the leghemoglobin solution over the anion-exchange resin. In some embodiments, the anion exchange resin is FFQ, DEAE, Amberlite IRA900, Dowex 22, or Dowex 1×4. Leghemoglobin purified either by ammonium sulfate fractionation (60% wt/v and 90% wt/v ammonium sulfate) or by anion-exchange chromatography is buffer exchanged into 20 mM potassium phosphate pH 7.4, 100 mM sodium chloride, and the solution is passed over one of the above mentioned anion-exchange resins. Flowthrough that contains leghemoglobin can be collected.

The heme-containing polypeptide can also be recombinantly produced as described in the Examples. For instance, a non-symbiotic hemoglobin from mung bean can be recombinantly expressed in E. coli and purified using the anion-exchange chromatography or cation-exchange chromatography. The cell lysate can be loaded over FF-Q resin on a fast protein liquid chromatography instrument (GE Healthcare). Mung bean non-symbiotic hemoglobin elutes in the flowthrough fractions. Purity (partial abundance) of mung bean non-symbiotic hemoglobin is analyzed by SDS-PAGE and can be determined to be as a fraction of total protein. Representative abundance and purity can be about 12% in E. coli lysate, and about 31% after purification on FFQ. UV-Vis analysis of purified protein shows spectra characteristic of heme-bound protein.

Alternatively, the mung bean cell lysate can be loaded over a FF-S resin on a fast protein liquid chromatography instrument (GE Healthcare). Mung bean non-symbiotic hemoglobin can bind the FF-S column, and the bound fractions can be eluted using sodium chloride gradient (50 mM-1000 mM). Purity (partial abundance) of mung bean non-symbiotic hemoglobin can be analyzed by SDS-PAGE and can be in the following range: E. coli lysate about 13%, after purification on FFQ about 35%. UV-Vis analysis of the purified protein can show spectra characteristic of heme bound protein.

In certain embodiments, standard polypeptide synthesis techniques (e.g., liquid-phase polypeptide synthesis techniques or solid-phase polypeptide synthesis techniques) can be used to produce heme-containing polypeptides synthetically. In some embodiments, cell-free translation techniques can be used to produce heme-containing proteins synthetically.

The present disclosure contemplates purification of monomeric heme-containing polypeptides (e.g., leghemoglobin) or multimeric heme-containing polypeptides (e.g., tetrameric hemoglobin). In some embodiments, each subunit of the tetrameric hemoglobin may be individually expressed and isolated from inclusion bodies in E. coli. The denatured β-subunits can be refolded and reconstituted with hemin in the presence of native reduced α-chains to produce functional, tetrameric hemoglobin (e.g., Fronticelli et al. (1991) J Protein Chem 10:495-501). Alternatively, the α and β subunits can be coexpressed to produce a large amount of intact, soluble tetrameric hemoglobin in E. Coli or P. pastoris (e.g., Hoffman et al. (1990) Proc Natl Acad Sci USA 87:8521-8525; Anwised et al. (2016) Protein J 35:256-268). In certain embodiments, a genes can be fused to create a di-α gene, which is inserted in an operon with a copy of the β-gene to express a tetramer that does not dissociate into α1β1 dimers under a physiological condition (e.g., Shen et al. (1993) Proc Natl Acad Sci USA 90:8108-8112).

In some embodiments, the heme-containing polypeptide is sterilized, e.g., prior to combining with other components of a growth media. Sterilization may be by Ultra High Temperature (UHT) processing, autoclaving or filtering. In some embodiments, the heme-containing polypeptide is autoclaved. In some embodiments, the heme-containing polypeptide is filtered, e.g., the heme-containing polypeptide is sterilized prior to use (e.g., prior to addition to growth media or with sterilization of the growth media) in methods to culture hemoglobin-dependent bacteria (e.g., for use in a bacterial composition (e.g., a pharmaceutical composition) and/or for therapeutic use). The heme-containing polypeptides provided herein can allow for large-scale manufacture and/or GMP grade manufacture of hemoglobin-dependent bacteria, e.g., for therapeutic use and/or in a bacterial composition (e.g., a pharmaceutical composition).

Growth Media

In some embodiments, provided herein is growth media comprising a heme-containing polypeptide (e.g., a heme-containing polypeptide not sourced from an animal) disclosed herein. In certain embodiments, the growth media comprises an amount of a heme-containing polypeptide disclosed herein sufficient to support growth of hemoglobin-dependent bacteria (e.g., for use in a bacterial composition (e.g., a pharmaceutical composition) and/or for therapeutic use). In certain embodiments, the growth medium comprises at least 0.001 g/L, at least 0.005 g/L, at least 0.01 g/L, at least 0.02 g/L, at least 0.03 g/L, at least 0.04 g/L, at least 0.05 g/L, at least 0.06 g/L, at least 0.07 g/L, at least 0.08 g/L, at least 0.09 g/L, at least 0.1 g/L, at least 0.2 g/L, at least 0.3 g/L, at least 0.4 g/L, at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of a heme-containing polypeptide. In some embodiments, the growth media comprises at least 0.005 g/L and no more than 1 g/L of a heme-containing polypeptide. In some embodiments, the growth medium comprises about 0.02 g/L of a heme-containing polypeptide. In some embodiments, the growth medium comprises about 0.05 g/L of a heme-containing polypeptide. In some embodiments, the growth medium comprises about 0.1 g/L of a heme-containing polypeptide. In some embodiments, the growth medium comprises about 0.2 g/L of a heme-containing polypeptide. In some embodiments, the growth medium comprises about 0.5 g/L of a heme-containing polypeptide. In some embodiments, the growth medium comprises about 1 g/L of a heme-containing polypeptide disclosed herein. In some embodiments, the growth medium comprises about 0.01 g/L to about 0.2 g/L of a heme-containing polypeptide. In certain embodiments, the growth medium comprises at least 0.001 g/L, at least 0.005 g/L, at least 0.01 g/L, at least 0.02 g/L, at least 0.03 g/L, at least 0.04 g/L, at least 0.05 g/L, at least 0.06 g/L, at least 0.07 g/L, at least 0.08 g/L, at least 0.09 g/L, at least 0.1 g/L, at least 0.2 g/L, at least 0.3 g/L, at least 0.4 g/L, at least 0.5 g/L, at least 0.75 g/L, at least 1 g/L, at least 1.25 g/L, at least 1.5 g/L, at least 1.75 g/L, at least 2 g/L, at least 2.25 g/L, at least 2.5 g/L, at least 2.75 g/L, at least 3 g/L, at least 3.25 g/L, at least 3.5 g/L, at least 3.75 g/L, at least 4 g/L, or at least 4.25 g/L of a soy leghemoglobin. In some embodiments, the growth medium comprises about 0.02 g/L of a soy leghemoglobin. In some embodiments, the growth medium comprises about 0.05 g/L of a soy leghemoglobin. In some embodiments, the growth medium comprises about 0.1 g/L of a soy leghemoglobin. In some embodiments, the growth medium comprises about 0.2 g/L of a soy leghemoglobin. In some embodiments, the growth medium comprises about 0.5 g/L of a soy leghemoglobin. In some embodiments, the growth medium comprises about 1 g/L of a soy leghemoglobin. In some embodiments, the growth medium comprises about 0.01 g/L to about 0.2 g/L of a soy leghemoglobin. In some embodiments of the methods and compositions provided herein, the growth media does not comprise hemoglobin or a derivative thereof (or other heme-containing polypeptide) that is sourced from an animal. In some embodiments, the growth media does not comprise animal products.

In some embodiments the growth media may contain sugar, yeast extracts, plant based peptones, buffers, salts, trace elements, surfactants, anti-foaming agents, and/or vitamins.

In some embodiments, the growth media comprise yeast extract, soy peptone A2SC 19649, Soy peptone E110 19885, dipotassium phosphate, monopotassium phosphate, L-cysteine-HCl, ammonium chloride, glucidex 21 D, and/or glucose.

In some embodiments, the growth media comprises 5 g/L to 15 g/L yeast extract 19512. In some embodiments, the growth media comprises 10 g/L yeast extract 19512.

In some embodiments, the growth media comprises 10 g/L to 15 g/L soy peptone A2SC 19649. In some embodiments, the growth media comprises 12.5 g/L soy peptone A2SC 19649. In some embodiments, the growth media comprises 10 g/L soy peptone A2SC 19649.

In some embodiments, the growth media comprises 10 g/L to 15 g/L Soy peptone E110 19885. In some embodiments, the growth media comprises 12.5 g/L Soy peptone E110 19885. In some embodiments, the growth media comprises 10 g/L soy peptone E110 19885.

In some embodiments, the growth media comprises 1 g/L to 3 g/L dipotassium phosphate. In some embodiments, the growth media comprises 1.59 g/L dipotassium phosphate. In some embodiments, the growth media comprises 2.5 g/L dipotassium phosphate.

In some embodiments, the growth media comprises 0 g/L to 1.5 g/L monopotassium phosphate. In some embodiments, the growth media comprises 0.91 g/L monopotassium phosphate. In some embodiments, the growth media does not comprise monopotassium phosphate.

In some embodiments, the growth media comprises 0.1 g/L to 1.0 g/L L-cysteine-HCl. In some embodiments, the growth media comprises 0.5 g/L L-cysteine-HCl.

In some embodiments, the growth media comprises 0 g/L to 1.0 g/L ammonium chloride. In some embodiments, the growth media comprises 0.5 g/L ammonium chloride. In some embodiments, the growth media does not comprise ammonium chloride.

In some embodiments, the growth media comprises 0 g/L to 30 g/L glucidex 21 D. In some embodiments, the growth media comprises 25 g/L glucidex 21 D. In some embodiments, the growth media does not comprise glucidex 21 D.

In some embodiments, the growth media comprises 5 g/L to 15 g/L glucose. In some embodiments, the growth media comprises 10 g/L glucose. In some embodiments, the growth media comprises 5 g/L glucose.

In certain embodiments, the growth media comprises a heme-containing polypeptide provided herein, about 10 g/L yeast extract 19512, about 12.5 g/L soy peptone A2SC 19649, about 12.5 g/L soy peptone E110 19885, about 1.59 g/L dipotassium phosphate, about 0.91 g/L monopotassium phosphate, about 0.5 g/L ammonium chloride, about 25 g/L glucidex 21 D, and/or about 10 g/L glucose. In some embodiments, the growth medium is the growth medium of Table 4.

In certain embodiments, the growth media comprises a heme-containing polypeptide provided herein, about 10 g/L yeast extract 19512, about 10 g/L soy peptone A2SC 19649, about 10 g/L soy peptone E110 19885, about 2.5 g/L dipotassium phosphate, about 0.5 g/L L-cysteine-HCl, and/or about 5 g/L glucose. In some embodiments, the growth medium is the growth medium of Table 5.

In certain embodiments, the growth media is at a pH of 5.5 to 7.5. In some embodiments, the growth media is at a pH of about 6.5.

In some embodiments, prior to being added to the growth media, a heme-containing polypeptide is prepared as a liquid mixture and sterilized by autoclaving or filtration. In some embodiments, the heme-containing polypeptide is added to the growth media, which is then sterilized as described below.

In some embodiments, the media is sterilized. Sterilization may be by Ultra High Temperature (UHT) processing, autoclaving or filtering. The UHT processing is performed at very high temperature for short periods of time. The UHT range may be from 135-180° C. For example, the medium may be sterilized from between 10 to 30 seconds at 135° C.

Culturing Methods

In certain aspects, provided herein are methods and/or compositions that facilitate the growth of hemoglobin-dependent bacteria (e.g., for use in a bacterial composition (e.g., a pharmaceutical composition) and/or for therapeutic use). Such methods may comprise incubating the hemoglobin-dependent bacteria in a growth media provided herein. The methods may comprise maintaining the temperature and pH of the growth media as disclosed herein. The culturing may begin in a relatively small volume of growth media (e.g., 1 L) where bacteria are allowed to reach the log phase of growth. Such culture may be transferred to a larger volume of growth media (e.g., 20 L) for further growth to reach a larger biomass. Depending on the need of the final amount of biomass, such transfer may be repeated more than once. The methods may comprise the incubation of the hemoglobin-dependent bacteria in bioreactors.

In certain aspects, the hemoglobin-dependent bacteria are incubated at a temperature of 35° C. to 39° C. In some embodiments, the hemoglobin-dependent bacteria are incubated at a temperature of about 37° C.

In certain embodiments, the methods and/or compositions provided herein increase the growth rate of hemoglobin-dependent bacteria such that hemoglobin-dependent bacteria grow at an increased rate in the growth media comprising a heme-containing polypeptide disclosed herein, compared to the rate at which the hemoglobin-dependent bacteria grow in the same growth media but without the heme-containing polypeptide disclosed herein. In some embodiments, the rate at which the hemoglobin-dependent bacteria grow in the growth media comprising a heme-containing polypeptide disclosed herein is higher than the rate at which the hemoglobin-dependent bacteria grow in the same growth media but without the heme-containing polypeptide disclosed herein by at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400%. In some embodiments, the growth rate is increased by about 200% to about 400%. The rate may be measured as the cell density (as measured by e.g., optical density at the wavelength of 600 nm (OD600)) reached within a given amount of time. In certain embodiments, such rate is measured and compared during the log phase (or exponential phase) of the bacterial growth, optionally wherein the log phase is early log phase.

In certain embodiments, the methods and/or compositions provided herein increase the bacterial cell density such that the hemoglobin-dependent bacteria grow to a higher bacterial cell density in the growth media comprising a heme-containing polypeptide disclosed herein, compared to the cell density to which the hemoglobin-dependent bacteria grow in the same growth media but without the heme-containing polypeptide disclosed herein. In some embodiments, the hemoglobin-dependent bacteria grow to a cell density in the growth media comprising a heme-containing polypeptide disclosed herein is higher than the cell density to which the hemoglobin-dependent bacteria grow in the same growth media but without the heme-containing polypeptide disclosed herein by at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, or at least 400%. In some embodiments, the bacterial cell density higher than about 200% to about 400%. The cell density may be measured (e.g., by OD600 or by cell counting) at the stationary phase of bacterial growth, optionally wherein the stationary phase is early stationary phase. In some embodiments, the stationary phase is determined as the phase where the growth rate is retarded followed by an exponential phase of growth (e.g., from a growth curve). In other embodiments, the stationary phase is determined by the low glucose level in the growth media.

In some embodiments, the methods provided herein comprise incubating the hemoglobin-dependent bacteria under anaerobic atmosphere. In certain aspects, provided herein are methods of culturing hemoglobin-dependent bacteria under anaerobic atmosphere comprising CO₂. In some embodiments, the anaerobic atmosphere comprises greater than 1% CO₂. In some embodiments, the anaerobic atmosphere comprises greater than 5% CO₂. In some embodiments, the anaerobic atmosphere comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the anaerobic atmosphere comprises at least 10% CO₂. In some embodiments, the anaerobic atmosphere comprises at least 20% CO₂. In some embodiments, the anaerobic atmosphere comprises from 10% to 40% CO₂. In some embodiments, the anaerobic atmosphere comprises from 20% to 30% CO₂. In some embodiments, the anaerobic atmosphere comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% CO₂. In some embodiments, the anaerobic atmosphere comprises about 25% CO₂.

In certain aspects, the anaerobic atmosphere comprises N₂. In some embodiments, the anaerobic atmosphere comprises less than 95% N₂. In some embodiments, the anaerobic atmosphere comprises less than 90% N₂. In some embodiments, the anaerobic atmosphere comprises less than 95%, less than 92%, less than 90%, less than 87%, less than 85%, less than 82%, less than 80%, less than 77% N₂. In some embodiments, the anaerobic atmosphere comprises less than 85% N₂. In some embodiments, the anaerobic atmosphere comprises less than 80% N₂. In some embodiments, the anaerobic atmosphere comprises from 65% to 85% N₂. In some embodiments, the anaerobic atmosphere comprises from 70% to 80% N₂. In some embodiments, the anaerobic atmosphere comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic atmosphere comprises about 75% N₂.

In some embodiments, the anaerobic atmosphere consists essentially of CO₂ and N₂. In some embodiments, the anaerobic atmosphere comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

Thus, in some embodiments provided herein are methods of culturing hemoglobin-dependent bacteria under anaerobic conditions comprising a greater level of CO₂ compared to conventional anaerobic culture conditions (e.g., at a level of greater than 1% CO₂, e.g., at a level of greater than 5% CO₂, such as at a level of about 25% CO₂). In certain embodiments, provided herein are bioreactors comprising hemoglobin-dependent bacteria being cultured under conditions comprising a greater level of CO₂ compared to conventional anaerobic culture conditions (e.g., at a level of greater than 1% CO₂, such as at a level of about 25% CO₂). In some embodiments, the methods and compositions provided herein result in increased bacterial yield compared to conventional culture conditions.

In certain aspects, provided herein are methods of culturing hemoglobin-dependent bacteria under anaerobic conditions comprising a lower level of N₂ compared to conventional anaerobic culture conditions (e.g., at a level of less than 95% N₂, e.g., at a level of less than 90% N₂, such as at a level of about 75% N₂). In certain embodiments, provided herein are bioreactors comprising hemoglobin-dependent bacteria being cultured under conditions comprising a lower level of N₂ compared to conventional anaerobic culture conditions (e.g., at a level of less than 95% N₂ such as at a level of about 75% N₂). In some embodiments, the methods and compositions provided herein result in increased bacterial yield compared to conventional culture conditions.

In certain aspects, provided herein are methods of culturing hemoglobin-dependent bacteria, the method comprises the steps of a) purging a bioreactor with an anaerobic gaseous mixture comprising greater than 1% CO₂; and b) culturing the hemoglobin-dependent bacteria in the bioreactor purged in step a). In some embodiments, the anaerobic gaseous mixture comprises greater than 1% CO₂. In some embodiments, the anaerobic gaseous mixture comprises at least about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% CO₂. In some embodiments, the anaerobic gaseous mixture comprises at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CO₂. In some embodiments, the anaerobic gaseous mixture comprises from 5% to 35% CO₂, 10% to 40% CO₂, 10% to 30% CO₂, 15% to 30% CO₂, 20% to 30% CO₂, 22% to 28% CO₂, or 24%, to 26% CO₂. In some embodiments, the anaerobic gaseous mixture comprises greater than 5% CO₂. In some embodiments, the anaerobic gaseous mixture comprises at least 10% CO₂. In some embodiments, the anaerobic gaseous mixture comprises at least 20% CO₂. In some embodiments, the anaerobic gaseous mixture comprises from 10% to 40% CO₂. In some embodiments, the anaerobic gaseous mixture comprises from 20% to 30% CO₂. In some embodiments, the anaerobic gaseous mixture comprises about 25% CO₂.

In certain aspects, provided herein are methods of culturing hemoglobin-dependent bacteria, the method comprises the steps of a) purging a bioreactor with an anaerobic gaseous mixture comprising less than 95% N₂; and b) culturing the hemoglobin-dependent bacteria in the bioreactor purged in step a). In some embodiments, the anaerobic gaseous mixture comprises less than 95% N₂. In some embodiments, the anaerobic gaseous mixture comprises less than 95%, less than 92%, less than 90%, less than 87%, less than 85%, less than 82%, less than 80%, less than 77% N₂. In some embodiments, the anaerobic gaseous mixture comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic gaseous mixture comprises less than 95% N₂. In some embodiments, the anaerobic gaseous mixture comprises less than 90% N₂. In some embodiments, the anaerobic gaseous mixture comprises from 65% to 85% N₂. In some embodiments, the anaerobic gaseous mixture comprises from 70% to 80% N₂CO₂. In some embodiments, the anaerobic gaseous mixture comprises about 75% N₂.

In some embodiments, the anaerobic gaseous mixture consists essentially of CO₂ and N₂. In some embodiments, the anaerobic gaseous mixture comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

In some embodiments, the anaerobic gaseous mixture comprises CO₂ and N₂ in a ratio of about 1:99, about 2:98, about 3:97, about 4:96, about 5:95, about 6:94, about 7:93, about 8:92, about 9:91, about 10:90, 11:89, about 12:88, about 13:87, about 14:86, about 15:85, about 16:84, about 17:83, about 18:82, about 19:81, about 20:80, 21:79, about 22:78, about 23:77, about 24:76, about 25:75, about 26:74, about 27:73, about 28:72, about 29:71, about 30:70, 31:69, about 32:68, about 33:67, about 34:66, about 35:65, about 36:64, about 37:63, about 38:62, about 39:61, or about 40:50 CO₂ to N₂. In some embodiments, the mixed gas composition provides an atmosphere in the bioreactor comprising CO₂ and N₂ in a ratio of about 25:75.

In some embodiments, an anaerobic gaseous mixture is continuously added to the bioreactor during culturing. In some embodiments, the continuously added anaerobic gaseous mixture is added at a rate of 0.01 to 0.1 vvm. In some embodiments the continuously added anaerobic gaseous mixture is added at a rate of 0.02 vvm. In some embodiments, the continuously added anaerobic gaseous mixture comprises any one of gaseous mixtures described above.

In some embodiments, the methods provided herein further comprises the step of inoculating a growth media with the hemoglobin-dependent bacteria, wherein the bacteria are cultured in the growth media according to the methods provided herein. In some embodiments, the volume of the inoculated hemoglobin-dependent bacteria is between 0.01% and 10% v/v of the growth media (e.g., about 0.1% v/v of the growth media, about 0.5% v/v of the growth media, about 1% v/v of the growth media, about 5% v/v of the growth media). In some embodiments, the volume of hemoglobin-dependent bacteria is about 1 mL.

In some embodiments, inoculum can be prepared in flasks or in smaller bioreactors where growth is monitored. For example, the inoculum size may be between approximately 0.1% v/v and 5% v/v of the total bioreactor volume. In some embodiments, the inoculum is 0.1-3% v/v, 0.1-1% v/v, 0.1-0.5% v/v, or 0.5-1% v/v of the total bioreactor volume. In some embodiments, the inoculum is about 0.1% v/v, about 0.2% v/v, about 0.3% v/v, about 0.4%, v/v, about 0.5% v/v, about 0.6% v/v, about 0.7% v/v, about 0.8% v/v, about 0.9% v/v, about 1% v/v, about 1.5% v/v, about 2% v/v, about 2.5% v/v, about 3% v/v, about 4%, v/v, or about 5% v/v of the total bioreactor volume.

In some embodiments, before the inoculation, the bioreactor is prepared with growth medium at desired pH and temperature. The initial pH of the culture medium may be different than the process set-point. pH stress may be detrimental at low cell concentration; the initial pH could be between pH 7.5 and the process set-point. For example, pH may be set between 4.5 and 8.0, preferably 6.5. During the fermentation, the pH can be controlled through the use of sodium hydroxide, potassium hydroxide, or ammonium hydroxide. The temperature may be controlled from 25° C. to 45° C., for example at 37° C.

In some embodiments, depending on strain and inoculum size, the bioreactor fermentation time can vary. For example, fermentation time can vary from 5 hours to 48 hours. In some embodiments, fermentation time may be from 5 hours to 24 hours, 8 hours to 24 hours, 8 hours to 18 hours, 8 hours to 16 hours, 8 hours to 14 hours, 10 hours to 24 hours, 10 hours to 18 hours, 10 hours to 16 hours, 10 hours to 14 hours, 10 hours to 12 hours, 12 hours to 24 hours, 12 hours to 18 hours, 12 hours to 16 hours, or 12 hours to 14 hours.

In some embodiments, culturing the hemoglobin-dependent bacteria comprises agitating the culture at a RPM of 50 to 300. In some embodiments, the hemoglobin-dependent bacteria is agitated at a RPM of about 150.

For example, in some embodiments, a culturing method comprises culturing the hemoglobin-dependent bacteria for at least 5 hours (e.g., at least 10 hours). In some embodiments, the hemoglobin-dependent bacteria is cultured for 10-24 hours. In some embodiments, the hemoglobin-dependent bacteria is cultured for 14 to 16 hours. In some embodiments, the method further comprises the step of inoculating about 5% v/v of the cultured bacteria in a growth media. In some embodiments, the growth media is about 20 L in volume. In some embodiments, the hemoglobin-dependent bacteria is cultured for 10-24 hours. In some embodiments, the hemoglobin-dependent bacteria is cultured for 12-14 hours. In some embodiments, the method further comprises the step of inoculating about 0.5% v/v of the cultured bacteria in a growth medium. In some embodiments, the growth medium is about 3500 L in volume. In some embodiments, the hemoglobin-dependent bacteria is cultured for 10-24 hours. In some embodiments, the hemoglobin-dependent bacteria is cultured for 12-14 hours. In some embodiments, the hemoglobin-dependent bacteria is cultured at least until a stationary phase is reached.

In certain embodiments, the culturing method further comprises the step of harvesting the cultured bacteria. The harvest time may be based on either glucose level being below 2 g/L or when stationary phase is reached. In some embodiments, the method further comprises the step of centrifuging the cultured bacteria after harvesting (e.g., to produce a cell paste). In some embodiments, the method further comprises diluting the cell paste with a stabilizer solution to produce a cell slurry. In some embodiments, the method further comprises the step of lyophilizing the cell slurry to produce a powder. In some embodiments, the method further comprises irradiating the powder with gamma radiation.

For example, in some embodiments, once fermentation complete, the culture is cooled (e.g., to 10° C.) and centrifuged collecting the cell paste. A stabilizer may be added to the cell paste and mixed thoroughly. Harvesting may be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets may be mixed with excipients and submerged in liquid nitrogen.

In certain embodiments, the cell slurry may be lyophilized. Lyophilization of material, including live bacteria, may begin with primary drying. During the primary drying phase, the ice is removed. Here, a vacuum is generated and an appropriate amount of heat is supplied to the material for the ice to sublime. During the secondary drying phase, product bound water molecules may be removed. Here, the temperature is raised higher than in the primary drying phase to break any physico-chemical interactions that have formed between the water molecules and the product material. The pressure may also be lowered further to enhance desorption during this stage. After the freeze-drying process is complete, the chamber may be filled with an inert gas, such as nitrogen. The product may be sealed within the freeze dryer under dry conditions, preventing exposure to atmospheric water and contaminants. The lyophilized material may be gamma irradiated (e.g., 17.5 kGy).

Bioreactors

In certain aspects, provided herein are bioreactors comprising growth media provided herein (i.e., a growth media comprising a heme-containing polypeptide disclosed herein (e.g., a heme-containing polypeptide not sourced from an animal)) and/or hemoglobin-dependent bacteria (e.g., for use in a bacterial composition (e.g., a pharmaceutical composition) and/or for therapeutic use) provided herein. In some embodiments, the hemoglobin-dependent bacteria are Prevotella bacteria (e.g., a Prevotella strain provided herein). In some embodiments, provided herein are methods of culturing bacteria in such bioreactors.

In certain embodiments, the bioreactor is under the anaerobic conditions mentioned above. In certain aspects, provided herein are bioreactors comprising hemoglobin-dependent bacteria under an anaerobic atmosphere disclosed above. In certain aspects, provided herein are bioreactors of various sizes. In some embodiments, the bioreactors are at least 1 L in volume, at least 5 L in volume, at least 10 L in volume, at least 15 L in volume, at least 20 L in volume, at least 30 L in volume, at least 40 L in volume, at least 50 L in volume, at least 100 L in volume, at least 200 L in volume, at least 250 L in volume, at least 500 L in volume, at least 750 L in volume, at least 1000 L in volume, at least 1500 L in volume, at least 2000 L in volume, at least 2500 L in volume, at least 3000 L in volume, at least 3500 L in volume, at least 4000 L in volume, at least 5000 L in volume, at least 7500 L in volume, at least 10,000 L in volume, at least 15,000 L in volume, or at least 20,000 L in volume. In some embodiments, the bioreactors are about 1 L in volume, about 5 L in volume, about 10 L in volume, about 15 L in volume, about 20 L in volume, about 30 L in volume, about 40 L in volume, about 50 L in volume, about 100 L in volume, about 200 L in volume, about 250 L in volume, about 500 L in volume, about 750 L in volume, about 1000 L in volume, about 1500 L in volume, about 2000 L in volume, about 2500 L in volume, about 3000 L in volume, about 3500 L in volume, about 4000 L in volume, about 5000 L in volume, about 7500 L in volume, about 10,000 L in volume, about 15,000 L in volume, or about 20,000 L in volume.

### EXEMPLARY EMBODIMENTS

According to exemplary embodiment 1, provided herein is a method of culturing hemoglobin-dependent bacteria, the method comprising incubating the hemoglobin-dependent bacteria in a growth medium that comprises a heme-containing polypeptide, wherein the heme-containing polypeptide is: (i) a piscine polypeptide; (ii) an avian polypeptide; or (iii) a non-animal-derived polypeptide.

According to exemplary embodiment 2, provided herein is the method of embodiment 1, wherein the heme-containing polypeptide is a hemoglobin.

According to exemplary embodiment 3, provided herein is the method of embodiment 2, wherein the hemoglobin is a symbiotic hemoglobin, non-symbiotic hemoglobin, and/or truncated hemoglobin.

According to exemplary embodiment 4, provided herein is the method of embodiment 1, wherein the heme-containing polypeptide is a leghemoglobin.

According to exemplary embodiment 5, provided herein is the method of embodiment 1, wherein the heme-containing polypeptide is a myoglobin.

According to exemplary embodiment 6, provided herein is the method of any one of embodiments 1-5, wherein the heme-containing polypeptide is a piscine polypeptide.

According to exemplary embodiment 7, provided herein is the method of embodiment 6, wherein the piscine polypeptide is purified from fish.

According to exemplary embodiment 8, provided herein is the method of embodiment 7, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 9, provided herein is the method of embodiment 6, wherein the piscine polypeptide is recombinantly expressed.

According to exemplary embodiment 10, provided herein is the method of any one of embodiments 1-5, wherein the heme-containing polypeptide is an avian polypeptide.

According to exemplary embodiment 11, provided herein is the method of embodiment 10, wherein the avian polypeptide is purified from a bird.

According to exemplary embodiment 12, provided herein is the method of embodiment 11, wherein the bird is of the genus Gallus, Meleagris, Anas, Anser, Branta, Chen, Agelastes, Numida, Guttera, Acryllium, or Columba.

According to exemplary embodiment 13, provided herein is the method of embodiment 10, wherein the avian polypeptide is recombinantly expressed.

In exemplary embodiment 14, provided herein is the method of any one of embodiments 1-5, wherein the heme-containing polypeptide is a non-animal-derived polypeptide.

In exemplary embodiment 15, provided herein is the method of embodiment 14, wherein the non-animal-derived polypeptide is purified from a plant (e.g., soy), bacteria, cyanobacteria, fungus, algae, grain, legume, and/or protozoa.

According to exemplary embodiment 16, provided herein is the method of embodiment 14 or 15, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 17, provided herein is the method of any one of embodiments 14-16, wherein the non-animal-derived polypeptide is a soy leghemoglobin.

According to exemplary embodiment 18, provided herein is the method of embodiment 17, wherein the soy leghemoglobin is purified from soy roots or soy root nodules.

According to exemplary embodiment 19, provided herein is the method of embodiment 14, wherein the non-animal-derived polypeptide is recombinantly expressed.

According to exemplary embodiment 20, provided herein is the method of any one of embodiments 1-5, 14, and 19, wherein the heme-containing polypeptide comprises an amino acid sequence with at least 70% identity to an amino acid sequence in any one of SEQ ID NOs: 1-29, 31, 33, 35, 37, 39, 41, 43, 114 (e.g., SEQ ID NO: 4 or 114) or a combination thereof.

According to exemplary embodiment 21, provided herein is the method of embodiment 20, wherein the heme-containing polypeptide comprises an amino acid sequence with at least 90% identity (or at least 99.5% or 100%) to an amino acid sequence in any one of SEQ ID NOs: 1-29, 31, 33, 35, 37, 39, 41, 43, 114 (e.g., SEQ ID NO: 4 or 114) or a combination thereof.

According to exemplary embodiment 22, provided herein is the method of any one of embodiments 9, 13, and 19, wherein the recombinantly expressed polypeptide comprises a heterologous polypeptide.

According to exemplary embodiment 23, provided herein is the method of embodiment 22, wherein the heterologous polypeptide comprises a histidine tag, TAP (tandem affinity purification) tag, TEV cleavage site, a FLAG tag, a GST tag, and/or an immunoglobulin domain.

According to exemplary embodiment 24, provided herein is the method of any one of embodiments 9, 13, and 19-23, wherein the recombinant polypeptide is expressed and purified from an exogenous nucleic acid in a host cell.

According to exemplary embodiment 25, provided herein is the method of embodiment 24, wherein the exogenous nucleic acid is in a vector.

According to exemplary embodiment 26, provided herein is the method of embodiment 25, wherein the vector is an expression vector.

According to exemplary embodiment 27, provided herein is the method of any one of embodiments 24-26, wherein the host cell is bacteria, yeast, insect, or mammalian cell lines.

According to exemplary embodiment 28, provided herein is the method of embodiment 27, wherein the host cell is Pichia Pastoris or Escherichia coli.

According to exemplary embodiment 29, provided herein is the method of any one of embodiments 19-28, wherein the heme-containing polypeptide is a soy leghemoglobin recombinantly expressed in Pichia pastoris.

According to exemplary embodiment 30, provided herein is the method of any one of embodiments 1-29, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

According to exemplary embodiment 31, provided herein is the method of any one of embodiments 1-29, wherein the hemoglobin-dependent bacteria are of the genus Prevotella.

According to exemplary embodiment 32, provided herein is the method of embodiment 31, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 33, provided herein is the method of embodiment 31, wherein the hemoglobin-dependent bacteria are of the species Prevotella histicola.

According to exemplary embodiment 34, provided herein is the method of embodiment 31, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 35, provided herein is the method of embodiment 31, wherein the Prevotella comprise at least 99.5% genomic, 16S and/or CRISPR sequence identity to the nucleotide sequence of the Prevotella Strain B 50329 (NRRL accession number B 50329) or Prevotella Strain C (PTA-126140).

According to exemplary embodiment 36, provided herein is the method of embodiment 31, wherein the Prevotella are Prevotella Strain B 50329 (NRRL accession number B 50329) or Prevotella Strain C (PTA-126140).

According to exemplary embodiment 37, provided herein is the method of any one of embodiments 31-36, wherein the hemoglobin-dependent bacteria are a strain of Prevotella bacteria comprising one or more proteins listed in Table 1.

According to exemplary embodiment 38, provided herein is the method of any one of embodiments 31-37, wherein the hemoglobin-dependent bacteria are from a strain of Prevotella substantially free of a protein listed in Table 2.

According to exemplary embodiment 39, provided herein is the method of any one of embodiments 1-38, wherein the hemoglobin-dependent bacteria grow at an increased rate in the growth medium comprising the heme-containing polypeptide compared to the rate at which the hemoglobin-dependent bacteria grow in the same growth medium but without the heme-containing polypeptide.

According to exemplary embodiment 40, provided herein is the method of embodiment 39, wherein the rate at which the hemoglobin-dependent bacteria grow in the growth medium comprising the heme-containing polypeptide is at least 50% higher than the rate at which the hemoglobin-dependent bacteria grow in the same growth medium but without the heme-containing polypeptide.

According to exemplary embodiment 41, provided herein is the method of embodiment 39, wherein the rate at which the hemoglobin-dependent bacteria grow in the growth medium comprising the heme-containing polypeptide is at least 100% higher than the rate at which the hemoglobin-dependent bacteria grow in the same growth medium but without the heme-containing polypeptide.

According to exemplary embodiment 42, provided herein is the method of embodiment 39, wherein the rate at which the hemoglobin-dependent bacteria grow in the growth medium comprising the heme-containing polypeptide is 200% to 400% higher than the rate at which the hemoglobin-dependent bacteria grow in the same growth medium but without the heme-containing polypeptide.

According to exemplary embodiment 43, provided herein is the method of embodiment 39, wherein the rate at which the hemoglobin-dependent bacteria grow in the growth medium comprising the heme-containing polypeptide is at least 300% higher than the rate at which the hemoglobin-dependent bacteria grow in the same growth medium but without the heme-containing polypeptide.

According to exemplary embodiment 44, provided herein is the method of any one of embodiments 1-43, wherein the hemoglobin-dependent bacteria grow to a higher cell density in the growth medium comprising the heme-containing polypeptide, compared to the cell density to which the hemoglobin-dependent bacteria grow in the same growth medium but without the heme-containing polypeptide.

According to exemplary embodiment 45, provided herein is the method of embodiment 44, wherein the hemoglobin-dependent bacteria grow to a cell density in the growth medium comprising the heme-containing polypeptide that is at least 50% higher than the cell density to which the hemoglobin-dependent bacteria grow in the same growth medium but without the heme-containing polypeptide.

According to exemplary embodiment 46, provided herein is the method of embodiment 44, wherein the hemoglobin-dependent bacteria grow to a cell density in the growth medium comprising the heme-containing polypeptide that is at least 100% higher than the cell density to which the hemoglobin-dependent bacteria grow in the same growth medium but without the heme-containing polypeptide.

According to exemplary embodiment 47, provided herein is the method of embodiment 44, wherein the hemoglobin-dependent bacteria grow to a cell density in the growth medium comprising the heme-containing polypeptide that is at 200% to 400% higher than the cell density to which the hemoglobin-dependent bacteria grow in the same growth medium but without the heme-containing polypeptide.

According to exemplary embodiment 48, provided herein is the method of embodiment 44, wherein the hemoglobin-dependent bacteria grow to a cell density in the growth medium comprising the heme-containing polypeptide that is at least 300% higher than the cell density to which the hemoglobin-dependent bacteria grow in the same growth medium but without the heme-containing polypeptide.

According to exemplary embodiment 49, provided herein is the method of any one of embodiments 1-48, wherein the method comprises incubating the hemoglobin-dependent bacteria under an anaerobic atmosphere comprising greater than 1% CO₂.

According to exemplary embodiment 50, provided herein is the method of embodiment 49, wherein the anaerobic atmosphere comprises at least 10% CO₂.

According to exemplary embodiment 51, provided herein is the method of embodiment 49, wherein the anaerobic atmosphere comprises at least 20% CO₂.

According to exemplary embodiment 52, provided herein is the method of embodiment 49, wherein the anaerobic atmosphere comprises from 10% to 40% CO₂.

According to exemplary embodiment 53, provided herein is the method of embodiment 49, wherein the anaerobic atmosphere comprises from 20% to 30% CO₂.

According to exemplary embodiment 54, provided herein is the method of embodiment 49, wherein the anaerobic atmosphere comprises about 25% CO₂.

According to exemplary embodiment 55, provided herein is the method of any one of embodiments 49-54, wherein the anaerobic atmosphere consists essentially of CO₂ and N₂.

According to exemplary embodiment 56, provided herein is the method of embodiment 49 wherein the anaerobic atmosphere comprises about 25% CO₂ and about 75% N₂.

According to exemplary embodiment 57, provided herein is the method of embodiment 46, wherein the method comprises the steps of a) purging a bioreactor with an anaerobic gaseous mixture comprising greater than 1% CO₂; and b) incubating the hemoglobin-dependent bacteria in the bioreactor purged in step a).

According to exemplary embodiment 58, provided herein is the method of embodiment 57, wherein the anaerobic gaseous mixture comprises at least 10% CO₂.

According to exemplary embodiment 59, provided herein is the method of embodiment 57, wherein the anaerobic gaseous mixture comprises at least 20% CO₂.

According to exemplary embodiment 60, provided herein is the method of embodiment 57, wherein the anaerobic gaseous mixture comprises from 10% to 40% CO₂.

According to exemplary embodiment 61, provided herein is the method of embodiment 57, wherein the anaerobic gaseous mixture comprises from 20% to 30% CO₂.

According to exemplary embodiment 62, provided herein is the method of embodiment 57, wherein the anaerobic gaseous mixture comprises about 25% CO₂.

According to exemplary embodiment 63, provided herein is the method of any one of embodiments 57-62, wherein the anaerobic gaseous mixture consists essentially of CO₂ and N₂.

According to exemplary embodiment 64, provided herein is the method of embodiment 57, wherein the anaerobic gaseous mixture comprises about 25% CO₂ and about 75% N₂.

According to exemplary embodiment 65, provided herein is the method of any one of embodiments 57-64, wherein the bioreactor is an about 1 L, about 20 L, about 3,500 L, or about 20,000 L bioreactor.

According to exemplary embodiment 66, provided herein is the method of any one of embodiments 57-65, wherein the method further comprises the step of inoculating a growth medium with hemoglobin-dependent bacteria, wherein the inoculation step precedes step b).

According to exemplary embodiment 67, provided herein is the method of embodiment 66, wherein the volume of hemoglobin-dependent bacteria is about 0.1% v/v of the growth medium.

According to exemplary embodiment 68, provided herein is the method of embodiment 66, wherein the growth medium is about 1 L in volume.

According to exemplary embodiment 69, provided herein is the method of embodiment 66, wherein the volume of hemoglobin-dependent bacteria is about 1 mL.

According to exemplary embodiment 70, provided herein is the method of any one of embodiments 57-69, wherein the hemoglobin-dependent bacteria is incubated for 10-24 hours.

According to exemplary embodiment 71, provided herein is the method of embodiment 70, wherein the hemoglobin-dependent bacteria is incubated for 14 to 16 hours.

According to exemplary embodiment 72, provided herein is the method of embodiment 70 or 71, wherein the method further comprises the step of inoculating about 5% v/v of the cultured bacteria in a growth medium.

According to exemplary embodiment 73, provided herein is the method of embodiment 72, wherein the growth medium is about 20 L in volume.

According to exemplary embodiment 74, provided herein is the method of embodiment 72 or 73, wherein the hemoglobin-dependent bacteria is incubated for 10-24 hours.

According to exemplary embodiment 75, provided herein is the method of embodiment 74, wherein the hemoglobin-dependent bacteria is incubated for 12 to 14 hours.

According to exemplary embodiment 76, provided herein is the method of embodiment 74 or 75, wherein the method further comprises the step of inoculating about 0.5% v/v of the cultured bacteria in a growth medium.

According to exemplary embodiment 77, provided herein is the method of embodiment 76, wherein the growth medium is about 3500 L in volume.

According to exemplary embodiment 78, provided herein is the method of embodiment 76 or 77, wherein the hemoglobin-dependent bacteria is incubated for 10-24 hours.

According to exemplary embodiment 79, provided herein is the method of embodiment 78, wherein the hemoglobin-dependent bacteria is incubated for 12 to 14 hours.

According to exemplary embodiment 80, provided herein is the method of any one of embodiments 1 to 79, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

According to exemplary embodiment 81, provided herein is the method of embodiment 80, wherein the growth medium comprises 5 g/L to 15 g/L yeast extract 19512.

According to exemplary embodiment 82, provided herein is the method of embodiment 80, wherein the growth medium comprises about 10 g/L yeast extract 19512.

According to exemplary embodiment 83, provided herein is the method of any one of embodiments 80 to 82, wherein the growth medium comprises 10 g/L to 15 g/L soy peptone A2SC 19649.

According to exemplary embodiment 84, provided herein is the method of embodiment 83, wherein the growth medium comprises about 12.5 g/L soy peptone A2SC 19649.

According to exemplary embodiment 85, provided herein is the method of embodiment 83, wherein the growth medium comprises about 10 g/L soy peptone A2SC 19649.

According to exemplary embodiment 86, provided herein is the method of any one of embodiments 80 to 85, wherein the growth medium comprises 10 g/L to 15 g/L Soy peptone E110 19885.

According to exemplary embodiment 87, provided herein is the method of embodiment 86, wherein the growth medium comprises about 12.5 g/L Soy peptone E110 19885.

According to exemplary embodiment 88, provided herein is the method of embodiment 86, wherein the growth medium comprises about 10 g/L soy peptone E110 19885.

According to exemplary embodiment 89, provided herein is the method of any one of embodiments 80 to 88, wherein the growth medium comprises 1 g/L to 3 g/L dipotassium phosphate.

According to exemplary embodiment 90, provided herein is the method of embodiment 89, wherein the growth medium comprises about 1.59 g/L dipotassium phosphate.

According to exemplary embodiment 91, provided herein is the method of embodiment 89, wherein the growth medium comprises about 2.5 g/L dipotassium phosphate.

According to exemplary embodiment 92, provided herein is the method of any one of embodiments 80 to 91, wherein the growth medium comprises 0.5 g/L to 1.5 g/L monopotassium phosphate.

According to exemplary embodiment 93, provided herein is the method of embodiment 92, wherein the growth medium comprises about 0.91 g/L monopotassium phosphate.

According to exemplary embodiment 94, provided herein is the method of any one of embodiments 80 to 93, wherein the growth medium comprises 0.1 g/L to 1.0 g/L L-cysteine-HCl.

According to exemplary embodiment 95, provided herein is the method of embodiment 94, wherein the growth medium comprises about 0.5 g/L L-cysteine-HCl.

According to exemplary embodiment 96, provided herein is the method of any one of embodiments 80 to 95, wherein the growth medium comprises 0.1 g/L to 1.0 g/L ammonium chloride.

According to exemplary embodiment 97, provided herein is the method of embodiment 96, wherein the growth medium comprises about 0.5 g/L ammonium chloride.

According to exemplary embodiment 98, provided herein is the method of any one of embodiments 80 to 97, wherein the growth medium comprises 20 g/L to 30 g/L glucidex 21 D.

According to exemplary embodiment 99, provided herein is the method of embodiment 98, wherein the growth medium comprises about 25 g/L glucidex 21 D.

According to exemplary embodiment 100, provided herein is the method of any one of embodiments 80 to 99, wherein the growth medium comprises 5 g/L to 15 g/L glucose.

According to exemplary embodiment 101, provided herein is the method of embodiment 100, wherein the growth medium comprises about 5 g/L glucose or about 10 g/L glucose.

According to exemplary embodiment 102, provided herein is the method of any one of embodiments 1-101, wherein the growth medium comprises at least 0.001 g/L of the heme-containing polypeptide.

According to exemplary embodiment 103, provided herein is the method of embodiment 102, wherein the growth medium comprises at least 0.005 g/L of the heme-containing polypeptide.

According to exemplary embodiment 104, provided herein is the method of embodiment 102, wherein the growth medium comprises at least 0.01 g/L the heme-containing polypeptide.

According to exemplary embodiment 105, provided herein is the method of embodiment 102, wherein the growth medium comprises at least 0.02 g/L of the heme-containing polypeptide.

According to exemplary embodiment 106, provided herein is the method of embodiment 102, wherein the growth medium comprises about 0.1 g/L of the heme-containing polypeptide.

According to exemplary embodiment 107, provided herein is the method of embodiment 102, wherein the growth medium comprises about 0.2 g/L of the heme-containing polypeptide.

According to exemplary embodiment 108, provided herein is the method of embodiment 102, wherein the growth medium comprises at least 0.001 g/L of a soy leghemoglobin.

According to exemplary embodiment 109, provided herein is the method of embodiment 108, wherein the growth medium comprises at least 0.005 g/L of a soy leghemoglobin.

According to exemplary embodiment 110, provided herein is the method of embodiment 108, wherein the growth medium comprises at least 0.01 g/L of a soy leghemoglobin.

According to exemplary embodiment 111, provided herein is the method of embodiment 108, wherein the growth medium comprises at least 0.02 g/L of a soy leghemoglobin.

According to exemplary embodiment 112, provided herein is the method of embodiment 108, wherein the growth medium comprises about 0.1 g/L of a soy leghemoglobin.

According to exemplary embodiment 113, provided herein is the method of embodiment 108, wherein the growth medium comprises about 0.2 g/L of a soy leghemoglobin.

According to exemplary embodiment 114, provided herein is the method of any one of embodiments 1-113, wherein the hemoglobin-dependent bacteria is incubated at a temperature of 35° C. to 39° C.

According to exemplary embodiment 115, provided herein is the method of embodiment 114, wherein the hemoglobin-dependent bacteria is incubated at a temperature of about 37° C.

According to exemplary embodiment 116, provided herein is the method of any one of embodiments 1-115, wherein the growth medium is at a pH of 5.5 to 7.5.

According to exemplary embodiment 117, provided herein is the method of embodiment 116, wherein the growth medium is at a pH of about 6.5.

According to exemplary embodiment 118, provided herein is the method of any one of embodiments 1-117, wherein incubating the hemoglobin-dependent bacteria comprises agitating the growth medium at a RPM of 50 to 300.

According to exemplary embodiment 119, provided herein is the method of embodiment 118, wherein the growth medium is agitated at a RPM of about 150.

According to exemplary embodiment 120, provided herein is the method of any one of embodiments 57-119, wherein the anaerobic gaseous mixture is continuously added during incubation.

According to exemplary embodiment 121, provided herein is the method of embodiment 120, wherein the anaerobic gaseous mixture is added at a rate of about 0.02 vvm.

According to exemplary embodiment 122, provided herein is the method of any one of embodiments 1-121, wherein the method further comprises the step of harvesting the hemoglobin-dependent bacteria when a stationary phase is reached.

According to exemplary embodiment 123, provided herein is the method of embodiment 122, further comprising the step of centrifuging the hemoglobin-dependent bacteria after harvesting to produce a cell paste.

According to exemplary embodiment 124, provided herein is the method of embodiment 123, further comprising diluting the cell paste with a stabilizer solution to produce a cell slurry.

According to exemplary embodiment 125, provided herein is the method of embodiment 124, further comprising the step of lyophilizing the cell slurry to produce a powder.

According to exemplary embodiment 126, provided herein is the method of embodiment 125, further comprising irradiating the powder with gamma radiation.

According to exemplary embodiment 127, provided herein is a method of culturing hemoglobin-dependent bacteria, the method comprising (a) adding a heme-containing polypeptide and hemoglobin-dependent bacteria to a growth medium; and (b) incubating the hemoglobin-dependent bacteria in the growth medium, wherein the heme-containing polypeptide is: (i) a piscine polypeptide; (ii) an avian polypeptide; or (iii) a non-animal-derived polypeptide.

According to exemplary embodiment 128, provided herein is the method of embodiment 127, wherein the heme-containing polypeptide is a hemoglobin.

According to exemplary embodiment 129, provided herein is the method of embodiment 128, wherein the hemoglobin is a symbiotic hemoglobin, non-symbiotic hemoglobin, and/or truncated hemoglobin.

According to exemplary embodiment 130, provided herein is the method of embodiment 127, wherein the heme-containing polypeptide is a leghemoglobin.

According to exemplary embodiment 131, provided herein is the method of embodiment 127, wherein the heme-containing polypeptide is a myoglobin.

According to exemplary embodiment 132, provided herein is the method of any one of embodiments 127-131, wherein the heme-containing polypeptide is a piscine polypeptide.

According to exemplary embodiment 133, provided herein is the method embodiment 132, wherein the piscine polypeptide is purified from fish.

According to exemplary embodiment 134, provided herein is the method of embodiment 133, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, Ictalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 135, provided herein is the method of embodiment 132, wherein the piscine polypeptide is recombinantly expressed.

According to exemplary embodiment 136, provided herein is the method of any one of embodiments 127-131, wherein the heme-containing polypeptide is an avian polypeptide.

According to exemplary embodiment 137, provided herein is the method of embodiment 136, wherein the avian polypeptide is purified from a bird.

According to exemplary embodiment 138, provided herein is the method of embodiment 137, wherein the bird is of the genus Gallus, Meleagris, Anas, Anser, Branta, Chen, Agelastes, Numida, Guttera, Acryllium, or Columba.

According to exemplary embodiment 139, provided herein is the method of embodiment 136, wherein the avian polypeptide is recombinantly expressed.

According to exemplary embodiment 140, provided herein is the method of any one of embodiments 127-131, wherein the heme-containing polypeptide is a non-animal-derived polypeptide.

According to exemplary embodiment 141, provided herein is the method of embodiment 140, wherein the non-animal-derived polypeptide is purified from plants, bacteria, cyanobacteria, fungus, algae, grain, legume, and/or protozoa.

According to exemplary embodiment 142, provided herein is the method of embodiment 140 or 141, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 143, provided herein is the method of any one of embodiments 140-142, wherein the non-animal-derived polypeptide is a soy leghemoglobin.

According to exemplary embodiment 144, provided herein is the method of embodiment 143, wherein the soy leghemoglobin is purified from soy roots or soy root nodules.

According to exemplary embodiment 145, provided herein is the method of embodiment 140, wherein the non-animal-derived polypeptide is recombinantly expressed.

According to exemplary embodiment 146, provided herein is the method of any one of embodiments 127-131, 140, and 145, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

According to exemplary embodiment 147, provided herein is the method of embodiment 146, wherein the heme-containing polypeptide comprises an amino acid sequence with at least 90% identity to an amino acid sequence in any one of SEQ ID NOs: 1-29, 31, 33, 35, 37, 39, 41, 43, 114, or a combination thereof.

According to exemplary embodiment 148, provided herein is the method of any one of embodiments 135, 139, and 145, wherein the recombinantly expressed polypeptide comprises a heterologous polypeptide.

According to exemplary embodiment 149, provided herein is the method of embodiment 148, wherein the heterologous polypeptide comprises a histidine tag, TAP (tandem affinity purification) tag, TEV cleavage site, a FLAG tag, a GST tag, and/or an immunoglobulin domain.

According to exemplary embodiment 150, provided herein is the method of any one of embodiments 135, 139, and 145-149, wherein the recombinant polypeptide is expressed and purified from an exogenous nucleic acid in a host cell.

According to exemplary embodiment 151, provided herein is the method of embodiment 150, wherein the exogenous nucleic acid is in a vector.

According to exemplary embodiment 152, provided herein is the method of embodiment 151, wherein the vector is an expression vector.

According to exemplary embodiment 153, provided herein is the method of any one of embodiments 150-152, wherein the host cell is bacteria, yeast, insect, or mammalian cell lines.

According to exemplary embodiment 154, provided herein is the method of embodiment 153, wherein the host cell is Pichia Pastoris or Escherichia coli.

According to exemplary embodiment 155, provided herein is the method of any one of embodiments 145-154, wherein the heme-containing polypeptide is a soy leghemoglobin recombinantly expressed in Pichia pastoris.

According to exemplary embodiment 156, provided herein is the method of embodiment 127-155, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

According to exemplary embodiment 157, provided herein is the method of embodiment 127-155, wherein the hemoglobin-dependent bacteria are of the genus Prevotella.

According to exemplary embodiment 158, provided herein is the method of embodiment 157, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 159, provided herein is the method of embodiment 157, wherein the hemoglobin-dependent bacteria are of the species Prevotella histicola.

According to exemplary embodiment 160, provided herein is the method of embodiment 157, wherein the Prevotella comprise at least 90% genomic, 16S and/or CRISPR sequence identity to the nucleotide sequence of the Prevotella Strain B 50329 (NRRL accession number B 50329) or Prevotella Strain C (PTA-126140).

According to exemplary embodiment 161, provided herein is the method of embodiment 157, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 162, provided herein is the method of embodiment 157, wherein the Prevotella are Prevotella Strain B 50329 (NRRL accession number B 50329) or Prevotella Strain C (PTA-126140).

According to exemplary embodiment 163, provided herein is the method of any one of embodiments 157-162, wherein the hemoglobin-dependent bacteria are a strain of Prevotella bacteria comprising one or more proteins listed in Table 1.

According to exemplary embodiment 164, provided herein is the method of any one of embodiments 157-163, wherein the hemoglobin-dependent bacteria are a strain of Prevotella substantially free of a protein listed in Table 2.

According to exemplary embodiment 165, provided herein is the method of any one of embodiments 127-164, wherein the hemoglobin-dependent bacteria grow at an increased rate in the growth medium comprising the heme-containing polypeptide compared to the rate at which the hemoglobin-dependent bacteria grow in the same growth medium but without the heme-containing polypeptide.

According to exemplary embodiment 166, provided herein is the method of embodiment 165, wherein the rate at which the hemoglobin-dependent bacteria grow in the growth medium comprising the heme-containing polypeptide is at least 50% higher than the rate at which the hemoglobin-dependent bacteria grow in the same growth medium but without the heme-containing polypeptide.

According to exemplary embodiment 167, provided herein is the method of embodiment 165, wherein the rate at which the hemoglobin-dependent bacteria grow in the growth medium comprising the heme-containing polypeptide is at least 100% higher than the rate at which the hemoglobin-dependent bacteria grow in the same growth medium but without the heme-containing polypeptide.

According to exemplary embodiment 168, provided herein is the method of embodiment 165, wherein the rate at which the hemoglobin-dependent bacteria grow in the growth medium comprising the heme-containing polypeptide is 200% to 400% higher than the rate at which the hemoglobin-dependent bacteria grow in the same growth medium but without the heme-containing polypeptide.

According to exemplary embodiment 169, provided herein is the method of embodiment 165, wherein the rate at which the hemoglobin-dependent bacteria grow in the growth medium comprising the heme-containing polypeptide is at least 300% higher than the rate at which the hemoglobin-dependent bacteria grow in the same growth medium but without the heme-containing polypeptide.

According to exemplary embodiment 170, provided herein is the method of any one of embodiments 127-169, wherein the hemoglobin-dependent bacteria grow to a higher cell density in the growth medium comprising the heme-containing polypeptide, compared to the cell density to which the hemoglobin-dependent bacteria grow in the same growth medium but without the heme-containing polypeptide.

According to exemplary embodiment 171, provided herein is the method of embodiment 170, wherein the hemoglobin-dependent bacteria grow to a cell density in the growth medium comprising the heme-containing polypeptide that is at least 50% higher than the cell density to which the hemoglobin-dependent bacteria grow in the same growth medium but without the heme-containing polypeptide.

According to exemplary embodiment 172, provided herein is the method of embodiment 170, wherein the hemoglobin-dependent bacteria grow to a cell density in the growth medium comprising the heme-containing polypeptide that is at least 100% higher than the cell density to which the hemoglobin-dependent bacteria grow in the same growth medium but without the heme-containing polypeptide.

According to exemplary embodiment 173, provided herein is the method of embodiment 170, wherein the hemoglobin-dependent bacteria grow to a cell density in the growth medium comprising the heme-containing polypeptide that is at 200% to 400% higher than the cell density to which the hemoglobin-dependent bacteria grow in the same growth medium but without the heme-containing polypeptide.

According to exemplary embodiment 174, provided herein is the method of embodiment 170, wherein the hemoglobin-dependent bacteria grow to a cell density in the growth medium comprising the heme-containing polypeptide that is at least 300% higher than the cell density to which the hemoglobin-dependent bacteria grow in the same growth medium but without the heme-containing polypeptide.

According to exemplary embodiment 175, provided herein is the method of any one of embodiments 127-174, wherein the method comprises incubating the hemoglobin-dependent bacteria under an anaerobic atmosphere comprising greater than 1% CO₂.

According to exemplary embodiment 176, provided herein is the method of embodiment 175, wherein the anaerobic atmosphere comprises at least 10% CO₂.

According to exemplary embodiment 177, provided herein is the method of embodiment 175, wherein the anaerobic atmosphere comprises at least 20% CO₂.

According to exemplary embodiment 178, provided herein is the method of embodiment 175, wherein the anaerobic atmosphere comprises from 10% to 40% CO₂.

According to exemplary embodiment 179, provided herein is the method of embodiment 175, wherein the anaerobic atmosphere comprises from 20% to 30% CO₂.

According to exemplary embodiment 180, provided herein is the method of embodiment 175, wherein the anaerobic atmosphere comprises about 25% CO₂.

According to exemplary embodiment 181, provided herein is the method of any one of embodiments 175-180, wherein the anaerobic atmosphere consists essentially of CO₂ and Nz.

According to exemplary embodiment 182, provided herein is the method of embodiment 175, wherein the anaerobic atmosphere comprises about 25% CO₂ and about 75% N₂.

According to exemplary embodiment 183, provided herein is the method of embodiment 175, wherein the method comprises the steps of a) purging a bioreactor with an anaerobic gaseous mixture comprising greater than 1% CO₂; and b) incubating the hemoglobin-dependent bacteria in the bioreactor purged in step a).

According to exemplary embodiment 184, provided herein is the method of embodiment 183, wherein the anaerobic gaseous mixture comprises at least 10% CO₂.

According to exemplary embodiment 185, provided herein is the method of embodiment 183, wherein the anaerobic gaseous mixture comprises at least 20% CO₂.

According to exemplary embodiment 186, provided herein is the method of embodiment 183, wherein the anaerobic gaseous mixture comprises from 10% to 40% CO₂.

According to exemplary embodiment 187, provided herein is the method of embodiment 183, wherein the anaerobic gaseous mixture comprises from 20% to 30% CO₂.

According to exemplary embodiment 188, provided herein is the method of embodiment 183, wherein the anaerobic gaseous mixture comprises about 25% CO₂.

According to exemplary embodiment 189, provided herein is the method of any one of embodiments 183-188, wherein the anaerobic gaseous mixture consists essentially of CO₂ and N₂.

According to exemplary embodiment 190, provided herein is the method of embodiment 183, wherein the anaerobic gaseous mixture comprises about 25% CO₂ and about 75% N₂.

According to exemplary embodiment 191, provided herein is the method of any one of embodiments 183-190, wherein the bioreactor is an about 1 L, about 20 L, about 3,500 L, or about 20,000 L bioreactor.

According to exemplary embodiment 192, provided herein is the method of any one of embodiments embodiment 183-191, wherein the method further comprises the step of inoculating a growth medium with hemoglobin-dependent bacteria, wherein the inoculation step precedes step b).

According to exemplary embodiment 193, provided herein is the method of embodiment 192, wherein the volume of hemoglobin-dependent bacteria is about 0.1% v/v of the growth medium.

According to exemplary embodiment 194, provided herein is the method of embodiment 192, wherein the growth medium is about 1 L in volume.

According to exemplary embodiment 195, provided herein is the method of embodiment 192, wherein the volume of hemoglobin-dependent bacteria is about 1 mL.

According to exemplary embodiment 196, provided herein is the method of any one of embodiments 183-195, wherein the hemoglobin-dependent bacteria is incubated for 10-24 hours.

According to exemplary embodiment 197, provided herein is the method of embodiment 196, wherein the hemoglobin-dependent bacteria is incubated for 14 to 16 hours.

According to exemplary embodiment 198, provided herein is the method of embodiment 196 or 197, wherein the method further comprises the step of inoculating about 5% v/v of the cultured bacteria in a growth medium.

According to exemplary embodiment 199, provided herein is the method of embodiment 198, wherein the growth medium is about 20 L in volume.

According to exemplary embodiment 200, provided herein is the method of embodiment 198 or 199, wherein the hemoglobin-dependent bacteria is incubated for 10-24 hours.

According to exemplary embodiment 201, provided herein is the method of embodiment 200, wherein the hemoglobin-dependent bacteria is incubated for 12 to 14 hours.

According to exemplary embodiment 202, provided herein is the method of embodiment 200 or 201, wherein the method further comprises the step of inoculating about 0.5% v/v of the cultured bacteria in a growth medium.

According to exemplary embodiment 203, provided herein is the method of embodiment 202, wherein the growth medium is about 3500 L in volume.

According to exemplary embodiment 204, provided herein is the method of embodiment 202 or 203, wherein the hemoglobin-dependent bacteria is incubated for 10-24 hours.

According to exemplary embodiment 205, provided herein is the method of embodiment 204, wherein the hemoglobin-dependent bacteria is incubated for 12 to 14 hours.

According to exemplary embodiment 206, provided herein is the method of any one of embodiments 127-205, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

According to exemplary embodiment 207, provided herein is the method of embodiment 206, wherein the growth medium comprises 5 g/L to 15 g/L yeast extract 19512.

According to exemplary embodiment 208, provided herein is the method of embodiment 206, wherein the growth medium comprises about 10 g/L yeast extract 19512.

According to exemplary embodiment 209, provided herein is the method of any one of embodiments 206 to 208, wherein the growth medium comprises 10 g/L to 15 g/L soy peptone A2SC 19649.

According to exemplary embodiment 210, provided herein is the method of embodiment 209, wherein the growth medium comprises about 12.5 g/L soy peptone A2SC 19649.

According to exemplary embodiment 211, provided herein is the method of embodiment 209, wherein the growth medium comprises about 10 g/L soy peptone A2SC 19649.

According to exemplary embodiment 212, provided herein is the method of any one of embodiments 206 to 211, wherein the growth medium comprises 10 g/L to 15 g/L Soy peptone E110 19885.

According to exemplary embodiment 213, provided herein is the method of embodiment 212, wherein the growth medium comprises about 12.5 g/L Soy peptone E110 19885.

According to exemplary embodiment 214, provided herein is the method of embodiment 212, wherein the growth medium comprises about 10 g/L soy peptone E110 19885.

According to exemplary embodiment 215, provided herein is the method of any one of embodiments 206 to 214, wherein the growth medium comprises 1 g/L to 3 g/L dipotassium phosphate.

According to exemplary embodiment 216, provided herein is the method of embodiment 215, wherein the growth medium comprises about 1.59 g/L dipotassium phosphate.

According to exemplary embodiment 217, provided herein is the method of embodiment 215, wherein the growth medium comprises about 2.5 g/L dipotassium phosphate.

According to exemplary embodiment 218, provided herein is the method of any one of embodiments 206 to 217, wherein the growth medium comprises 0.5 g/L to 1.5 g/L monopotassium phosphate.

According to exemplary embodiment 219, provided herein is the method of embodiment 218, wherein the growth medium comprises about 0.91 g/L monopotassium phosphate.

According to exemplary embodiment 220, provided herein is the method of any one of embodiments 206 to 219, wherein the growth medium comprises 0.1 g/L to 1.0 g/L L-cysteine-HCl.

According to exemplary embodiment 221, provided herein is the method of embodiment 220, wherein the growth medium comprises about 0.5 g/L L-cysteine-HCl.

According to exemplary embodiment 222, provided herein is the method of any one of embodiments 206 to 221, wherein the growth medium comprises 0.1 g/L to 1.0 g/L ammonium chloride.

According to exemplary embodiment 223, provided herein is the method of embodiment 222, wherein the growth medium comprises about 0.5 g/L ammonium chloride.

According to exemplary embodiment 224, provided herein is the method of any one of embodiments 206 to 223, wherein the growth medium comprises 20 g/L to 30 g/L glucidex 21 D.

According to exemplary embodiment 225, provided herein is the method of embodiment 224, wherein the growth medium comprises about 25 g/L glucidex 21 D.

According to exemplary embodiment 226, provided herein is the method of any one of embodiments 206 to 225, wherein the growth medium comprises 5 g/L to 15 g/L glucose.

According to exemplary embodiment 227, provided herein is the method of embodiment 226, wherein the growth medium comprises about 5 g/L glucose or about 10 g/L glucose.

According to exemplary embodiment 228, provided herein is the method of any one of embodiments 127-227, wherein the growth medium comprises at least 0.001 g/L of the heme-containing polypeptide.

According to exemplary embodiment 229, provided herein is the method of embodiment 228, wherein the growth medium comprises at least 0.005 g/L of the heme-containing polypeptide.

According to exemplary embodiment 230, provided herein is the method of embodiment 228, wherein the growth medium comprises at least 0.01 g/L the heme-containing polypeptide.

According to exemplary embodiment 231, provided herein is the method of embodiment 228, wherein the growth medium comprises at least 0.02 g/L of the heme-containing polypeptide.

According to exemplary embodiment 232, provided herein is the method of embodiment 228, wherein the growth medium comprises about 0.1 g/L of the heme-containing polypeptide.

According to exemplary embodiment 233, provided herein is the method of embodiment 228, wherein the growth medium comprises about 0.2 g/L of the heme-containing polypeptide.

According to exemplary embodiment 234, provided herein is the method of embodiment 228, wherein the growth medium comprises at least 0.001 g/L of a soy leghemoglobin.

According to exemplary embodiment 235, provided herein is the method of embodiment 234, wherein the growth medium comprises at least 0.005 g/L of a soy leghemoglobin.

According to exemplary embodiment 236, provided herein is the method of embodiment 234, wherein the growth medium comprises at least 0.01 g/L of a soy leghemoglobin.

According to exemplary embodiment 237, provided herein is the method of embodiment 234, wherein the growth medium comprises at least 0.02 g/L of a soy leghemoglobin.

According to exemplary embodiment 238, provided herein is the method of embodiment 234, wherein the growth medium comprises about 0.1 g/L of a soy leghemoglobin.

According to exemplary embodiment 239, provided herein is the method of embodiment 234, wherein the growth medium comprises about 0.2 g/L of a soy leghemoglobin.

According to exemplary embodiment 240, provided herein is the method of any one of embodiments 127-239, wherein the hemoglobin-dependent bacteria is incubated at a temperature of 35° C. to 39° C.

According to exemplary embodiment 241, provided herein is the method of embodiment 240, wherein the hemoglobin-dependent bacteria is incubated at a temperature of about 37° C.

According to exemplary embodiment 242, provided herein is the method of any one of embodiments 127-241, wherein the growth medium is at a pH of 5.5 to 7.5.

According to exemplary embodiment 243, provided herein is the method of embodiment 242, wherein the growth medium is at a pH of about 6.5.

According to exemplary embodiment 244, provided herein is the method of any one of embodiments 127-243, wherein incubating the hemoglobin-dependent bacteria comprises agitating the growth medium at a RPM of 50 to 300.

According to exemplary embodiment 245, provided herein is the method of embodiment 244, wherein the growth medium is agitated at a RPM of about 150.

According to exemplary embodiment 246, provided herein is the method of any one of embodiments 183-245, wherein the anaerobic gaseous mixture is continuously added during incubation.

According to exemplary embodiment 247, provided herein is the method of embodiment 246, wherein the anaerobic gaseous mixture is added at a rate of about 0.02 vvm.

According to exemplary embodiment 248, provided herein is the method of any one of embodiments 127-247, wherein the method further comprises the step of harvesting the hemoglobin-dependent bacteria when a stationary phase is reached.

According to exemplary embodiment 249, provided herein is the method of embodiment 248, further comprising the step of centrifuging the hemoglobin-dependent bacteria after harvesting to produce a cell paste.

According to exemplary embodiment 250, provided herein is the method of embodiment 249, further comprising diluting the cell paste with a stabilizer solution to produce a cell slurry.

According to exemplary embodiment 251, provided herein is the method of embodiment 250, further comprising the step of lyophilizing the cell slurry to produce a powder.

According to exemplary embodiment 252, provided herein is the method of embodiment 251, further comprising irradiating the powder with gamma radiation.

According to exemplary embodiment 253, provided herein is a bioreactor comprising hemoglobin-dependent bacteria in a growth medium comprising a heme-containing polypeptide, wherein the heme-containing polypeptide is: (i) a piscine polypeptide; (ii) an avian polypeptide; or (iii) a non-animal-derived polypeptide.

According to exemplary embodiment 254, provided herein is the bioreactor of embodiment 253, wherein the heme-containing polypeptide is a hemoglobin.

According to exemplary embodiment 255, provided herein is the bioreactor of embodiment 254, wherein the hemoglobin is a symbiotic hemoglobin, non-symbiotic hemoglobin, and/or truncated hemoglobin.

According to exemplary embodiment 256, provided herein is the bioreactor of embodiment 253, wherein the heme-containing polypeptide is a leghemoglobin.

According to exemplary embodiment 257, provided herein is the bioreactor of embodiment 253, wherein the heme-containing polypeptide is a myoglobin.

According to exemplary embodiment 258, provided herein is the bioreactor of any one of embodiments 253-257, wherein the heme-containing polypeptide is a piscine polypeptide.

According to exemplary embodiment 259, provided herein is the bioreactor embodiment 258, wherein the piscine polypeptide is purified from fish.

According to exemplary embodiment 260, provided herein is the bioreactor of embodiment 259, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 261, provided herein is the bioreactor of embodiment 258, wherein the piscine polypeptide is recombinantly expressed.

According to exemplary embodiment 262, provided herein is the bioreactor of any one of embodiments 253-257, wherein the heme-containing polypeptide is an avian polypeptide.

According to exemplary embodiment 263, provided herein is the bioreactor of embodiment 262, wherein the avian polypeptide is purified from a bird.

According to exemplary embodiment 264, provided herein is the bioreactor of embodiment 263, wherein the bird is of the genus Gallus, Meleagris, Anas, Anser, Branta, Chen, Agelastes, Numida, Guttera, Acryllium, or Columba.

According to exemplary embodiment 265, provided herein is the bioreactor of embodiment 262, wherein the avian polypeptide is recombinantly expressed.

According to exemplary embodiment 266, provided herein is the bioreactor of any one of embodiments 253-257, wherein the heme-containing polypeptide is a non-animal-derived polypeptide.

According to exemplary embodiment 267, provided herein is the bioreactor of embodiment 266, wherein the non-animal-derived polypeptide is purified from plants, bacteria, cyanobacteria, fungus, algae, grain, legume, and/or protozoa.

According to exemplary embodiment 268, provided herein is the bioreactor of embodiment 266 or 267, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 269, provided herein is the bioreactor of any one of embodiments 266-268, wherein the non-animal-derived polypeptide is a soy leghemoglobin.

According to exemplary embodiment 270, provided herein is the bioreactor of embodiment 269, wherein the soy leghemoglobin is purified from soy roots or soy root nodules.

According to exemplary embodiment 271, provided herein is the bioreactor of embodiment 266, wherein the non-animal-derived polypeptide is recombinantly expressed.

According to exemplary embodiment 272, provided herein is the bioreactor of any one of embodiments 253-257, 266, and 271, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

According to exemplary embodiment 273, provided herein is the bioreactor of embodiment 272, wherein the heme-containing polypeptide comprises an amino acid sequence with at least 90% identity to an amino acid sequence in any one of SEQ ID NOs: 1-29, 31, 33, 35, 37, 39, 41, 43, 114, or a combination thereof.

According to exemplary embodiment 274, provided herein is the bioreactor of any one of embodiments 261, 265, and 271, wherein the recombinantly expressed polypeptide comprises a heterologous polypeptide.

According to exemplary embodiment 275, provided herein is the bioreactor of embodiment 274, wherein the heterologous polypeptide comprises a histidine tag, TAP (tandem affinity purification) tag, TEV cleavage site, a FLAG tag, a GST tag, and/or an immunoglobulin domain.

According to exemplary embodiment 276, provided herein is the bioreactor of any one of embodiments 261, 265, and 271-275, wherein the recombinant polypeptide is expressed and purified from an exogenous nucleic acid in a host cell.

According to exemplary embodiment 277, provided herein is the bioreactor of embodiment 276, wherein the exogenous nucleic acid is in a vector.

According to exemplary embodiment 278, provided herein is the bioreactor of embodiment 277, wherein the vector is an expression vector.

According to exemplary embodiment 279, provided herein is the bioreactor of any one of embodiments 276-278, wherein the host cell is bacteria, yeast, insect, or mammalian cell lines.

According to exemplary embodiment 280, provided herein is the bioreactor of embodiment 279, wherein the host cell is Pichia Pastoris or Escherichia coli.

According to exemplary embodiment 281, provided herein is the bioreactor of any one of embodiments 271-280, wherein the heme-containing polypeptide is a soy leghemoglobin recombinantly expressed in Pichia pastoris.

According to exemplary embodiment 282, provided herein is the bioreactor of any one of embodiments 253-281, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

According to exemplary embodiment 283, provided herein is the bioreactor of any one of embodiments 253-281, wherein the hemoglobin-dependent bacteria are of the genus Prevotella.

According to exemplary embodiment 284, provided herein is the bioreactor of embodiment 283, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 285, provided herein is the bioreactor of embodiment 283, wherein the hemoglobin-dependent bacteria are of the species Prevotella histicola.

According to exemplary embodiment 286, provided herein is the bioreactor of embodiment 283, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 287, provided herein is the bioreactor of embodiment 283, wherein the Prevotella comprise at least 99.5% genomic, 16S and/or CRISPR sequence identity to the nucleotide sequence of the Prevotella Strain B 50329 (NRRL accession number B 50329) or Prevotella Strain C (PTA-126140).

According to exemplary embodiment 288, provided herein is the bioreactor of embodiment 283, wherein the Prevotella are Prevotella Strain B 50329 (NRRL accession number B 50329) or Prevotella Strain C (PTA-126140).

According to exemplary embodiment 289, provided herein is the bioreactor of any one of embodiments 283-288, wherein the hemoglobin-dependent bacteria are a strain of Prevotella bacteria comprising one or more proteins listed in Table 1.

According to exemplary embodiment 290, provided herein is the bioreactor of any one of embodiments 283-289, wherein the hemoglobin-dependent bacteria are from a strain of Prevotella substantially free of a protein listed in Table 2.

According to exemplary embodiment 291, provided herein is the bioreactor of any one of embodiments 253-290, wherein the hemoglobin-dependent bacteria grow at an increased rate in the growth medium comprising the heme-containing polypeptide compared to the rate at which the hemoglobin-dependent bacteria grow in the same growth medium but without the heme-containing polypeptide.

According to exemplary embodiment 292, provided herein is the bioreactor of embodiment 291, wherein the rate at which the hemoglobin-dependent bacteria grow in the growth medium comprising the heme-containing polypeptide is at least 50% higher than the rate at which the hemoglobin-dependent bacteria grow in the same growth medium but without the heme-containing polypeptide.

According to exemplary embodiment 293, provided herein is the bioreactor of embodiment 291, wherein the rate at which the hemoglobin-dependent bacteria grow in the growth medium comprising the heme-containing polypeptide is at least 100% higher than the rate at which the hemoglobin-dependent bacteria grow in the same growth medium but without the heme-containing polypeptide.

According to exemplary embodiment 294, provided herein is the bioreactor of embodiment 291, wherein the rate at which the hemoglobin-dependent bacteria grow in the growth medium comprising the heme-containing polypeptide is 200% to 400% higher than the rate at which the hemoglobin-dependent bacteria grow in the same growth medium but without the heme-containing polypeptide.

According to exemplary embodiment 295, provided herein is the bioreactor of embodiment 291, wherein the rate at which the hemoglobin-dependent bacteria grow in the growth medium comprising the heme-containing polypeptide is at least 300% higher than the rate at which the hemoglobin-dependent bacteria grow in the same growth medium but without the heme-containing polypeptide.

According to exemplary embodiment 296, provided herein is the bioreactor of any one of embodiments 253-295, wherein the hemoglobin-dependent bacteria grow to a higher cell density in the growth medium comprising the heme-containing polypeptide, compared to the cell density to which the hemoglobin-dependent bacteria grow in the same growth medium but without the heme-containing polypeptide.

According to exemplary embodiment 297, provided herein is the bioreactor of embodiment 296, wherein the hemoglobin-dependent bacteria grow to a cell density in the growth medium comprising the heme-containing polypeptide that is at least 50% higher than the cell density to which the hemoglobin-dependent bacteria grow in the same growth medium but without the heme-containing polypeptide.

According to exemplary embodiment 298, provided herein is the bioreactor of embodiment 296, wherein the hemoglobin-dependent bacteria grow to a cell density in the growth medium comprising the heme-containing polypeptide that is at least 100% higher than the cell density to which the hemoglobin-dependent bacteria grow in the same growth medium but without the heme-containing polypeptide.

According to exemplary embodiment 299, provided herein is the bioreactor of embodiment 296, wherein the hemoglobin-dependent bacteria grow to a cell density in the growth medium comprising the heme-containing polypeptide that is at 200% to 400% higher than the cell density to which the hemoglobin-dependent bacteria grow in the same growth medium but without the heme-containing polypeptide.

According to exemplary embodiment 300, provided herein is the bioreactor of embodiment 296, wherein the hemoglobin-dependent bacteria grow to a cell density in the growth medium comprising the heme-containing polypeptide that is at least 300% higher than the cell density to which the hemoglobin-dependent bacteria grow in the same growth medium but without the heme-containing polypeptide.

According to exemplary embodiment 301, provided herein is the bioreactor of any one of embodiments 253-300, wherein the hemoglobin-dependent bacteria are under an anaerobic atmosphere comprising at least about 1% CO₂.

According to exemplary embodiment 302, provided herein is the bioreactor of embodiment 301, wherein the anaerobic atmosphere comprises at least 10% CO₂.

According to exemplary embodiment 303, provided herein is the bioreactor of embodiment 301, wherein the anaerobic atmosphere comprises at least 20% CO₂.

According to exemplary embodiment 304, provided herein is the bioreactor of embodiment 301, wherein the anaerobic atmosphere comprises from 10% to 40% CO₂.

According to exemplary embodiment 305, provided herein is the bioreactor of embodiment 301, wherein the anaerobic atmosphere comprises from 20% to 30% CO₂.

According to exemplary embodiment 306, provided herein is the bioreactor of embodiment 301, wherein the anaerobic atmosphere comprises about 25% CO₂.

According to exemplary embodiment 307, provided herein is the bioreactor of any one of embodiments 301-306, wherein the anaerobic atmosphere consists essentially of CO₂ and N₂.

According to exemplary embodiment 308, provided herein is the bioreactor of embodiment 301, wherein the anaerobic atmosphere comprises about 25% CO₂ and about 75% N₂.

According to exemplary embodiment 309, provided herein is the bioreactor of any one of embodiments embodiment 301-308, wherein bioreactor is a 1 L, 20 L, 3500 L or 20,000 L bioreactor.

According to exemplary embodiment 310, provided herein is the bioreactor of any one of embodiments 253-309, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

According to exemplary embodiment 311, provided herein is the bioreactor of embodiment 310, wherein the growth medium comprises 5 g/L to 15 g/L yeast extract 19512.

According to exemplary embodiment 312, provided herein is the bioreactor of embodiment 310, wherein the growth medium comprises about 10 g/L yeast extract 19512.

According to exemplary embodiment 313, provided herein is the bioreactor of any one of embodiments 310 to 312, wherein the growth medium comprises 10 g/L to 15 g/L soy peptone A2SC 19649.

According to exemplary embodiment 314, provided herein is the bioreactor of embodiment 313, wherein the growth medium comprises about 12.5 g/L soy peptone A2SC 19649.

According to exemplary embodiment 315, provided herein is the bioreactor of embodiment 313, wherein the growth medium comprises about 10 g/L soy peptone A2SC

According to exemplary embodiment 316, provided herein is the bioreactor of any one of embodiments 310 to 315, wherein the growth medium comprises 10 g/L to 15 g/L Soy peptone E110 19885.

According to exemplary embodiment 317, provided herein is the bioreactor of embodiment 316, wherein the growth medium comprises about 12.5 g/L Soy peptone E110 19885.

According to exemplary embodiment 318, provided herein is the bioreactor of embodiment 316, wherein the growth medium comprises about 10 g/L soy peptone E110 19885.

According to exemplary embodiment 319, provided herein is the bioreactor of any one of embodiments 310-318, wherein the growth medium comprises 1 g/L to 3 g/L dipotassium phosphate.

According to exemplary embodiment 320, provided herein is the bioreactor of embodiment 319, wherein the growth medium comprises about 1.59 g/L dipotassium phosphate.

According to exemplary embodiment 321, provided herein is the bioreactor of embodiment 319, wherein the growth medium comprises about 2.5 g/L dipotassium phosphate.

According to exemplary embodiment 322, provided herein is the bioreactor of any one of embodiments 310-321, wherein the growth medium comprises 0.5 g/L to 1.5 g/L monopotassium phosphate.

According to exemplary embodiment 323, provided herein is the bioreactor of embodiment 322, wherein the growth medium comprises about 0.91 g/L monopotassium phosphate.

According to exemplary embodiment 324, provided herein is the bioreactor of any one of embodiments 310-323, wherein the growth medium comprises 0.1 g/L to 1.0 g/L L-cysteine-HCl.

According to exemplary embodiment 325, provided herein is the bioreactor of embodiment 324, wherein the growth medium comprises about 0.5 g/L L-cysteine-HCl.

According to exemplary embodiment 326, provided herein is the bioreactor of any one of embodiments 310-325, wherein the growth medium comprises 0.1 g/L to 1.0 g/L ammonium chloride.

According to exemplary embodiment 327, provided herein is the bioreactor of embodiment 326, wherein the growth medium comprises about 0.5 g/L ammonium chloride.

According to exemplary embodiment 328, provided herein is the bioreactor of any one of embodiments 310-327, wherein the growth medium comprises 20 g/L to 30 g/L glucidex 21 D.

According to exemplary embodiment 329, provided herein is the bioreactor of embodiment 328, wherein the growth medium comprises about 25 g/L glucidex 21 D.

According to exemplary embodiment 330, provided herein is the bioreactor of any one of embodiments 310-329, wherein the growth medium comprises 5 g/L to 15 g/L glucose.

According to exemplary embodiment 331, provided herein is the bioreactor of embodiment 330, wherein the growth medium comprises about 5 g/L glucose or about 10 g/L glucose.

According to exemplary embodiment 332, provided herein is the bioreactor of any one of embodiments 253-331, wherein the growth medium comprises at least 0.001 g/L of the heme-containing polypeptide.

According to exemplary embodiment 333, provided herein is the bioreactor of embodiment 332, wherein the growth medium comprises at least 0.005 g/L of the heme-containing polypeptide.

According to exemplary embodiment 334, provided herein is the bioreactor of embodiment 332, wherein the growth medium comprises at least 0.01 g/L the heme-containing polypeptide.

According to exemplary embodiment 335, provided herein is the bioreactor of embodiment 332, wherein the growth medium comprises at least 0.02 g/L of the heme-containing polypeptide.

According to exemplary embodiment 336, provided herein is the bioreactor of embodiment 332, wherein the growth medium comprises about 0.1 g/L of the heme-containing polypeptide.

According to exemplary embodiment 337, provided herein is the bioreactor of embodiment 332, wherein the growth medium comprises about 0.2 g/L of the heme-containing polypeptide.

According to exemplary embodiment 338, provided herein is the bioreactor of embodiment 332, wherein the growth medium comprises at least 0.001 g/L of a soy leghemoglobin.

According to exemplary embodiment 339, provided herein is the bioreactor of embodiment 338, wherein the growth medium comprises at least 0.005 g/L of a soy leghemoglobin.

According to exemplary embodiment 340, provided herein is the bioreactor of embodiment 338, wherein the growth medium comprises at least 0.01 g/L of a soy leghemoglobin.

According to exemplary embodiment 341, provided herein is the bioreactor of embodiment 338, wherein the growth medium comprises at least 0.02 g/L of a soy leghemoglobin.

According to exemplary embodiment 342, provided herein is the bioreactor of embodiment 338, wherein the growth medium comprises about 0.1 g/L of a soy leghemoglobin.

According to exemplary embodiment 343, provided herein is the bioreactor of embodiment 338, wherein the growth medium comprises about 0.2 g/L of a soy leghemoglobin.

According to exemplary embodiment 344, provided herein is the bioreactor of any one of embodiments 253-343, wherein the bioreactor is at a temperature of 35° C. to 39° C.

According to exemplary embodiment 345, provided herein is the bioreactor of embodiment 344, wherein the a bioreactor is at a temperature of 37° C.

According to exemplary embodiment 346, provided herein is the bioreactor of any one of embodiments 253-345, wherein the growth medium is at a pH of 5.5 to 7.5.

According to exemplary embodiment 347, provided herein is the bioreactor of embodiment 346, wherein the growth medium is at a pH of about 6.5.

According to exemplary embodiment 348, provided herein is a method of culturing hemoglobin-dependent bacteria in the bioreactor of any one of embodiments 253-347, provided herein is the method comprises incubating the hemoglobin-dependent bacteria in the bioreactor.

According to exemplary embodiment 349, provided herein is the method of embodiment 348, wherein the hemoglobin-dependent bacteria are incubated in an anaerobic gaseous mixture comprising greater than 1% CO₂.

According to exemplary embodiment 350, provided herein is the method of embodiment 348, wherein the anaerobic gaseous mixture comprises at least 10% CO₂.

According to exemplary embodiment 351, provided herein is the method of embodiment 348, wherein the anaerobic gaseous mixture comprises at least 20% CO₂.

According to exemplary embodiment 352, provided herein is the method of embodiment 348, wherein the anaerobic gaseous mixture comprises from 10% to 40% CO₂.

According to exemplary embodiment 353, provided herein is the method of embodiment 348, wherein the anaerobic gaseous mixture comprises from 20% to 30% CO₂.

According to exemplary embodiment 354, provided herein is the method of embodiment 348, wherein the anaerobic gaseous mixture comprises about 25% CO₂.

According to exemplary embodiment 355, provided herein is the method of any one of embodiments 348-354, wherein the anaerobic gaseous mixture consists essentially of CO₂ and N₂.

According to exemplary embodiment 356, provided herein is the method of embodiment 355, wherein the anaerobic gaseous mixture comprises about 25% CO₂ and about 75% N₂.

According to exemplary embodiment 357, provided herein is the method of any one of embodiments embodiment 348-356, wherein the method further comprises the step of inoculating the growth medium with the hemoglobin-dependent bacteria prior to incubation.

According to exemplary embodiment 358, provided herein is the method of embodiment 357, wherein the volume of hemoglobin-dependent bacteria inoculated is about 0.1% v/v of the growth medium.

According to exemplary embodiment 359, provided herein is the method of embodiment 357, wherein the growth medium is about 1 L in volume.

According to exemplary embodiment 360, provided herein is the method of embodiment 357, wherein the volume of hemoglobin-dependent bacteria inoculated is about 1 mL.

According to exemplary embodiment 361, provided herein is the method of any one of embodiments 348-360, wherein the hemoglobin-dependent bacteria is cultured for 10-24 hours.

According to exemplary embodiment 362, provided herein is the method of embodiment 361, wherein the hemoglobin-dependent bacteria is incubated for 14 to 16 hours.

According to exemplary embodiment 363, provided herein is the method of embodiment 361 or 362, wherein the method further comprises the step of inoculating about 5% v/v of the cultured bacteria in a growth medium.

According to exemplary embodiment 364, provided herein is the method of embodiment 363, wherein the growth medium is about 20 L in volume.

According to exemplary embodiment 365, provided herein is the method of embodiment 363 or 364, wherein the hemoglobin-dependent bacteria is incubated for 10-24 hours.

According to exemplary embodiment 366, provided herein is the method of embodiment 365, wherein the hemoglobin-dependent bacteria is incubate for 12 to 14 hours.

According to exemplary embodiment 367, provided herein is the method of embodiment 365 or 366, wherein the method further comprises the step of inoculating about 0.5% v/v of the cultured bacteria in a growth medium.

According to exemplary embodiment 368, provided herein is the method of embodiment 367, wherein the growth medium is about 3500 L in volume.

According to exemplary embodiment 369, provided herein is the method of embodiment 367 or 368, wherein the hemoglobin-dependent bacteria is incubated for 10-24 hours.

According to exemplary embodiment 370, provided herein is the method of embodiment 369, wherein the hemoglobin-dependent bacteria is incubated for 12 to 14 hours.

According to exemplary embodiment 371, provided herein is the method of any one of embodiments 348-370, wherein the hemoglobin-dependent bacteria is incubated at a temperature of 35° C. to 39° C.

According to exemplary embodiment 372, provided herein is the method of embodiment 371, wherein the hemoglobin-dependent bacteria is incubated at a temperature of 37° C.

According to exemplary embodiment 373, provided herein is the method of any one of embodiments 348-372, wherein incubating the hemoglobin-dependent bacteria comprises agitating the growth medium at a RPM of 50 to 300.

According to exemplary embodiment 374, provided herein is the method of embodiment 373, wherein the growth medium is agitated at a RPM of 150.

According to exemplary embodiment 375, provided herein is the method of any one of embodiments 348-374, wherein the anaerobic gaseous mixture is continuously added during incubation.

According to exemplary embodiment 376, provided herein is the method of embodiment 375, wherein the anaerobic gaseous mixture is added at a rate of 0.02 vvm.

According to exemplary embodiment 377, provided herein is the method of any one of embodiments 348-376, wherein the method further comprises the step of harvesting the hemoglobin-dependent bacteria when a stationary phase is reached.

According to exemplary embodiment 378, provided herein is the method of embodiment 377, further comprising the step of centrifuging the hemoglobin-dependent bacteria after harvesting to produce a cell paste.

According to exemplary embodiment 379, provided herein is the method of embodiment 378, further comprising diluting the cell paste with a stabilizer solution to produce a cell slurry.

According to exemplary embodiment 380, provided herein is the method of embodiment 379, further comprising the step of lyophilizing the cell slurry to produce a powder.

According to exemplary embodiment 381, provided herein is the method of embodiment 380, further comprising irradiating the powder with gamma radiation.

According to exemplary embodiment 382, A composition comprising a) hemoglobin-dependent bacteria, and b) a growth medium comprising a heme-containing polypeptide, wherein the heme-containing polypeptide is: (i) a piscine polypeptide; (ii) an avian polypeptide; or (iii) a non-animal-derived polypeptide.

According to exemplary embodiment 383, provided herein is the composition of embodiment 382, wherein the heme-containing polypeptide is a hemoglobin.

According to exemplary embodiment 384, provided herein is the composition of embodiment 383, wherein the hemoglobin is a symbiotic hemoglobin, non-symbiotic hemoglobin, and/or truncated hemoglobin.

According to exemplary embodiment 385, provided herein is the composition of embodiment 382, wherein the heme-containing polypeptide is a leghemoglobin.

According to exemplary embodiment 386, provided herein is the composition of embodiment 382, wherein the heme-containing polypeptide is a myoglobin.

According to exemplary embodiment 387, provided herein is the composition of any one of embodiments 382-386, wherein the heme-containing polypeptide is a piscine polypeptide.

According to exemplary embodiment 388, provided herein is the composition of embodiment 387, wherein the piscine polypeptide is purified from fish.

According to exemplary embodiment 389, provided herein is the composition of embodiment 388, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 390, provided herein is the composition of embodiment 387, wherein the piscine polypeptide is recombinantly expressed.

According to exemplary embodiment 391, provided herein is the composition of any one of embodiments 382-386, wherein the heme-containing polypeptide is an avian polypeptide.

According to exemplary embodiment 392, provided herein is the composition of embodiment 391, wherein the avian polypeptide is purified from a bird.

According to exemplary embodiment 393, provided herein is the composition of embodiment 392, wherein the bird is of the genus Gallus, Meleagris, Anas, Anser, Branta, Chen, Agelastes, Numida, Guttera, Acryllium, or Columba.

According to exemplary embodiment 394, provided herein is the composition of embodiment 391, wherein the avian polypeptide is recombinantly expressed.

According to exemplary embodiment 395, provided herein is the composition of any one of embodiments 382-386, wherein the heme-containing polypeptide is a non-animal-derived polypeptide.

According to exemplary embodiment 396, provided herein is the composition of embodiment 395, wherein the non-animal-derived polypeptide is purified from plants, bacteria, cyanobacteria, fungus, algae, grain, legume, and/or protozoa.

According to exemplary embodiment 397, provided herein is the composition of embodiment 395 or 396, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 398, provided herein is the composition of any one of embodiments 395-397, wherein the non-animal-derived polypeptide is a soy leghemoglobin.

According to exemplary embodiment 399, provided herein is the composition of embodiment 398, wherein the soy leghemoglobin is purified from soy roots or soy root nodules.

According to exemplary embodiment 400, provided herein is the composition of embodiment 395, wherein the non-animal-derived polypeptide is recombinantly expressed.

According to exemplary embodiment 401, provided herein is the composition of any one of embodiments 382-386, 395, and 400, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

According to exemplary embodiment 402, provided herein is the composition of embodiment 401, wherein the heme-containing polypeptide comprises an amino acid sequence with at least 90% identity to an amino acid sequence in any one of SEQ ID NOs: 1-29, 31, 33, 35, 37, 39, 41, 43, 114, or a combination thereof.

According to exemplary embodiment 403, provided herein is the composition of any one of embodiments 390, 394, and 400, wherein the recombinantly expressed polypeptide comprises a heterologous polypeptide.

According to exemplary embodiment 404, provided herein is the composition of embodiment 403, wherein the heterologous polypeptide comprises a histidine tag, TAP (tandem affinity purification) tag, TEV cleavage site, a FLAG tag, a GST tag, and/or an immunoglobulin domain.

According to exemplary embodiment 405, provided herein is the composition of any one of embodiments 390, 394, and 400-404, wherein the recombinant polypeptide is expressed and purified from an exogenous nucleic acid in a host cell.

According to exemplary embodiment 406, provided herein is the composition of embodiment 405, wherein the exogenous nucleic acid is in a vector.

According to exemplary embodiment 407, provided herein is the composition of embodiment 406, wherein the vector is an expression vector.

According to exemplary embodiment 408, provided herein is the composition of any one of embodiments 405-407, wherein the host cell is bacteria, yeast, insect, or mammalian cell lines.

According to exemplary embodiment 409, provided herein is the composition of embodiment 408, wherein the host cell is Pichia Pastoris or Escherichia coli.

According to exemplary embodiment 410, provided herein is the composition of any one of embodiments 400-409, wherein the heme-containing polypeptide is a soy leghemoglobin recombinantly expressed in Pichia pastoris.

According to exemplary embodiment 411, provided herein is the composition of any one of embodiments 382-410, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

According to exemplary embodiment 412, provided herein is the composition of any one of embodiments 382-410, wherein the hemoglobin-dependent bacteria are of the genus Prevotella.

According to exemplary embodiment 413, provided herein is the composition of embodiment 412, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 414, provided herein is the composition of embodiment 412, wherein the hemoglobin-dependent bacteria are of the species Prevotella histicola.

According to exemplary embodiment 415, provided herein is the composition of embodiment 412, wherein the Prevotella comprise at least 90% genomic, 16S and/or CRISPR sequence identity to the nucleotide sequence of the Prevotella Strain B 50329 (NRRL accession number B 50329) or Prevotella Strain C (PTA-126140).

According to exemplary embodiment 416, provided herein is the composition of embodiment 412, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 417, provided herein is the composition of embodiment 412, wherein the Prevotella are Prevotella Strain B 50329 (NRRL accession number B 50329) or Prevotella Strain C (PTA-126140).

According to exemplary embodiment 418, provided herein is the composition of any one of embodiments 412-417, wherein the hemoglobin-dependent bacteria are a strain of Prevotella bacteria comprising one or more proteins listed in Table 1.

According to exemplary embodiment 419, provided herein is the composition of any one of embodiments 412-418, wherein the hemoglobin-dependent bacteria are a strain of Prevotella substantially free of a protein listed in Table 2.

According to exemplary embodiment 420, provided herein is the composition of any one of embodiments 382-419, wherein the hemoglobin-dependent bacteria grow at an increased rate in the growth medium comprising the heme-containing polypeptide compared to the rate at which the hemoglobin-dependent bacteria grow in the same growth medium but without the heme-containing polypeptide.

According to exemplary embodiment 421, provided herein is the composition of embodiment 420, wherein the rate at which the hemoglobin-dependent bacteria grow in the growth medium comprising the heme-containing polypeptide is at least 50% higher than the rate at which the hemoglobin-dependent bacteria grow in the same growth medium but without the heme-containing polypeptide.

According to exemplary embodiment 422, provided herein is the composition of embodiment 420, wherein the rate at which the hemoglobin-dependent bacteria grow in the growth medium comprising the heme-containing polypeptide is at least 100% higher than the rate at which the hemoglobin-dependent bacteria grow in the same growth medium but without the heme-containing polypeptide.

According to exemplary embodiment 423, provided herein is the composition of embodiment 420, wherein the rate at which the hemoglobin-dependent bacteria grow in the growth medium comprising the heme-containing polypeptide is 200% to 400% higher than the rate at which the hemoglobin-dependent bacteria grow in the same growth medium but without the heme-containing polypeptide.

According to exemplary embodiment 424, provided herein is the composition of embodiment 420, wherein the rate at which the hemoglobin-dependent bacteria grow in the growth medium comprising the heme-containing polypeptide is at least 300% higher than the rate at which the hemoglobin-dependent bacteria grow in the same growth medium but without the heme-containing polypeptide.

According to exemplary embodiment 425, provided herein is the composition of any one of embodiments 382-424, wherein the hemoglobin-dependent bacteria grow to a higher cell density in the growth medium comprising the heme-containing polypeptide, compared to the cell density to which the hemoglobin-dependent bacteria grow in the same growth medium but without the heme-containing polypeptide.

According to exemplary embodiment 426, provided herein is the composition of embodiment 425, wherein the hemoglobin-dependent bacteria grow to a cell density in the growth medium comprising the heme-containing polypeptide that is at least 50% higher than the cell density to which the hemoglobin-dependent bacteria grow in the same growth medium but without the heme-containing polypeptide.

According to exemplary embodiment 427, provided herein is the composition of embodiment 425, wherein the hemoglobin-dependent bacteria grow to a cell density in the growth medium comprising the heme-containing polypeptide that is at least 100% higher than the cell density to which the hemoglobin-dependent bacteria grow in the same growth medium but without the heme-containing polypeptide.

According to exemplary embodiment 428, provided herein is the composition of embodiment 425, wherein the hemoglobin-dependent bacteria grow to a cell density in the growth medium comprising the heme-containing polypeptide that is at 200% to 400% higher than the cell density to which the hemoglobin-dependent bacteria grow in the same growth medium but without the heme-containing polypeptide.

According to exemplary embodiment 429, provided herein is the composition of embodiment 425, wherein the hemoglobin-dependent bacteria grow to a cell density in the growth medium comprising the heme-containing polypeptide that is at least 300% higher than the cell density to which the hemoglobin-dependent bacteria grow in the same growth medium but without the heme-containing polypeptide.

According to exemplary embodiment 430, provided herein is the composition of any one of embodiments 382-429, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

According to exemplary embodiment 431, provided herein is the composition of embodiment 430, wherein the growth medium comprises 5 g/L to 15 g/L yeast extract 19512.

According to exemplary embodiment 432, provided herein is the composition of embodiment 430, wherein the growth medium comprises about 10 g/L yeast extract 19512.

According to exemplary embodiment 433, provided herein is the composition of any one of embodiments 430-432, wherein the growth medium comprises 10 g/L to 15 g/L soy peptone A2SC 19649.

According to exemplary embodiment 434, provided herein is the composition of embodiment 433, wherein the growth medium comprises about 12.5 g/L soy peptone A2SC 19649.

According to exemplary embodiment 435, provided herein is the composition of embodiment 433, wherein the growth medium comprises about 10 g/L soy peptone A2SC 19649.

According to exemplary embodiment 436, provided herein is the composition of any one of embodiments 430-435, wherein the growth medium comprises 10 g/L to 15 g/L Soy peptone E110 19885.

According to exemplary embodiment 437, provided herein is the composition of embodiment 436, wherein the growth medium comprises about 12.5 g/L Soy peptone E110 19885.

According to exemplary embodiment 438, provided herein is the composition of embodiment 436, wherein the growth medium comprises about 10 g/L soy peptone E110 19885.

According to exemplary embodiment 439, provided herein is the composition of any one of embodiments 430-438, wherein the growth medium comprises 1 g/L to 3 g/L dipotassium phosphate.

According to exemplary embodiment 440, provided herein is the composition of embodiment 439, wherein the growth medium comprises about 1.59 g/L dipotassium phosphate.

According to exemplary embodiment 441, provided herein is the composition of embodiment 439, wherein the growth medium comprises about 2.5 g/L dipotassium phosphate.

According to exemplary embodiment 442, provided herein is the composition of any one of embodiments 430-441, wherein the growth medium comprises 0.5 g/L to 1.5 g/L monopotassium phosphate.

According to exemplary embodiment 443, provided herein is the composition of embodiment 442, wherein the growth medium comprises about 0.91 g/L monopotassium phosphate.

According to exemplary embodiment 444, provided herein is the composition of any one of embodiments 430-441, wherein the growth medium comprises 0.1 g/L to 1.0 g/L L-cysteine-HCl.

According to exemplary embodiment 445, provided herein is the composition of embodiment 444, wherein the growth medium comprises about 0.5 g/L L-cysteine-HCl.

According to exemplary embodiment 446, provided herein is the composition of any one of embodiments 430-445, wherein the growth medium comprises 0.1 g/L to 1.0 g/L ammonium chloride.

According to exemplary embodiment 447, provided herein is the composition of embodiment 446, wherein the growth medium comprises about 0.5 g/L ammonium chloride.

According to exemplary embodiment 448, provided herein is the composition of any one of embodiments 430-447, wherein the growth medium comprises 20 g/L to 30 g/L glucidex 21 D.

According to exemplary embodiment 449, provided herein is the composition of embodiment 448, wherein the growth medium comprises about 25 g/L glucidex 21 D.

According to exemplary embodiment 450, provided herein is the composition of any one of embodiments 430-416449 wherein the growth medium comprises 5 g/L to 15 g/L glucose.

According to exemplary embodiment 451, provided herein is the composition of embodiment 450, wherein the growth medium comprises about 5 g/L glucose or about 10 g/L glucose.

According to exemplary embodiment 452, provided herein is the composition of any one of embodiments 382-451, wherein the growth medium comprises at least 0.001 g/L of the heme-containing polypeptide.

According to exemplary embodiment 453, provided herein is the composition of embodiment 452, wherein the growth medium comprises at least 0.005 g/L of the heme-containing polypeptide.

According to exemplary embodiment 454, provided herein is the composition of embodiment 453, wherein the growth medium comprises at least 0.01 g/L the heme-containing polypeptide.

According to exemplary embodiment 455, provided herein is the composition of embodiment 453, wherein the growth medium comprises at least 0.02 g/L of the heme-containing polypeptide.

According to exemplary embodiment 456, provided herein is the composition of embodiment 453, wherein the growth medium comprises about 0.1 g/L of the heme-containing polypeptide.

According to exemplary embodiment 457, provided herein is the composition of embodiment 453, wherein the growth medium comprises about 0.2 g/L of the heme-containing polypeptide.

According to exemplary embodiment 458, provided herein is the composition of embodiment 453, wherein the growth medium comprises at least 0.001 g/L of a soy leghemoglobin.

According to exemplary embodiment 459, provided herein is the composition of embodiment 458, wherein the growth medium comprises at least 0.005 g/L of a soy leghemoglobin.

According to exemplary embodiment 460, provided herein is the composition of embodiment 458, wherein the growth medium comprises at least 0.01 g/L of a soy leghemoglobin.

According to exemplary embodiment 461, provided herein is the composition of embodiment 458, wherein the growth medium comprises at least 0.02 g/L of a soy leghemoglobin.

According to exemplary embodiment 462, provided herein is the composition of embodiment 458, wherein the growth medium comprises about 0.1 g/L of a soy leghemoglobin.

According to exemplary embodiment 463, provided herein is the composition of embodiment 458, wherein the growth medium comprises about 0.2 g/L of a soy leghemoglobin.

According to exemplary embodiment 464, provided herein is the composition of any one of embodiments 382-463, wherein the growth medium is at a pH of 5.5 to 7.5.

According to exemplary embodiment 465, provided herein is the composition of embodiment 464, wherein the growth medium is at a pH of about 6.5.

According to exemplary embodiment 466, provided herein is a growth medium for use in culturing hemoglobin-dependent bacteria, the growth medium comprising a heme-containing polypeptide, wherein the heme-containing polypeptide is: (i) a piscine polypeptide; (ii) an avian polypeptide; or (iii) a non-animal-derived polypeptide.

According to exemplary embodiment 467, provided herein is the growth medium of embodiment 466, wherein the heme-containing polypeptide is a hemoglobin.

According to exemplary embodiment 468, provided herein is the growth medium of embodiment 467, wherein the hemoglobin is a symbiotic hemoglobin, non-symbiotic hemoglobin, and/or truncated hemoglobin.

According to exemplary embodiment 469, provided herein is the growth medium of embodiment 466, wherein the heme-containing polypeptide is a leghemoglobin.

According to exemplary embodiment 470, provided herein is the growth medium of embodiment 466, wherein the heme-containing polypeptide is a myoglobin.

According to exemplary embodiment 471, provided herein is the growth medium of any one of embodiments 466-470, wherein the heme-containing polypeptide is a piscine polypeptide.

According to exemplary embodiment 472, provided herein is the growth medium embodiment 471, wherein the piscine polypeptide is purified from fish.

According to exemplary embodiment 473, provided herein is the growth medium of embodiment 472, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 474, provided herein is the growth medium of embodiment 471, wherein the piscine polypeptide is recombinantly expressed.

According to exemplary embodiment 475, provided herein is the growth medium of any one of embodiments 466-470, wherein the heme-containing polypeptide is an avian polypeptide.

According to exemplary embodiment 476, provided herein is the growth medium of embodiment 475, wherein the avian polypeptide is purified from a bird.

According to exemplary embodiment 477, provided herein is the growth medium of embodiment 476, wherein the bird is of the genus Gallus, Meleagris, Anas, Anser, Branta, Chen, Agelastes, Numida, Guttera, Acryllium, or Columba.

According to exemplary embodiment 478, provided herein is the growth medium of embodiment 475, wherein the avian polypeptide is recombinantly expressed.

According to exemplary embodiment 479, provided herein is the growth medium of any one of embodiments 466-470, wherein the heme-containing polypeptide is a non-animal-derived polypeptide.

According to exemplary embodiment 480, provided herein is the growth medium of embodiment 479, wherein the non-animal-derived polypeptide is purified from plants, bacteria, cyanobacteria, fungus, algae, grain, legume, and/or protozoa.

According to exemplary embodiment 481, provided herein is the growth medium of embodiment 479 or 480, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 482, provided herein is the growth medium of any one of embodiments 479-481, wherein the non-animal-derived polypeptide is a soy leghemoglobin.

According to exemplary embodiment 483, provided herein is the growth medium of embodiment 482, wherein the soy leghemoglobin is purified from soy roots or soy root nodules.

According to exemplary embodiment 484, provided herein is the growth medium of embodiment 479, wherein the non-animal-derived polypeptide is recombinantly expressed.

According to exemplary embodiment 485, provided herein is the growth medium of any one of embodiments 466-470, 479, and 484, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

According to exemplary embodiment 486, provided herein is the growth medium of embodiment 485, wherein the heme-containing polypeptide comprises an amino acid sequence with at least 90% identity to an amino acid sequence in any one of SEQ ID NOs: 1-29, 31, 33, 35, 37, 39, 41, 43, 114, or a combination thereof.

According to exemplary embodiment 487, provided herein is the growth medium of any one of embodiments 474, 478, and 484, wherein the recombinantly expressed polypeptide comprises a heterologous polypeptide.

According to exemplary embodiment 488, provided herein is the growth medium of embodiment 487, wherein the heterologous polypeptide comprises a histidine tag, TAP (tandem affinity purification) tag, TEV cleavage site, a FLAG tag, a GST tag, and/or an immunoglobulin domain.

According to exemplary embodiment 489, provided herein is the growth medium of any one of embodiments 474, 478, and 484-488, wherein the recombinant polypeptide is expressed and purified from an exogenous nucleic acid in a host cell.

According to exemplary embodiment 490, provided herein is the growth medium of embodiment 489, wherein the exogenous nucleic acid is in a vector.

According to exemplary embodiment 491, provided herein is the growth medium of embodiment 490, wherein the vector is an expression vector.

According to exemplary embodiment 492, provided herein is the growth medium of any one of embodiments 489-491, wherein the host cell is bacteria, yeast, insect, or mammalian cell lines.

According to exemplary embodiment 493, provided herein is the growth medium of embodiment 492, wherein the wherein the host cell is Pichia Pastoris or Escherichia coli.

According to exemplary embodiment 494, provided herein is the growth medium of any one of embodiments 484-493, wherein the heme-containing polypeptide is a soy leghemoglobin recombinantly expressed in Pichia pastoris.

According to exemplary embodiment 495, provided herein is the growth medium of embodiment 466-494, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

According to exemplary embodiment 496, provided herein is the growth medium of any one of embodiments 466-494, wherein the hemoglobin-dependent bacteria are of the genus Prevotella.

According to exemplary embodiment 497, provided herein is the growth medium of embodiment 496, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 498, provided herein is the growth medium of embodiment 496, wherein the hemoglobin-dependent bacteria are of the species Prevotella histicola.

According to exemplary embodiment 499, provided herein is the growth medium of embodiment 496, wherein the Prevotella comprise at least 90% genomic, 16S and/or CRISPR sequence identity to the nucleotide sequence of the Prevotella Strain B 50329 (NRRL accession number B 50329) or Prevotella Strain C (PTA-126140).

According to exemplary embodiment 500, provided herein is the growth medium of embodiment 496, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 501, provided herein is the growth medium of embodiment 496, wherein the Prevotella are Prevotella Strain B 50329 (NRRL accession number B 50329) or Prevotella Strain C (PTA-126140).

According to exemplary embodiment 502, provided herein is the growth medium of any one of embodiments 496-501, wherein the hemoglobin-dependent bacteria are a strain of Prevotella bacteria comprising one or more proteins listed in Table 1.

According to exemplary embodiment 503, provided herein is the growth medium of any one of embodiments 496-502, wherein the hemoglobin-dependent bacteria are a strain of Prevotella substantially free of a protein listed in Table 2.

According to exemplary embodiment 504, provided herein is the growth medium of any one of embodiments 466-503, wherein the hemoglobin-dependent bacteria grow at an increased rate in the growth medium comprising the heme-containing polypeptide compared to the rate at which the hemoglobin-dependent bacteria grow in the same growth medium but without the heme-containing polypeptide.

According to exemplary embodiment 505, provided herein is the growth medium of embodiment 504, wherein the rate at which the hemoglobin-dependent bacteria grow in the growth medium comprising the heme-containing polypeptide is at least 50% higher than the rate at which the hemoglobin-dependent bacteria grow in the same growth medium but without the heme-containing polypeptide.

According to exemplary embodiment 506, provided herein is the growth medium of embodiment 504, wherein the rate at which the hemoglobin-dependent bacteria grow in the growth medium comprising the heme-containing polypeptide is at least 100% higher than the rate at which the hemoglobin-dependent bacteria grow in the same growth medium but without the heme-containing polypeptide.

According to exemplary embodiment 507, provided herein is the growth medium of embodiment 504, wherein the rate at which the hemoglobin-dependent bacteria grow in the growth medium comprising the heme-containing polypeptide is 200% to 400% higher than the rate at which the hemoglobin-dependent bacteria grow in the same growth medium but without the heme-containing polypeptide.

According to exemplary embodiment 508, provided herein is the growth medium of embodiment 504, wherein the rate at which the hemoglobin-dependent bacteria grow in the growth medium comprising the heme-containing polypeptide is at least 300% higher than the rate at which the hemoglobin-dependent bacteria grow in the same growth medium but without the heme-containing polypeptide.

According to exemplary embodiment 509, provided herein is the growth medium of any one of embodiments 466-508, wherein the hemoglobin-dependent bacteria grow to a higher cell density in the growth medium comprising the heme-containing polypeptide, compared to the cell density to which the hemoglobin-dependent bacteria grow in the same growth medium but without the heme-containing polypeptide.

According to exemplary embodiment 510, provided herein is the growth medium of embodiment 509, wherein the hemoglobin-dependent bacteria grow to a cell density in the growth medium comprising the heme-containing polypeptide that is at least 50% higher than the cell density to which the hemoglobin-dependent bacteria grow in the same growth medium but without the heme-containing polypeptide.

According to exemplary embodiment 511, provided herein is the growth medium of embodiment 509, wherein the hemoglobin-dependent bacteria grow to a cell density in the growth medium comprising the heme-containing polypeptide that is at least 100% higher than the cell density to which the hemoglobin-dependent bacteria grow in the same growth medium but without the heme-containing polypeptide.

According to exemplary embodiment 512, provided herein is the growth medium of embodiment 509, wherein the hemoglobin-dependent bacteria grow to a cell density in the growth medium comprising the heme-containing polypeptide that is at 200% to 400% higher than the cell density to which the hemoglobin-dependent bacteria grow in the same growth medium but without the heme-containing polypeptide.

According to exemplary embodiment 513, provided herein is the growth medium of embodiment 509, wherein the hemoglobin-dependent bacteria grow to a cell density in the growth medium comprising the heme-containing polypeptide that is at least 300% higher than the cell density to which the hemoglobin-dependent bacteria grow in the same growth medium but without the heme-containing polypeptide.

According to exemplary embodiment 514, provided herein is the growth medium of any one of embodiments 466-513, wherein the growth medium comprises yeast extract, soy peptone A2SC 19649, Soy peptone E110 19885, dipotassium phosphate, monopotassium phosphate, L-cysteine-HCl, ammonium chloride, glucidex 21 D, and glucose.

According to exemplary embodiment 515, provided herein is the growth medium of embodiment 514, wherein the growth medium comprises 5 g/L to 15 g/L yeast extract 19512.

According to exemplary embodiment 516, provided herein is the growth medium of embodiment 514, wherein the growth medium comprises about 10 g/L yeast extract 19512.

According to exemplary embodiment 517, provided herein is the growth medium of any one of embodiments 514-516, wherein the growth medium comprises 10 g/L to 15 g/L soy peptone A2SC 19649.

According to exemplary embodiment 518, provided herein is the growth medium of embodiment 517, wherein the growth medium comprises about 12.5 g/L soy peptone A2SC 19649.

According to exemplary embodiment 519, provided herein is the growth medium of embodiment 517, wherein the growth medium comprises about 10 g/L soy peptone A2SC 19649.

According to exemplary embodiment 520, provided herein is the growth medium of any one of embodiments 514-519, wherein the growth medium comprises 10 g/L to 15 g/L Soy peptone E110 19885.

According to exemplary embodiment 521, provided herein is the growth medium of embodiment 5205, wherein the growth medium comprises about 12.5 g/L Soy peptone E110 19885.

According to exemplary embodiment 522, provided herein is the growth medium of embodiment 520, wherein the growth medium comprises about 10 g/L soy peptone E110 19885.

According to exemplary embodiment 523, provided herein is the growth medium of any one of embodiments 514-522, wherein the growth medium comprises 1 g/L to 3 g/L dipotassium phosphate.

According to exemplary embodiment 524, provided herein is the growth medium of embodiment 523, wherein the growth medium comprises about 1.59 g/L dipotassium phosphate.

According to exemplary embodiment 525, provided herein is the growth medium of embodiment 523, wherein the growth medium comprises about 2.5 g/L dipotassium phosphate.

According to exemplary embodiment 526, provided herein is the growth medium of any one of embodiments 514-525, wherein the growth medium comprises 0.5 g/L to 1.5 g/L monopotassium phosphate.

According to exemplary embodiment 527, provided herein is the growth medium of embodiment 526, wherein the growth medium comprises about 0.91 g/L monopotassium phosphate.

According to exemplary embodiment 528, provided herein is the growth medium of any one of embodiments 514-527, wherein the growth medium comprises 0.1 g/L to 1.0 g/L L-cysteine-HCl.

According to exemplary embodiment 529, provided herein is the growth medium of embodiment 528, wherein the growth medium comprises about 0.5 g/L L-cysteine-HCl.

According to exemplary embodiment 530, provided herein is the growth medium of any one of embodiments 514-529, wherein the growth medium comprises 0.1 g/L to 1.0 g/L ammonium chloride.

According to exemplary embodiment 531, provided herein is the growth medium of embodiment 530, wherein the growth medium comprises about 0.5 g/L ammonium chloride.

According to exemplary embodiment 532, provided herein is the growth medium of any one of embodiments 514-531, wherein the growth medium comprises 20 g/L to 30 g/L glucidex 21 D.

According to exemplary embodiment 533, provided herein is the growth medium of embodiment 532, wherein the growth medium comprises about 25 g/L glucidex 21 D.

According to exemplary embodiment 534, provided herein is the growth medium of any one of embodiments 514-533, wherein the growth medium comprises 5 g/L to 15 g/L glucose.

According to exemplary embodiment 535, provided herein is the growth medium of embodiment 534, wherein the growth medium comprises about 5 g/L glucose or about 10 g/L glucose.

According to exemplary embodiment 536, provided herein is the growth medium of any one of embodiments 466-535, wherein the growth medium comprises at least 0.001 g/L of the heme-containing polypeptide.

According to exemplary embodiment 537, provided herein is the growth medium of embodiment 536, wherein the growth medium comprises at least 0.005 g/L of the heme-containing polypeptide.

According to exemplary embodiment 538, provided herein is the growth medium of embodiment 536, wherein the growth medium comprises at least 0.01 g/L the heme-containing polypeptide.

According to exemplary embodiment 539, provided herein is the growth medium of embodiment 536, wherein the growth medium comprises at least 0.02 g/L of the heme-containing polypeptide.

According to exemplary embodiment 540, provided herein is the growth medium of embodiment 536, wherein the growth medium comprises about 0.1 g/L of the heme-containing polypeptide.

According to exemplary embodiment 541, provided herein is the growth medium of embodiment 536, wherein the growth medium comprises about 0.2 g/L of the heme-containing polypeptide.

According to exemplary embodiment 542, provided herein is the growth medium of embodiment 536, wherein the growth medium comprises at least 0.001 g/L of a soy leghemoglobin.

According to exemplary embodiment 543, provided herein is the growth medium of embodiment 542, wherein the growth medium comprises at least 0.005 g/L of a soy leghemoglobin.

According to exemplary embodiment 544, provided herein is the growth medium of embodiment 542, wherein the growth medium comprises at least 0.01 g/L of a soy leghemoglobin.

According to exemplary embodiment 545, provided herein is the growth medium of embodiment 542, wherein the growth medium comprises at least 0.02 g/L of a soy leghemoglobin.

According to exemplary embodiment 546, provided herein is the growth medium of embodiment 542, wherein the growth medium comprises about 0.1 g/L of a soy leghemoglobin.

According to exemplary embodiment 547, provided herein is the growth medium of embodiment 542, wherein the growth medium comprises about 0.2 g/L of a soy leghemoglobin.

According to exemplary embodiment 548, provided herein is the growth medium of any one of embodiments 466-547, wherein the growth medium is at a pH of 5.5 to 7.5.

According to exemplary embodiment 549, provided herein is the growth medium of embodiment 548, wherein the growth medium is at a pH of about 6.5.

According to exemplary embodiment 550, provided herein is a heme-containing polypeptide for use as a supplement in a growth medium for facilitating the growth of hemoglobin-dependent bacteria, wherein the heme-containing polypeptide is: (i) a piscine polypeptide; (ii) an avian polypeptide; or (iii) a non-animal-derived polypeptide.

According to exemplary embodiment 551, provided herein is the heme-containing polypeptide of embodiment 550, wherein the heme-containing polypeptide is a hemoglobin.

According to exemplary embodiment 552, provided herein is the heme-containing polypeptide of embodiment 551, wherein the hemoglobin is a symbiotic hemoglobin, non-symbiotic hemoglobin, and/or truncated hemoglobin.

According to exemplary embodiment 553, provided herein is the heme-containing polypeptide of embodiment 550, wherein the heme-containing polypeptide is a leghemoglobin.

According to exemplary embodiment 554, provided herein is the heme-containing polypeptide of embodiment 550, wherein the heme-containing polypeptide is a myoglobin.

According to exemplary embodiment 555, provided herein is the heme-containing polypeptide of any one of embodiments 550-554, wherein the heme-containing polypeptide is a piscine polypeptide.

According to exemplary embodiment 556, provided herein is the heme-containing polypeptide embodiment 555, wherein the piscine polypeptide is purified from fish.

According to exemplary embodiment 557, provided herein is the heme-containing polypeptide of embodiment 556, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 558, provided herein is the heme-containing polypeptide of embodiment 555, wherein the piscine polypeptide is recombinantly expressed.

According to exemplary embodiment 559, provided herein is the heme-containing polypeptide of any one of embodiments 550-554, wherein the heme-containing polypeptide is an avian polypeptide.

According to exemplary embodiment 560, provided herein is the heme-containing polypeptide of embodiment 559, wherein the avian polypeptide is purified from a bird.

According to exemplary embodiment 561, provided herein is the heme-containing polypeptide of embodiment 560, wherein the bird is of the genus Gallus, Meleagris, Anas, Anser, Branta, Chen, Agelastes, Numida, Guttera, Acryllium, or Columba.

According to exemplary embodiment 562, provided herein is the heme-containing polypeptide of embodiment 559, wherein the avian polypeptide is recombinantly expressed.

According to exemplary embodiment 563, provided herein is the heme-containing polypeptide of any one of embodiments 550-554, wherein the heme-containing polypeptide is a non-animal-derived polypeptide.

According to exemplary embodiment 564, provided herein is the heme-containing polypeptide of embodiment 563, wherein the non-animal-derived polypeptide is purified from plants, bacteria, cyanobacteria, fungus, algae, grain, legume, and/or protozoa.

According to exemplary embodiment 565, provided herein is the heme-containing polypeptide of embodiment 563 or 564, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 566, provided herein is the heme-containing polypeptide of any one of embodiments 563-565, wherein the non-animal-derived polypeptide is a soy leghemoglobin.

According to exemplary embodiment 567, provided herein is the heme-containing polypeptide of embodiment 566, wherein the soy leghemoglobin is purified from soy roots or soy root nodules.

According to exemplary embodiment 568, provided herein is the heme-containing polypeptide of embodiment 563, wherein the non-animal-derived polypeptide is recombinantly expressed.

According to exemplary embodiment 569, provided herein is the heme-containing polypeptide of any one of embodiments 550-554, 563, and 568, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

According to exemplary embodiment 570, provided herein is the heme-containing polypeptide of embodiment 569, wherein the heme-containing polypeptide comprises an amino acid sequence with at least 90% identity to an amino acid sequence in any one of SEQ ID NOs: 1-29, 31, 33, 35, 37, 39, 41, 43, 114, or a combination thereof.

According to exemplary embodiment 571, provided herein is the heme-containing polypeptide of any one of embodiments 558, 562, and 568, wherein the recombinantly expressed polypeptide comprises a heterologous polypeptide.

According to exemplary embodiment 572, provided herein is the heme-containing polypeptide of embodiment 571, wherein the heterologous polypeptide comprises a histidine tag, TAP (tandem affinity purification) tag, TEV cleavage site, a FLAG tag, a GST tag, and/or an immunoglobulin domain.

According to exemplary embodiment 573, provided herein is the heme-containing polypeptide of any one of embodiments 558, 562, and 568-572, wherein the recombinant polypeptide is expressed and purified from an exogenous nucleic acid in a host cell.

According to exemplary embodiment 574, provided herein is the heme-containing polypeptide of embodiment 573, wherein the exogenous nucleic acid is in a vector.

According to exemplary embodiment 575, provided herein is the heme-containing polypeptide of embodiment 574, wherein the vector is an expression vector.

According to exemplary embodiment 576, provided herein is the heme-containing polypeptide of any one of embodiments 573-575, wherein the host cell is bacteria, yeast, insect, or mammalian cell lines.

According to exemplary embodiment 577, provided herein is the heme-containing polypeptide of embodiment 576, wherein the host cell is Pichia Pastoris or Escherichia coli.

According to exemplary embodiment 578, provided herein is the heme-containing polypeptide of any one of embodiments 568-577, wherein the heme-containing polypeptide is a soy leghemoglobin recombinantly expressed in Pichia pastoris.

According to exemplary embodiment 579, provided herein is the heme-containing polypeptide of any one of embodiments 550-578, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

According to exemplary embodiment 580, provided herein is the heme-containing polypeptide of any one of embodiments 550-578, wherein the hemoglobin-dependent bacteria are of the genus Prevotella.

According to exemplary embodiment 581, provided herein is the heme-containing polypeptide of embodiment 580, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella orails, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 582, provided herein is the heme-containing polypeptide of embodiment 580, wherein the hemoglobin-dependent bacteria are of the species Prevotella histicola.

According to exemplary embodiment 583, provided herein is the heme-containing polypeptide of embodiment 580, wherein the Prevotella comprise at least 90% genomic, 16S and/or CRISPR sequence identity to the nucleotide sequence of the Prevotella Strain B 50329 (NRRL accession number B 50329) or Prevotella Strain C (PTA-126140).

According to exemplary embodiment 584, provided herein is the heme-containing polypeptide of embodiment 580, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 585, provided herein is the heme-containing polypeptide of embodiment 580, wherein the Prevotella are Prevotella Strain B 50329 (NRRL accession number B 50329) or Prevotella Strain C (PTA-126140).

According to exemplary embodiment 586, provided herein is the heme-containing polypeptide of any one of embodiments 580-585, wherein the hemoglobin-dependent bacteria are a strain of Prevotella bacteria comprising one or more proteins listed in Table 1.

According to exemplary embodiment 587, provided herein is the heme-containing polypeptide of any one of embodiments 580-586, wherein the hemoglobin-dependent bacteria are a strain of Prevotella substantially free of a protein listed in Table 2.

According to exemplary embodiment 588, provided herein is a bacterial composition comprising (a) hemoglobin-dependent bacteria, and (b) a heme-containing polypeptide, wherein the heme-containing polypeptide is: (i) a piscine polypeptide; (ii) an avian polypeptide; or (iii) a non-animal-derived polypeptide.

According to exemplary embodiment 589, provided herein is the bacterial composition of embodiment 588, wherein the heme-containing polypeptide is a hemoglobin.

According to exemplary embodiment 590, provided herein is the bacterial composition of embodiment 589, wherein the hemoglobin is a symbiotic hemoglobin, non-symbiotic hemoglobin, and/or truncated hemoglobin.

According to exemplary embodiment 591, provided herein is the bacterial composition of embodiment 588, wherein the heme-containing polypeptide is a leghemoglobin.

According to exemplary embodiment 592, provided herein is the bacterial composition of embodiment 588, wherein the heme-containing polypeptide is a myoglobin.

According to exemplary embodiment 593, provided herein is the bacterial composition of any one of embodiments 588-592, wherein the heme-containing polypeptide is a piscine polypeptide.

According to exemplary embodiment 594, provided herein is the bacterial composition embodiment 593, wherein the piscine polypeptide is purified from fish.

According to exemplary embodiment 595, provided herein is the bacterial composition of embodiment 594, wherein the fish is of the genus Ctenopharyngodon, Engraulis, Hypophthalmichthys, Cyprinus, Theragra, Oreochromis, Hypophthalmichthys nobilis, Katsuwonus, Catla, Carassius, Salmo, Clupea, Scomber, Labeo, Trichiurus, Gadus, Sardina, Mallotus, Chanos, Sconmber, Oncorhynchus, Clupea, Procambarus, Brevoortia, Sardinella, Mylopharyngodon, Channa, Gadus, Cololabis, Trachurus, Larimichthys, Melanogrammus, Silurus, Sprattus, Cirrhinus, ktalurus, Micromesistius, Tenualosa, Muraenesox, Sardinops, Cetengraulis, Pollachius, Euthynnus, Rastrelliger, Monopterus, Merluccius, Rastrelliger, Misgurnus, Siniperca, Lates, Sardinops, Harpadon, Scomberomorus, Ethmalosa, Oreochromis, Brevoortia, Opisthonema, Selar, Selaroides, or Ammodytes.

According to exemplary embodiment 596, provided herein is the bacterial composition of embodiment 593, wherein the piscine polypeptide is recombinantly expressed.

According to exemplary embodiment 597, provided herein is the bacterial composition of any one of embodiments 588-592, wherein the heme-containing polypeptide is an avian polypeptide.

According to exemplary embodiment 598, provided herein is the bacterial composition of embodiment 597, wherein the avian polypeptide is purified from a bird.

According to exemplary embodiment 599, provided herein is the bacterial composition of embodiment 598, wherein the bird is of the genus Gallus, Meleagris, Anas, Anser, Branta, Chen, Agelastes, Numida, Guttera, Acryllium, or Columba.

According to exemplary embodiment 600, provided herein is the bacterial composition of embodiment 597, wherein the avian polypeptide is recombinantly expressed.

According to exemplary embodiment 601, provided herein is the bacterial composition of any one of embodiments 588-592, wherein the heme-containing polypeptide is a non-animal-derived polypeptide.

According to exemplary embodiment 602, provided herein is the bacterial composition of embodiment 601, wherein the non-animal-derived polypeptide is purified from plants, bacteria, cyanobacteria, fungus, algae, grain, legume, and/or protozoa.

According to exemplary embodiment 603, provided herein is the bacterial composition of embodiment 601 or 602, wherein the non-animal-derived polypeptide is purified from at least one organism of the genus Arabidopsis, Nicotiana, Acidovorax, Aquifex, Thermophilus, Bacillus, Escherichia, Brevibacillus, Corynebacterium, Frigoribacterium, Methylacidiphilum, Rhizobium, Synechococcus, Synechocystis, Nostoc, Fusarium, Aspergillus, Saccharomyces, Pichia, Schizosaccharomyces, Trichoderma, Myceliopthera, Kluyvera, Chlamydomonas, Oryza, Magnaporthe, Zea, Hordeum, Glycine, Cicer, Phaseolus, Lupinus, Medicago, Brassica, Triticum, Gossypium, Zizania, Helianthus, Beta, Pennisetum, Chenopodium, Sesamum, Linum, Vigna, Ricinus, Pisum, Tetrahymena, or Paramecium.

According to exemplary embodiment 604, provided herein is the bacterial composition of any one of embodiments 601-603, wherein the non-animal-derived polypeptide is a soy leghemoglobin.

According to exemplary embodiment 605, provided herein is the bacterial composition of embodiment 604, wherein the soy leghemoglobin is purified from soy roots or soy root nodules.

According to exemplary embodiment 606, provided herein is the bacterial composition of embodiment 601, wherein the non-animal-derived polypeptide is recombinantly expressed.

According to exemplary embodiment 607, provided herein is the bacterial composition of any one of embodiments 588-592, 601, and 606, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

According to exemplary embodiment 608, provided herein is the bacterial composition of embodiment 607, wherein the heme-containing polypeptide comprises an amino acid sequence with at least 90% identity to an amino acid sequence in any one of SEQ ID NOs: 1-29, 31, 33, 35, 37, 39, 41, 43, 114, or a combination thereof.

According to exemplary embodiment 609, provided herein is the bacterial composition of any one of embodiments 596, 600, and 606, wherein the recombinantly expressed polypeptide comprises a heterologous polypeptide.

According to exemplary embodiment 610, provided herein is the bacterial composition of embodiment 609, wherein the heterologous polypeptide comprises a histidine tag, TAP (tandem affinity purification) tag, TEV cleavage site, a FLAG tag, a GST tag, and/or an immunoglobulin domain.

According to exemplary embodiment 611, provided herein is the bacterial composition of any one of embodiments 596, 600, 606, 609, and 555, wherein the recombinant polypeptide is expressed and purified from an exogenous nucleic acid in a host cell.

According to exemplary embodiment 612, provided herein is the bacterial composition of embodiment 611, wherein the exogenous nucleic acid is in a vector.

According to exemplary embodiment 613, provided herein is the bacterial composition of embodiment 612, wherein the vector is an expression vector.

According to exemplary embodiment 614, provided herein is the bacterial composition of any one of embodiments 611-613, wherein the host cell is bacteria, yeast, insect, or mammalian cell lines.

According to exemplary embodiment 615, provided herein is the bacterial composition of embodiment 614, wherein the host cell is Pichia Pastoris or Escherichia coli.

According to exemplary embodiment 616, provided herein is the bacterial composition of any one of embodiments 606-615, wherein the heme-containing polypeptide is a soy leghemoglobin recombinantly expressed in Pichia pastoris.

According to exemplary embodiment 617, provided herein is the bacterial composition of any one of embodiments 588-616, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

According to exemplary embodiment 618, provided herein is the bacterial composition of any one of embodiments 588-616, wherein the hemoglobin-dependent bacteria are of the genus Prevotella.

According to exemplary embodiment 619, provided herein is the bacterial composition of embodiment 618, wherein the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

According to exemplary embodiment 620, provided herein is the bacterial composition of embodiment 618, wherein the hemoglobin-dependent bacteria are of the species Prevotella histicola.

According to exemplary embodiment 621, provided herein is the bacterial composition of embodiment 618, wherein the Prevotella comprise at least 90% genomic, 16S and/or CRISPR sequence identity to the nucleotide sequence of the Prevotella Strain B 50329 (NRRL accession number B 50329) or Prevotella Strain C (PTA-126140).

According to exemplary embodiment 622, provided herein is the bacterial composition of embodiment 618, wherein the Prevotella comprise at least 99% genomic, 16S and/or CRISPR sequence identity to the nucleotide sequence of the Prevotella Strain B 50329 (NRRL accession number B 50329) or Prevotella Strain C (PTA-126140).

According to exemplary embodiment 623, provided herein is the bacterial composition of embodiment 618, wherein the Prevotella are Prevotella Strain B 50329 (NRRL accession number B 50329) or Prevotella Strain C (PTA-126140).

According to exemplary embodiment 624, provided herein is the bacterial composition of any one of embodiments 618-623, wherein the hemoglobin-dependent bacteria are a strain of Prevotella bacteria comprising one or more proteins listed in Table 1.

According to exemplary embodiment 625, provided herein is the bacterial composition of any one of embodiments 618-624, wherein the hemoglobin-dependent bacteria are a strain of Prevotella substantially free of a protein listed in Table 2.

### EXAMPLES

### Example 1: Exemplary Purification of Heme-containing Polypeptides

All steps are carried out at 4° C. or room temperature. Centrifugation steps are at 8000×g for 20 mins, 4° C. or room temperature.

The clarified tissue lysates or protein solutions prepared as described below are subjected to ammonium sulfate precipitation. Briefly, the clarified tissue lysates or protein solutions are buffered with 1M HEPES, pH 7.4 or 1 M Tris-HCl, pH 7.4, to a final concentration of 50 mM. Proteins are precipitated by addition of ammonium sulfate in at least two steps at 50% and 80% saturation. The lysate with ammonium sulfate is stirred gently for a minimum of 4 hours and a maximum of 16 hours at 4° C. The solution is centrifuged at 25,000×g for 1 hour at 4° C.

Upon fractionation, all ammonium sulfate precipitate fractions of interest are stored at −20° C. until further use. Prior to their use in experiments, the precipitates are resuspended in 10 volumes of 50 mM potassium phosphate buffer, pH 7.4, containing 0.5 M NaCl. The suspensions are centrifuged and the supernatants are microfiltered through a 0.2 micron PES membrane. The filtrates are concentrated by ultrafiltration on a 3 kDa, 5 kDa, or 10 kDa molecular weight cutoff PES membrane on a Spectrum Labs KrosFlo hollow fiber tangential flow filtration system. The protein composition at individual fractionation step is monitored by SDS-PAGE and protein concentrations are measured by standard UV-Vis methods. Hemoglobin, myoglobin, or other heme-containing polypeptide is further separated by size exclusion chromatography (Sephacryl S-100 HR, GE Healthcare) based on the molecular size of the protein. The fraction is optionally further purified using an ion exchange chromatography (High Prep Q; Prep S; High Prep DEAE, GE Healthtcare). The fraction containing the heme-containing polypeptide is determined by SDS-PAGE and/or mass spectrometry.

Heme-Containing Polypeptide from Fish

Carps are coarsely chopped and quickly frozen in liquid nitrogen. The frozen tissue is ground in liquid nitrogen with mortar and pestle until fine powder is formed. The powder is stored in −80° C. until further use.

The ground carp powder or commercially available fish protein hydrolysates are suspended in 10 volumes of 50 mM potassium phosphate buffer, pH 8.0 and 0.4 M sodium chloride and stirred for 1 hour at 4° C. Soluble proteins are separated from the rest by centrifugation. The supernatant is subjected to ammonium sulfate precipitation described above.

Heme-Containing Polypeptide from Birds

Chicken meat is coarsely chopped and frozen in liquid nitrogen. The frozen tissue is ground in liquid nitrogen with mortar and pestle until fine powder is formed. The powder is stored in −80° C. until further use.

The ground chicken powder or commercially available chicken meals are suspended in 10 volumes of 50 mM potassium phosphate buffer, pH 8.0 and 0.4 M sodium chloride. The lysate is clarified by centrifugation. The supernatant is used for ammonium sulfate precipitation described above.

Heme-Containing Polypeptide from Non Animal Source

Commercially available legume flour is suspended in 10 volumes of 50 mM potassium phosphate buffer, pH 8.0 and 0.4 M sodium chloride. The lysate is clarified by centrifugation. The supernatant is used for ammonium sulfate precipitation described above.

Pea total proteins: Dry green or yellow pea flour is used to extract total pea proteins. The flour is suspended in 10 volumes of 20 mM potassium phosphate buffer pH 8 and 100 mM sodium chloride and stirred for 1 hour. Soluble protein is separated from pea seed debris by centrifugation. The supernatant is collected and filtered through a 0.2 micron membrane and concentrated using a 10 KDa cutoff PES membrane.

Lentil total proteins: Air-classified lentil flour is used to extract crude mixture of lentil proteins. Flour is suspended in 5 volumes of 20 mM potassium phosphate buffer pH 7.4 and 0.5 M sodium chloride and stirred for 1 hour. Soluble protein is separated from the unextracted protein and lentil seed debris by centrifugation (8000 g, 20 minutes). The supernatant is collected and filtered through a 0.2 micron membrane and concentrated using a 10 KDa cutoff PES membrane.

Chickpea/Garbanzo bean total proteins: Garbanzo bean flour is suspended in 5 volumes of 20 mM potassium phosphate buffer pH 7.4 and 0.5 M sodium chloride and stirred for 1 hour. Soluble protein is separated from the unextracted protein and chickpea seed debris by centrifugation (8000 g, 20 minutes). The supernatant is collected and filtered through a 0.2 micron membrane and concentrated using a 10 KDa cutoff PES membrane.

Soybean proteins: Soybean proteins are also extracted by suspending the defatted soy flour in 4-15 volumes (e.g., 5 volumes) of 20 mM sodium carbonate, pH 9 (or water, pH adjusted to 9 after addition of the flour) or 20 mM potassium phosphate buffer pH 7.4 and 100 mM sodium chloride. The slurry is stirred for one hour and centrifuged at 8000×g for 20 minutes. The extracted proteins are ultrafiltered and then processed as above or alternatively, the supernatant is collected and filtered through a 0.2 micron membrane and concentrated using a 10 KDa cutoff PES membrane.

Amaranth flour dehydrins: Amaranth flour is suspended in 5 volumes of 0.5 M sodium chloride, pH 4.0 and stirred for 1 hour. Soluble protein is separated from the unextracted protein and debris by centrifugation (8000×g, 20 minutes). The supernatant is collected and filtered through a 0.2 micron membrane and concentrated using a 3 KDa cutoff PES membrane. Further enrichment of dehydrins from this fraction is obtained by boiling the concentrated protein material, spinning at 8000×g for 10 minutes, and collecting the supernatant.

Pea globulins: Dry green pea flour is used to extract pea globulin proteins. The flour is suspended in 10 volumes of 50 mM potassium phosphate buffer pH 8 and 0.4 M sodium chloride and stirred for 1 hour. Soluble protein is separated from pea seed debris by centrifugation. The supernatant is subjected to ammonium sulfate fractionation in two steps at 50% and 80% saturation. The 80% pellet containing globulins of interest is stored at −20° C. until further use. Protein is recovered from the pellet and prepared for use as described above.

Soybean 7S and 11S globulins: Globulins from soybean flour are isolated by first suspending lowfat/defatted soy flour in 4-15 volumes of 20 mM potassium phosphate pH 7.4. The slurry is centrifuged at 8000×g for 20 mins or clarified by 5 micron filtration and the supernatant is collected. The crude protein extract contains both the 7S and 11S globulins. The solution is filtered using a 0.2 micron filter and concentrated using a 10 kDa molecular weight cutoff PES membrane on a Spectrum Labs KrosFlo hollow fiber tangential flow filtration system or by passing over the anion-exchange resin prior to use in experiments. The 11S globulins are separated from the 7S proteins by isoelectric precipitation. The pH of the crude protein extract is adjusted to 6.4 with dilute HCl, stirred for 30 min-1 hour and then centrifuged to collect the 11S precipitate and 7S proteins in the supernatant. The 11S fraction is resuspended with 10 mM potassium phosphate pH 7.4 and the protein fractions are micro-filtered and concentrated prior to use.

Mung bean 8S globulins: Mung bean flour is used to extract 8S globulins by first suspending the flour in 4 volumes of 50 mM potassium phosphate buffer pH 7, 0.5M NaCl. After centrifugation, proteins in the supernatant are fractionated by addition of ammonium sulfate in 2 steps at 50% and 90% saturation respectively. The precipitate from the 90% fraction contains the 8S globulins, which is saved at −20° C. until further use. 8S globulins are recovered from the pellet and prepared for use as described above.

Mung bean globulins: Mung bean globins are also extracted by suspending the flour in 4 volumes of 20 mM sodium carbonate buffer, pH 9 (or water adjusted to pH 9 after addition of the mung bean flour). The slurry is centrifuged (or filtered) to remove solids, ultrafiltered, and then processed as described above.

Leghemoglobin. Soy root nodules are suspended and lysed in 20 mM potassium phosphate pH 7.4, 100 mM potassium chloride and 5 mM EDTA using a grinder-blender. During this process, leghemoglobin is released into the buffer. Root-nodule lysate containing leghemoglobin is cleared from cell debris by filtration through 5 micron filter. Filtration is followed by centrifugation (7000×g, 20 min). Clarified lysate containing leghemoglobin is then filtered through 0.2 micron filter and applied onto an anion-exchange chromatography column (High Prep Q; High Prep DEAE, GE Healthtcare) on a fast protein liquid chromatography instrument (GE Healthcare). Leghemoglobin is collected in the flowthrough fraction and concentrated over 3 kDa molecular weight cutoff PES membrane on a Spectrum Labs KrosFlo hollow fiber tangential flow filtration system to a desired concentration. Purity (partial abundance) of purified leghemoglobin is analyzed by SDS-PAGE gel. In lysate, leghemoglobin is typically present at 20-40%, while after anion-exchange purification, it can be present at 70-80%. The soybean leghemoglobin flowthrough from anion-exchange chromatography is optionally applied onto size-exclusion chromatography (Sephacryl S-100 HR, GE Healthcare). Soybean leghemoglobin elutes as two fractions corresponding to dimeric and monomeric species. Purity (partial abundance) of leghemoglobin is analyzed by SDS-PAGE and can be in the range of ˜90-100%. Analysis of UV-VIS spectra (250-700 nm) can reveal spectral signature consistent with heme loaded leghemoglobin.

### Example 2: Exemplary Recombinant Heme-Containing Polypeptide Expression and Purification

Monomeric Heme-Containing Polypeptide

Non-symbiotic hemoglobin from mung bean is cloned into pJexpress401 vector (DNA2.0) and transformed into E. coli BL21. Cells are grown in LB media containing soytone instead of tryptone, kanamycin, 0.1 mM ferric chloride and 10 μg/ml 5-aminolevulinic acid. Expression is induced by 0.2 mM IPTG and cells are grown at 30° C. for 20 hours. E. coli cells expressing a mung bean non-symbiotic hemoglobin are collected and resuspended in 20 mM MES buffer pH 6.5, 50 mM NaCl, 1 mM MgCl₂, 1 mM CaCl₂), DNAase I, and protease inhibitors. Cells are lysed by sonication. Lysate is cleared from cell debris by centrifugation at 16,000×g for 20 min, followed by filtration over 200 nm filter. The cell lysate is then loaded over a FF-S column on a fast protein liquid chromatography instrument (GE Healthcare). Mung bean non-symbiotic hemoglobin binds to the FF-S column and the bound protein is eluted using a sodium chloride gradient (50 mM-1000 mM). Purity (partial abundance) of mung bean non-symbiotic hemoglobin is analyzed by SDS-PAGE. In a typical case, the purity (partial abundance) in E. coli lysate is about 13%, and after purification on FF-Q is about 35%. UV-Vis analysis of the purified protein can show spectra characteristic of heme bound protein.

Monomeric heme-containing polypeptides are synthesized with an N-terminal His6 epitope tag (SEQ ID NO: 115) and a TEV cleavage site, cloned into pJexpress401 vector (DNA2.0), and transformed into E. coli BL21. Transformed cells are grown in LB media containing soytone instead of tryptone, kanamycin, 0.1 mM ferric chloride and 10 g/ml 5-aminolevulinic acid. Expression is induced by 0.2 mM IPTG and cells are grown at 30° C. for 20 hours. E. coli cells expressing heme proteins are collected and resuspended in 50 mM potassium phosphate pH 8, 150 mM NaCl, 10 mM imidazole, 1 mM MgCl₂, 1 mM CaCl₂), DNAase I, and protease inhibitors. Cells are lysed by sonication and clarified by centrifugation at 9000×g. Lysate is incubated with NiNTA resin (MCLAB), which is subsequently washed with 5 column volumes (CV) of 50 mM potassium phosphate pH 8, 150 mM NaCl, 10 mM imidazole. The bound proteins are eluted with 50 mM potassium phosphate pH 8, 150 mM NaCl, 500 mM imidazole. SDS-PAGE and UV-vis spectra can confirm the expected molecular weights as well as heme-loading, respectively.

In some cases, the transformed cells are grown in seed media comprising 10 g/L glucose monohydrate, 8 g/L monopotassium phosphate, 2.5 g/L Sensient Amberferm 6400, 2.5 g/L Sensient Tastone 154, 2 g/L diammonium phosphate, 1 mL/L trace metals mixture (Teknova 1000×trace metals mixture Cat. No. T1001), 1 g/L magnesium sulfate, 0.25 mL 0.1 M solution ferric chloride, 0.5 mL/L Sigma anti-foam 204, 1 mL/L kanamycin sulfate 1000×solution. 250 mL of media is used in four 1 L baffled shakeflasks, inoculated with 0.25 mL each from a single vial of glycerol stock culture. Shakeflasks are grown for 5.5 hours, with 250 RPM agitation at 37° C. 40 L of seed media is steam-sterilized in a 100 L bioreactor, cooled to 37° C., pH-adjusted to 7.0, and is inoculated with 800 mL of shakeflask culture, once a shakeflask OD of 2.5 is achieved. Aeration to the bioreactor is supplied at 40 L/m and the agitation is at 250 RPM. After 2.2 hours of growth, or when an OD of 2.2 is reached, the 22 L of culture is transferred a bioreactor. The starting media for the final bioreactor comprises of the following components steamed-in-place: 1775 L deionized water, 21.75 kg monopotassium phosphate, 2.175 kg diammonium phosphate, 4.35 kg ammonium ferric citrate, 8.7 kg ammonium sulfate, 10.875 kg Sensient Amberferm 6400, and 10.875 kg Sensient Tastone 154. After 30 minutes of steaming, the media components are cooled to 37° C. and post-sterilization additions are made: 2.145 L of 0.1 M ferric chloride solution, 59.32 kg 55% w/w glucose monohydrate, 3.9 L of trace metals mixture (Teknova 1000×trace metals mixture Cat. No. T1001), 10.88 L of 200 g/L diammonium phosphate, 36.14 L 1 M magnesium sulfate, and 2.175 L Sigma anti-foam 204, 2.175 L kanamycin sulfate 1000×solution. pH is controlled at 7.0 via addition of 30% ammonium hydroxide. Aeration is supplied at 2.175 m³/min, and the dissolved oxygen is controlled at 25% by varying agitation between 60-150 RPM. At two time points, bolus additions of additional nutrients are supplied. Each addition adds 5.5 kg of Sensient Amberferm 6400, 5.5 kg of Sensient Tastone 154 and 4.4 kg of diammonium phosphate, in autoclaved solutions (100 g/L solution for Amberferm and Tastetone, 200 g/L for diammonium phosphate). A sterile glucose solution of 55% w/w glucose monohydrate is fed into the bioreactor to maintain a level of residual glucose of 2-5 g/L. Once an OD of 2.5 is reached, the temperature is reduced to 25° C. and the culture is induced with 0.648 L of 1 M Isopropyl β-D-1-thiogalactopyranoside. The culture is allowed to grow for a total time of 25 hours, at which point the culture is diluted 1:1 with deionized water, then centrifuged, concentrating the centrate to 50% v/v solids content. Cell centrate is frozen at −20° C. Centrate is thawed to 4° C. and diluted in 20 mM potassium phosphate p 7.8, 00 mM NaCl, 0 mM imidazole, and homogenized at 15,000 PSI. Homogenized cells are 0.2 μm filtered by tangential flow filtration (TFF) and filtered lysate is loaded directly onto a zinc-charged IMAC column (GE). Bound proteins are washed with 10 column volumes (CV) 20 mM potassium phosphate pH 7.4, 100 mM NaCl, 5 mM histidine and eluted with 10 CV 500 mM potassium phosphate monobasic, 100 mM NaCl. Eluted leghemoglobin is concentrated and diafiltered using a 3 kDa molecular weight cutoff PES membrane and TFF. The concentrated sample is reduced with 20 mM sodium dithionite and desalted using a G-20 resin (GE). Desalted leghemoglobin samples are frozen in liquid nitrogen and stored at −20° C. Leghemoglobin concentration and purity are determined by SDS-PAGE and UV-vis analysis.

A non-limiting example of cloning, expression, and reconstitution of a myoglobin with heme includes Varadaraj an et al. (1985) Proc Natl Acad Sci USA 82:5681-5684. Heme-containing polypeptides can be readily prepared in large scale using these methods.

Tetrameric Heme-Containing Polypeptide

Expression and Purification in E. Coli

The plasmid vector pCold TFTM DNA (TaKaRa, Bio Inc, Japan) is used for soluble protein expression in E. coli. Chicken hemoglobin subunits α and β are cloned into the expression vector as described for co-expression (e.g., Aniwised et al. (2013) Protein J 32:172-182). Alternatively, constructs co-expressing the subunits are synthesized (Blue Heron Biotech, Bothell, Wash., USA).

The overexpression of globin genes induced at 37° C. for 5 hours results in aggregation of the expressed proteins to form inclusion bodies. To avoid this, overexpression of globin genes is induced at 15° C. overnight. Specifically, the transformed E. coli JM109 cells are cultivated at 37° C. in LB media containing 50 μg/mL of ampicillin until the optical density at 600 nm reaches 0.4-0.5. Expression is then induced by adding 1 mM isopropyl-β-D-thiogalactopyranoside (IPTG) at 15° C. overnight. Cells from 100 mL culture (˜0.9 g) are centrifuged at 6000 rpm for 10 min at 4° C. To prepare the crude extract, the cells are resuspended in 10 mL of lysis buffer (10 mM Tris-HCl, pH 8.0), then 0.1 mg/mL of lysozyme is added to the crude extract solution, followed by incubation on ice for 30 minutes.

The cells are disrupted by sonication and centrifuged at 12,000 rpm for 10 minutes at 4° C. The soluble proteins are loaded onto a Co²⁺-charged Talon resin column (Clontech, Palo Alto, Calif., USA), equilibrated with 10 mM Tris-HCl (pH 8.0), 150 mM NaCl. After washing the column with the same buffer, the protein is eluted from the column by applying a stepwise gradient of 50 to 500 mM imidazole eluent. The protein bands on the SDS-PAGE gel are visualized by staining with Coomassie brilliant blue R250.

The target fusion protein is eluted by adding 300 mM imidazole and dialyzed against 10 mM Tris-HCl buffer, pH 8.0 overnight at 4° C. The purified fusion protein is concentrated using a centricon filter (30,000 MW cut off; Millipore Ireland, Cork, Ireland). To cleave the fusion protein, 30 units of HRV3C protease (Nacalai tesque, Inc. Japan) is employed. All cleavage reactions are conducted at 4° C. overnight and further analyzed by SDS-PAGE and native-PAGE. The cleaved protein solution is then loaded on an immobilized metal affinity chromatography (Co²⁺-IMAC) column (10×40 mm, Clontech, Palo Alto, Calif., USA), equilibrated with 10 mM Tris-HCl (pH 8.0) containing 150 mM NaCl. After washing the column with the same buffer, the protein is eluted from the column in 2 mL fractions using 0.3 M imidazole in the elution buffer. The eluted proteins are visualized by SDS-PAGE.

Expression and Purification in P. Pastoris (Secretion of Recombinant Protein)

Expression constructs for P. pastoris: The plasmid pPIC9K vector (Invitrogen, Carlsbad, Calif., USA) is used for direct secretion of a recombinant protein in yeast. P. pastoris GS115 strain, genotype his4 (Invitrogen, Carlsbad, Calif., USA), is used for yeast transformation and protein expression. Chicken hemoglobin subunits α and β are cloned into the expression vector as described (e.g., Aniwised et al. (2013) Protein J32:172-182). Alternatively, constructs co-expressing the subunits are synthesized via a commercial vendor (e.g., Blue Heron Biotech, Bothell, Wash., USA).

P. pastoris/GS115 transformation, designated as recombinant cHb (pPIC9K(His6×)-cHb), is processed according to the method described by the Invitrogen protocol. Prior to transformation, pPIC9K(His6×)-cHb recombinant plasmids are extracted from E. coli. Briefly, the E. coli cells are cultivated at 37° C. overnight in 40 mL of LB containing 50 μg/mL of ampicillin. The recombinant plasmids are extracted using an alkaline extraction method, and linearized with a restriction enzyme (e.g., SalI), then transformed to P. pastoris/GS115 by a lithium chloride method according to the manufacturer's instructions (Invitrogen). Yeast cells are grown in minimal dextrose (MD) medium at 30° C. for 2-3 days, following the manufacturer's recommendations.

Positive multiple copy colonies are cultivated in 2 mL of BMGY medium (2% peptone, 1% yeast extract, 40 mM (NH₄)₂SO₄, 100 mM potassium phosphate, pH 6.0, 4×10⁻⁵% biotin and 1% glycerol), and incubated at 30° C. for 24 hours using a shaking incubator (160 rpm). The cells are collected by centrifugation at 8000 rpm for 5 minutes at ambient temperature and resuspended in the same medium, except 1% methanol is added to the medium instead of glycerol. The homogenized cultures are transferred into 1 mL of fresh BMMY broth. In order to enhance the expression level of recombinant pPIC9K(His6×)-cHb in P. pastoris, temperature (25 and 30° C.) and methanol concentration (0.5, 1, 1.5, and 2%) are optimized. The culture is maintained for 72 hours. After 3 days, the culture medium is collected by centrifugation at 12,000 rpm and 4° C. for 10 minutes. The secreted recombinant protein in culture medium is monitored using SDS-PAGE.

After 72 hours of culturing, the entire medium is harvested by centrifugation at 12,000 rpm and 4° C. for 20 minutes, and the supernatant is concentrated and washed using a centricon filter (Millipore Ireland, Cork, Ireland). Then, the crude protein is loaded on an affinity chromatography (Co²⁺-IMAC) column (10×40 mm, Clontech, Palo Alto, Calif., USA) pre-equilibrated with 10 mM Tris-HCl (pH 8.0) containing 150 mM NaCl. After washing the column with the same buffer, the bound protein is eluted from the column in 2 mL fractions by 0.5 M imidazole in the elution buffer. The eluted proteins are analyzed by SDS-PAGE.

### Example 3: Determining Proper Incorporation of the Heme Group in Purified Heme-Containing Polypeptides

Recombinant heme-containing protein produced from any organism (e.g., E. coli or P. pastoris) is analyzed for proper incorporation of the heme group using the pyridine hemochromogen method (Oinuma et al., (2003) J Mol Chem 278:29600-29608) using the molecular extinction coefficient of the α-peak at 557 nm of the hemochromogen of protoheme IX (ε=34.4 mM⁻¹ cm⁻¹). Briefly, 200 μL of 0.5 M NaOH is added to 500 μL of purified protein and then 200 μL of pyridine and 5 μL of 0.1 M K₃Fe(CN)₆ are added to the mixture. Then, 10 μL of 0.5 M Na₂S₂O₄ is added to heme-containing protein. The absorption spectrum of the sample is monitored from 340 to 700 nm using a Halo DB-20 UV-Vis spectrophotometer (Dynamica Ltd., VIC, AU). The heme content is reported as mol of heme per mol of globin, and the molar extinction coefficient of heme is calculated by substitution in the Beer-Lambert law. Furthermore, the iron content of the recombinant α-globin is measured with an atomic absorption flame emission spectrophotometer (PerkinElmer, Analyst 800, USA). The typical protein concentration used in UV-VIS experiments is 8-9 μM purified recombinant α-globin.

### Example 4: Exemplary Manufacturing Process of Hemoglobin-Dependent Bacteria

A hemoglobin solution is also prepared by dissolving a piscine hemoglobin, an avian hemoglobin, a fungal hemoglobin, a plant hemoglobin, or a bacterial hemoglobin in 0.01 M NaOH. The solution is sterilized by autoclaving. A working concentration of 0.02 g/L or 0.2 g/L is used. A myoglobin solution, a leghemoglobin solution, or other heme-containing polypeptide solution (e.g., peroxidase, cytochrome c) is prepared similarly in phosphate buffer saline, pH 7.4, or 0.01 M NaOH. The solution is sterilized by autoclaving, and is added to the growth media at various working concentrations (e.g., 0.02 g/L, 0.05 g/L, 0.1 g/L, 0.2 g/L, or 0.5 g/L).

An exemplary manufacturing process of hemoglobin-dependent bacteria, e.g., Prevotella histicola is presented herein. In this exemplary method, the hemoglobin-dependent bacteria are grown in growth media comprising the components listed in Table 4. The media is filter sterilized prior to use.

TABLE 4
Exemplary Growth Media

| Component | g/L |
|---|---|
| Yeast Extract 19512 | 10 |
| Soy Peptone A2SC 19649 | 12.5 |
| Soy Peptone E110 19885 | 12.5 |
| Dipotassium Phosphate K2HPO4 | 1.59 |
| Monopotassium phosphate | 0.91 |
| L-Cysteine-HCl | 0.5 |
| Ammonium chloride | 0.5 |
| Glucidex 21 D (Maltodextrin) | 25 |
| Glucose | 10 |
| Heme-containing polypeptide | 0.02 |

TABLE 5
Another Exemplary Growth Media

| Component | g/L |
|---|---|
| Yeast Extract 19512 Organotechnie S.A.S. | 10 |
| Soy Peptone A2SC 19649 Organotechnie S.A.S. | 10 |
| Soy Peptone E110 19885 Organotechnie S.A.S. | 10 |
| Dipotassium Phosphate K2HPO4 | 2.5 |
| L-Cysteine-HCl | 0.5 |
| Glucose | 5 |
| Heme-containing polypeptide | 1 |

Briefly, a 1 L bottle is inoculated with a 1 mL of a cell bank sample that had been stored at −80° C. This inoculated culture is incubated in an anaerobic chamber at 37° C., pH=6.5 due to sensitivity of this strain to aerobic conditions. When the bottle reaches log growth phase (after approximately 14 to 16 hours of growth), the culture is used to inoculate a 20 L bioreactor at 5% v/v. During log growth phase (after approximately 10 to 12 hours of growth), the culture is used to inoculate a 3500 L bioreactor at 0.5% v/v.

Fermentation culture is continuously mixed with addition of a mixed gas at 0.02 VVM with a composition of 25% CO₂ and 75% N₂. pH is maintained at 6.5 with ammonium hydroxide and temperature is controlled at 37° C. Harvest time is based on when the stationary phase is reached (after approximately 12 to 14 hours of growth).

Once fermentation completes, the culture is cooled to 10° C., centrifuged, and the resulting cell paste is collected. 10% Stabilizer is added to the cell paste and mixed thoroughly (Stabilizer Concentration (in slurry): 1.5% Sucrose, 1.5% Dextran, 0.03% Cysteine).

For other growth conditions that can be used, see, e.g., WO 2019/051381, the disclosure of which is hereby incorporated by reference.

TABLE 6
Stabilizer Formulation

| Component | g/kg |
|---|---|
| Sucrose | 200 |
| Dextran 40k | 200 |
| Cysteine HCl | 4 |
| Water | 596 |

Growth Analysis

Four replicates are performed for each growth analysis. 0.1% inoculum from a frozen cell bank is used for each culture. Bacteria are grown in the media of Table 5 as described above. Kinetics of bacterial growth are measured by measuring the optical density (OD600) every 30 minutes on a plate reader for 48 hours while culturing in the anaerobic environment at 37° C.

### Example 5: Isolation and Purification of Soy Leghemoglobin

A nucleic acid encoding Glycine max leghemoglobin C2 (Uniprot KB P02236) with an N-terminal His6 epitope tag (SEQ ID NO: 115) and a TEV cleavage site is cloned into the pJexpress401 vector (DNA2.0), and transformed into E. coli BL21. Transformed cells are grown by fed-batch fermentation supplemented with kanamycin, 0.1 mM ferric chloride and 10 ug/ml 5-aminolevulinic acid. Expression is induced by 0.3 mM isopropyl β-D-1-thiogalactopyranoside (IPTG) and cells are grown at 30 degrees ° C. for 24 hr. Cells are concentrated by centrifugation and resuspended in 20 mM potassium phosphate pH 7.8, 100 mM NaCl. Cells are lysed by high-pressure homogenization and clarified by centrifugation and microfiltration. Leghemoglobin is purified from the soluble lysate using zinc-charged IMAC sepharose fast flow resin (GE Healthcare). Bound leghemoglobin is eluted off the resin with 500 mM potassium phosphate monobasic, 100 mM NaCl. Purified leghemoglobin is neutralized and concentrated using ultrafiltration. Concentrated leghemoglobin is reduced with 20 mM Na dithionite. Na dithionite is removed by diafiltration. Leghemoglobin concentration is determined by soret peak absorbance and adjusted to 60-70 mg/ml. The final leghemoglobin product is frozen in liquid nitrogen, lyophilized, and stored at −20 degrees C. Purity (partial abundance) of leghemoglobin is analyzed by SDS-PAGE and determined to be about 80%. Analysis of UV-VIS spectra (250-700 nm) reveals spectral signature consistent with heme-loaded leghemoglobin.

Glycine max leghemoglobin C2 and eight Pichia pastoris heme biosynthesis genes (listed in Table 7) are cloned into the Pichia pastoris expression vector pJA (BioGrammatics Inc.; Carlsbad, Calif.) under the control of the pAOX1 methanol inducible promoter. Pichia pastoris strain Bg11 (BioGrammatics, Inc.) is transformed with linearized plasmids, and stable integrants are selected by antibiotic resistance.

TABLE 7

| | | | UniprotKB |
|---|---|---|---|
| Gene | Species | Function | # |
| Leghemoglobin C2 | Glycine max | Leghemoglobin | P02236 |
| | | production | |
| ALA synthase | Pichia pastoris | Heme enzyme- | F2QS71 |
| | | step 1 | |
| ALA dehydratase | Pichia pastoris | Heme enzyme- | F2QZA1 |
| | | step 2 | |
| Porphobilinogen | Pichia pastoris | Heme enzyme- | F2QP90 |
| deaminase | | step 3 | |
| Uroporphyrinogen | Pichia pastoris | Heme enzyme- | F2QSR5 |
| III synthase | | step 4 | |
| Uroporphyrinogen | Pichia pastoris | Heme enzyme- | F2QUW1 |
| III decarboxylase | | step 5 | |
| Coproporphyrinogen | Pichia pastoris | Heme enzyme- | F2QUX3 |
| oxidase | | step 6 | |
| Protoporphyrinogen | Pichia pastoris | Heme enzyme- | F2R0D |
| III oxidase | | step 7 | |
| Ferrochelatase | Pichia pastoris | Heme enzyme- | F2QWX6 |
| | | step 8 | |
| Sh bleomycin | Streptoalloteichus | Resistance to | P17493 |
| | hindustanus | Zeocin | |
| Beta lactamase | E. coli | Resistance to | Q9L5C7 |
| | | ampicillin | |
| Hygromycin | E. coli | Resistance to | P00557 |
| | | hygromycin | |
| NatR | Streptomyces | Nourseothricin | O33583 |
| | noursei | resistance | |
| Neomycin resistance | Synthetic | Resistance to | n/a |
| | bacterial | geniticin | |
| | transposon Tn5 | (G418) | |

Transformed Pichia cells are grown by fed-batch fermentation and leghemoglobin expression is induced with methanol for 120 hours at 30 degrees ° C. Cells are concentrated by centrifugation, resuspended in water, and lysed by high pressure homogenization. Solids are removed by treatment with Tramfloc 863A, centrifugation, and 0.2 micron microfiltration (Koch Membrane Systems). The soluble lysate is concentrated and diafiltered with water using 3 kDa ultrafiltration (Spectrum Laboratories). The formulated lysate is partially purified using HPA25 L anion exchange resin (Mitsubishi) to a final purity of about 40%. The partially purified leghemoglobin solution is re-formulated by concentration and water diafiltration using 3 kDa ultrafiltration (Spectrum Laboratories) and further purified using Q Fast Flow anion exchange resin (GE Lifesciences). The final leghemoglobin product is concentrated using 3 kD ultrafiltration and frozen at −20 degrees C. The final product is about 80% pure and contains 80 g/L leghemoglobin.

See U.S. Pat. No. 10,798,958.

### Example 6: Testing Soy Leghemoglobin as a Substitute for Animal-Sourced Hemoglobin in Growth Media

A sample of Impossible™ meat acquired from a commercial grocer was used to generate solutions containing different concentrations of soy leghemoglobin as listed in Table 7.

TABLE 8
Soy leghemoglobin Solutions

| Name | Solution |
|---|---|
| IM1 | 1% solution of soy leghemoglobin in 0.01M NaOH |
| IM2 | 0.5% solution of soy leghemoglobin in 0.01M NaOH |
| IM3 | 0.25% solution of soy leghemoglobin in water |
| IM4 | 0.25% solution of soy leghemoglobin in 0.01M NaOH |

Solutions IM1 and IM2 were prepared by resuspending the Impossible™ meat in distilled water and adding 0.01 M NaOH to achieve a concentration similar to the concentration used for the animal-sourced hemoglobin or spirulina stock solutions. After resuspension, solids were removed from the solutions by centrifugation. Solutions IM3 and IM4 were prepared by resuspending the Impossible™ meat in either water or 0.01 M NaOH solution followed by heating until boiling. Solids were then removed by double filtration through a gauze filter. All solutions were autoclaved following solid removal and then added to growth media to test a range of 3 concentrations: 20 mg/L final concentration of soy leghemoglobin (this is the concentration currently used for animal-sourced hemoglobin); 100 mg/L final concentration of soy leghemoglobin; and 200 mg/L final concentration of soy leghemoglobin.

As a positive control, a spirulina solution at 1 g/L concentration or an animal-sourced hemoglobin solution at 20 mg/L concentration was used. The SPY media (Table 8) with glucose without any other additives was used as a negative control.

The following recipe of the growth media was used:

TABLE 9
SPY Media

| | g/L |
|---|---|
| Component | SPY |
| Yeast Extract 19512 Organotechnie S.A.S. | 10 |
| Soy Peptone A2 SC 19649 Organotechnie S.A.S. | 10 |
| Soy Peptone El10 19885 Organotechnie S.A.S. | 10 |
| Dipotassium Phosphate K2HPO4 | 2.5 |
| L-Cysteine-HCl | 0.5 |

5 g/L of glucose was used as a carbon source for all growth media.

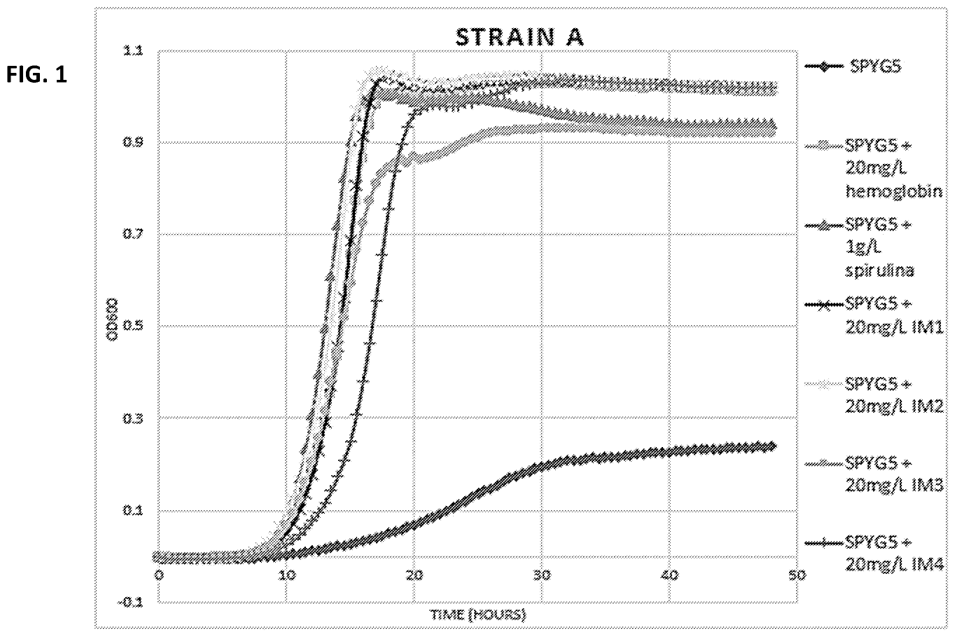

For all 4 IM solutions (IM1-IM4), growth of Strain A (Prevotella histicola strain B 50329 (NRRL accession number B 50329)) was restored to levels similar to when cultured with animal-sourced hemoglobin or spirulina (FIG. 1).

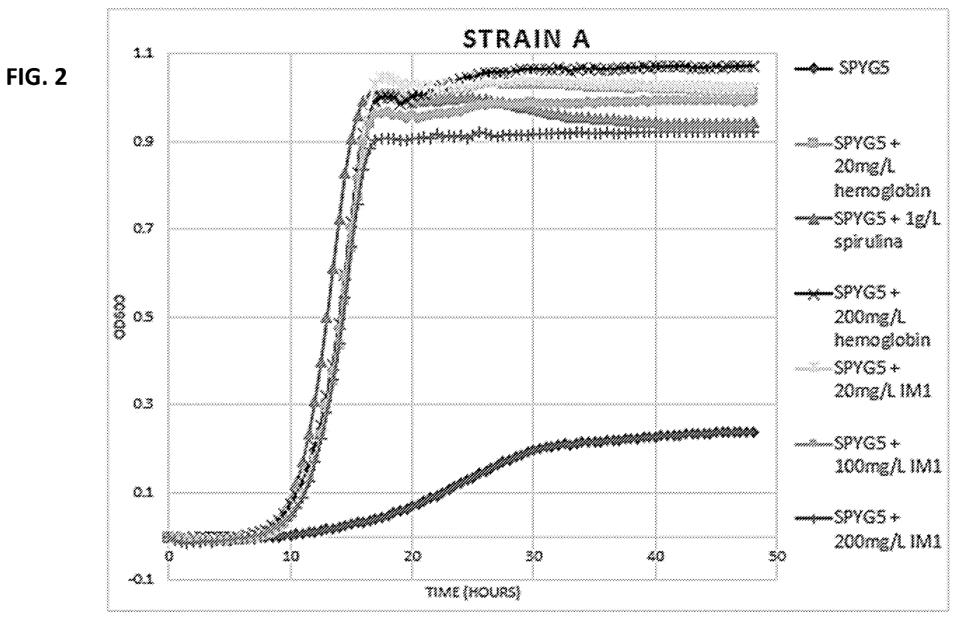

The higher concentrations of soy leghemoglobin did not improve the growth of Strain A (Prevotella histicola strain B 50329 (NRRL accession number B 50329)) beyond the positive controls and showed similar results as the lower concentration of soy leghemoglobin (FIG. 2), thereby demonstrating that the 20 mg/L concentration provides sufficient leghemoglobin to support the growth. Animal-sourced hemoglobin and spirulina solution provided consistent growth support for Strain A (Prevotella histicola strain B 50329 (NRRL accession number B 50329)) (FIG. 2).

### Example 7: Testing Soy Leghemoglobin to Replace Animal Source Hemoglobin in Different Microbes

To confirm that soy leghemoglobin will be sufficient to support growth of hemoglobin-dependent microbes, a sample of Impossible™ Meat was purchased and a soy leghemoglobin-containing solution was prepared using this sample.

Solution Prepared:

TABLE 10
Soy leghemoglobin Solution
IM - 0.25% solution in 0.01M NaOH

Percentage of the solution was calculated based on estimation that the Impossible™ Meat contains 2% of soy leghemoglobin (the package states 2% or less). The actual concentration of soy leghemoglobin in the solution is expected to be equal or less than the calculated estimate.

Solutions were prepared by resuspending the Impossible™ Meat in 0.01M NaOH solution, followed by heating until boiling and then clearing the Impossible™ Meat solids out by double filtering through a gauze filter. Final solution was autoclaved and then added to the growth media to test a range of 2 concentrations: 20 mg/L of soy leghemoglobin (IM) final concentration; and 200 mg/L of soy leghemoglobin final concentration.

As positive control, animal (porcine) sourced hemoglobin solution at 20 mg/L concentration was used. SPY base with glucose without any other additives was used as a negative control.

Growth media used for the experiment was SPY, according to the following recipe:

TABLE 11
SPY Media

| | g/L |
|---|---|
| Component | SPY |
| Yeast Extract 19512 Organotechnie S.A.S. | 10 |
| Soy Peptone A2 SC 19649 Organotechnie | 10 |
| S.A.S. | |
| Soy Peptone E110 19885 Organotechnie | 10 |
| S.A.S. | |
| Dipotassium Phosphate K2HPO4 | 2.5 |
| L-Cysteine-HCl | 0.5 |

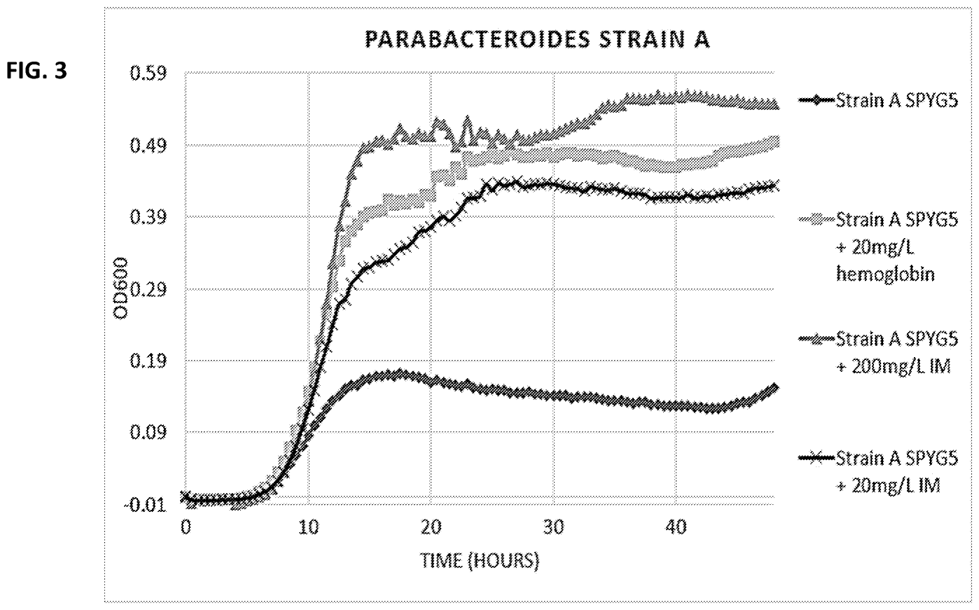

5 g/L of glucose was used as a carbon source for all media compositions. Complete media with glucose added is referred to as SPYG5 in FIGS. 3-5.

The growth dynamics test was performed in a BioTek Epoch2 platereader under anaerobic conditions for 48 hours.

As can be seen in the growth dynamics graph in FIG. 3, the negative control, SPYG5 media without any additives, was unable to support growth of a Parabacteroides strain. In both IM concentrations, the growth of a Parabacteroides strain, Parabacteroides Strain A, was restored to the similar levels as animal source hemoglobin. The higher concentration of IM even exceeded the growth with animal sourced hemoglobin, therefore showing that soy leghemoglobin can be used to support full growth of this microbe.

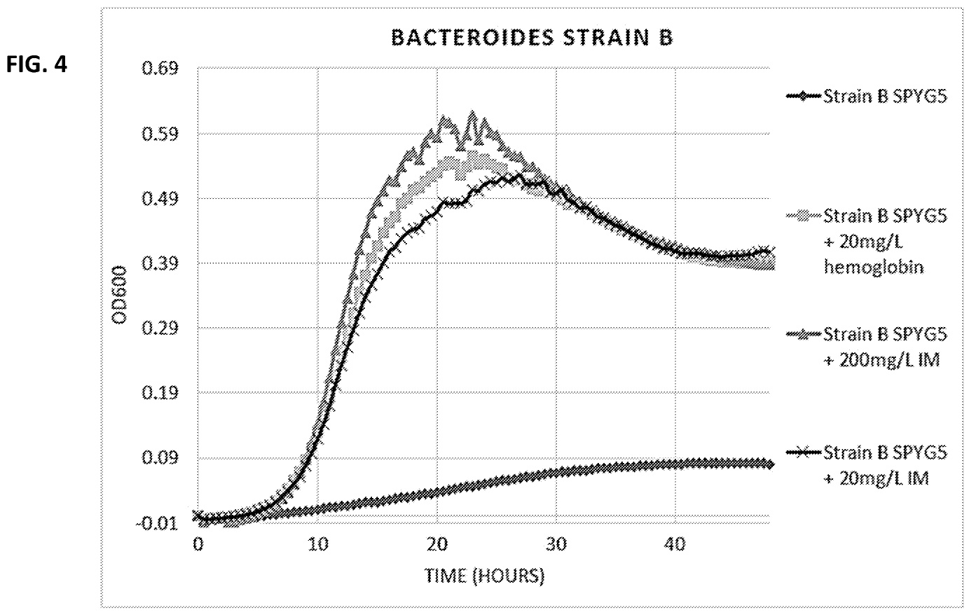

Similar to FIG. 3, in the growth dynamics graph in FIG. 4, the negative control, SPYG5 media without any additives, was unable to support growth of a Bacteroides strain. In both IM concentrations here, the growth of a Bacteroides strain, Bacteroides Strain B, was restored to the same level as with positive control—animal source hemoglobin. Soy leghemoglobin can be used to fully support growth of this microbe.

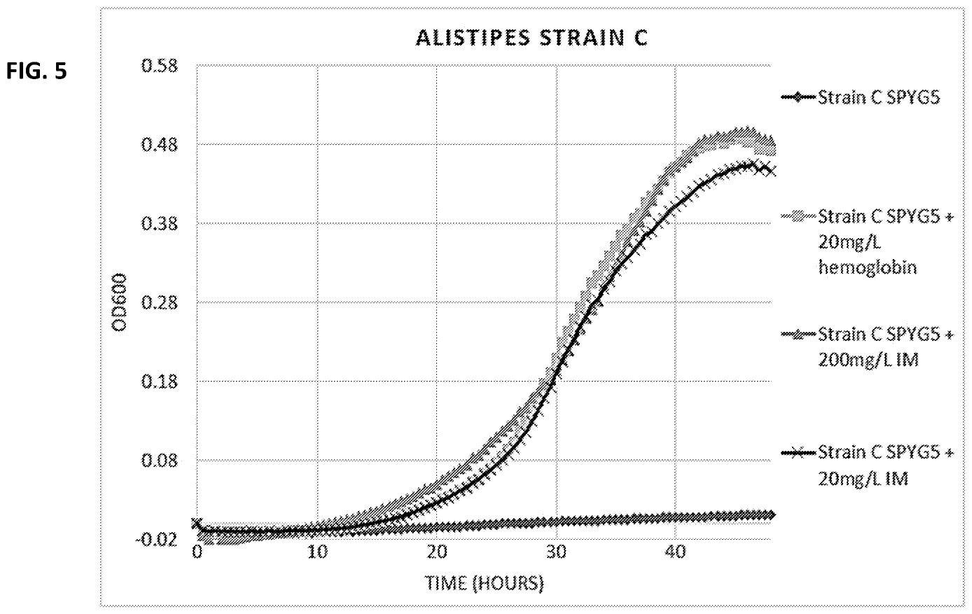

In the growth dynamics graph in FIG. 5 for an Alistipes strain, Alistipes Strain C, the negative control, SPYG5 media without any additives, was unable to support growth of this microbe. In both IM concentrations, the growth of Alistipes Strain C was restored to the same level as with positive control—animal sourced hemoglobin.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of culturing hemoglobin-dependent bacteria, the method comprising incubating the hemoglobin-dependent bacteria in a growth medium that comprises a heme-containing polypeptide, wherein the heme-containing polypeptide is:
(i) a piscine polypeptide;
(ii) an avian polypeptide; or
(iii) a non-animal-derived polypeptide.

2. The method of claim 1, wherein the heme-containing polypeptide is a hemoglobin.

3. (canceled)

4. The method of claim 1, wherein the heme-containing polypeptide is a leghemoglobin.

5. The method of claim 1, wherein the heme-containing polypeptide is a myoglobin.

6. The method of claim 1, wherein the heme-containing polypeptide is a piscine polypeptide.

7-8. (canceled)

9. The method of claim 1, wherein the heme-containing polypeptide is an avian polypeptide.

10-11. (canceled)

12. The method of claim 1, wherein the non-animal-derived polypeptide is purified from a plant, bacteria, cyanobacteria, fungus, algae, grain, legume, and/or protozoa.

13-18. (canceled)

19. The method of claim 1, wherein the heme-containing polypeptide comprises an amino acid sequence with at least 70% identity to an amino acid sequence in any one of SEQ ID NOs: 1-29, 31, 33, 35, 37, 39, 41, 43, 114, or a combination thereof.

20. (canceled)

21. The method of claim 1, wherein the heme-containing polypeptide comprises an amino acid sequence with at least 70% identity to the amino acid sequence of SEQ ID NO: 4.

22-23. (canceled)

24. The method of claim 1, wherein the heme-containing polypeptide comprises an amino acid sequence with at least 70% identity to the amino acid sequence of SEQ ID NO: 114 or UniProtKB—P02236 (LGB2_SOYBN).

25-33. (canceled)

34. The method of claim 12, wherein the heme-containing polypeptide is a soy leghemoglobin recombinantly expressed in Pichia pastoris.

35. The method of claim 1, wherein the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

36. The method of claim 1, wherein the hemoglobin-dependent bacteria are of the genus Prevotella.

37-38. (canceled)

39. The method of claim 36, wherein the Prevotella comprise at least 99% genomic, 16S and/or CRISPR sequence identity to the nucleotide sequence of the Prevotella Strain B 50329 (NRRL accession number B 50329).

40. (canceled)

41. The method of claim 36, wherein the Prevotella comprise at least 99% genomic, 16S and/or CRISPR sequence identity to the nucleotide sequence of the Prevotella Strain C (PTA-126140).

42. (canceled)

43. The method of claim 36, wherein the hemoglobin-dependent bacteria are a strain of Prevotella bacteria comprising one or more proteins listed in Table 1.

44. The method of claim 36, wherein the hemoglobin-dependent bacteria are from a strain of Prevotella substantially free of a protein listed in Table 2.

45-48. (canceled)

49. A method of culturing hemoglobin-dependent bacteria, the method comprising (a) adding a heme-containing polypeptide and hemoglobin-dependent bacteria to a growth medium; and (b) incubating the hemoglobin-dependent bacteria in the growth medium, wherein the heme-containing polypeptide is:
(i) a piscine polypeptide;
(ii) an avian polypeptide; or
(iii) a non-animal-derived polypeptide.

50. A bioreactor comprising hemoglobin-dependent bacteria in a growth medium comprising a heme-containing polypeptide, wherein the heme-containing polypeptide is:
(i) a piscine polypeptide;
(ii) an avian polypeptide; or
(iii) a non-animal-derived polypeptide.

51-52. (canceled)

53. A composition comprising
a) hemoglobin-dependent bacteria, and
b) a growth medium comprising a heme-containing polypeptide, wherein the heme-containing polypeptide is:
(i) a piscine polypeptide;
(ii) an avian polypeptide; or
(iii) a non-animal-derived polypeptide.

54. A growth medium for use in culturing hemoglobin-dependent bacteria, the growth medium comprising a heme-containing polypeptide, wherein the heme-containing polypeptide is:
(i) a piscine polypeptide;
(ii) an avian polypeptide; or
(iii) a non-animal-derived polypeptide.

55. A heme-containing polypeptide for use as a supplement in a growth medium for facilitating the growth of hemoglobin-dependent bacteria, wherein the heme-containing polypeptide is:
(i) a piscine polypeptide;
(ii) an avian polypeptide; or
(iii) a non-animal-derived polypeptide.

56. A bacterial composition comprising
(a) hemoglobin-dependent bacteria, and
(b) a heme-containing polypeptide, wherein the heme-containing polypeptide is:
(i) a piscine polypeptide;
(ii) an avian polypeptide; or
(iii) a non-animal-derived polypeptide.


SEQUENCE LISTING (115 sequences)

SEQ ID NO: 1 - protein, 161 aa, Vigna radiate
MTTTLERGFTEEQEALVVKSWNVMKKNSGELGLKFFLKIFEIAPSAQKLFSFLRDSTVPLEQNPKLKPHAVSVFVMTCDSAVQLRKAGKVTVRESNLKKLGATHFRTGVANEHFEVTKFALLETIKEAVPEMWSPAMKNAWGEAYDQLVDAIKYEMKPPSS

SEQ ID NO: 2 - protein, 133 aa, Methylacidiphilum infernorum
MIDQKEKELIKESWKRIEPNKNEIGLLFYANLFKEEPTVSVLFQNPISSQSRKLMQVLGILVQGIDNLEGLIPTLQDLGRRHKQYGVVDSHYPLVGDCLLKSIQEYLGQGFTEEAKAAWTKVYGIAAQVMTAE

SEQ ID NO: 3 - protein, 139 aa, Aquifex aeolicus
MLSEETIRVIKSTVPLLKEHGTEITARMYELLFSKYPKTKELFAGASEEQPKKLANAIIAYATYIDRLEELDNAISTIARSHVRRNVKPEHYPLVKECLLQAIEEVLNPGEEVLKAWEEAYDFLAKTLITLEKKLYSQP

SEQ ID NO: 4 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 5 - protein, 162 aa, Hordeum vulgare
MSAAEGAVVFSEEKEALVLKSWAIMKKDSANLGLRFFLKIFEIAPSARQMFPFLRDSDVPLETNPKLKTHAVSVFVMTCEAAAQLRKAGKITVRETTLKRLGGTHLKYGVADGHFEVTRFALLETIKEALPADMWGPEMRNAWGEAYDQLVAAIKQEMKPAE

SEQ ID NO: 6 - protein, 1153 aa, Pyricularia oryzae
MDGAVRLDWTGLDLTGHEIHDGVPIASRVQVMVSFPLFKDQHIIMSSKESPSRKSSTIGQSTRNGSCQADTQKGQLPPVGEKPKPVKENPMKKLKEMSQRPLPTQHGDGTYPTEKKLTGIGEDLKHIRGYDVKTLLAMVKSKLKGEKLKDDKTMLMERVMQLVARLPTESKKRAELTDSLINELWESLDHPPLNYLGPEHSYRTPDGSYNHPFNPQLGAAGSRYARSVIPTVTPPGALPDPGLIFDSIMGRTPNSYRKHPNNVSSILWYWATIIIHDIFWTDPRDINTNKSSSYLDLAPLYGNSQEMQDSIRTFKDGRMKPDCYADKRLAGMPPGVSVLLIMFNRFHNHVAENLALINEGGRFNKPSDLLEGEAREAAWKKYDNDLFQVARLVTSGLYINITLVDYVRNIVNLNRVDTTWTLDPRQDAGAHVGTADGAERGTGNAVSAEFNLCYRWHSCISEKDSKFVEAQFQNIFGKPASEVRPDEMWKGFAKMEQNTPADPGQRTFGGFKRGPDGKFDDDDLVRCISEAVEDVAGAFGARNVPQAMKVVETMGIIQGRKWNVAGLNEFRKHFHLKPYSTFEDINSDPGVAEALRRLYDHPDNVELYPGLVAEEDKQPMVPGVGIAPTYTISRVVLSDAVCLVRGDRFYTTDFTPRNLTNWGYKEVDYDLSVNHGCVFYKLFIRAFPNHFKQNSVYAHYPMVVPSENKRILEALGRADLFDFEAPKYIPPRVNITSYGGAEYILETQEKYKVTWHEGLGFLMGEGGLKFMLSGDDPLHAQQRKCMAAQLYKDGWTEAVKAFYAGMMEELLVSKSYFLGNNKHRHVDIIRDVGNMVHVHFASQVFGLPLKTAKNPTGVFTEQEMYGILAAIFTTIFFDLDPSKSFPLRTKTREVCQKLAKLVEANVKLINKIPWSRGMFVGKPAKDEPLSIYGKTMIKGLKAHGLSDYDIAWSHVVPTSGAMVPNQAQVFAQAVDYYLSPAGMHYIPEIHMVALQPSTPETDALLLGYAMEGIRLAGTFGSYREAAVDDVVKEDNGRQVPVKAGDRVFVSFVDAARDPKHFPDPEVVNPRRPAKKYIHYGVGPHACLGRDASQIAITEMFRCLFRRRNVRRVPGPQGELKKVPRPGGFYVYMREDWGGLFPFPVTMRVMWDDE

SEQ ID NO: 7 - protein, 530 aa, Fusarium oxysporum
MKGSATLAFALVQFSAASQLVWPSKWDEVEDLLYMQGGFNKRGFADALRTCEFGSNVPGTQNTAEWLRTAFHDAITHDAKAGTGGLDASIYWESSRPENPGKAFNNTFGFFSGFHNPRATASDLTALGTVLAVGACNGPRIPFRAGRIDAYKAGPAGVPEPSTNLKDTFAAFTKAGFTKEEMTAMVACGHAIGGVHSVDFPEIVGIKADPNNDTNVPFQKDVSSFHNGIVTEYLAGTSKNPLVASKNATFHSDKRIFDNDKATMKKLSTKAGFNSMCADILTRMIDTVPKSVQLTPVLEAYDVRPYITELSLNNKNKIHFTGSVRVRITNNIRDNNDLAINLIYVGRDGKKVTVPTQQVTFQGGTSFGAGEVFANFEFDTTMDAKNGITKFFIQEVKPSTKATVTHDNQKTGGYKVDDTVLYQLQQSCAVLEKLPNAPLVVTAMVRDARAKDALTLRVAHKKPVKGSIVPRFQTAITNFKATGKKSSGYTGFQAKTMFEEQSTYFDIVLGGSPASGVQFLTSQAMPSQCS

SEQ ID NO: 8 - protein, 358 aa, Fusarium graminearum
MASATRQFARAATRATRNGFAIAPRQVIRQQGRRYYSSEPAQKSSSAWIWLTGAAVAGGAGYYFYGNSASSATAKVFNPSKEDYQKVYNEIAARLEEKDDYDDGSYGPVLVRLAWHASGTYDKETGTGGSNGATMRFAPESDHGANAGLAAARDFLQPVKEKFPWITYSDLWILAGVCAIQEMLGPAIPYRPGRSDRDVSGCTPDGRLPDASKRQDHLRGIFGRMGFNDQEIVALSGAHALGRCHTDRSGYSGPWTFSPTVLTNDYFRLLVEEKWQWKKWNGPAQYEDKSTKSLMMLPSDIALIEDKKFKPWVEKYAKDNDAFFKDFSNVVLRLFELGVPFAQGTENQRWTFKPTHQE

SEQ ID NO: 9 - protein, 121 aa, Chlamydomonas moewusii
SLFAKLGGREAVEAAVDKFYNKIVADPTVSTYFSNTDMKVQRSKQFAFLAYALGGASEWKGKDMRTAHKDLVPHLSDVHFQAVARHLSDTLTELGVPPEDITDAMAVVASTRTEVLNMPQQ

SEQ ID NO: 10 - protein, 164 aa, Chlamydomonas moewusii
MMRTVQLRTLRPCIRAQQQPVRPSTSATAAAATAPAPARKCPSSLFAKLGGREAVEAAVDKFYNKIVADPTVSTYFSNTDMKVQRSKQFAFLAYALGGASEWKGKDMRTAHKDLVPHLSDVHFQAVARHLSDTLTELGVPPEDITDAMAVVASTRTEVLNMPQQ

SEQ ID NO: 11 - protein, 121 aa, Tetrahymena pyriformis
MNKPQTIYEKLGGENAMKAAVPLFYKKVLADERVKHFFKNTDMDHQTKQQTDFLTMLLGGPNHYKGKNMTEAHKGMNLQNLHFDAIIENLAATLKELGVTDAVINEAAKVIEHTRKDMLGK

SEQ ID NO: 12 - protein, 117 aa, Paramecium caudatum
MSLFEQLGGQAAVQAVTAQFYANIQADATVATFFNGIDMPNQTNKTAAFLCAALGGPNAWTGRNLKEVHANMGVSNAQFTTVIGHLRSALTGAGVAAALVEQTVAVAETVRGDVVTV

SEQ ID NO: 13 - protein, 412 aa, Aspergillus niger
  MOD_RES (29)..(29): Any amino acid
MPLTPEQIKIIKATVPVLQEYGTKITTAXYMNMSTVHPELNAVFNTANQVKGHQARALAGALFAYASHIDDLGALGPAVELICNKHASLYIQADEYKIVGKYLLEAMKEVLGDACTDDILDAWGAAYWALADIMINREAALYKQSQGWTNWRQFRISKKVPESDEITSFYLEPVDGKPLPAFRPGQYISVSVQVPEEDPQARQYSLSDTSRSDYYRISVKKETGLDPRAPGAKRHPGYVSNVLHDMIKEGDLIDVSHPYGDFFLSTAEATHPIVLLSAGVGMTPMMSILNTITKKSNRKIHFIHGSRTTEARAFKSHVQKLEKEIPNMQVTYFLSRPGDSDQLGVDYHHAGRIDLQKLDGPSHLYLDNPSTEYYICGPDTFMTQMEEALKAYGVGDDRIKMELFGTGGVPHN

SEQ ID NO: 14 - protein, 165 aa, Zea mays
MALAEADDGAVVFGEEQEALVLKSWAVMKKDAANLGLRFFLKVFEIAPSAEQMFSFLRDSDVPLEKNPKLKTHAMSVFVMTCEAAAQLRKAGKVTVRETTLKRLGATHLRYGVADGHFEVTGFALLETIKEALPADMWSLEMKKAWAEAYSQLVAAIKREMKPDA

SEQ ID NO: 15 - protein, 169 aa, Oryza sativa
MALVEGNNGVSGGAVSFSEEQEALVLKSWAIMKKDSANIGLRFFLKIFEVAPSASQMFSFLRNSDVPLEKNPKLKTHAMSVFVMTCEAAAQLRKAGKVTVRDTTLKRLGATHFKYGVGDAHFEVTRFALLETIKEAVPVDMWSPAMKSAWSEAYNQLVAAIKQEMKPAE

SEQ ID NO: 16 - protein, 160 aa, Arabidopsis thaliana
MESEGKIVFTEEQEALVVKSWSVMKKNSAELGLKLFIKIFEIAPTTKKMFSFLRDSPIPAEQNPKLKPHAMSVFVMCCESAVQLRKTGKVTVRETTLKRLGASHSKYGVVDEHFEVAKYALLETIKEAVPEMWSPEMKVAWGQAYDHLVAAIKAEMNLSN

SEQ ID NO: 17 - protein, 147 aa, Pisum sativum
MGFTDKQEALVNSSWESFKQNLSGNSILFYTIILEKAPAAKGLFSFLKDTAGVEDSPKLQAHAEQVFGLVRDSAAQLRTKGEVVLGNATLGAIHVQRGVTDPHFVVVKEALLQTIKKASGNNWSEELNTAWEVAYDGLATAIKKAMT

SEQ ID NO: 18 - protein, 145 aa, Vigna unguiculata
MVAFSDKQEALVNGAYEAFKANIPKYSVVFYTTILEKAPAAKNLFSFLANGVDATNPKLTGHAEKLFGLVRDSAAQLRASGGVVADAALGAVHSQKAVNDAQFVVVKEALVKTLKEAVGDKWSDELGTAVELAYDELAAAIKKAY

SEQ ID NO: 19 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 20 - protein, 154 aa, Sus scrofa
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 21 - protein, 154 aa, Equus caballus
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 22 - protein, 152 aa, Nicotiana tabacum
MSSFTEEQEALVLKSWDSMKKNAGEWGLKLFLKIFEIAPSAKKLFSFLKDSNVPLEQNAKLKPHAKSVFVMTCEAAVQLRKAGKVVVRDSTLKKLGAAHFKYGVADEHFEVTKFALLETIKEAVPDMWSVDMKNAWGEAFDQLVNGIKTEMK

SEQ ID NO: 23 - protein, 132 aa, Bacillus sp.
MGQSFNAPYEAIGEELLSQLVDTFYERVASHPLLKPIFPSDLTETARKQKQFLTQYLGGPPLYTEEHGHPMLRARHLPFPITNERADAWLSCMKDAMDHVGLEGEIREFLFGRLELTARHMVNQTEAEDRSS

SEQ ID NO: 24 - protein, 131 aa, Corynebacterium glutamicum
MTTSENFYDSVGGEETFSLIVHRFYEQVPNDDILGPMYPPDDFEGAEQRLKMFLSQYWGGPKDYQEQRGHPRLRMRHVNYPIGVTAAERWLQLMSNALDGVDLTAEQREAIWEHMVRAADMLINSNPDPHA

SEQ ID NO: 25 - protein, 124 aa, Synechocystis sp.
MSTLYEKLGGTTAVDLAVDKFYERVLQDDRIKHFFADVDMAKQRAHQKAFLTYAFGGTDKYDGRYMREAHKELVENHGLNGEHFDAVAEDLLATLKEMGVPEDLIAEVAAVAGAPAHKRDVLNQ

SEQ ID NO: 26 - protein, 183 aa, Synechococcus sp.
MDVALLEKSFEQISPRAIEFSASFYQNLFHHHPELKPLFAETSQTIQEKKLIFSLAAIIENLRNPDILQPALKSLGARHAEVGTIKSHYPLVGQALIETFAEYLAADWTEQLATAWVEAYDVIASTMIEGADNPAAYLEPELTFYEWLDLYGEESPKVRNAIATLTHFHYGEDPQDVQRDSRG

SEQ ID NO: 27 - protein, 118 aa, Nostoc commune
MSTLYDNIGGQPAIEQVVDELHKRIATDSLLAPVFAGTDMVKQRNHLVAFLAQIFEGPKQYGGRPMDKTHAGLNLQQPHFDAIAKHLGERMAVRGVSAENTKAALDRVTNMKGAILNK

SEQ ID NO: 28 - protein, 136 aa, Bacillus megaterium
MREKIHSPYELLGGEHTISKLVDAFYTRVGQHPELAPIFPDNLTETARKQKQFLTQYLGGPSLYTEEHGHPMLRARHLPFEITPSRAKAWLTCMHEAMDEINLEGPERDELYHRLILTAQHMINSPEQTDEKGFSH

SEQ ID NO: 29 - protein, 143 aa, Salmo salar
MSLTARDKSVVNAFWGKIKGKADVVGAEALGRMLTAYPQTKTYFSHWADLSPGSAPVKKHGGVIMGAIGNAVGLMDDLVGGMSGLSDLHAFKLRVDPGNFKILSHNILVTLAIHFPADFTPEVHIAVDKFLAALSAALADKYR

SEQ ID NO: 30 - DNA, 556 bp, Salmo salar
ttcttcagttcatttctcgtagatttaatacaggtcaatcaaaggagcaaaatgagtctgacagcaagggacaaatctgtggtcaatgccttctggggcaagattaaaggaaaggcagatgtcgtcggcgctgaggctttgggaaggatgctgactgcttacccccagactaagacctacttctcccactgggctgacctgagccccggctctgccccagtcaagaagcatggaggcgtcatcatgggtgcaattggtaatgctgtcggactgatggacgacctcgtggggggaatgagtggtctcagcgatctgcacgccttcaagctgcgcgttgaccctggaaacttcaagattctgtcccacaacatccttgtcaccctggctattcacttccctgcggatttcactcccgaagtgcacattgctgtggataaattccttgcagccttgtccgctgccctggctgacaaatacagataagaccatcatgaagtccaaaattggactccagttcctgctctgttgttatcacaaaataaacaggcaatgaatg

SEQ ID NO: 31 - protein, 147 aa, Salmo salar
MVDWTDAEKSTISAVWGKVDINEVGPLALARVLIVYPWTQRYFGSFGDVSTPAAIMGNPKVAAHGKVVCGALDKAVKNMGNILATYKSLSETHANKLFVDPDNFRVLADVLTIVVAAKFGASFTPEIQATWQKFMKVVVAAMGSRYF

SEQ ID NO: 32 - DNA, 545 bp, Salmo salar
tttaatcttgttgcaaaaccggagatcaaacactgacaacatggttgactggacagacgccgagaagagcaccatcagtgctgtctggggaaaagtagatatcaatgaggtcggaccactggctctggcaagagtcctgatcgtctacccctggactcagcgttatttcggctctttcggagatgtgtctactcccgcagcaatcatgggcaaccccaaagttgctgctcacggcaaggtcgtgtgtggagctctggataaagctgtgaagaacatgggcaacatcttggccacatacaagtcactgagcgagacacacgccaacaaactcttcgtcgaccctgacaatttcagggtgttggctgacgtcctcacaattgtcgttgccgccaagttcggagcctctttcactcctgaaatccaggcaacctggcagaagttcatgaaagtggttgtcgcagccatgggcagccggtacttctaaatgcacacgagacaagtgattatgttctgacatccctgcctgagtgaaaataaagacgttg

SEQ ID NO: 33 - protein, 143 aa, Cyprinus carpio
MSLSDKDKAAVKALWAKISPKADDIGAEALGRMLTVYPQTKTYFAHWADLSPGSGPVKKHGKVIMGAVGDAVSKIDDLVGGLAALSELHAFKLRVDPANFKILAHNVIVVIGMLYPGDFPPEVHMSVDKFFQNLALALSEKYR

SEQ ID NO: 34 - DNA, 600 bp, Cyprinus carpio
aaaatcctcttttcgccaacatctttttctgaagacaatctaacttgagaaaaagaagacgcagcaatgagtctctctgataaggacaaggctgctgtgaaagccctatgggctaagatcagccccaaagccgatgatatcggcgctgaagctctcggcagaatgctgaccgtctaccctcagaccaagacctacttcgctcactgggctgacctgagccctgggtccggtcctgtgaagaagcatggcaaggttatcatgggtgcagtcggcgatgccgtttcaaaaatagacgaccttgtgggaggtctggctgccctgagcgaacttcatgctttcaagctgcgtgttgacccggccaacttcaagatcctcgcacacaatgtcatcgtggtcatcggcatgctctaccctggagacttccccccagaggttcacatgtcagttgacaagtttttccagaacttggctctggctctctctgagaagtaccgctaaatttccggtgggcattcatacgcgacactgtgcggcactcctgaccaactcaagtgtgatgtctgaataaaatttctcaataaaaggcagagaattaaatga

SEQ ID NO: 35 - protein, 148 aa, Cyprinus carpio
MVEWTDAERSAIIGLWGKLNPDELGPQALARCLIVYPWTQRYFASFGNLSSPAAIMGNPKVAAHGRTVMGGLERAIKNMDNIKATYAPLSVMHSEKLHVDPDNFRLLADCITVCAAMKFGPSGFNADVQEAWQKFLCVVVSALCRQYH

SEQ ID NO: 36 - DNA, 630 bp, Cyprinus carpio
actccggtttctgaatttctactagtgattcctactggacttaaacaagaacctaaggacaccctaaaacgtagacatggttgagtggacagatgctgagcgaagtgccatcattggcctgtggggaaagctcaatcccgatgaactcggacctcaggccctggccaggtgtctgatcgtgtatccctggactcagagatatttcgcctccttcggaaacctgtcaagccccgctgcgatcatgggtaaccccaaggtggcagctcacgggaggactgtgatgggaggtctggagagagccatcaagaacatggacaacatcaaggccacctatgcgccactcagtgtgatgcactctgagaaattgcatgtggatcccgacaacttcaggctcctggctgattgcatcaccgtgtgcgctgccatgaagtttggcccctctggtttcaatgctgacgtccaggaggcctggcagaagtttctgtgtgtcgtcgtttccgctctgtgcagacagtaccactagattctcatcaccgatgaacaccagctgtattgcagaagattgcgggagctgttcacacaaagatctaactcttataaaataagatcaataaaatattgttaaaagca

SEQ ID NO: 37 - protein, 142 aa, Meleagris gallopavo
MVLSAADKNNVKGIFTKIAGHAEEYGAETLERMFITYPPTKTYFPHFDLSHGSAQIKGHGKKVVAALIEAVNHIDDIAGTLSKLSDLHAHKLRVDPVNFKLLGQCFLVVVAIHHPAALTPEVHASLDKFLCAVGTVLTAKYR

SEQ ID NO: 38 - DNA, 579 bp, Meleagris gallopavo
agcagactgcaggggcacccgtgctgggggctgccaacgcggaggtgcaaccatggtgctgtccgctgctgacaagaacaacgtcaagggcatcttcaccaaaatcgccggccatgctgaggagtatggcgccgagaccctggagaggatgttcatcacctaccccccgaccaagacctacttcccccactttgatctgtcacacggctccgctcagatcaaggggcacggcaagaaggtagtggctgccctgatcgaggctgtcaaccacattgatgacatcgcaggcaccctctccaagctcagcgacctccatgcccacaagctccgcgtggaccctgtcaacttcaaactcctgggccaatgcttcctggtggtggtggccatccaccaccctgctgccctgaccccagaggtccatgcttccctggacaagttcttgtgcgctgtgggcaccgtgctgactgccaagtaccgttaagacagcatggcagctagagctggggccaacccatcgccagccttccaacagcgagcagccaaatgagatgaaataaaatctgttgcatttgtgctcca

SEQ ID NO: 39 - protein, 147 aa, Meleagris gallopavo
MVHWSAEEKQLITGLWGKVNVADCGAEALARLLIVYPWTQRFFASFGNLSSPTAILGNPMVRAHGKKVLTSFGDAVKNLDNIKNTFSQLSELHCDKLHVDPENFRLLGDILIIVLAAHFSKDFTPECQAAWQKLVRVVAHALARKYH

SEQ ID NO: 40 - DNA, 644 bp, Meleagris gallopavo
acggccgctcaccagcgtgcgatccccgcgggagcaagagcccagacctcctccgtaccgacagccacacgctaccctccaaccgccgccatggtgcactggtctgctgaggagaagcagctcatcaccggcctctggggcaaggtcaatgtggccgactgcggggccgaagccctggccaggctgctgattgtctacccctggacccagaggttctttgcgtcctttgggaacctctccagccccaccgccatccttggcaaccccatggtccgtgcccatggcaagaaagtgctcacctcctttggggatgctgtgaagaacctggacaacatcaagaacaccttctcccaactgtccgagctgcactgtgacaagctgcatgtggaccccgagaacttcaggctcctgggtgacatcctcatcatcgtcctggccgcccacttcagcaaggacttcactcctgaatgccaggctgcctggcagaagctggtccgtgtggtggcccatgccctggctcgcaagtaccactaagcaccagcagcaaagatcccggagcgccctacagcaattgtatgcacctgcagaaatgctccgaggctgacagcttgtgacaggcaaataaagttcattcagtgacactc

SEQ ID NO: 41 - protein, 142 aa, Gallus gallus
MVLSAADKNNVKGIFTKIAGHAEEYGAETLERMFTTYPPTKTYFPHFDLSHGSAQIKGHGKKVVAALIEAANHIDDIAGTLSKLSDLHAHKLRVDPVNFKLLGQCFLVVVAIHHPAALTPEVHASLDKFLCAVGTVLTAKYR

SEQ ID NO: 42 - DNA, 554 bp, Gallus gallus
ggggctgccaacacagaggtgcaaccatggtgctgtccgctgctgacaagaacaacgtcaagggcatcttcaccaaaatcgccggccatgctgaggagtatggcgccgagaccctggaaaggatgttcaccacctaccccccaaccaagacctacttcccccacttcgatctgtcacacggctccgctcagatcaaggggcacggcaagaaggtagtggctgccttgatcgaggctgccaaccacattgatgacatcgccggcaccctctccaagctcagcgacctccatgcccacaagctccgcgtggaccctgtcaacttcaaactcctgggccaatgcttcctggtggtggtggccatccaccaccctgctgccctgaccccggaggtccatgcttccctggacaagttcttgtgcgccgtgggcactgtgctgaccgccaagtaccgttaagacggcacggtggctagagctggggccaacccatcgccagccctccgacagcgagcagccaaatgagatgaaataaaatctgttgcatttgtgctccag

SEQ ID NO: 43 - protein, 147 aa, Gallus gallus
MVHWTAEEKQLITGLWGKVNVAECGAEALARLLIVYPWTQRFFASFGNLSSPTAILGNPMVRAHGKKVLTSFGDAVKNLDNIKNTFSQLSELHCDKLHVDPENFRLLGDILIIVLAAHFSKDFTPECQAAWQKLVRVVAHALARKYH

SEQ ID NO: 44 - DNA, 632 bp, Gallus gallus
tccccacgggagcaagagcccagacctcctccgtaccgacagccacacgctaccctccaaccgccgccatggtgcactggactgctgaggagaagcagctcatcaccggcctctggggcaaggtcaatgtggccgaatgtggggccgaagccctggccaggctgctgatcgtctacccctggacccagaggttctttgcgtcctttgggaacctctccagccccactgccatccttggcaaccccatggtccgcgcccacggcaagaaagtgctcacctcctttggggatgctgtgaagaacctggacaacatcaagaacaccttctcccaactgtccgaactgcattgtgacaagctgcatgtggaccccgagaacttcaggctcctgggtgacatcctcatcattgtcctggccgcccacttcagcaaggacttcactcctgaatgccaggctgcctggcagaagctggtccgcgtggtggcccatgccctggctcgcaagtaccactaagcaccagcaccaaagatcacggagcacctacaaccattgcatgcacctgcagaaatgctccggagctgacagcttgtgacaaataaagttcattcagtgacactcaaaaaaaaaaaaaaa

SEQ ID NO: 45 - protein, 770 aa, Prevotella histicola
MNLKTFTKTVLCFALFAVSAITAKAADHLAIVGEAVWGGWDLVKATAMVKSPNNPDVFMATVHLNAGKGFKFLTEREWGKLEYRSGASDVVLKSGIRYKLYASIGASEDGKFKVSESANYEIICDLARKTVEVKKVAYQAKEIRYAALWMIGDATAGDWDYNNGVLLSQDSGNPTCYTATVELKEGEFKFTTNKQWGYDHSVYIFRDVNDQNKIVFGGEDNKWRITEDGMYNVTVDVPTKTISIKQIDDPAGHKPQFGNDVILVGDATIAGWNLDNAIYLEHTGQAGRVFKTTTYLEAGKGFKFLSMLSYDDIDYRPANNTVLNPGVPGTFVPSLPSSTDTKFSVERSGNYDIVCNMNNRTVVVTLSENQVLVNYPALWLIGSATSAGWNPGKAVELKRSEADPAVYTARVQLKKGEFKILTSKNVGFDQPTYYRDSTNEHRIVFGVDGDEVAKKDCKWTLSENAEGTYDVTVDIEAMTIFCDKVNMDEPSVESTDKELILIGDATYSAWDLPKSIVMTPVGPTTFKAVTHLEAGKEFKFLTELAWKRYEYRAESLRKELQEGSMSMLVPYRYTNDKDDKDHDFKFVVKESGNYEIVCDLYIPALIIRKVRYQDTPVTYSSLWIVGSATPGGWTIERGIKMTQDENYPTKFTAKANLVPGELKFATNKFADFTQDFFFRGKDDYTAVLGGNDNKWNITEAGTYSVTIDVASKRVTITKPARNAPTGISTVDSSDEAPAEYFTLNGIKVTTPSSGIYIKRQGGRTTKVVMK

SEQ ID NO: 46 - protein, 193 aa, Salmonella typhimurium
MDTYQILDIIGCIVGLIYIYQEYKASIWLWMTGIIMPVIYMFVYYEAGLYADFGMQIYYTLAAIYGYLYWKLGKKKGTEDKEIPITHFPRRYIIPAIIVFFVLWIALYYILICFTNSTVPVLDSFGNALSFIGLWALAKKYLEQWWIWIVVDAELSALYIYKGIPFTAMLYALYTVIAVAGYFKWRRYIKQQK

SEQ ID NO: 47 - protein, 544 aa, Bacillus licheniformis
MRVRLYKNILLFLFLWVNTLACVSADTSRTVESQPIENGLIITESKGWLETIYAKWKPVAEADGYYVYVKGGQYADYSKVDSELIRVYNGYVRVDIPGLKAGTYSLKIVAVKGGKETQSSEVTGLKVLNYVREGFAHKNYSGVGAYNDDGTLKSGAVVIYVNKDNAKTVSAHLGKTTFIGLQAILNAYQKGNITTPLSVRILGLLRNGDTDTFGSSTEGIQIKGKQADSEMNITIEGIGEDASIYGFGFLVRNAKSVEFRNLGIMRAMDDGVSLDTNNSNIWIHHMDLFYGKASGGDHIKGDGSIDVKTDSKYVTIDNCHFWDTGKTSMCGMKKETGPNYITYHHNWFDHSDSRHARVRTMSVHLWNNYYDGCAKYGIGATMGCSVFSENNYFRATKNPILISKQGSDAKGTGKFSGEPGGMVKEYGSLFTEKGAESTYTPISYADNNSSFDFYHAISRNEKVPASVKTLNGGNIYNNFDTDAALMYSYTPDATALVPSQVTGFYGAGRLNHGSLQFKFNNAVEDTNSTPIPALEALIDAYSGK

SEQ ID NO: 48 - protein, 366 aa, Streptococcus gordonii
MKYNIAYCIEGFYNHGGMERILSVCANLLSDIYSITIIVANQRGREHAYNLAQNVNVVDLGVSCKNYKEEYKKSLTRYLQDHQFSVVISLAGLELFFLPQIKDGSKKVMWFHFAFDVSKMFLSERFHGWKLNLLYYIHTIRRIYFAKKFDTIVVLSKSDCDSWSRFCNNVKYIYNPITIDRKVISNLSEESVIAVGRLGWQKGFDFLIDSWVLVDDKHPDWHLDIFGEGPDRLELQHQIDRKGLHDKVRLCGVTKQIEEEYGKHSIYVMSSRAEGFPLALLEASSCGLPMISFNCHQGPNEIIQEGENGFLVDKVGDIYTLSDRICKLIEDNNLRNMMGKKALDSSFRFEGEVIKKDWISLLKQLI

SEQ ID NO: 49 - protein, 158 aa, Prevotella histicola
MKRLFFMFLFLGTITMNSLAQEEKPIKYETKNFSLPDKMPLYPGGDGALRAFLSLNLHYPEKAQAFGVEGRSLMKFCVSSDGSIKDISAVDCKITNYNRTEFNKLPLSKQESLKKECAKAFAKEAARVIRLMPKWEPAELNGKKMNVYYSLPFTFKLR

SEQ ID NO: 50 - protein, 415 aa, Prevotella histicola
MNYPLFIARKIYNGGDRTRKVSKPAIRIATIGVAIGLAVMIISVGVVLGFKHTIRNKVVGFGSDITVANFLTLQSSEQYPIQITDSLVKSLQITPGIKHVQRYDYTQGILKTDNDFLGVLLKGVGPDFDSTFIHENMVEGSLPHFHDNESQQKIVISKTIADKLNLKVGQRIFAYFINKQGVRTRKFTITGIYATNMKQFDSQICFTDIYTTNKLNGWEPDQYSGAELQVDNFSQLTPISMRVLNKVKNTVDHYGGTYSSENIIEQNPQIFSWLDLMDMNVWIILALMISVAGVTMISGLLIIILERTQMIGILKALGSRNRQIRHIFLWFATFIIGKGLLWGNIIGLGCILFQSWTGLVKLDPQTYYVNTVPVEINIPLIIALNMVTMLVCLVILIAPSYLISHIHPAKSMHYE

SEQ ID NO: 51 - protein, 736 aa, Mycobacterium tuberculosis
MEDKFIYTDKERKLSYQILDELKDTLDKSFLENDLPMLQVQLKDSVAKNTIHRNVFGLNPILCSLQTAAIAVKDIGLKRDSVIAILLHQSVQDGYITLEDIDNRFGKSVAKIIHGLIRIQTLYQKNPIIESENFRNLLLSFAEDMRVILIMIADRVNLMRQIRDAEDKEAQHKVAEEASYLYAPLAHKLGLYQLKRELEDLSLKYLEHDAYYLIKDKLNATKASRDAYINQFIAPVRERLTAGGLRFHIKGRTKSIHSIWQKMKKQKCGFEGIYDLFAIRIILDAPLEKEKIQCWQAYSIVTDMYQPNPKRLRDWLSVPKSNGYECLHITVLGPEKKWVEVQIRTERMDEIAEHGLAAHWRYKGIKEEGGLDDWLASIRAALEAGDNLEVMDQFKSDLYEKEIYVFTPKGDLLKFPKGATILDFAYHIHSKVGNQCVGGKINAKNVSLRTELHSGDTVEILTSATQKPKAEWLKIVKSSRAKAKIRLALKETQIKDGLYAKELLERRFKNKKIEIEESTMGHLLRKLGFKEVSEFYKQVADEKLDPNYIIEEYQKVYNHDHNLNQPKETESAENFEFENPTNEFLKKNDDVLVIDKNLKGLDFSLAKCCHPIYGDPVFGFVTVNGGIKIHRTDCPNAPEMRKRFGYRIVKARWSGKGSSQYAITLRVIGNDDIGIVSNITNVISKDEKIVMRSINIDSHDGLFSGNLVVLLDDNSKLNMLIKKLRTVKGVKQVTRI

SEQ ID NO: 52 - protein, 336 aa, Escherichia coli
MKRRIFLFVALSVSIVILFGLNLIIGSVHIPLSDILTILSGSFTGKESWRFIIWDSRLPQALTAMLCGSSLAVCGLMLQTAFRNPLAGPDVFGISSGASLGVALVMLLLGGTVETSMFTASGFLAILIVAFAGAILVTAFILFLSSVVRNSVLLLIVGIMVGYVASSAVTLLNFFSSEDGVKGYIVWGMGNFGGVSMSHIPLFAFLCLAGIIASFLLVKPLNILLLGPQYAESLGISIRRIRNILLVVVGILTAVTTAFCGPISFIGLAAPHVARLLFRTENHQKLLPGTLLVGTVVALLCNLICFLPRESGMIPLNAVTPLIGAPIIIYVIMKRH

SEQ ID NO: 53 - protein, 524 aa, Escherichia coli
MKLENKEFGFDSFATEMARLKNEKHFDYLVTVVGEDFGTEEGLGCIYILENTSTHERCSVKQLAKKVGEEFVIPSVIKLWADADLLEREVYDFYGIKFLGHPDMRRLFLRNDFKGYPLRKDYDMDPAKNMYTTEDDVELDTTTEWNLDKNGELVGTQHALFTDDNFVVNIGPQHPSTHGVLRLQTVLDGETVTNIYPHLGYIHRGIEKLCEQFTYPQTLALTDRMNYLSAMMNRHALVGVIEEGMGIELSERILYIRTIMDELQRIDNHLLYTACCAQDLGALTAFLYGMRDREHVLNVMEETTGGRLIQNYYRIGGLQADIDPNFVSNVKELCKYLRPMIQEYVDVFGDNVITHQRFEGVGVMDEKDCISYGVTGPAGRASGWKNDVRKYHPYAMYDKVNFEEITLTNGDSMDRYFCHIKEIYQSLNIIEQLIDNIPEGEFYIKQKPIIKVPEGQWYFSVEGASGEFGAYLDSRGDKTAYRLKFRPMGLTLVGAMDKMLRGQKIADLVTTGAALDFVIPDIDR

SEQ ID NO: 54 - protein, 142 aa, Escherichia coli
MRTSTQSKDMGKKQEYKLRNEEFLHNISKKDSIKTLPHGIFYEIIKEGSGEGTVQPRSIVICNYRGSLISGQVFDDSWQKPTPEAFRLNELITGLQIALCAMHKGDSWRIYIPYQEGYGSKRNADIPAFSTLIFDIELINIA

SEQ ID NO: 55 - protein, 196 aa, Mycobacterium tuberculosis
MADNKIAKESVKREVIAGERLYTLLVYSENVAGVLNQIAAVFTRRQVNIESLNVSASSIEGIHKYTITAWSDAATIEKITKQVEKKIDVIKADYYEDSDLFIHEVGLYKIATPILLENAEVSRAIRKRNARMMEVNPTYSTVLLAGMTDEVTALYHDLKNFDCLLQYSRSGRVAVTRGFSEPVSDFLKSEEESSVL

SEQ ID NO: 56 - protein, 390 aa, Escherichia coli
MKKKVKIGLLPRVIIAILLGIFFGYFMPTPLARVFLTFNGIFSQFLGFMIPLIIIGLVTPAIADIGKGAGKLLLVTVIIAYVDTVVAGGLAYGTGLCLFPSMIASTGGAMPHIDKATELAPYFSINIPAMADVMSGLVFSFMLGLGIAYGGLTATKNIFNEFKYVIEKVIAKAIIPLLPLYIFGVFLNMAHNGQAQQILLVFSQIIIVILVLHVFILVYQFCIAGAIIRRNPFRLLWNMMPAYLTALGTSSSAATIPVTLEQTMKNGVGKEIAGFVVPLCATIHLSGSAMKITACALTICLLVGLPHDPALFIYFILMLSIIMVAAPGVPGGAIMAALAPLASILGFNSEAQALMIALYIAMDSFGTACNVTGDGAIALVVNKMFGKKER

SEQ ID NO: 57 - protein, 162 aa, Prevotella histicola
MKKLLLLVCAAVMSLSASAQAGDKALGAQLVFGSETNSLGFGVKGQYYFTDHIRGEGSFDYFLKNKGISMWDINANVHYLFDVADKFKVYPLAGLGYTNWSYKYEYAGAPVVEGSDGRLAVNLGGGVEYELTKNLNVNAEAKYQIISNYNQLVLGVGVAYKF

SEQ ID NO: 58 - protein, 84 aa, Nostoc sp.
MHFYCTKSSLDTMSERYVKRMIAKLASQGKTVISIAHRFSTIMDAKHIILLAKGKVVAEGTHQELLKTSEDYRKLWSDQNDEID

SEQ ID NO: 59 - protein, 254 aa, Pseudomonas aeruginosa
MKNVYFLSDAHLGSLAIAHRRTQERRLVRFLDSIKHKASAVYLLGDMFDFWDEYKYVVPKGFTRFLGKVSELTDMGVEVHFFTGNHDLWTYGYLEEECGVILHRKPVTMEIYGKVFYLAHGDGLGDPDPMFQFLRKVFHNRVCQRLLNFFHPWWGMQLGLNWAKKSRLKRADGKEMPYLGEDKEYLVRYTKDYMRSHKDIDYYIYGHRHIELDLTLSGKVRMLILGDWIWQFTYAVFDGEHMFLEEYIEGESKP

SEQ ID NO: 60 - protein, 532 aa, Escherichia coli
MNSKQNDNYDVIIIGGGITGAGTARDCALRGLKVLLVEKFDFTNGATGRNHGLLHSGARYAVTDPESATECIKENMVLRRIAKHCIEETDGLFITLPEDDINYQKTFVEACARAGISANIISPEEALRLDPSVNPDLLGAVRVPDASVDPFHLTTANVLDARQHGADVLTYHEVVAILTSNGRVEGVRLRNNHTGEEIEKHAVLVINAAGIWGHDIAKMADIKINMFPAKGTLLVFGHRVNKMVINRCRKPANADILVPDDAVCVIGTTSDRVPYDTVDNLKITSEEVDTLIREGEKLAPSLATTRILRAYAGVRPLVAADNDPTGRSISRGIVCLDHEKRDGLTGMITITGGKMMTYRLMAEQATDLACKKLGINKTCETATTPLPGTAGKDSDNPHHTYSTAHKAAKGRQGNRVKEIDERTEDDRALICECEEVSVGEAKYAIEELHVHDLLNLRRRTRVGMGTCQGELCACRAAGVMCENGVKVDKAMTDLTKFINERWKGMRPVAWGSTLDEAQLTTIIYQGLCGLGI

SEQ ID NO: 61 - protein, 416 aa, Escherichia coli
MRYDTIIIGGGLSGLTAGITLAKAGQKVCIVSAGQSSLHFHSGSFDLLGYDADGEVVTHPLQAIADLKAEHPYSKIGISNIEHLASQAKTLLCEAGISVMGNYEQNHYRVTPLGTLKPAWLTTEGYAMIDDPEILPWKKVELLNIQGFMDFPTQFIAENLRMMGVECQIKTFTTDELSTARQSPTEMRATNIAKVLANKDALSKVSERINAISGDPDALLLPAVLGFSNAESLDEMKQWIKKPVQYIATLPPSVSGVRTTILLKRLFAQAGGTLLIGDSATTGQFSGNHLVSITTDHLPDEKLYADHFILASGSFMSHGIRSNYAGVYEPVFKLDVDAAEKRDDWSVTNAFEAQPYMEFGVHTDKDFHATKDGKNIENLYAIGSVLSGHNSIKHADGTGVSLLTALYVAKKITGKG

SEQ ID NO: 62 - protein, 416 aa, Escherichia coli
MAEGIQLKNISGNNLEQCLKCSICTAYCPVSAVEPKYPGPKQSGPDQERYRLKDSKFFDEALKMCLNCKRCEVACPSGVRIADIIQASRITYSTHRPIPRDIMLANTDFVGTMANMVAPIVNATLGLKPVKAVLHGVMGIDKHRTFPAYSSQKFETWYKRMAAKKQDSYSKHVSYFHGCYVNYNFPQLGKDLVKIMNAVGYGVHLLEKEKCCGVALIANGLSGQARRQGKVNIRSIRKAAEQNRIVLTTSSTCTFTMRDEYEHLLDIKTDDVRENITLATRFLYRLIEKGDIKLAFRKDFKMRTAYHSACHMEKMGWIIYSTELLKMIPGLELIMLDSQCCGIAGTYGFKKENYQRSQEIGEGLFKQIKELNPDCVSTDCETCKWQIEMSTGYEVKNPISILADALDVEETIKLNQ

SEQ ID NO: 63 - protein, 270 aa, Bacillus subtilis
MMIKNIVLSIPISLIIYLNHLIMEYSMTTQFLMELIGTLILVLFGDGVCACVTLNKSKGQKAGWVVITIAWGLAVCMGVLVAGPYTGAHLNPAVSIGLAVAGMFPWSSVPYYIVAQMIGGFLGGLLVWFFYKDHYDATDDEAAKLGTFCTSPAIRNYKMNFLSEVIATLVLVFIIISFSVDGNTGDAEHFKFGLAALGPIPVTLLIIALGMSLGGTTGYAMNPARDLSPRLAHAVCMKGDNDWSYSWIPVLGPIIGAIIAGFCGAALLLV

SEQ ID NO: 64 - protein, 398 aa, Streptococcus pneumoniae
MSEKIIPSNEPAQAASEPIKASYTEYTVIPSQGYCQFVKCKKGDQPVVLKGLKEAYRERVLLRNALKREFKQCQRLNHPGIVRYQGLVDVEGYGLCIEEEYVDGRTLQAYLKESHTDDEKITIVNQIADALRYAHQQGVAHRNLKPSNILITKQGDHVKLIDFNVLSLDDVKPTADTTRFMAPELKDETMTADGTADIYSLGTIMKVMGLTLAYSEVIKRCCAFKRSDRYSDIDEFLADFNHDGSSFSMPKIGKGTVVIGFIAVVVIALAALAYNYGGALVDQVGKIDVTSIFKSDAETAPEDSAMVKSVEQNNNDSVADEAPATGKLAFMNTMKPALYKDLDRLFAKHSDDRAKLNRAIKVYYRGLIQANDTLDNEQRAELDRVFGNYVKQKKAALK

SEQ ID NO: 65 - protein, 62 aa, Prevotella histicola
MLVAQLFVGVLQAQKPVQNRRQAVGQSMERQGLVNVKAVVPSIKVALMYARTDNFCHRMALS

SEQ ID NO: 66 - protein, 440 aa, Escherichia coli
MITGLVIIQLLIVLALIFIGARVGGIGLGIYGMIGVFILVYGFGLAPGSAPIDVMMIIVAVITAASALQASGGLEYLVGVAAKFLQKHPDHITYFGPITCWLFCVVAGTAHTSYSLMPIIAEIAQTNKIRPERPLSLSVIAASLGITCSPVSAATAALISQDLLGAKGIELGTVLMICIPTAFISILVAAFVENHIGKELEDDPEYKRRVAAGLINPEAACEEVQKAENEHDPSAKHAVWAFLFGVALVILFGFLPQLRPEGVSMSQTIEMIMMSDAALILLVGKGKVGDAVNGNIFKAGMNAVVAIFGIAWMGNTFYVGNEKILDAALSSMISSTPILFAVALFLLSIMLFSQAATVTTLYPVGIALGINPLLLIAMFPACNGYFFLPNYPTEVAAIDFDRTGTTRVGKYVINHSFQIPGFITTIVSILLGVLMVQFFR

SEQ ID NO: 67 - protein, 351 aa, Escherichia coli
MRILKITFVTVLALVMSTVVFAQKPKIRIIATGGTIAGVSASATSSAYGAGQVGVQTLIDAVPQIKDIADVSGEQLVNIGSQDMNDEVWLKLAKRINDLLNKEGYDGVLITHGTDTMEETAYFLSLTVHTDKPVVMVGSMRPSTAISADGPANLYNGICTLVDPSSKGHGVMVCMNNELFEAKSVIKTHTTDVSTFKGGLYGEMGYVYNGKPYFLHKPVAKQGLTSEFNVDNLTSLPKVGIVYGYANCSPLPIQAFVNAKFDGIVLAGVGDGNFYKDVFDVALKAQNSGIQIVRSSRVPFGPTNLNGEVDDAKYHFVASLNLNPQKARVLLMLALTKTKDWQKIQQYFNEY

SEQ ID NO: 68 - protein, 574 aa, Mycobacterium tuberculosis
MALACAMTMSASAQMGTNPKWLGDAIFYQIYPSSYMDTDGNGIGDLPGITQKLDYIKSLGVNAIWLNPVFESGWFDGGYDVIDFYKIDPRFGTNTDMVNLVKEAHKRGIKVCLDLVAGHTSTKCPWFKESANGDRNSRYSDYFIWTDSISEADKKEIAERHKEANPASSTHGRYVEMNAKRGKYYEKNFFECQPALNYGFAKPDPNQPWEQPVTAPGPQAVRREMRNIMAFWFDKGVDGFRVDMASSLVKNDWGKKEVSKLWNEMREWKDKNYPECVLISEWSDPAVAIPAGFNIDFMIHFGIKGYPSLFFDRNTPWGKPWPGQDISKDYKFCYFDKAGKGEVKEFVDNFSEAYNATKNLGYIAIPSANHDYQRPNIGTRNTPEQLKVAMTFFLTMPGVPFIYYGDEIGMKYQMDLPSKEGSNERAGTRTPMQWTSGPTAGFSTCNPSQLYFPVDTEKGKLTVEAQQNDPRSLLNYTRELTRLRHSQPALRGNGEWILVSKESQPYPMVYKRTSGGETVVVAINPSDKKVSANIAHLGKAKSLIMTGKASYKTGKTEDAVELNGVSAAVFKIAE

SEQ ID NO: 69 - protein, 244 aa, Listeria monocytogenes
MNIAVIFAGGSGLRMHTKSRPKQFLDLNGKPIIIYTLELFDNHPGIDAIVVACIESWIPFLEKQLRKFEINKVVKIVPGGESGQASIYNGLCAAEAYIKSKNVASEDTTVLIHDGVRPLITEETITDNINKVAEVGSCITCIPATETLVVKQHDGSLEIPSRADSLIARAPQSFLLSDILTAHRRAIDEKKNDFIDSCTMMSHYGYRLGTIIGPMENIKITTPTDFFVLRAMVKVHEDQQIFGL

SEQ ID NO: 70 - protein, 327 aa, Escherichia coli
MTEKKSVSIVLCTYNGTKYLQEQLDSILAQTYPLHEIIIQDDGSTDNTWQILEKYEEKYPLIHIYHNEGTHGVNANFLSAMHRTTGDFIAIADQDDIWETDKIANQMTTIGNKLLCSGLTRPFSSDGSFAYFDNRPRNVSIFRMMFLGLPGHTMLFRRELLRMMPPVTHSFFNVSLYDAALSILAASHDSIAFCNKVLVNFRRHADATTYNDYSRSLPSWQNGLYELLWGLRHYHQARSIALPIYRGKLALMEGITTNYHDFIEAKAIMRLETQKGLWAFLRLQYLLTKNHQRLFQTSGGSFIKMIRAWLYPVMQLYMYHHALRRCK

SEQ ID NO: 71 - protein, 437 aa, Enterobacter cloacae
MESFIIEGGHRLSGTIAPQGAKNEALEVICATLLTTEEVIIRNIPNILDVNNLIKLLQDIGVKVKKLGANDFSFQADEVKLDYLESIDFVKKCSSLRGSVLMIGPLLGRFGKATIAKPGGDKIGRRRLDTHFLGFKNLGARFVRIEDRDVYEIQADKLVGDYMLLDEASVTGTANIIMSAVMAEGTTTIYNAACEPYIQQLCHLLNAMGAKITGIASNLITIEGVTSLHGAEHRILPDMIEVGSFIGMAAMVGDGVRIKDVSIPNLGLILDTFRRLGVQIIEDEDDLIIPRQDHYVIDSFIDGTIMTISDAPWPGLTPDLISVLLVVATQAQGSVLFHQKMFESRLFFVDKLIDMGAQIILCDPHRAVVVGHDHAKKLRAGRMSSPDIRAGIALLIAALTAEGTSRIDNIAQIDRGYENIEGRLNALGAKVQRVEIC

SEQ ID NO: 72 - protein, 772 aa, Escherichia coli
MERSGNFYKAIRLGYILISILIGCMAYNSLYEWQEIEALELGNKKIDELRKEINNINIQMIKFSLLGETILEWNDKDIEHYHARRMAMDSMLCRFKATYPAERIDSVRHLLEDKERQMCQIVQILEQQQAINDKITSQVPVIVQKSVQEQPKKSKRKGFLGIFGKKEEAKPTVTTTMHRSFNRNMRTEQQAQSRRLSVHADSLAARNAELNRQLQGLVVQIDGKVQTDLQKREAEITAMRERSFIQIGGLTGFVILLLVISYIIIHRNANRIKRYKQETADLIERLQQMAKRNEALITSRKKAVHTITHELRTPLTAITGYAGLIQKNFNADKTGMYIRNIQQSSDRMREMLNTLLSFFRLDDGKEQPNFSTCRISSIAHTLESEFMPIAINKGLALTVTNHTDAVVLTDKERILQIGNNLLSNAIKFTENGAVSLTMGYDNGMLKLIVKDTGSGMTEEEQQRVFGAFERLSNAAAKDGFGLGLSIVQRIVTMLGGTIQLKSEKGKGSRFTVEIPMQSAEELPERINKTQIHHNRTLHDIVAIDNDKVLLLMLKEMYAQEGIHCDTCTNAAELMEMIRRKEYSLLLTDLNMPDINGFELLELLRTSNVGNSRIIPIIVTTASGSCNREELLERGFSDCLLKPFSISELMEVSDKCAMKGKQNEKPDFSSLLSYGNESVMLDKLIAETEKEMQSVRDGEQRKDFQELDALTHHLRSSWEILRADQPLRELYKQLHGSAVPDYEALNNAVTAVLDKGSEIIRLAKEERRKYENG

SEQ ID NO: 73 - protein, 321 aa, Staphylococcus aureus
MKRSRFYITVGLILSLTLLMSACGQKKAKDGRTDTPTSGTIKFASDESFSPIVEELLQNYQFRYPQAHLLPIYTDDNTGMKLLLDQKVNLFITSHAMTKGEDAILRGKGPIPEVFPIGYDGIAFIVNRSNPDSCITVDDVKKILQGKIAKWNQLNPKNNRGSIEVVFDNKASATLHYVVDSILGGKNIKSENIVAAKNSKSVIDYVNKTPNAIGVIGSNWLNDHRDTTNTTFKKDVTVASISKATVASPSNSWQPYQAYLLDGRYPFVRTIYALLADPHKALPYAFANYIANPIGQMIIFKAGLLPYRGNINIREVEVKNQ

SEQ ID NO: 74 - protein, 198 aa, Mycobacterium tuberculosis
MAGTKRIKTALISVFHKDGLDDLLKKLDEEGVQFLSTGGTQQFIESLGYECQKVEDVTSYPSILGGRVKTLHPKIFGGILARRDNEEDQKQMVEYTIPAIDLVIVDLYPFEQTVASGASAQDIIEKIDIGGISLIRAGAKNFKDVVIVPSKAEYPVLLQLLNTKGAETEIEDRKMFAERAFGVSSHYDTAIHSWFAAE

SEQ ID NO: 75 - protein, 472 aa, Escherichia coli
MEEEKGGRIGQRPYILKIITERNYIIIIDMKKAKILLFVTALVAVLTSCGGGQKGLPTSDEYPVITIGASNAQLKTTYPATIKGVQDVEVRPKVSGFITKLNIHEGEYVHAGQVLFVIDNSTYQAAVRQAQAQVNSAQSAVAQAKANVVQANASLNSANAQAATSRLTYNNSQNLYNNKVIGDYELQSAKNTYETAQASVRQAQSGIASAQAAVKQAEAGVRQAQAMLSTAKDNLGFCYVKSPASGYVGSLPFKEDALVSASSAQPVTTISNTSTIEVYFSMTEADVLKLSRTDDGLSNAIKKFPAVSLLLADGSTYNHEGAIVKTSGMIDATTGTINVIARFPNPEHLLKSGGSGKIVIAKNNNRALLIPQEAVTQVQNKMFVYKVDAKDKVHYSEITVDPQNDGINYIVTSGLKMGERIVSKGVSSLEDGAKIKALTPAEYEEAIKKAEKLGENQSSASGFLKTMKGDSK

SEQ ID NO: 76 - protein, 292 aa, Bacillus anthracis
MAKRRNKARSHHSLQVVTLCISTAMVLILIGMVVLTVFTSRNLSSYVKENLTVTMILQPDMSTEESAALCQRIRSLHYINSLNFISKEQALKEGTRELGANPAEFAGQNPFTGEIELQLKANYANNDSIKNIERELRTYRGVSDITYPQNLVESVNHTLGKISLVLLVIAILLTIVSFSLMNNTIRLSIYARRFSIHTMKLVGASWGFIRAPFLRRAVMEGLVSALLAIAVLGVGLCLLYDYEPDITKVLSWDVLVITAGVMLAFGVLIATFCSWLSVNKFLRMKAGDLYKI

SEQ ID NO: 77 - protein, 828 aa, Campylobacter jejuni
MKLSDLKTGETGVIVKVLGHGGFRKRIIEMGFIQGKQVEVLLNAPLRDPVKYKIMGYEVSLRHSEADQIEVISAEEARQLEQAKADNEPQQGALSNNIPDESDHALTPFELTDAANRKSKVINVALVGNPNCGKTSLFNFASGAHERVGNYSGVTVDAKVGRANYEGYEFHLVDLPGTYSLSAYSPEELYVRKQLVEKTPDVVINVIDASNLERNLYLTTQLIDMHVRMVCALNMFDETEQRGDNIDYQKISELFGIPMVPTVFTNGRGVKELFHQVIAVYEGKEDETSQFRHIHINHGHELEGGIKNIQEHLRAYPDICQRYSTRYLAIKLLEHDKDVEELIKPLKDSDEIFKHRDIAAQRVKEETGNESETAIMDAKYGFIHGALEEADYSTGQKKDTYQTTHFIDQILTNKYFGFPIFFLILFIMFTATFVIGQYPMDWIDGGVSWLGDFISSNMPDGPVKDMLVDGIIGGVGAVIVFLPQILILYFFISYMEDSGYMARAAFIMDKLMHKMGLHGKSFIPLIMGFGCNVPAVMATRTIESRRSRLVTMLILPLMSCSARLPIYVMITGSFFALKYRSLAMLSLYVIGILMSVIMSRVFSRFLVKGEDTPFVMELPPYRFPTWKAIGRHTWEKGKQYLKKMGGIILVASIIVWALGYFPLPDKPDMGQQERQEHSFIGQIGHAVEPVFRPQGFNWKLDVGLLAGVGAKEIVASTMGVLYSNDDSFKDDNSFSSEGGKYVKLHKQITQDVANLHGVSYNEAEPIATLTAFCFLLFVLLYFPCIATIAAIKGETGSWGWALFAAGYTTLLAWVVSAIVFQVGMLFIG

SEQ ID NO: 78 - protein, 566 aa, Streptococcus pneumoniae
MKKNLLKAVLPASLALFAVTFGSCSQDGQLTGTKEDTGERVLDNTREIQNYLRTLPLAPMMSRASDPVPSDDGTTVPVDEGTSKTEEKGVLNGIPGSWVKTTRRYKMTQAFDESFLFDPTSDIVYPGCVLKGGTIANGTYAIITSHETGDVTFSINLSPANPQEARETSATVHNIRKSEYQEVWNKWANMQWKESPITTIESVEKINSQEELATKLGVAVNSPVANGSLNFGFNFNKKKNHILARLIQKYFSVSTDAPKKGNIFESIDKEALDGYQPVYISNINYGRIIYLSVESDEDEKVVDEAINFAMNQIKGVDVSVSADQSLHYRKVLANCDIRITVLGGGQTIQKEVLKGDIDSFQRFLNADIPMEQMSPISFSLRYAVDNSQARVVTSNEFTVTQRDFVPEFKKVRMQLQVLGFSGTNTGPFPNLDREAGLWGSISLSLNGQDNELVKISQSNPFFFNYREKKETMHPIGFGGIVTVEFDKDPNESLEDFVDHQKMTFVSDLHSTRSIYNYNFGRTTFTHTLGTLYTKYKGDDPIFVLESNNKNVKIHTYVKVLDMKFFN

SEQ ID NO: 79 - protein, 922 aa, Prevotella histicola
MTKFIYAMSLFLLAAISIKAQPIQKTSGCLLHGSVVSSTDATAIAGATVRLYQLKKLVGGTVSDASGNFDVKCPSSGSLQLRITAVGFKEVDTTLNVPTVTPLSIYMRAGKHAMDEVTVTASEKRGMTSTTVIGQTAMEHLQPSSFADLLALLPGGMTKIPALGSANVITLREAGPPSSQYATSSLGTKFVIDGQAIGTDANMQYIAGSFQGDADNSRNHVSYGVDMREIPTDNIEKVEVVRGIPSVKYGELTSGLINITRKRSQSPLLLRLKADEYGKLVSVGKGFLLSGKWNLNVDGGLLDARKEPRNRFETYRRLTFSARLRRKWNLGERYVLEWSGATDYSLNIDNVKTDPEIQIHREDSYRSSYLKMGMNHRLLLRRKALVGLQSVSLAYSASLASDRIHQTEAVALQRDYVVPLAYEGGEYDGLFLPMQYLCDYRVEGKPFYSTLRGETEWLARTSFISHHITAGGEFLLNKNYGRGQIFDITKPLHASTARRPRSYKDIPATDILSFYAEDKATMPIGKHQLTVMAGLRTTQMLNIPASYAVHGKLFTDTRVNVQWDFPSFLGFKSFVSGGLGMMTKMPTVLDLYPDYVYKDITEMNYWDIRPAYKRIHIRTYKLNQVNPDLRPARNKKWEIRLGMDKGAHHFSVTYFHEDMKDGFRSTTTMRPFIYKRYDTSVINPSALTGPPSLASLPVVTDTLLDGYGRTENGSRITKQGIEFQYSSPRIPVIQTRITVNGAWFRTLYENSIPLFRSAPNVVVGTVAIADRYAGYYMSTDKYDKQIFTSNFIFDSYVDKLGLILSATAECFWMSNTKRPATSSTPMGYMDITGTVHPYVEADQSDPYLRWLVLTGTAGQDMDYRERSYMLVNFKATKRFGRHLSLSFFADRVFYVAPDYEVNGFIVRRTFSPYFGMEIGLKI

SEQ ID NO: 80 - protein, 257 aa, Escherichia coli
MLIDFKKVNIYQDERLILKDIDFQATEGEFIYLIGRVGSGKSSLLKTFYGELDIDQEDAEKAEVLGESVLDIKQKRIPALRRQMGIIFQDFQLLHDRSVAKNLKFVLQATGWKDKEKIKQRIKEVLEQVGMIDKAAKMPSELSGGEQQRIAIARAFLNNPKIILADEPTGNLDPETASNIVSILKDTCKNGTTVIMSTHNINLLSQFPGKVYRCMEQALVPVTNEAQTKDLEEDSTSVEPLIEPVLEEEAQAEDSKE

SEQ ID NO: 81 - protein, 508 aa, Lactococcus lactis
MFENQPKALYALALANTGERFGYYTMIAVFALFLRANFGLEPGTAGLIYSIFLGLVYFLPLIGGIMADKFGYGKMVTIGIIVMFAGYLFLSVPLGGGTVAFGAMLAALLLISFGTGLFKGNLQVMVGNLYDTPELASKRDSAFSIFYMAINIGALFAPTAAVKIKEWAETSLGYAGNDAYHFSFAVACVSLIVSMGIYYAFRSTFKHVEGGTKKTEKAAAAAVEELTPQQTKERIVALCLVFAVVIFFWMAFHQNGLTLTYFADEFVSPTSTGVQSMAFDVVNLVMIVFIVYSIMALFQSKTTKAKGIACAVILAAIAVLAYKYMNVNGQVEVSAPIFQQFNPFYVVALTPISMAIFGSLAAKGKEPSAPRKIAYGMIVAGCAYLLMVLASQGLLTPHEQKLAKAAGETVPFASANWLIGTYLVLTFGELLLSPMGISFVSKVAPPKYKGAMMGGWFVATAIGNILVSVGGYLWGDLSLTVVWTVFIVLCLVSASFMFLMMKRLEKVA

SEQ ID NO: 82 - protein, 492 aa, Myroides odoratus
MKKILIFVAGLCMSLAASAQIQRPKLVVGLVVDQMRWDYLYYYYNEYGTDGLRRLVDNGFSFENTHINYAPTVTAIGHSSVYTGSVPAITGIAGNYFFQDDKNVYCCEDPNVKSVGSDSKEGQMSPHRLLASTIGDELQISNDFRSKVIGVALKDRASILPAGHAADAAYWWDTSAGHFVTSTFYTDHLPQWVIDFNEKNHTAPNFNIKTSTQGVTMTFKMAEAALKNENLGKGKETDMLAVSISSTDAIGHVYSTRGKENHDVYMQLDKDLAHFLKTLDEQVGKGNYLLFLTADHGAAHNYNYMKEHRIPAGGWDYRQSVKDLNGYLQGKFGIAPVMAEDDYQFFLNDSLIAASGLKKQQIIDESVEYLKKDPRYLYVFDEERISEVTMPQWIKERMINGYFRGRSGEIGVVTRPQVFGAKDSPTYKGTQHGQPFPYDTHIPFLLYGWNVKHGATTQQTYIVDIAPTVCAMLHIQMPNGCIGTARNMALGN

SEQ ID NO: 83 - protein, 1138 aa, Staphylococcus epidermidis
MDRQVFQTDSRQRWNRFKWTLRVLITIAILLGVVFVAMFALEGSPQMPFRHDYRSVVSASEPLLKDNKRAEVYKSFRDFFKEQKMHSNYAKVAARQHRFVGHTDNVTQKYIKEWTDPRMGIRSAWYVNWDKHAYISLKNNLKNLNMVLPEWYFINPKTDRIEARIDQRALKLMRRAHIPVLPMLTNNYNSAFRPEAIGRIMRDSTKRMGMINELVAACKHNGFAGINLDLEELNINDNALLVTLVKDFARVFHANGLYVTQAVAPFNEDYDMQELAKYDDYLFLMAYDEYNAGSQAGPVSSQRWVEKATDWAAKNVPNDKIVLGMATYGYNWAQGQGGTTMSFDQTMATALNAGAKVNFNDDTYNLNFSYQDEDDGTLHQVFFPDAVTTFNIMRFGATYHLAGFGLWRLGTEDSRIWKYYGKDLSWESAARMPIAKIMQLSGTDDVNFVGSGEVLNVTSEPHAGRIGIVLDKDNQLIIEERYLSLPATYTVQRLGKCKEKQLVLTFDDGPDSRWTPKVLSILKHYKVPAAFFMVGLQIEKNIPIVKDVFNQGCTIGNHTFTHHNMIENSDRRSFAELKLTRMLIESITGQSTILFRAPYNADADPTDHEEIWPMIIASRRNYLFVGESIDPNDWQQGVTADQIYKRVLDGVHQEYGHIILLHDAGGDTREPTVTALPRIIETLQREGYQFISLEKYLGMSRQTLMPPIKKGKEYYAMQANLSLAELIYHISDFLTALFLVFLVLGFMRLVFMYVLMIREKRAENRRNYAPIDPLTAPAVSIIVPAYNEEVNIVRTISNLKEQDYPSLKIYLVDDGSKDNTLQRVREVFENDDKVVIISKKNGGKASALNYGIAACSTDYIVCVDADTQLYKDAVSKLMKHFIADKTGKLGAVAGNVKVGNQRNMLTYWQAIEYTTSQNFDRMAYSNINAITVIPGAIGAFRKDVLEAVGGFTTDTLAEDCDLTMSINEHGYLIENENYAVAMTEAPESLRQFIKQRIRWCFGVMQTFWKHRASLFAPSKGGFGMWAMPNMLIFQYIIPTFSPIADVLMLFGLFSGNASQIFIYYLIFLLVDASVSIMAYIFEHESLWVLLWIIPQRFFYRWIMYYVLFKSYLKAIKGELQTWGVLKRTGHVKGAQTIS

SEQ ID NO: 84 - protein, 508 aa, Propionigenium modestum
MSQINGRISQIIGPVIDVYFDTKGENPEKVLPNIYDALRVKKADGQDLIIEVQQQIGEDTVRCVAMDNTDGLQRGLEVVPTGSPIVMPAGEQIKGRMMNVIGQPIDGMSALQMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDSEELQKSQATLVYGQMNEPPGARASVALSGLTVAEEFRDHGGKNGEAADIMFFIDNIFRFTQAGSEVSALLGRMPSAVGYQPTLASEMGAMQERITSTKHGSITSVQAVYVPADDLTDPAPATTFTHLDATTELSRKITELGIYPAVDPLGSTSRILDPLIVGKEHYDCAQRVKQLLQKYNELQDIIAILGMDELSDDDKLVVNRARRVQRFLSQPFTVAEQFTGVKGVMVPIEETIKGFNAILNGEVDDLPEQAFLNVGTIEDVKEKAKQLLEATKA

SEQ ID NO: 85 - protein, 984 aa, Prevotella histicola
MNPIYKIITSILFCVLSINTMAQDLTGHVTSKADDKPIAYATVTLKENRLYAFTDEKGNYTIKNVPKGKYTVVFSCMGYASQTVVVMVNAGGATQNVRLAEDNLQLDEVQVVAHRKKDEITTSYTIDRKTLDNQQIMTLSDIAQLLPGGKSVNPSLMNDSKLTLRSGTLERGNASFGTAVEVDGIRLSNNAAMGETAGVSTRSVSASNIESVEVVPGIASVEYGDLTNGVVKVKTRRGSSPFIVEGSINQHTRQIALHKGVDLGGNVGLLNFSIEHARSFLDAASPYTAYQRNVLSLRYMNVFMKKSLPLTLEVGLNGSIGGYNSKADPDRSLDDYNKVKDNNVGGNIHLGWLLNKRWITNVDLTAAFTYADRLSESYTNESSNATQPYIHTLTEGYNIAEDYDRNPSANIILGPTGYWYLRGFNDSKPLNYSLKMKANWSKAFGKFRNRLLVGGEWTSSMNRGRGTYYADMRYAPSWREYRYDALPSLNNIAIYAEDKLSMDVNERQNAELTAGIREDITSIPGSEYGSVGSFSPRMNARYVFRFGQNSWLNSMTLHAGWGRSVKIPSFQVLYPSPSYRDMLAFASTSDADNRSYYAYYTYPSMARYNANLKWQRADQWDLGVEWRTKIADVSLSFFRSKVSNPYMATDVYTPFTYKYTSPAMLQRSGIAVADRRFSIDPQTGIVTVSDASGVKSPVTLGYEERNTYVTNTRYVNADALQRYGLEWIVDFKQIKTLRTQVRLDGKYYHYKAQDETLFADVPVGLNTRQSDGRLYQYVGYYRGGAATTTNYTANASASNGSVSGQVDLNATITTHIPKIRLIVALRLESSLYAFSRATSSRGYVVSSGNEYFGVPYDDKTENQTVIVYPEYYSTWDAPDVLIPFAEKLRWAETNDRGLFNDLAQLVVRTNYPYTLNPNRLSAYWSANLSVTKEIGRHVSVSFYANNFFNTLSQVHSTQTGLETSLFGSGYVPSFYYGLSLRLKI

SEQ ID NO: 86 - protein, 273 aa, Escherichia coli
MERIDISVLMAVYKKDNPAFLRESLESIFSQTVEAAEVVLLEDGPLTDALYDVIKSYEAIYSTLKVVSYPENRGLGKTLNDGLLLCKYNLVARMDADDICKPNRLEMEYNWLKSHEDYDVIGSWVDEFTDNKTRVKSIRKVPEAYDEIKNYAQYRCPINHPTAMYRKAAVLAVGGYLTEYFPEDYFLWLRMLNNGSKFYNIQESLLWFRYSEETVAKRGGWAYACDEVRILVRMLKMGYIPFHVFCQSVVIRFTTRVMPLPIRQRLYNLIRKT

SEQ ID NO: 87 - protein, 507 aa, Paracoccus denitrificans
MSQINGRISQIIGPVIDVYFDTKGENPEKVLPKIHDALRVKRANGQDLIIEVQQHIGEDTVRCVAMDNTDGLQRNLEVVPTGSPIVMPAGDQIKGRMMNVIGQPIDGMEALSMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDQEELQKSQATLVYGQMNEPPGARASVALSGLTVAEEFRDHGGKNGEAADIMFFIDNIFRFTQAGSEVSALLGRMPSAVGYQPTLASEMGTMQERITSTKHGSITSVQAVYVPADDLTDPAPATTFTHLDATTELSRKITELGIYPAVDPLGSTSRILDPLIVGKDHYECAQRVKQLLQHYNELQDIIAILGMDELSDEDKLVVNRARRVQRFLSQPFTVAEQFTGVKGVMVPIEETIKGFNAILNGEVDDLPEQAFLNVGTIEDVKEKAKRLLEATK

SEQ ID NO: 88 - protein, 268 aa, Mycobacterium tuberculosis
MPIGNGQKYQLTIINHTEIIMLIDYKKVNIYQDERLILKDVDFQAETGEFIYLIGRVGSGKSSLLKTIYGELDIDSEDAEKAVVLDESMPNIKRSRIPALRKQMGIIFQDFQLLHDRSVAKNLKFVLQATGWTSKQKIERRIEEVLAQVGMTDKKNKMPSELSGGEQQRIAIARALLNTPKIIIADEPTGNLDPETAANIVSILKDSCQAGTTVIMSTHNINLIDQFPGKVYRCHEGELHQLTDKKEVSELAEETAPVETIDEPEQND

SEQ ID NO: 89 - protein, 336 aa, Yersinia pestis
MKRNILLFICLATSILLLFGLNLTTGSVQIPFADILDILCGRFIGKESWEYIILENRLPQTLTAILCGASLSVCGLMLQTAFRNPLAGPDVFGISSGAGLGVALVMLLLGGTVSTSIFTVSGFLAILTAAFVGAIAVTALILFLSTLVRNSVLLLIVGIMVGYVSSSAVSLLNFFASEEGVKSYMVWGMGNFGAVSMNHIPLFSILCLIGIIASFLLVKPLNILLLGPQYAESLGISTRQIRNILLVVVGLLTAITTAFCGPISFIGLAIPHIARLLFRTENHQILLPGIVLSGAAIALLCNFICYLPGESGIIPLNAVTPLIGAPIIIYVIIQRR

SEQ ID NO: 90 - protein, 408 aa, Bacillus subtilis
MKKYYPWVLVALLWFVALLNYMDRQMLSTMQEAMKVDIAELNHAEAFGALMAVFLWIYGIVSPFAGIIADRVNRKWLVVGSIFVWSAVTYLMGYAESFDQLYWLRAFMGISEALYIPAALSLIADWHEGKSRSLAIGIHMTGLYVGQAVGGFGATLAAMFSWHAAFHWFGIIGIVYSLVLLLFLKENPKHGQKSVLQGETKPSKNPFRGLSIVFSTWAFWVILFYFAVPSLPGWATKNWLPTLFANSLDIPMSSAGPMSTITIAVSSFIGVIMGGVISDRWVQRNLRGRVYTSAIGLGLTVPALMLLGFGHSLVSVVGAGLCFGIGYGMFDANNMPILCQFISSKYRSTAYGIMNMTGVFAGAAVTQVLGKWTDGGNLGNGFAILGGIVVLALVLQLSCLKPTTDNME

SEQ ID NO: 91 - protein, 694 aa, Mycobacterium tuberculosis
MVTKKTTTKKAPVKKTSAKTTKVKEPSHIGLVKNDAYLAPYEDAIRGRHEHALWKMNQLTQNGKLTLSDFANGHNYYGLHQTADGWVFREWAPNATEIYLVGDFNGWNEQEAYQCHRIEGTGNWELTLPHDAMQHGQYYKMRVHWEGGEGERIPAWTQRVVQDEASKIFSAQVWAPAEPYVWEKKTFKPQTSPLLIYECHIGMAQDEEKVGTYNEFREKVLPRIIKDGYNAIQIMAIQEHPYYGSFGYHVSSFFAASSRFGTPEELKALIDEAHKNGIAVIMDIVHSHAVKNEVEGLGNLAGDPNQYFYPGERHEHPAWDSLCFDYGKDEVLHFLLSNCKYWLEEYHFDGFRFDGVTSMLYYSHGLGEAFCNYADYFNGHQDDNAICYLTLANCLIHEVNKNAVTIAEEVSGMPGLAAKFKDGGYGFDYRMAMNIPDYWIKTIKELPDEAWKPSSIFWEIKNRRSDEKTISYCESHDQALVGDKTIIFRLVDADMYWHFRKGDETEMTHRGIALHKMIRLATIAAINGGYLNFMGNEFGHPEWIDFPREGNGWSHKYARRQWNLVDNEELCYHLLGDFDRKMLEVITSEKKFNETPIQEIWHNDGDQILAFSRGELVFVFNFSPSHSYSDYGFLVPEGSYNVVLNTDAREFGGFGFADDTVEHFTNSDPLYEKDHKGWLKLYIPARSAVVLRKK

SEQ ID NO: 92 - protein, 448 aa, Prevotella melaninogenica
MKIDIERIKYFLTVGMFMKTEHSSKRRNMLIRQFQKFYLTVKFFFVRDHAASTAQLSFSTIMAIVPIASMIFAIANGFGFGQFLEKQFREMLSAQPEAATWLLKLTQSYLVHAKTGLFIGIGLMIMLYSVFSLIRTVETTFDNIWQVKDSRPISRIVIDYTALMFLVPISIIILSGLSIYFYSFVENLNGLRFLGTIASFSLRYLVPWAILTLMFIVLYVFMPNAKVKITKTVAPAMIASIAMLCLQAVYIHGQIFLTSYNAIYGSFAALPLFMLWILASWYICLFCAELCYFNQNLEYYECLIDTEDICHNDLLILCATVLSHICQRFANDQKPQTALQIKTETHIPIRVMTDILYRLKEVNLISENFSPTSDEVTYTPTHDTNNITVGEMIARLESTPASDFALLGFSPKKAWNHDIYDRVGSIREIYLNELKSINIKELISYSEN

SEQ ID NO: 93 - protein, 326 aa, Bacillus anthracis
MMKRPSIARVVKVIICLLTPILLSFSGIGDNDIDKKKSTSKEVDDTLRIVITGDLLLDRGVRQKIDMAGVDALFSPTIDSLFHSSNYVIANLECPVTKIRERVFKRFIFRGEPEWLPTLRRHGITHLNLANNHSIDQGRNGLLDTQEQIKKAGMIPIGAGKNMEEAAEPVLISTSPRHVWVISSLRLPLENFLYLPQKPCVSQESIDSLIMRVKRLRATDKNCYILLILHWGWEHHFRATPQQREDAHKLIDAGADAIVGHHSHTLQTIETYRGKPIYYGIGNFIFDQRKPMNSRACLVELSITAEKCKAKALPIEIKNCTPYLSK

SEQ ID NO: 94 - protein, 259 aa, Helicobacter pylori
MILLSFDTEEFDVPREHGVDFSLEEGMKVSIEGTNRILDILKANNVCATFFCTGNFAELAPEVMERIKNEGHEVACHGVDHWQPKPEDVFRSKEIIERVTGVKVAGYRQPRMFPVSDEDIEKAGYLYNSSLNPAFIPGRYMHLTTSRTWFMQGKVMQIPASVSPHLRIPLFWLSMHNFPEWFYLRLVRQVLRHDGYFVTYFHPWEFYDLKSHPEFKMPFIIKNHSGHELEQRLDRFIKAMKADKQEFITYVDFVNRQKK

SEQ ID NO: 95 - protein, 252 aa, Escherichia coli
MAKNISFTIKYWKQNGPQDQGHFDTHEMKNIPDDTSFLEMLDILNEELIAAGDEPFVFDHDCREGICGMCSLYINGTPHGKTERGATTCQLYMRRFNDGDVITVEPWRSAGFPVIKDCMVDRTAFDKIIQAGGYTTIRTGQAQDANAILISKDNADEAMDCATCIGCGACVAACKNGSAMLFVSSKVSQLALLPQGKPEAAKRAKAMVAKMDEVGFGNCTNTRACEAVCPKNEKIANIARLNREFIKAKFAD

SEQ ID NO: 96 - protein, 398 aa, Mycobacterium tuberculosis
MSENKLSTNEQAQTADAPVKASYTEYKVIPSQGYCMIVKCRKGDQTVVLKTLKEEYRERVLLRNALKREFKQCQRLNHSGIVRYQGLVEVDGYGLCIEEEYVEGRTLQAYLKENHTDDEKIAIINQIADALRYAHQQGVIHRNLKPSNVLVTTQGDYVKLIDFSVLSPEDVKPTAETTRFMAPEMKDETLTADATADIYSLGTIMKVMGLTLAYSEVIKRCCAFKRSDRYSNVDELLADLNNEGSSFSMPKIGKGTVVLGLIIAVVIGIGALLYNYGGALIDQVGKIDVSSVFSSDAETAPEDTVKVNTAEQSDSLSTEAEAPAIGKLAFMNRMKPALYKDLDNIFEKNSADKAKLTKAIKTYYRGLIQANDTLDNEQRAEVDRVFGDYVKQKKAALN

SEQ ID NO: 97 - protein, 522 aa, Bacillus subtilis
MRKYICLLLFYLFTFLPLSAQQGNDSPLRKLQLAEMAIKNFYVDSVNEQKLVEDGIRGMLEKLDPHSTYTDAKETKAMNEPLQGDFEGIGVQFNMIEDTLVVIQPVVNGPSQKVGILAGDRIVSVNDSTIAGVKMARIDIMKMLRGKKGTKVKLGVVRRGVKGVLTFVVTRAKIPVHTINASYMIRPNVGYIRIESFGMKTHDEFMSAVDSLKKKGMKTLLLDLQDNGGGYLQSAVQISNEFLKNNDMIVYTEGRRARRQNFKAIGNGRLQDVKVYVLVNELSASAAEIVTGAIQDNDRGTVVGRRTFGKGLVQRPFDLPDGSMIRLTIAHYYTPSGRCIQKPYTKGDLKDYEMDIEKRFKHGELTNPDSIQFSDSLKYYTIRKHRVVYGGGGIMPDNFVPLDTTKFTRYHRMLAAKSIIINAYLKYADANRQALKAQYSSFDAFNKGYVVPQSLLDEIVAEGKKEKIEPKDAAELKATLPNIALQIKALTARDIWDMNEYFRVWNTQSDIVNKAVALATGK

SEQ ID NO: 98 - protein, 348 aa, Prevotella melaninogenica
MKLTEQRSSMLHGVLLITLFACAAFYIGDMGWVKALSLSPMVVGIILGMLYANSLRNNLPDTWVPGIAFCGKRVLRFGIILYGFRLTFQDVVAVGFPAIIVDAIIVSGTILLGVLVGRLLKMDRSIALLTACGSGICGAAAVLGVDGAIRPKPYKTAVAVATVVIFGTLSMFLYPILYRAGIFDLSPDAMGIFAGSTIHEVAHVVGAGNAMGAAVSNSAIIVKMIRVMMLVPVLLVIAFFVAKNVAERDDEAGGSRKINIPWFAILFLVVIGFNSLNLLPKELVDFINTLDTFLLTMAMSALGAETSIDKFKKAGFKPFLLAAILWCWLIGGGYCLAKYLVPVLGVAC

SEQ ID NO: 99 - protein, 833 aa, Prevotella sp.
MNKQFLLAALWLSPLGLYAHKANGIGAVTWKNEAPKERMIRGIDEDKTHQRFTLSGYVKDRNGEPLINATIYDLTTRQGTMTNAYGHFSLTLGEGQHEIRCSYVGYKTLIETIDLSANQNHDIILQNEAQLDEVVVTTDLNSPLLKTQTGKLSLSQKDIKTEYALLSSPDVIKTLQRTSGVADGMELASGLYVHGGNGDENLFLLDGTPLYHTNHSLGLFSSFNADVVKNVDFYKSGFPARYGGRLSSVIDVRTADGDLYKTHGSYRIGLLDGAFHIGGPIRKGKTSYNFGLRRSWMDLLTRPAFAIMNHKSDNEDKLSMSYFFHDLNFKLTNIFNERSRMSLSVYSGEDRLDAKDEWHSNNSSGYNDVDIYVNRFHWGNFNAALDWNYQFSPKLFANFTAVYTHNRSTVSSSDEWRFTRPGEKEQLTLTSHGYRSSIDDIGYRAAFDFRPSPRHHIRFGQDYTYHRFQPQTYNRFDNYQTNSEAKADTIATHSYNKNVAHQLTFYAEDEMTLNEKWSLNGGVNADVFHISGKTFATLSPRLSMKFQPTERLSLKASYTLMSQFVHKIANSFLDLPTDYWVPTTARLHPMRSWQVAAGAYMKPNKHWLLSLEAYYKRSSHILQYSSWAGLEPPAANWDYMVMEGDGRSYGVELDADYNVSNLTLHGSYTLSWTQKKFDDFYDGWYYDKFDNRHKLTLTGRWNITKKIAAFAAWTFRTGNRMTIPTQYIGLPDVPAQEQGGLTFNSSDDNTLNFAYEKPNNVILPAYHRLDIGFDFHHTTKKGHERIWNLSFYNAYCHLNSLWVRVKIDSNNQMKIRNIAFIPVIPSFSYTFKF

SEQ ID NO: 100 - protein, 1133 aa, Escherichia coli
MSKQVFQTDSRQRWSYFKWTLRVILTILSLLGIVFLAMFALEGSPQMPFRHDYRNAVTAASPYTKDNKTAKLYKSFRDFFKEKKMHNNYAKATIKKQRFIGKADSVTQKYFREWDDPRIGVRSAWYVNWDKHAYISLKNNIKHLNMVLPEWFFINPKTDKVEYRIDKQALRLMRRTGIPVLPMLTNNYNSDFHPEAIGRIMRDEKKRMALINEMVRTCRHYGFAGINLDLEELNIQDNDLLVELLKDFSRVFHANGLYVTQAVAPFNEDYNMQELAKYNDYLFLMAYDEHNIESQPGAVSSQRWVEKATDWAAKNVPNDKIVLGMATYGYDWANGEGGTTVSFDQTMAIAQDADAKVKFDDDTYNVNFSYQNTDDGKIHHVFFTDAATTFNIMRFGAEYHLAGYGLWRLGTEDKRIWRFYGKDMSWENVARMSVAKLMQLNGTDDVNFVGSGEVLEVTTEPHPGDISIRIDKDNRLISEEYYRALPSTYTIQRLGKCKDKQLVITFDDGPDSRWTPTVLSTLKKYNVPAAFFMVGLQMEKNLPLVKQVYEDGHTIGNHTFTHHNMIENSDRRSYAELKLTRMLIESVTGHSTILFRAPYNADADPTEHEEIWPMIVASRRNYLFVGESIDPNDWEPNVTSDQIYQRVIDGVHHEDGHIILLHDAGGSSRKPTLDALPRIIETLQHEGYQFISLEQYLGMGKQTLMPEINKGKAYYAMQTNLWLAEMIYHVSDFLTALFLVFLALGMMRLIFMYVLMIREKRAENRRNYAPIDAATAPAVSIIVPGYNEEVNIVRTITTLKQQDYPNLHIYFVDDGSKDHTLERVHEAFDNDDTVTILAKKNGGKASALNYGIAACRSEYVVCIDADTQLKNDAVSRLMKHFIADTEKRVGAVAGNVKVGNQRNMLTYWQAIEYTSSQNFDRMAYSNINAITVVPGAIGAFRKEVIEAVGGFTTDTLAEDCDLTMSINEHGYIIENENYAVALTEAPETLRQFVKQRIRWCFGVMQAFWKHRSSLFAPSKKGFGLWAMPNMLIFQYIIPTFSPLADVLMLIGLFTGNALQIFFYYLIFLVIDASVSIMAYIFEGERLWVLLWVIPQRFFYRWIMYYVLFKSYLKAIKGELQTWGVLKRTGHVKG

SEQ ID NO: 101 - protein, 292 aa, Bacillus subtilis
MAKKRNKARSRHSLQVVTLCISTAMVLMLIGIVVLTGFTSRNLSSYVKENLTITMILQPDMNTEESAALCERIRTLHYINSLNFISKEQALKDGTKELGANPAEFAGENPFTGEIEVQLKANYANNDSIRNIVQQLRTYRGVSDITYPQSLVESVNQTLGKISLVLLVIAVLLTIISFSLINNTIRLSIYAHRFSIHTMKLVGGSWSFIRAPFLRRAVLEGLVSALLAIAVLGIGICLLYEKEPEITKLLSWDALIITAIVMLAFGVIIATFCAWLSVNKFLRMKAGDLYKI

SEQ ID NO: 102 - protein, 254 aa, Haemophilus influenzae
MKNIYFLSDAHLGSLAIDHRRTHERRLVRFLDSIKHKAAAVYLLGDMFDFWNEYKYVVPKGFTRFLGKISELTDMGVEVHFFTGNHDLWTYGYLEKECGVILHRKPITTEIYDKVFYLAHGDGLGDPDPMFRFLRKVFHNRFCQRLLNFFHPWWGMQLGLNWAKRSRLKRKDGKEVPYLGEDKEYLVQYTKEYMSTHKDIDYYIYGHRHIELDLTLSRKARLLILGDWIWQFTYAVFDGEHMFLEEYVEGESKP

SEQ ID NO: 103 - protein, 388 aa, Escherichia coli
MVGLDVLCYFIHAKGREKECYFERIIYQITCHSRTKCYLCNIMKYSIIVPVFNRPDEVEELLESLLSQEEKDFEVVIVEDGSQIPCKEVCDKYADKLDLHYYSKENSGPGQSRNYGAERAKGEYLLILDSDVVLPKGYICAVSEELKREPADAFGGPDCAHESFTDTQKAISYSMTSFFTTGGIRGGKKKLDKFYPRSFNMGIRRDVYQELGGFSKMRFGEDIDFSIRIFKAGKRCRLFPEAWVWHKRRTDFRKFWKQVYNSGIARINLYKKYPESLKLVHLLPMVFTVGTALLVLMILFGLFLQLFPIINVFGSVFIMMGLMPLVLYSVIICVDSTMQNNSLNIGLLSIEAAFIQLTGYGCGFISAWWKRCVCGMDEFAAYEKNFYK

SEQ ID NO: 104 - protein, 435 aa, Streptococcus mutans
MKIEKVHAREIMDSRGNPTVEVEVTLENGVMGRASVPSGASTGENEALELRDGDKNRFLGKGVLKAVENVNNLIAPALKGDCVLNQRAIDYKMLELDGTPTKSKLGANAILGVSLAVAQAAAKALNIPLYRYIGGANTYVLPVPMMNIINGGAHSDAPIAFQEFMIRPVGAPSEKEGIRMGAEVFHALAKLLKKRGLSTAVGDEGGFAPKFDGIEDALDSIIQAIKDAGYEPGKDVKIAMDCAASEFAVCEDGKWFYDYRQLKNGMPKDPNGKKLSADEQIAYLEHLITKYPIDSIEDGLDENDWENWVKLTSAIGDRCQLVGDDLFVTNVKFLEKGIKMGAANSILIKVNQIGSLTETLEAIEMAHRHGYTTVTSHRSGETEDTTIADIAVATNSGQIKTGSMSRTDRMAKYNQLIRIEEELGACAKYGYAKLK

SEQ ID NO: 105 - protein, 501 aa, Brucella suis
MKKLFTIAMLLGVTLGIHAQEVYSLQKCRELALQNNRQLKVSRMTVDVAENTRKAAKTKYLPRVDALAGYQHFSREISLLSDDQKNAFSNLGTNTFGQLGGQIGQNLTSLAQQGILSPQMAQQLGQLFSNVATPLTQVGNNIGQSINDAFRSNTKNVYAGGIVVNQPIYMGGAIKAANDMAAIGEQVAQNNISLKRQLVLYGVDNAYWLAISLKKKEALAIRYRDLAQKLNEDVKKMIREGVATRADGLKVEVAVNTADMQIARIQSGVSLAKMALCELCGLELNGDIPLSDEGDADLPPTPSTQFDNYTVSSSDTTGLNEARPELRLLQNAVDLSIQNTKLIRSLYMPHVLLTAGYSVSNPNLFNGFQKRFTDLWNIGITVQVPVWNWGENKYKVRASKTATTIAQLEMDDVRKKIDLEIEQNRLRLKDANKQLATSQKNMAAAEENLRCANVGFKEGVMTVTEVMAAQTAWQTSRMAIIDAEISVKLAQTGLQKALGGL

SEQ ID NO: 106 - protein, 577 aa, Salmonella typhimurium
MKRTFVTKMVKPIEENSLFFMFMLLVGAFTNVSHRNVFGYIELIADVYIICFLLSLCQRTIRQGLVIMLSSVIYVVAIIDTCCKTLFDTPITPTMLLLAQETTGREATEFFLQYLNLKLFFSAADIILFLAFCHIVMAVKKMKFSTSYLKQPFVAFVLMFTIFVGMALSIYDKVQLYTVKNLSGLEVAVTNGFAHLYHPVERIVYGLYSNHLIAKQVDGVIMANQQIKVDSCSFTSPTIVLVIGESANRHHSQLYGYPLPTTPYQLAMKNGKDSLAVFTNVVSPWNLTSKVFKQIFSLQSVDEKGDWSKYVLFPAVFKKAGYHVSFLSNQFPYGINYTPDWTNNLVGGFFLNHPQLNKQMFDYRNVTIHNYDEDLLNDYKEIISYKKPQLIIFHLLGQHFQYSLRCKSNMKKFGIKDYKRMDLTDKEKQTIADYDNATLYNDFVLNKIVEQFRNKDAIIVYLSDHGEDCYGKDVNMAGRLTEVEQINLKKYHEEFEIPFWIWCSPIYKQRHRKIFTETLMARNNKFMTDDLPHLLLYLAGIKTKDYCEERNVISPSFNNNRRRLVLKTIDYDKALYQ

SEQ ID NO: 107 - protein, 517 aa, Escherichia coli
MFKNHPKGLLQAAFSNMGERFGYYIMNAVLALFLCSKFGLSDETSGLIASLFLAAIYVMSLVGGVIADRTQNYQRTIESGLVVMALGYVALSIPVLATPENNSYLLAFTIFALVLIAVGNGLFKGNLQAIVGQMYDDFETEAAKVSPERLKWAQGQRDAGFQIFYVFINLGALAAPFIAPVLRSWWLGRNGLTYDAALPQLCHKYINGTIGDNLGNLQELATKVGGNSADLASFCPHYLDVFNTGVHYSFIASVVTMLISLIIFMSSKKLFPMPGKKEQIVNVEYTDEEKASMAKEIKQRMYALFAVLGISVFFWFSFHQNGQSLSFFARDFVNTDSVAPEIWQAVNPFFVISLTPLIMWVFAYFTKKGKPISTPRKIAYGMGIAGFAYLFLMGFSLVHNYPSAEQFTSLEPAVRATMKAGPMILILTYFFLTVAELFISPLGLSFVSKVAPKNLQGLCQGLWLGATAVGNGFLWIGPLMYNKWSIWTCWLVFAIVCFISMVVMFGMVKWLERVTKS

SEQ ID NO: 108 - protein, 395 aa, Pseudomonas aeruginosa
MQKKIKIGLLPRVIIAILLGLFLGYYLPDPAVRVFLTFNSIFSQFLGFMIPLIIIGLVTPAIAGIGKGAGKLLLATVAIAYVDTIVAGGLSYGTGTWLFPSMIASTGGAIPHIDKATELTPYFTINIPAMVDVMSSLVFSFIAGLGIAYGGLRTMENLFNEFKTVIEKVIEKAIIPLLPLYIFGVFLSMTHNGQARQVLLVFSQIIIVILVLHVLILIYEFCIAGAIVKHNPFRLLWNMLPAYLTALGTSSSAATIPVTLKQTVKNGVSEEVAGFVVPLCATIHLSGSAMKITACALTICMLTDLPHDPGLFIYFILMLAIIMVAAPGVPGGAIMAALAPLSSILGFNEEAQALMIALYIAMDSFGTACNVTGDGAIALAVNKFFGKKKETSILS

SEQ ID NO: 109 - protein, 208 aa, Escherichia coli
MISVYSIKPQFQRVLTPILELLHRAKVTANQITLWACVLSLVIGILFWFAGDVGTWLYLCLPVGLLIRMALNALDGMMARRYNQITRKGELLNEVGDVVSDTIIYFPLLKYHPESLYFIVAFIALSIINEYAGVMGKVLSAERRYDGPMGKSDRAFVLGLYGVVCLFGINLSGYSVYIFGVIDLLLVLSTWIRIKKTLKVTRNSQTPE

SEQ ID NO: 110 - protein, 582 aa, Escherichia coli
MKLSTILLSIMLGLSSSTMAQQKDVTIKLIETTDVHGSFFPYDFITRKPKSGSMARVYTLVEELRKKDGKDNVYLLDNGDILQGQPISYYYNYVAPEKTNIAASVLNYMGYDVATVGNHDIETGHKVYDKWFKELKFPILGANIIDTKTNKPYILPYYTIKKKNGIKVCVIGMLTPAIPNWLKESIWSGLRFEEMVSCAKRTMAEVKTQEKPDVIVGLFHSGWDGGIKTPEYDEDASKKVAKEVPGFDIVFFGHDHTPHSSIEKNIVGKDVICLDPANNAQRVAIATLTLRPKTVKGKRQYTVTKATGELVDVKELKADDAFIQHFQPEIDAVKAWSDQVIGRFENTIYSKDSYFGNSAFNDLILNLELEITKADIAFNAPLLFNASIKAGPITVADMFNLYKYENNLCTMRLTGKEIRKHLEMSYDLWCNTMKSPEDHLLLLSSTQNDAQRLGFKNFSFNFDSAAGIDYEVDVTKPDGQKVRILRMSNGEPFDENKWYTVAVNSYRANGGGELLTKGAGIPRDSLKSRIIWESPKDQRHYLMEEIKKAGVMNPQPNHNWKFIPETWTVPAAARDRKLLFGE

SEQ ID NO: 111 - protein, 826 aa, Escherichia coli
MKLSELKTGETGVIVKVSGHGGFRKRIIEMGFIKGKTVEVLLNAPLQDPVKYKIMGYEVSLRHSEADQIEVLSDVKTHSVGNEEEQEDNQLEMDSTTYDSTDKELTPEKQSDAVRRKNHTINVALVGNPNCGKTSLFNFASGAHERVGNYSGVTVDAKVGRAEFDGYVFNLVDLPGTYSLSAYSPEELYVRKQLVDKTPDVVINVIDSSNLERNLYLTTQLIDMHIRMVCALNMFDETEQRGDHIDAQKLSELFGVPMIPTVFTNGRGVKELFRQIIAVYEGKEDESLQFRHIHINHGHEIENGIKEMQEHLKKYPELCHRYSTRYLAIKLLEHDKDVEQLVSPLGDSIEIFNHRDTAAARVKEETGNDSETAIMDAKYGFINGALKEANFSTGDKKDTYQTTHVIDHVLTNKYFGFPIFFLVLLVMFTATFVIGQYPMDWIEAGVGWLGEFISKNMPAGPVKDMIVDGIIGGVGAVIVFLPQILILYFFISYMEDCGYMSRAAFIMDRLMHKMGLHGKSFIPLIMGFGCNVPAVMATRTIESRRSRLITMLILPLMSCSARLPIYVMITGSFFALKYRSLAMLSLYIIGVLMAVAMSRLFSAFVVKGEDTPFVMELPPYRFPTWKAIGRHTWEKGKQYLKKMGGIILVASIIVWALGYFPLPDDPNMDNQARQEQSYIGRIGKAVEPVFRPQGFNWKLDVGLLSGMGAKEIVASTMGVLYSNDGSFSDDNGYSSETGKYSKLHNLITKDVATMHHISYEEAEPIATLTAFSFLLFVLLYFPCVATIAAIKGETGSWGWALFAAGYTTALAWIVSAVVFQVGMLFM

SEQ ID NO: 112 - protein, 437 aa, Mycobacterium tuberculosis
MESFIIEGGHQLSGTIAPQGAKNEALEVICATLLTSEEVIIRNVPDILDVNNLIKLLQDIGVKVKKLAPNEFSFQADEVNLDYLESSDFVKKCSSLRGSVLMIGPLLGRFGKATIAKPGGDKIGRRRLDTHFLGFKNLGAHFGRVEDRDVYEIQADKLVGTYMLLDEASITGTANIIMAAVLAEGTTTIYNAACEPYIQQLCKMLNAMGAKISGIASNLITIEGVKELHSADHRILPDMIEVGSFIGIAAMIGDGVRIKDVSVPNLGLILDTFHRLGVQIIVDNDDLIIPRQDHYVIDSFIDGTIMTISDAPWPGLTPDLISVLLVVATQAQGSVLFHQKMFESRLFFVDKLIDMGAQIILCDPHRAVVVGHDNAKKLRAGRMSSPDIRAGIALLIAALTAQGTSRIDNIVQIDRGYENIEGRLNALGAKIQRAEVC

SEQ ID NO: 113 - protein, 244 aa, Bacillus spizizenii
MNIAVIFAGGSGLRMHTKSRPKQFLDLNGKPIIIYTLELFDNHPNIDAIVVACIESWIPFLEKQLRKFEINKVVKIIPGGKSGQESIYKGLCAAEEYAQSKGVSNEETTVLIHDGVRPLITEETITDNIKKVEEVGSCITCIPATETLIVKQADDALEIPSRADSFIARAPQSFRLIDIITAHRRSLAEGKADFIDSCTMMSHYGYKLGTIIGPMENIKITTPTDFFVLRAMVKVHEDQQIFGL

SEQ ID NO: 114 - protein, 145 aa, Glycine max; identical to SEQ ID NO: 4

SEQ ID NO: 115 - protein, 6 aa, Artificial Sequence
  Description of Artificial Sequence Synthetic Peptide
HHHHHH